SEQUENCE LISTING (50 sequences)

SEQ ID NO: 1 - DNA, 1524 bp, synthetic construct
  source 1..1524: mol_type = other DNA
agcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctg

SEQ ID NO: 2 - RNA, 2384 nt, synthetic construct
  source 1..2384: mol_type = other RNA
tattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaacataggcctatcccactggtgggatacgagccaattcgaaagaaaagtcagtcatgtgcttttcagaggatgaaagcttaagataaagactaaaagtgtttgatgctggaggtgggagtggtattatataggtctcagccaagacatgtgataatcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttatttt

SEQ ID NO: 3 - DNA, 1580 bp, synthetic construct
  source 1..1580: mol_type = other DNA
cccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagctttcagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagggtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcaaaaaaaaaaaaaaaaaaaaaaaggccaca

SEQ ID NO: 4 - DNA, 1629 bp, synthetic construct
  source 1..1629: mol_type = other DNA
gttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacacaaaaaaaaaaaaaaaaaaaaaaaaaaaa

SEQ ID NO: 5 - DNA, 1725 bp, synthetic construct
  source 1..1725: mol_type = other DNA
atggagctgctgatccacaggttaagtgcaatcttcctaactcttgctattaatgcattgtacctcacctcaagtcagaacataactgaggagttttaccaatcgacatgtagtgcagttagcagaggttattttagtgctttaagaacaggttggtataccagtgtcataacaatagaattaagtaatataaaagaaaccaaatgcaatggaactgacactaaagtaaaacttataaaacaagaattagataagtataagaatgcagtgacagaattacagctacttatgcaaaacacaccagctgccaacaaccgggccagaagagaagcaccacagtatatgaactatacaatcaataccactaaaaacctaaatgtatcaataagcaagaagaggaaacgaagatttctgggcttcttgttaggtgtaggatctgcaatagcaagtggtatagctgtatccaaagttctacaccttgaaggagaagtgaacaagatcaaaaatgctttgttatctacaaacaaagctgtagtcagtctatcaaatggggtcagtgttttaaccagcaaagtgttagatctcaagaattacataaataaccaattattacccatagtaaatcaacagagctgtcgcatctccaacattgaaacagttatagaattccagcagaagaacagcagattgttggaaatcaacagagaattcagtgtcaatgcaggtgtaacaacacctttaagcacttacatgttaacaaacagtgagttactatcattgatcaatgatatgcctataacaaatgatcagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttctttccacaggctgacacttgtaaagtacagtccaatcgagtattttgtgacactatgaacagtttgacattaccaagtgaagtcagcctttgtaacactgacatattcaattccaagtatgactgcaaaattatgacatcaaaaacagacataagcagctcagtaattacttctcttggagctatagtgtcatgctatggtaaaactaaatgcactgcatccaacaaaaatcgtgggattataaagacattttctaatggttgtgactatgtgtcaaacaaaggagtagatactgtgtcagtgggcaacactttatactatgtaaacaagctggaaggcaagaacctttatgtaaaaggggaacctataataaattactatgaccctctagtgtttccttctgatgagtttgatgcatcaatatctcaagtcaatgaaaaaatcaatcaaagtttagcttttattcgtagatctgatgaattactacataatgtaaatactggcaaatctactacaaatattatgataactacaattattatagtaatcattgtagtattgttatcattaatagctattggtttgctgttgtattgcaaagccaaaaacacaccagttacactaagcaaagaccaactaagtggaatcaataatattgcattcagcaaatag

SEQ ID NO: 6 - DNA, 3681 bp, synthetic construct
  source 1..3681: mol_type = other DNA
gtgagcagaatccatgtgcaaggagagcaggcagttcaggacgagggtgagctggtctctgcaggtttagtgctgtggcactgtgcctggtatatgctcccggcaacttctcctgactctgccttcagacgagacttggaagacagtcacatctcagcagctcctctgccgttatccagcctgcctctgacaagaacccaatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttgacctcaaaaccaagaaaaggtgggcgggggagggggccaaaaccatgagactgaggctgtgggggcaaggaggcaagtcctacgtgtacctatggaaaccgggcgatgatgtgcctgctatcagggcctctgctccctatctagctgccctcctagatcatatcatctgccttacagctgagaggggtgccaatcccagcctagcccctagttccaacctagccccaagatgaactttccagtcaaagagccctcacaaccagctatacatatctgccttggccactgccaagcagaaagatgacagacaccatcctaatatttactcaacccaaaccctaaaacatgaagagcctgccttggtacattcgtgaactttcaaagttagtcatgcagtcacacatgactgcagtcctactgactcacaccccaaagcactcacccacaacatctggaaccacgggcactatcacacataggtgtatatacagacccttacacagcaacagcactggaaccttcacaattacatccccccaaaccacacaggcataactgatcatacgcagcctcaagcaatgcccaaaatacaagtcagacacagcttgtcagaacacgctcgtgtgcacgtacacacatgcagcccctccactctatctcctgagttccatgaatacacaccgactctccaagatgtaccccacgtctcacttgccactgaccccagttccctacccacaagccccaatccatgcctaagcgtggcccacagaagaacttctcttttatttgggatccaaggcccctggcccccagtgcccatccaataaactgtggtcagctggacaatcaccctgatcagatatgggaacatataagcagacagctgggtttaagatcccagcaggagaaagcggataccaaatgaaagagagtgctagaacaggtgcctcagcactgtctccagcaccccaaattcctgcctgtggttaggagacatccatcagggctctaggcctctcggacccggcccaagaggccagcattctcctggcgaagggctcggtagtcctcacagatcttctccaggttgctcaaagtcttcttgcccatctctgtctcaatctaagaaaacaggatgcacacttcttcagcccctgcaggctgcccctctactgaactcctccctgctcctcctattcccgtaacagcagcctgttccttcccatcactgggcttctgggtatgtccttccctccactccacctaaagcagcaacttctgccatgggctctgggaggcattaggagccgcaagctaaaagccagggctcagagtaggctactggctagcttcaggtcccaggcacagtgggcacgaaggcaaagcctctagctgttagttgtctggtttcaaagactctcagcgcaaaacaaggaactatcccctggcctgtctccattccccttaccagtcccaggtctcacctgctcctcaagatctcgaacttccctcatgatagtgcctgtgtcctcaatggtctggatgagctgactgcaattctggagacagcaagaatacaaggcttgcacctatgctggccctctccagccaacccaccaggcacatggctcccctcacctcatgcagggcagctaggtacttgtaggctttccgaacagcatcatccttcttagcatcctgataagacaaaggggatctccgagatatcagcaagccattcccccttttccactactctatgcccctataagaccaccctttactagtactttgccttcatcctccacagagcaaagctaggccccaagcaacagtgcacctaaaggactcacagaggggcaggcaacaactcagtcccgcctccaccctcccggaggccagcctgctccataccttgaacacaagctcatcagtcactgcaaatgtccggtcgagcttcccagagagagagttgatttccttctgcagttcctttgtgtccgacaagatctggtagaaaccagggtaactatcagtgcacatcttgggcaaggtagctgatcagtgataacactcacgtgcctatacttacatccagtcagggcccatgtcgctgtgttggggtgactattatgtgttggagtgtgcctgaacagctctgcctagtagtgagcataaagtccctgtgt

SEQ ID NO: 7 - DNA, 11664 bp, synthetic construct
  source 1..11664: mol_type = other DNA
ggtaccatgtctatcctgaccctaagattagttcctcgggtttgaggattgcagcaacactgaccgttcaggccctggtcaaggtggggctgctgcttcttccttggcttctttccaaggagccaccaagaggcagaaagaaatgaagagacacaaagcaaggcagaatagcacttcggatgacactgtccgcattgcccgacagatatggcactagactgcagccaaaggactctctgaaactattaacaaggttgtgagaacttgtgaccagtctgtgaggtgctgtgtctgggtgtcatgtcactggggacatcatcagtgtcaccagtggcacagtggaatgcctggtgagctgagacacacaaatgaggcaggcgtggtgggcacacacctgtaatcccaacggaacgtaaacctggtatggcagtgctcacctgtatgtggtggcactcaggaagtggaggcaggagcattaggagtctaaggtcatcctcagctacattgacaaatctgagaccagcctgggcgacatgagtccttgtgttaaaaaaaaaaaaaaaaaaaaaaaaaaaaaaagagagagagagagagagagagagaatcagacagtggtggtgcatgcttttaatccagcacttgggaggcagaggcaaacagattactgtgagatcaaggccagcctggtcttcagagcaagttccaggacaagcagggctacatagaaaaaacccgtctctataaacaaacaaaacaaaacaaaacaaaaaaaagcagatctcgtgactctctgaagaaggccatttcccgccagtccttggggttagccgtaagtagcaggctgtagtgtctcgaggccacaaaaactaggagaaccctgggaccacttccagggtgtcgttttacatcacatgtccaactatttaccttcatcttggggctagctcccaccccatacagcctgtgagtgctggaggactttctagggagcctccgtaggaaaggcactggcaggtctcagaaaaggatcggggtcctgatgggggggcgggggtcagtagtgcctaatgcactcagacaagcaccggcgctgcagccagccctgaactgctttttctctaagcccagccaggtgtggacatagcctcagaggaccacgtgtcagctgaatcccatctcatgcccaggaggggtgactgggagagatgggcatctgcttctgggtaaagctacctaagagccacaggggacacagaaatctcagcctcacagggcactttcctgtttgtctaatgctcctctccctagcaccagccaggagtctatagaatcagaggattttaaagtaaggggggagtgggaggtcggttggccccaggagcaccctaagtgtgcccttccggcacttaccctgcgtcaagagccaggaaggaagctctcaagggcgttgcataagagtagaggattgagaagcctggggtggggctagagaggctcattctgaccccactcagcatcccttgcacagtccagagcgtggggatcaaacgagacccccttgtttgacggtgaacaaagtcaggctgagggggttcgggaagggggtaaaggactaggaaccgacatcggccagcacacgggaggtggacaggggtgtccctgctgagaagacctggagggctctcaagacacaggcaaacactgaggtcagcctgttcccatggagtccagcccccaggtcctctcccctactataagagcccatgactcaagtagggtactaagcagtaggcagccatggccaagtcgcacctactgcagtggctactgctgcttcctaccctctgctgcccaggtgcaggtgagtccccggcctccctcacagaggcctctccagcacttactgagtcagctccgtgcccagaaagaccccagtctgcacataatccagaatttaaacgccagttagctgaggcacagagaagtcctagggcctcatccaaggtcacagttagtggatggatgttgaagcaggaggactcagagctgcctggcagaagcaatggccactcctttgcaatgaaactgggttggaggtggggtggaggcagggtgccgagtgtatgctggatcctgatgagagttgctctgaccccaactccagctatcacgtcggcctcatccctggagtgtgcacaaggccctcaattctggtgccaaagcctggagcatgcagtgcagtgcagagccctggggcactgcctgcaggaagtctgggggcatgcaggagctgtgagtagcaccaagcgggcactggaaatccagggaggaggaactggggtggattctgagcggaccttaggaaattggagttcccacaaggctggggtggcagggaatgatggaatggtatagtgtgacaggaaatggtgggcagagtacaatagaaggaaacatggtggaatgagatgaatggggtgggcatggtgggtaagacagggtggatgtgtgggtaagacagggtggatgtggtgggtaagacagggtagatgtggtgggtaagagggggtgagcatgtgggtaagatggggtggctggggtgagatggacaagatggaatagaacagggtggatcaagtgggtggcacagaatgggatggaatttgcacaatgggatgagatgggatgatgggtgggtagccttaaggtacctgtcagcctgtgtctgagaaagcctcaatccctggagttaggagcatgcccccaactcattagcctcacttgagaccctttcttccagaatgacctgtgccaagagtgtgaggatattgtccacctcctcacaaagatgaccaaggaagatgctttccaggtaatgggaaacggtacagtgtgatctggtagaggcctggcgtcaggggactctggtgggggcagacctcagaaagaccaggctaatcctcccttctctgctctcccaggaagcaatccggaagttcctggaacaagaatgtgatatccttcccttgaagctgcttgtgccccggtgtcgccaagtgcttgatgtctacctgcccctggttattgactacttccagagccagattgtgaggaccctgacctacctgccgcacagtgcatgtgcctaagtggccacttacctatataagtggcaccccaacacatgcacacacacacatacacacccacagacgcaataagacacacacacacacacgtacacacacacacacacacacacacacacacacacacttcccactacagccacaggaagctcagtctcttcatccagatacccaaatcagagcctgcctgctcagcatactacagacattgagacccgccctccatcccctcacccacacatgcccacattcttattgtcacacaatatgctcacacacactcactctttccagacacatgctcccaggccctacacagccccatctctctgtctttgtccctttcatagtgtcctaagatgcagtacttcacccagcctgctccccataaccccaggctcaaagactgtggcccttgtccctgaatatgaacctgggcagagaggggttccctccttaccctaaaacccctcacctgttccatgccctagaaccccaaagccatctgcaatcatgtaggcctgtgcccacgtgggcaggctaagccagaacagaatccagggatgccggatgccgttccaaaccctctgctggacaagctggtcctccctgtgctgccaggagccctcttggcaaggcctgggcctcacactcaggtaagccagtccattcccagcagctgctgggaatccagaaggctagcatggccgctgagacgcgtgggcacccagagaggctgagctcaaactaggaggcagagatggcaaggtcaggcaaggtcacacaaccgaggtagctcccagcctaaccacacttcaccgcttccttcctcaggacttctctgagcaacagctccccattcccctgcccttctgctggctttgcagaactctgatcaagcgggttcaagccgtgatccccaaggtaaggaccacacagagctcagaggggcccccaatagctggcaccttcctccacctcaacactccaagaaggctgtgaggagttagatgaggagacacccacacattgctcctacccaaggaaccttgaggctcaggtatgggaggttaggtcagagccaccttctcttccaacagatcaccatcggaaggctgagaagcactggttgtcactgtaggaaaaaagtacattaatttctcaaaaaaaaaaaaaaacagttcatcaatagtaagcatctcttctgtcctccaaatccatggtagcctctgccagtgccttgtcagatgaggattgttctccccacaaatggtcatggcctatcaacactaacactaagcccacatcagtcataaagacaacagggcacacagtcaagcctttctgaagcctgtgtgatggaaggaacgtgcagactatagagcaggatgagctgaggggtcgcacagataaaaatggtaacagacaggtcagccagggagaggctctgaagagggtaacaactaagccaagatctaggagaaaacaaggtccccaggggccaaggacatccatccatcaataaaaaatgagctcaatcagatgttggagggagggactctgtaaggagggaccaggagcagggggcagcgtttggggtgtaaatgatagataaatgcctttaaaatgagctcagagggctaggaagatggctcggtgagtacagtccttgctgaacctgagttcagatacttgcaccctcataaaagttggggggtgggctggagacatggctcagtagttaagagcactgactgctcttctagaggtcctgagttcaattcccagcaaccacttggtggctcacaaccacctataatgggatctgatgccctcttctggtgtgtctgaacttacatacataaaataaaaataaaagttgggggttgctcacagtcagctaatggatggatcatagggctcccaatggaggagctagagaaagtagccaaggagctaaagggatctgcaaccctataggtggaacaacattatgagctaaccagtaccccggagctcttgactctagctgcatatatatcaaaagatggcctagtcggccatcactggaaagagaggcccattggacttgcaaactttatatgccccagtacaggggaataccagggccaaaaagggggagtgggtgggcaggggagtgggggtgggtggatatgggggacttttggtatagcattggaaatgtaaatgagttaaatacctaataaaaaatggaaaaaaaaaaagttgggggttagcaatgaacatttgtaaccctacacactaggtagtcagaaataggcagatccctagagcatgctggccagccagtctagccaaatggatgagcttcagggttagtgtgagaccttgtctcaaaaaaaaaaaaaaaaaaaaaaaaaatggacggcctgaagattcggatcgacagttaggaacatttgctgcttttcagaagagtgagttgggtacccagcaccactgtcaggcagctcacaaccccctgtaactgctgctctagggaatccaatgccctcttctggcagccaagggcaccagcacatatgtggcattcatatactcagatacacagacatatgtaaaaataaaaataaatctttagaaaataattaggtagggagtgaagtgactaaggaagacactcaatcttggctctggcctccacacacatgtgcacatgtacttaaacatctacgtgcaaaacaaacaaacaaacacccagccgtatcaatgtgaacatcactgaggaccgaaggcatgagcaagactgttaagagacaatgtatagacagatggagatggcatcagaattgctgagaggggacaggcagccaacgggggaccgtgctgcattgccagggaagccaagagagaagggtgtttgactgattgaaaggcagctgaaccatcaggcagggtgagagttaggcaggggatgtggaagtgttccaaaaaggggagcaggcatggtgaggcttcctaaggtcagaagccattctagcgtgttctccaggcagcagggaccagagagaggataaggccagggaaagaggcatgggtggaggtaatccaggagtgaagaccatttcaccaatgagcagcttggtcattgactacagtgactattgatttacatcaccatgacaggagagccatgtgtgggtcaatgataacaggtgggtctcttaagtgaagtgccccatttgggagccatcacactccaggggtgtccatattctgagtcctccccctgcctcaacctcctggcactggggctagctggtcacatgggctgaataaggagtaaaggaaaaagccacaccctggtgacctctgtcacccttcagctagagcctgcttggaattggagttgaggtaggagatgtgctggctttcccaggggttccaaaagccaaagacatgtcagctctgggggccagcagaaggaactgcctgtcttcctgatgcataagcatgggaaggtaggtggccctcggtcagggaatgggtttgaattgggtcaggctgttagatgccatggccttgcagccccctttcaaatgactcaagcctttagagctagatctatatttggtgtcaactgcagattctctcagtgactccgggtgcacctgagacccctgctgtcttggatgctcagtgacctgtggacagaactgctctttcctagaagggagaaaggggatgcatctggggtgcccactcagttgggcacagtgacatcgtgccagaagaaggttctatggttgtcctttctccaccttcaccccagggtgtgctggctgtggctgtgtcccaggtgtgccacgtggtacccctggtggtgggtggcatctgccagtgcctggctgagcgctacacagttctcctgctagacgcactgctgggccgtgtggtgccccagctagtctgtggccttgtcctccgatgttccactgaggatgccatgggccctggtaagacttgcccgtcccctccccctccccaactcacatccctccagtgcacatgggagggaacatggacaaggtggggttcaggaaccaacactttttttaaactatttatttctatggatatggctgcttttatttatatagctgaggctggctttgaactcctaatttcccttcctcagccattcaaatgttaggaaaggctagcaatgactgtactcagcttctagctctctccaagtggacttctcccagttgagttaaagagtgatgggggaggggtggggaacagggcaggaccctgggagaaggctaagttctttttttgctccagcttggacatctatataccccatgtatgcctggctcccacagaggccataaaggatgtcaaatcccctagaattggaataactgacagttatgagccatcatgtggggctctgggaatcgaacctcagccctctggaagagcagccagtgctcttaaccacggaaccatctctccagccccagaaccaacacttgtacaagacagtcctgggggaaagattaaaacagagtcttactacatagcacaggttggcctcgagcttggtgcaatcctcctgcctcagcctctcaaatactggcatgacaaggtatgtgcctccatacccagcttgctggacaattctaactgctttctctttagccctccctgctgtggagcctctgatagaagaatggccactacaggacactgagtgccatttctgcaagtctgtgatcaaccaggcctggaacaccagtgaacaggctatgccacaggcaatgcaccaggcctgccttcgcttctggctagacaggcaaaaggtagggggcccacgggttggatgtatgtcatatgtgtgatggtgccgagctagaagagactttgtagctagacacacgcacgatgctggttcccagcctggtggacaggcatgtgggtcagacaatgatgggattgtaacaaatttaactggctaggagacatcatggacccaaggctttggactatggaacatcagcaggccttctttatggactaagcacaagaaaagtcctgttagtcccaacaggaaagggtcatactgccctttcttggtttcactcgatggtgtgtttgccacactgttctcccagtgtgccatgtcacccccatgatgggtggtagcatttgacagtacctagcaggcaccagaaaatgagaaaagccagggtcagctggagcagaaaaagaacttagccttttcccagggtcctgttctgccccaccctgctcactctgtagaagtcctgcaggagagagctggaagctggtaccatagtgctagcctgtaattctaacatttggaaaggctgaagaaggagaaatgggagttcaaagccagcctcagctatataaataatgagttcagggtcagcctgggctacatgagaccctgtctggtgaaaggagacagagataggaaagaacatgaggcttgggtaaggctcactggcatggccacaaccaagtttgatccctgggatccgtatggtaaacaaagagaatcaactcctgtaaactttccttatgaacacacacacacacgaaaacataattttgaagccaggctgtggtggtgcacacctttagtcctagcccttgggaggcagaagcagatggatctaagtttgagcccagcctggtctacagtgtgagctccaggacagccagggttacacagagaaaccctgtctcacaaaaccaaaaagaaatcaacaaccacaaagaactgaacagatagttccttaagcctgtgatgaatcccctcactacagtgggactttctttagagagggtcctatgtaacttaaaccgcctccacctcctttgtactgagactacaggcaggtaccactactgagtttcatgtagttctgaagttgaaactaaaggtttcatgcatgctaggcaaccatgagacgatgctaagctgcaagcctgctccagctccaaggccctggcttcctccaaagcctggtttcagccaaacttagatagagtccctttttttaagactcattttatttgtgtttttagtgcatgtatgtatggacatcatgtgtgtgtggtgccggggggagggggtcagaagaggccatcagattccctggaactggagttgagtggttataagccgtccttcctgtcctccaaagagcagcaagtgcctaacccccgagccatcagccattcagcccttcggttgagtctttaatggtcagccaggcactgatggaaaaacacaaacccacagtccggagtggcagagtgaggtagaacgccagatctgcaggttaagttctctcctagaggggggtctacatattgtgtctttcctcagtgtgaacagtttgtggaacagcacatgccccagctgctggccctggtgcctaggagccaggatgcccacatcacctgccaggtatgcccactcttcagctggtcccaggagtcccctctgctcccacagtcccaccctccttggtctatgatcctcaagagccccatttcttggatccaggaagcctagggctcagaagcccagaactaagtgtacccatagaacaggctttggacttggagcagaaaagaacacatactgattaggtgggaggggcaagttcatgatggatgggcagctgggggctggggtatgatgctccttattgcatgtggtgtgtttagtgaccagtttgttctatggtggggctatagtatgaggtgggggtcccactaagtcccaaggccattgacttagggaatggcacaaggggttctgaaggtgaaggtgaagtgagagttgtctccatagccttgagaattagacgtagaaagctgaggcccacgtgctgtctccaacaggcccttggcgtatgtgaggccccggctagccctctgcagtgcttccaaaccccacacctctgagaacgcggtctccaggtgagtccagcctcctggggagagagaggaatgggtctttgcttgctaaggtttgggaacaagatggtcatcctgcccacttctgtggactgtgtcatcctacctctgccaggcacagttccaggctcctcggggtctccagtggttccatcaggaaaaggcagtcttttggacctatcgtcactccttgctctcccaccccatccagccctccacagcttctatctaaggcttcatcacatctgagctgcctgaccttaaagatactccatgttcgagcaatggccaacatttcttacttcactgtctcggctgtctctccctcagatgccagcagcaccatggtcacctgacctcaccctgcccaggctccctgttttctaagccagaaatagctctgacaccagagtcaggaaatgacatggggagtgtggggcgagaaaggcaacagtctctcaagtgaccctgacagtaatctggtccaggtcacaatgtacttaaagccagcgctcgctgggtagtcatttatccatttgttcccatttgtgaaaatctgctggtgtgcacagctggcccaccacttctaatgcgaggaaggaccccagcactgtcacagccactgtgggcagaggggcacttcaagtcagtaagtcccttgggggccaatttaatgtctcccctcccatcccccatcaagtccatctgggtgcgcgaagggaggcaatccaggagtcacctttttctagctctcagggctctaggccttgcctgctgaagaaggaattgtgagagactccctgagttctggtcccaactctgctatcaacagtcagtggatcccccgggaaaatcgcacagcccccaccctttgcgatatcactaaactagctgcaagtagcccaatgaagggaacttcggcacttatgaactgtcaccatcacagtgacagtgaccctactcccaccagtagctactctccttgaaaaagaccttacctcccaccctaatgctacttcctttcccacagcagcctgctccagaggacaagcctcagcctgcaccctcagccctgtgtcagtctgtatgtcccagctctaactacagaccaccaccaccaccaccaccaccaccaccaccaccaccaccaccaccaccaccaccaccaccaccaccacagtggtttctggctccccccggtgatggggggcggcaggcccacgtcctctggaagccttcagaaggggcttcgggccttcgcctccaccagagccaagccagctcccatagctcccacagcccacagggactgagaagaactgttgtggctccaagaagacatcgggtagaagctgggtatagccacaccaaccccttgctaacatttctatgaaatccaaacttgagaagaataaagaatgggaacatggagcattattctaagggctgtgggcgaggcgcagtgacagggcactttcctagcaagcaggaaacctgggttggatcc

SEQ ID NO: 8 - DNA, 1083 bp, synthetic construct
  source 1..1083: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 9 - DNA, 954 bp, synthetic construct
  source 1..954: mol_type = other DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 10 - DNA, 1440 bp, synthetic construct
  source 1..1440: mol_type = other DNA
atgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 11 - DNA, 1365 bp, synthetic construct
  source 1..1365: mol_type = other DNA
ctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 12 - DNA, 4107 bp, synthetic construct
  source 1..4107: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 13 - DNA, 4104 bp, synthetic construct
  source 1..4104: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagctgaacgtcatcctatttttggaaatatagtagatgaagttgcttatcatgagaaatatccaactatctacatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactctatccatcaatccatcactggtctttatgaaacacgcattgatttgagtcagctaggaggtgactga

SEQ ID NO: 14 - protein, 1054 aa, synthetic construct
MKEKYILGLDLGITSVGYGIINFETKKIIDAGVRLFPEANVDNNEGRRSKRGSRRLKRRRIHRLERVKLLLTEYDLINKEQIPTSNNPYQIRVKGLSEILSKDELAIALLHLAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLQNANKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKKPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 15 - protein, 1370 aa, synthetic construct
MNKPYSIGLDIGTNSVGWSIITDDYKVPAKKMRVLGNTDKEYIKKNLIGALLFDGGNTASDRRLKRTARRRYTRRRNRILYLQEIFAEEMSKVDDSFFHRLEDSFLVEDDKRGSKYPIFATMQEEKDYHEKFPTIYHLRKELADKKEKADLRLFYLALAHIIKFRGHFLIEDDSFDVRNTDIQRQYQAFLEIFDTTFENNHLLSQNIDVEGILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHREDLKQLKQFVKASLPEKYQEIFTDSSKDGYAGYIEGKTNQGAFYKYLSKLLTKQEGSEYFLEKIKNEDFLRKQRTFDNGSIPHQVHLTELKAIIRRQSEYYPFLKENLDRIEKILTFRIPYYVGPLAREKSDFAWMTRKTDDSIRPWNFEELVDKEASAEAFIHRMTNNDLYLPEEKVLPKHSLIYEKFTVYNELTKVRYKNEQGETYFFDSNIKQEIFDGVFKEHRKVSKKKLLDFLAKEYEEFRIVDVIGLDKENKAFNASLGTYHDLKKILDKDFLDNPDNESILEDIVQTLTLFEDREMIKKRLENYKDLFTESQLKKLYRRHYTGWGRLSAKLINGIRDKESQKTILDYLIDDGKSNRNFMQLIHDDGLSFKSIISKAQAGSHSDNLKEVVGELAGSPAIKKGILQSLKIVDELVKVMGYEPEQIVVEMARENQTTNQGRRNSRQRYKLLDDGVKNLASDLNGNILKEYPTDNQALQNERLFLYYLQNGRDMYTGKALDIDNLSQYDIDHIIPQAFIKDDSIDNRVLVSSAKNRGKSDDVPSLEIVKDCKVFWKKLLDAKLMSQRKYDNLTKAERGGLTSDDKARFIQRQLVETRQITKHVARILDERFNNELDSKGRRIRKVKIVTLKSNLVSNFRKEFGFYKIREVNNYHHAHDAYLNAVVAKAILTKYPQLEPEFVYGDYPKYNSYKTRKSATEKLFFYSNIMNFFKTKVTLADGTVVVKDDIEVNNDTGEIVWDKKKHFATVRKVLSYPQVNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNELKGKPEEIEQKQEFVVQHVSYFDDILQIINDFSNRVILADANLEKINKLYQDNKENISVDELANNIINLFTFTSLGAPAAFKFFDKIVDRKRYTSTKEVLNSTLIHQSITGLYETRIDLGKLGED

SEQ ID NO: 16-19 - skipped (4 entries)

SEQ ID NO: 20 - protein, 621 aa, Varicella Zoster
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 21 - DNA, 1872 bp, Varicella Zoster
  source 1..1872: mol_type = other DNA
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtga

SEQ ID NO: 22 - protein, 676 aa, Ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETRYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSLRPPITKPGPDNSTHNTPVYKLDISEATQVEQHHRRTDNASTTSDTPPATTAAGPLKAENTNTSKGTDLLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 23 - DNA, 2446 bp, Ebolavirus
  source 1..2446: mol_type = other DNA
gcgatgaagattaagccgacagtgagcgtaatcttcatctctcttagattatttgtcctccagagcagggatcgtcaggttcttttcaatcgtataaccaaaataaacttcactagaaggatattgtggggcaacaacacaatgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacgaatcaattgagatcagttggactgaatctcgaagggaatggagtggcaactgacgtgccatctgcaactaaaagatggggcttcaggtccggtgtcccaccaaaagtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgatcgacttgcttccacagttatctaccgaggaacgactttcgctgaaggtgtcgtggcatttctgatactgccccaagctaagaaggacttcttcagctcacaccccttgagagagccggtcaatgcaacggaggacccgtctagtggctactattctaccacaattagatatcaggctaccggctttggaaccaatgagacagagtatttgttcgaggttgacaatttgacctacgtccaacttgaatcaagattcacaccacagtttctgctccagctgaatgagacaagatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcttcgaccccccataaccaaaccaggtccggacaacagcacccacaatacacccgtgtataaacttgacatctctgaggcaactcaagttgaacaacatcaccgcagaacagacaacgccagcacaacctccgacactccccccgccacgaccgcagccggacccctaaaagcagagaacaccaacacgagcaagggcactgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactattcctgagagccacaaccgagctacgcaccttttcaatcctcaaccgtaaggcaattgatttcttgctgcagcgatggggcggcacatgccacattttgggaccggactgctgtatcgaaccacatgattggactaagaacataacggacaaaattgatcagattattcatgattttgttgataaaacccttccggaccagggggacaatgacaattggtggacaggatggagacagtggataccggcaggtattggagttacaggcgttataattgcagttatcgctttattctgtatatgcaaatttgtcttttagtttttcttcagattgcttcatggcaaagctcagcctcaaatcaatgaaaccaggatttaattatatggattacttgaatctaagattacttgacaaatgataatataatacactggagctttaaacatagccaatgtgattctaactcttttaaactcacagttaatcataaataaggtttgacatcaatctagttatctctttgagaatgataaacttgatgaagattaagaaaaaggtaatctttcgattatctttaatcttcatccttgatt

SEQ ID NO: 24 - protein, 1273 aa, SARS Covid
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - DNA, 4440 bp, SARS Covid
  source 1..4440: mol_type = other DNA
tgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaag

SEQ ID NO: 26 - protein, 3392 aa, Dengue virus
MINQRKKTGRPSFNMLKRARNRVSTVSQLAKRFSKGLLSGQGPMKLVMAFIAFLRFLAIPPTAGILARWGSFKKNGAIKVLRGFKREISNMLNIMNRRKRSVTMLLMLLPTALAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIAQYENLKYSVIVTVHTGDQHQVGNETTEHGTTATITPQAPTSEIQLTDYGTLTLDCSPRTGLDFNEMVLLTMKKKSWLVHKQWFLDLPLPWTSGALTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSVMCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDFCEGTTVVVDEHCGNRGPSLRTTTVTGKIIHEWCCRSCTLPPLRFKGEDGCWYGMEIRPVKDKEENLVKSMVSAGSGEVDSFSLGLLCISIMIEEVMRSRWSKKMLMTGTLAVFLLLIMGQLTWSDLIRLCIMVGANASDKMGMGTTYLALMATFKMRPMFAVGLLFRRLTSREVLLLTIGLSLVASVELPSSLEELGDGLAIGIMMLKLLTDFQSHQLWATLLSLTFIKTTFSLHYAWKTMAMVLSIVSLFPLCLSTTSQKTTWLPVLLGSLGCKPLPMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAVSGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNTGEEVQVIAVEPGKNPKNVQTTPGTFKTPEGEVGAIALDFKPGTSGSPIVNREGKIVGLYGNGVVTTSGTYVSAIAQAKASQEGPLPEIEDKVFRKRNLTIMDLHPGSGKTRRYLPAIVREAIKRKLRTLILAPTRVVASEMAEALKGVPIRYQTTAVKSEHTGKEIVDLMCHATFTMRLLSPVRVPNYNMIIMDEAHFTDPASIAARGYISTRVGMGEAAAIFMTATPPGSVEAFPQSNAIIQDEERDIPERSWNSGYDWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYIYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVMASSALLWMASVEPHWIAASIILEFFLMVLLIPEPDRQRTPQDNQLAYVVIGLLFVILTVAANEMGLLETTKKDLGIGHVAAENHHHATMLDVDLHPASAWTLYAVATTIITPMMRHTIENTTANISLTAIANQAAILMGLDKGWPISKMDIGVPLLALGCYSQVNPLTLIAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIMEVDRSEAKEGLKRGETTKHAVSRGTAKLRWFVERNLVKPEGKVIDLGCGRGGWSYYCAGLKKVTEVKGYTKGGPGHEEPIPMATYGWNLVKLHSGKDVFFIPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWELVHRERELHKQGKCATCVYNMMGKREKKLGEFGKAKGSRAIWYMWLGARFLEFEALGFMNEDHWFSRENSLSGVEGEGLHKLGYILRDISRIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFLPSELETPNLAGRVLDWLEKYGVERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEEVPYLGKREDQWCGSLIGLTARATWATNIQVAINQVRRLIGNENYLDYMTSMKRFKNESDPEGALW

SEQ ID NO: 27 - DNA, 10735 bp, Dengue virus
  source 1..10735: mol_type = other DNA
agttgttagtctacgtggaccgacaagaacagtttcgaatcggaagcttgcttaacgtagttctaacagttttttattagagagcagatctctgatgatcaaccaacgaaaaaagacgggtcgaccgtctttcaatatgctgaaacgcgcgagaaaccgcgtgtcaactgtttcacagttggcgaagagattctcaaaaggattgctctcaggccaaggacccatgaaattggtgatggctttcatagcattcttaagatttctagccatacccccaacagcaggaattttggctagatggggctcattcaagaagaatggagcgattaaagtgttacggggtttcaagagagaaatctcaaacatgctaaacataatgaacaggaggaaaagatccgtgaccatgctccttatgctgctgcccacagccctggcgttccatctgacgacacgagggggagagccgcatatgatagttagcaagcaggaaagaggaaagtcacttttgttcaagacctctgcaggtgtcaacatgtgcaccctcattgcgatggatttgggagagttgtgtgaggacacgatgacctacaaatgcccccggatcactgaggcggaaccagatgacgttgactgttggtgcaatgccacggacacatgggtgacctatggaacgtgctctcaaactggcgaacaccgacgagacaaacgttccgtcgcattggccccacacgtggggcttggcctagaaacaagagccgaaacgtggatgtcctctgaaggtgcttggaaacagatacaaaaagtagagacttgggctctgagacatccaggattcacggtgatagccctttttctagcacatgccataggaacatccatcacccagaaagggatcattttcattttgctgatgctggtaacaccatctatggccatgcgatgcgtgggaataggcaacagagacttcgtggaaggactgtcaggagcaacatgggtggatgtggtactggagcatggaagttgcgtcaccaccatggcaaaaaacaaaccaacactggacattgaactcttgaagacggaggtcacaaaccctgcagttctgcgtaaattgtgcattgaagctaaaatatcaaacaccaccaccgattcgagatgtccaacacaaggagaagccacactggtggaagaacaagacgcgaactttgtgtgccgacgaacgttcgtggacagaggctggggcaatggctgtgggctattcggaaaaggtagtctaataacgtgtgccaagtttaagtgtgtgacaaaactagaaggaaagatagctcaatatgaaaacctaaaatattcagtgatagtcaccgtccacactggagatcagcaccaggtgggaaatgagactacagaacatggaacaactgcaaccataacacctcaagctcctacgtcggaaatacagctgaccgactacggaacccttacattagattgttcacctaggacagggctagattttaacgagatggtgttgctgacaatgaaaaagaaatcatggcttgtccacaaacagtggtttctagacttaccactgccttggacctctggggctttaacatcccaagagacttggaacagacaagatttactggtcacatttaagacagctcatgcaaagaagcaggaagtagtcgtactaggatcacaagaaggagcaatgcacactgcgctgactggagcgacagaaatccaaacgtcaggaacgacaacaattttcgcaggacacctaaaatgcagactaaaaatggacaaactaactttaaaagggatgtcatatgtgatgtgcacaggctcattcaagttagagaaagaagtggctgagacccagcatggaactgttctggtgcaggttaaatatgaaggaacagacgcaccatgcaagattcccttttcgacccaagatgagaaaggagcaacccagaatgggagattaataacagccaaccccatagtcactgacaaagaaaaaccagtcaatattgaggcagaaccaccctttggtgagagctacatcgtggtaggagcaggtgaaaaagctttgaaactaagctggttcaagaaaggaagcagcatagggaaaatgtttgaagcaactgcccgaggagcacgaaggatggccattctgggagacaccgcatgggacttcggttctataggaggagtgttcacgtctatgggaaaactggtacaccaggtttttggaactgcatatggagttttgtttagcggagtttcttggaccatgaaaataggaatagggattctgctgacatggctaggattaaattcaaggaacacgtccctttcggtgatgtgcatcgcagttggcatggtcacactgtacctaggagtcatggttcaggcagattcgggatgtgtaatcaactggaaaggcagagaacttaaatgtggaagcggcatttttgtcactaatgaagttcacacttggacagagcaatacaaattccaggctgactcccccaagagactatcagcagccattgggaaggcatgggaggagggtgtgtgtggaatccgatcagccactcgtctcgagaacatcatgtggaaacaaatatcaaatgaattgaaccacatcctacttgaaaatgacatgaaatttacagtggtcgtgggagacgttagtggaatcttggcccaagggaaaaaaatgattaggccacaacccatggaacacaaatactcgtggaaaagctggggaaaagctaaaatcataggagcggatgtacagaacaccaccttcatcatcgacggcccaaacaccccagaatgccctgacaatcaaagagcatggaatatttgggaagtagaggactatggatttgggattttcacgacaaacatatggttgaaattgcgtgactcctacacccaagtatgtgaccaccggctgatgtcagctgccattaaggacagcaaggcagtccatgctgacatggggtactggatagaaagtgaaaagaacgagacatggaagttggcgagagcctcctttatagaagttaagacatgcatctggccaaaatcccacactctatggagcaatggagttctggaaagtgaaatgataattccaaagatatatggaggaccaatatctcagcacaactacagaccaggatatttcacacaaacagcagggccgtggcacctaggcaagttggaactagatttcgatttttgtgaaggtaccacagttgttgtggatgaacattgtggaaatcgaggaccatctctcagaaccacaacagtcacaggaaagataatccatgaatggtgctgcagatcttgtacgctaccccccctacgtttcaaaggggaagacgggtgttggtacggcatggaaatcagaccagtgaaggacaaggaagagaacctggtcaagtcaatggtctctgcagggtcaggagaagtggacagcttttcactaggactgctatgcatatcaataatgattgaagaagtgatgagatccagatggagcaaaaaaatgctgatgactggaacactggctgtgttcctccttcttataatgggacaattgacatggagtgatctgatcaggttatgtattatggttggagccaacgcttcagacaagatggggatgggaacaacgtacctagctttaatggccactttcaaaatgagaccaatgttcgccgtcgggctattatttcgcagactaacatctagagaagttcttcttcttacaattggcttgagcctggtggcatccgtggagctaccaagttccctagaggagctgggggatggacttgcaataggcatcatgatgttgaaattattgactgattttcagtcacaccagctatgggctactctgctatccttgacatttattaaaacaactttttcattgcactatgcatggaagacaatggctatggtactgtcaattgtatctctcttccctttatgcctgtccacgacctctcaaaaaacaacatggcttccggtgctgttgggatctcttggatgcaaaccactacccatgtttcttataacagaaaacaaaatctggggaaggaagagttggcccctcaatgaaggaattatggctgttggaatagttagtattctactaagttcacttttaaaaaatgatgtgccgctagccggcccattaatagctggaggcatgctaatagcatgttatgtcatatccggaagctcagctgatttatcactggagaaagcggctgaggtctcctgggaggaagaagcagaacactcaggcgcctcacacaacatactagtagaggttcaagatgatggaaccatgaagataaaagatgaagagagagatgacacgctcaccattctccttaaagcaactctgctggcagtctcaggggtgtacccaatgtcaataccagcgaccctttttgtgtggtatttttggcagaaaaagaaacagagatcaggagtgctatgggacacacccagccccccagaagtggaaagagcagttcttgatgatggcatctatagaattttgcaaagaggactgttgggcaggtcccaagtaggagtaggagttttccaagaaggcgtgttccacacaatgtggcacgtcactaggggagctgtcctcatgtatcaaggaaaaaggctggaaccaagctgggccagtgtcaaaaaagacttgatctcatatggaggaggttggaggtttcaaggatcctggaacacgggagaagaagtacaggtgattgctgttgaaccgggaaaaaaccccaaaaatgtacaaacaacgccgggtaccttcaagacccctgaaggcgaagttggagccatagccttagactttaaacctggcacatctggatctcccatcgtaaacagagagggaaaaatagtaggtctttatggaaatggagtggtgacaacaagcggaacttacgttagtgccatagctcaagctaaggcatcacaagaagggcctctaccagagattgaggacaaggtgtttaggaaaagaaacttaacaataatggacctacatccaggatcgggaaaaacaagaagataccttccagccatagtccgtgaggccataaaaaggaagctgcgcacgctaatcctagctcccacaagagttgtcgcttctgaaatggcagaggcactcaagggagtgccaataaggtatcagacaacagcagtgaagagtgaacacacaggaaaggagatagttgaccttatgtgccacgccactttcaccatgcgcctcctgtctcccgtgagagttcccaattataacatgattatcatggatgaagcacacttcaccgatccagccagcatagcagccagagggtacatctcaacccgagtgggtatgggtgaagcagctgcgatctttatgacagccactcccccaggatcggtggaggcctttccacagagcaatgcaattatccaagatgaggaaagagacattcctgagagatcatggaactcaggctatgactggatcactgattttccaggtaaaacagtctggtttgttccaagcatcaaatcaggaaatgacattgccaactgtttaagaaaaaacgggaaacgggtgatccaattgagcagaaaaacctttgacactgagtaccagaaaacaaaaaacaacgactgggactatgtcgtcacaacagacatttccgaaatgggagcaaatttccgggccgacagggtaatagacccaaggcggtgtctgaaaccggtaatactaaaagatggtccagagcgcgtcattctagccggaccgatgccagtgactgtggccagtgccgcccagaggagaggaagaattggaaggaaccaaaacaaggaaggtgatcagtatatttacatgggacagcctttaaacaatgatgaggaccacgctcattggacagaagcaaagatgctccttgacaatataaacacaccagaagggattatcccagccctctttgagccggagagagaaaagagtgcagctatagacggggaatacagactgcggggtgaagcaaggaaaacgttcgtggagctcatgagaagaggggatctaccagtctggctatcctacaaagttgcctcagaaggcttccagtactccgacagaaggtggtgcttcgatggggaaaggaacaaccaggtgttggaggagaacatggacgtggagatctggacaaaagaaggagaaagaaagaaactacgacctcgctggttggacgccagaacatactctgacccactggctctgcgcgagtttaaagagtttgcagcaggaagaagaagcgtctcaggtgacctaatattagaaatagggaaacttccacaacatttgacgcaaagggcccagaatgctttggacaacttggtcatgttgcacaattccgaacaaggaggaaaagcctatagacatgctatggaagaactgccagacacaatagaaacgttgatgctcctagccttgatagctgtgttgactggtggagtgacgctgttcttcctatcaggaagaggtctaggaaaaacatctatcggcttactctgcgtgatggcctcaagcgcactgttatggatggccagtgtggagccccattggatagcggcctccatcatactggagttctttctgatggtactgcttattccagagccagacagacagcgcactccacaggacaaccagctagcatatgtggtgataggtctgttattcgtgatattgacagtggcagccaatgagatgggattattggaaaccacaaagaaagacctggggattggccatgtagctgctgaaaaccaccaccatgctacaatgctggacgtagacctacatccagcttcagcctggaccctctatgcagtggccacaacaatcatcactcctatgatgagacacacaattgaaaacacaacggcaaatatttccctgacagccatcgcaaaccaagcagctatattgatgggacttgacaagggatggccaatatcgaagatggacataggagttccacttctcgccttggggtgctattcccaagtgaatccgctgacactgatagcggcagtattgatgctagtagctcattacgccataattggacctggactgcaagcaaaagctactagagaagctcaaaaaagaacagcggctggaataatgaaaaatccaactgtcgacgggattgttgcaatagacttagatcccgtggtttacgatgcaaaatttgaaaaacagctaggccaaataatgttgttgatactttgcacatcacagattcttttgatgcggactacatgggccttgtgtgaatccatcacattggctactggacctctgaccactctttgggagggatctccaggaaaattctggaacaccacaatagcggtatccatggcaaacattttcagggggagttatctagcaggagcaggtctggccttctcattaatgaaatctctaggaggaggtaggagaggcacgggagcccaaggggaaacactgggagaaaaatggaaaagacaactaaaccaactgagcaagtcagaattcaatacttacaagaggagtgggattatggaggtggatagatccgaagccaaagagggactgaaaagaggagaaacaaccaaacacgcagtatcgagaggaacggccaaactgaggtggttcgtggagaggaaccttgtgaaaccagaagggaaagtcatagacctcggttgtggaagaggtggctggtcatattattgcgctgggctgaagaaagtcacagaagtgaaaggatacacaaaaggaggacctggacatgaggaaccaatcccaatggcgacctatggatggaacctagtaaagctgcactccggaaaagatgtattttttataccacctgagaaatgtgacacccttttgtgtgatattggtgagtcctctccgaacccaactatagaggaaggaagaacgttacgtgttctgaaaatggtggaaccatggctcagaggaaaccaattttgcataaaaattctaaatccctatatgccgagcgtggtagaaactctggaacaaatgcaaagaaaacatggaggaatgctagtgcgaaacccactctcaagaaattccacccatgaaatgtactgggtttcatgtggaacaggaaacattgtgtcagcagtaaacatgacatctagaatgttgctaaatcggttcacaatggctcacaggaagccaacatatgaaagagacgtggacttaggcgctggaacaagacatgtggcagtagaaccagaggtagccaacctagatatcattggccagaggatagagaatataaaaaatgaacataagtcaacatggcattatgatgaggacaatccatacaaaacatgggcctatcatggatcatatgaggttaagccatcaggatcggcctcatccatggtcaatggcgtggtgagattgctcaccaaaccatgggatgttatccccatggtcacacaaatagccatgactgataccacaccctttggacaacagagggtgtttaaagagaaagttgacacgcgcacaccaaaagcaaaacgtggcacagcacaaattatggaagtgacagccaggtggttatggggtttcctttctagaaacaaaaaacccagaatttgcacaagagaggagtttacaagaaaagttaggtcaaacgcagctattggagcagtgttcgttgatgaaaatcaatggaactcggcaaaagaagcagtggaagacgaacggttctgggaacttgtccacagagagagggagcttcataaacaggggaaatgtgccacgtgtgtctacaatatgatggggaagagagagaaaaaattaggagagttcggaaaggcaaaaggaagtcgtgcaatatggtacatgtggttgggagcacgcttcctagagtttgaagcccttggtttcatgaatgaagatcactggttcagtagagagaattcactcagtggagtggaaggagaaggactccacaaacttggatacatactcagagacatatcaaggattccaggggggaacatgtatgcagatgacacagccggatgggacacaagaataacagaggatgatctccagaatgaggctaaaatcactgacatcatggagcccgaacatgccctgctggctacgtcaatctttaagctgacctaccaaaataaggtggtaagggtgcagagaccagcaaaaaatggaaccgtgatggatgttatatccagacgtgaccagagaggcagtggacaggttggaacttatggcttaaacactttcaccaacatggaggcccaactgataagacaaatggagtctgagggaatctttttacccagcgaattggaaaccccaaatctagccggaagagttctcgactggttggaaaaatatggtgtcgaaaggctgaaaagaatggcaatcagcggagatgactgtgtggtgaaaccaattgatgacaggttcgcaacagccttaacagctttgaatgacatgggaaaagtaagaaaagacataccacaatgggaaccttcaaaaggatggaatgattggcaacaagtgcctttctgttcacaccacttccaccagctaattatgaaggatgggagggagatagtggtgccatgccgcaaccaagatgaacttgtggggagggccagagtatcacaaggcgccggatggagcctgagagaaaccgcatgcctaggcaagtcatatgcacaaatgtggcagctgatgtatttccacaggagagacctgagactggcggctaacgctatttgttcagccgttccagttgattgggtcccaaccagccgcaccacctggtcgatccatgcccatcaccaatggatgacaacagaagacatgttatcagtatggaatagggtctggatagaggaaaacccatggatggaggataagactcatgtgtccagttgggaagaagttccatacctaggaaagagggaagatcagtggtgtggatccctgataggcttaacagcaagggccacctgggccactaatatacaagtggccataaaccaagtgagaaggctcattgggaatgagaattatctagattacatgacatcaatgaagagattcaagaatgagagtgatcccgaaggggcactctggtaagtcaacacattcacaaaataaaggaaaataaaaaatcaaatgaggcaagaagtcaggccagattaagccatagtacggtaagagctatgctgcctgtgagccccgtccaaggacgtaaaatgaagtcaggccgaaagccacggtttgagcaagccgtgctgcctgtggctccatcgtggggatgtaaaaacccgggaggctgcaacccatggaagctgtacgcatggggtagcagactagtggttagaggagacccctcccaagacacaacgcagcagcggggcccaacaccaggggaagctgtaccctggtggtaaggactagaggttagaggagaccccccgcgtaacaataaacagcatattgacgctgggagagaccagagatcctgctgtctctacagcatcattccaggcacagaacgccagaaaatggaatggtgctgttgaatcaacaggttct

SEQ ID NO: 28 - protein, 748 aa, HIV
MKVMGRRRNCQQWWELSSLCLWVVIIGSVAGNLWVTVYYGVPVWKEAKTTLFCASDAKAYKKEVHNIWATHACVPTDPSPQEILLENVTENFNMWKNDMVDQMHEDVVSLWDQSLKPCVKLTPLCVTLHCVNATYKIVNDTKVNATYNDTGVGELKNCSFNMTTEVRDQKQTRYALFYKLDIVPLSNDHNHSNYSNHSNYRLISCKTSTLTQACPKVTFDPLPIHYCAPAGYAILKCNNKTFNGTGPCTNVSTVQCTHGIKPVVSTQLLLNGSLSEGEIIIRSENLTNNVKTIIVHLNVSVEINCSRPNNNTRKSVRIGPGQTFYAMGDIIGDIRAAHCKISDKAWSETLQNVTKKLREHFPNKTIKFEPSSGGDLEITTHSFNCKGEFFYCNTSKLFNRTYWPNGTHWSNSTDNSSIIMLPCRIKQIINMWQKVGRAMYAPPIAGKLTCNSNITGLLLVRDGGRENNTNNTETFRPGGGNMKDNWRSELYKYKVVEIKPLGVAPTQARRMVVTREKRAVGIGAVLLGFLGAAGSTMGAASITLTAQARQVLSGIVQQQSNLLRAIEAQQHLLQLTVWGIKQLQTRVLAIERYLKDQQLLGLWGCSGKLICTTSVPWNSSWSNKSQTDIWNNMTWMQWDREISNYTSTIYKLLEDSQIQQDKNEKDLLALDSWKNLWSWFDISNWLWYIKIFIMIVGGLIGLRIVFAVLSIVNRVRQGYSPLSFQTLTPNQIPGGLDRLGRIEEEGGE

SEQ ID NO: 29 - DNA, 2619 bp, HIV
  source 1..2619: mol_type = other DNA
atgaaagtgatggggagacggaggaattgtcaacaatggtgggaattgagcagcttatgcttgtgggtggtgataattggtagtgtggcaggaaacttgtgggtcacagtctattatggggtacctgtgtggaaagaagcaaaaactactctattctgtgcatcagatgctaaagcctataagaaagaagtgcataatatatgggctacacatgcctgtgtacccacagaccccagcccacaagaaatactcttggaaaatgtaacagagaattttaacatgtggaaaaatgacatggtggatcagatgcatgaggatgtagttagtttatgggatcaaagtctaaagccatgtgtaaagttaactccactctgtgtcactttacactgtgtcaatgctacatacaaaattgtcaatgacactaaagtcaatgctacatacaatgatacaggggttggagaactaaaaaattgctctttcaatatgacaacagaagtaagagatcagaaacagactaggtatgcacttttttataaacttgatatagtaccacttagtaatgaccataaccatagtaactatagtaaccatagcaactatagattaataagttgtaagacctcaaccctaacacaagcctgtccaaaggtcacttttgacccacttcctatacattattgtgctccagctggttatgcgattctaaaatgtaataataagacattcaatgggacaggaccatgcactaatgtcagcacagtacaatgtacacatggaattaagccagtggtatcaactcaattactgttaaatggtagcctatcagaaggagagataataattagatctgaaaatttgacaaacaatgtcaaaacaataatagtacatcttaatgtatctgtagaaattaattgctcaaggcccaacaataatacaaggaaaagtgtaaggataggaccaggacaaacattttatgcaatgggtgacataataggagatataagagcagcacattgtaaaattagcgacaaagcatggtctgaaactttacaaaatgtaacaaaaaaattaagagaacacttccctaataagacaataaaatttgaaccatcctcaggaggggacctagaaattacaacacatagctttaattgtaagggagaatttttctattgtaatacatcaaagctgtttaataggacatactggcctaatggcacacactggtctaatagtactgataactcctccatcatcatgctcccatgcagaataaaacaaattataaacatgtggcagaaggtagggcgagcaatgtatgcccctcccattgcagggaaactaacatgtaattcaaatatcacaggactactattagtacgtgatggaggaagagaaaacaacacaaacaacacagagacattcagacctggaggaggaaatatgaaggataattggagaagtgaattatacaaatataaagtggtagaaattaagccattgggagtagcacccacccaggcacggaggatggtggtgacgagggaaaaaagagcagtaggaataggagctgtactccttgggttcttgggagctgcaggaagcactatgggcgcggcgtcaataacgctgacggcacaggccagacaagtattgtctggtatagtgcaacagcaaagcaatttgctgagagctatagaggcgcaacaacatctgttgcaactcacagtctggggcattaaacagctccagacaagagtcctggctatagaaagatacctaaaggatcaacagctcctagggctttggggctgctctggaaagctcatctgcaccacctctgtgccttggaactccagttggagtaataaatctcaaacagacatttggaataacatgacctggatgcagtgggatagagaaattagtaactacacaagtacaatatacaagctacttgaagactcgcaaatccagcaggataaaaatgaaaaagacttattagcattggacagttggaaaaatctgtggagttggtttgacatatcaaactggctgtggtatataaaaatattcatcatgatagtaggaggtttgataggtttgagaatagtttttgctgtactgtctatagtgaatagagttaggcagggatactcacctttgtcgtttcagacccttaccccgaaccagatcccgggaggactcgacaggctcggaagaatcgaagaagaaggtggagagtaagacagaaggagatccattcgattagtgagcggattcttgtctcttgcctgggaagacttgaggagcctgtgcctcttcagctaccaccgattgagagacttcgcattgattgtaacgagagcagtggaacttctgggacgcagcagcctcaggggactacagagggggtgggaggcccttaaatatctgggaggcctcgtgcagtattggggtctggaactaaaaaagagtgctattagtctgcttaataccatagcaatagtagtagctgaaggaacagataggattatagaactagtacaaagaatttgtagggctatcctcaatatacctagaagagtaagacaaggctttgaagccgcgttactataa

SEQ ID NO: 30 - protein, 912 aa, Bordetella pertussis
MNMSLSRIVKAAPLRRTTLAMALGALGAAPAAHADWNNQSIVKTGERQHGIHIQGSDPGGVRTASGTTIKVSGRQAQGILLENPAAELQFRNGSVTSSGQLFDDGIRRFLGTVTVKAGKLVADHATLANVGDTWDDDGIALYVAGEQAQASIADSTLQGAGGVQIERGANVTVQRSAIVDGGLHIGALQSLQPEDLPPSRVVLRDTNVTAVPASGAPAAVSVLGASELTLDGGHITGGRAAGVAAMQGAVVHLQRATIRRGDAPAGGGVPGGAVPGGAVPGGAVPGGFGPGGFGPVLDGWYGVDVSGSSVELAQSIVEAPELGAAIRVGRGARVTVSGGSLFAPHGNVIETGGARRFAPQAAPLSITLQAGAHAQGKALLYRVLPEPVKLTLTGGADAQGDIVATELPSIPGTSIGPLDVALASQARWTGATRAVDSLSIDNATWVMTDNSNVGALRLASDGSVDFQQPAEAGRFKVLTVNTLAGSGLFRMNVFADLGLSDKLVVMQDASGQHRLWVRNSGSEPASANTLLLVQTPRGSAATFTLANKDGKVDIGTYRYRLAANGNGQWSLVGAKAPPAPKPAPQPGPQPPQPQPEAPAPQPPAGRELSAAANAAVNTGGVGLASTLWYAESNALSKRLGELRLNPDAGGAWGRGFAQRQQLDNRAGRRFDQKVAGFELGADHAVAVAGGRWHLGGLAGYTRGDRGFTGDGGGHTDSVHVGGYATYIADSGFYLDATLRASRLENDFKVAGSDGYAVKGKYRTHGVGASLEAGRRFTHADGWFLEPQAELAVFRAGGGAYRAANGLRVRDEGGSSVLGRLGLEVGKRIELAGGRQVQPYIKASVLQEFDGAGTVRTNGIAHRTELRGTRAELGLGMAAALGRGHSLYASYEYSKGPKLAMPWTFHAGYRYSW

SEQ ID NO: 31 - DNA, 2739 bp, Bordetella pertussis
  source 1..2739: mol_type = other DNA
atgaacatgtctctgtcacgcattgtcaaggcggcgcccctgcgccgcaccacgctggccatggcgctgggcgcgctgggcgccgccccggcggcgcatgccgactggaacaaccagtccatcgtcaagaccggtgagcgccagcatggcatccatatccagggctccgacccgggcggcgtacggaccgccagcggaaccaccatcaaggtaagcggccgtcaggcccagggcatcctgctagaaaatcccgcggccgagctgcagttccggaacggcagtgtcacgtcgtcgggacagttgttcgacgatggcatccggcgctttctgggcaccgtcaccgtcaaggccggcaagctggtcgccgatcacgccacgctggccaacgtcggcgacacctgggacgacgacggcatcgcgctctatgtggccggcgaacaggcccaggccagcatcgccgacagcaccctgcagggcgctggcggcgtgcagatcgagcgcggcgccaatgtcacggtccaacgcagcgccatcgtcgacgggggcttgcatatcggcgccctgcagtcattgcagccggaagaccttccgcccagccgggtggtgctgcgcgacaccaacgtgaccgccgtgcccgccagcggcgcgcccgcggcggtgtctgtgttgggggccagtgagcttacgctcgacggcgggcacatcaccggcgggcgggcagcgggggtggcggccatgcaaggggcggtcgtgcatctgcagcgcgcgacgatacggcgcggggacgcgcctgccggcggtggggttcccggcggtgcggttcccggcggtgcggttcccggtggtgcggttcccggcggcttcggtcccggcggcttcggtcccgtcctcgacggctggtatggcgtggacgtatcgggctccagcgtggagctcgcccagtcgatcgtcgaggcgccggagctgggcgccgcaatccgggtgggccgcggcgccagggtgacggtgtcgggcggcagcttgttcgcaccgcacggcaatgtcatcgagaccggcggcgcgcgtcgctttgcgcctcaagccgcgcccctgtcgatcaccttgcaggccggcgcgcatgcccaggggaaagcgctgctgtaccgggtcctgccggagcccgtgaagctgacgctgaccgggggcgccgatgcgcagggcgacatcgtcgcgacggagctgccctctattcccggcacgtcgatcgggccgctcgacgtggcgctggccagccaggcccgatggacgggcgctacccgcgcggtcgactcgctgtccatcgacaacgccacctgggtcatgacggacaactcgaacgtcggtgcgctacggctggccagcgacggcagcgtcgatttccagcagccggccgaagctgggcggttcaaggtcctgacggtcaatacgctggcgggttcggggctgttccgcatgaatgtcttcgcggacctggggctgagcgacaagctggtcgtcatgcaggacgccagcggccagcacaggctgtgggtccgcaacagcggcagcgagccggccagcgccaacaccctgctgctggtgcagacgccacgaggcagcgcggcgacctttacccttgccaacaaggacggcaaggtcgatatcggtacctatcgctatcgattggccgccaacggcaatgggcagtggagcctggtgggcgcgaaggcgccgccggcgcccaagcccgcgccgcagccgggtccccagccgccgcagccgcagccggaagcgccggcgccgcaaccgccggcgggcagggagttgtccgccgccgccaacgcggcggtcaacacgggtggggtgggcctggccagcacgctctggtacgccgaaagcaatgcgttgtccaagcgcctgggcgagttgcgcctgaatccggacgccggcggcgcctggggccgcggcttcgcgcaacgccagcagctggacaaccgcgccgggcggcgcttcgaccagaaggtggccggcttcgagctgggcgccgaccacgcggtggcggtggccggcggacgctggcacctgggcgggctggccggctatacgcgcggcgaccgcggcttcaccggcgacggcggcggccacaccgacagcgtgcatgtcgggggctatgccacatatatcgccgacagcggtttctacctggacgcgacgctgcgcgccagccgcctggagaatgacttcaaggtggcgggcagcgacgggtacgcggtcaagggcaagtaccgcacccatggggtgggcgcctcgctcgaggcgggccggcgctttacccatgccgacggctggttcctcgagccgcaggccgagctggcggtattccgggccggcggcggtgcgtaccgcgcggccaacggcctgcgggtgcgcgacgaaggcggcagctcggtgctgggtcgcctgggcctggaggtcggcaagcgcatcgaactggcaggcggcaggcaggtgcagccatacatcaaggccagcgtgctgcaggagttcgacggcgcgggtacggtacgcaccaacggcatcgcgcaccgcaccgaactgcgcggcacgcgcgccgaactgggcctgggcatggccgccgcgctgggccgcggccacagcctgtatgcctcgtacgagtactccaagggcccgaagctggccatgccgtggaccttccacgcgggctaccggtacagctggtaa

SEQ ID NO: 32 - protein, 432 aa, Plasmodium sp.
KKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNDAGNAAGNAAGNAAGNAAGNAAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRARRKVDAKNKKPAELVLSDLETEICSLDKCSSTFNVVSNSLGIVLVLVLILFH

SEQ ID NO: 33 - DNA, 1302 bp, Plasmodium sp.
  source 1..1302: mol_type = other DNA
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtacatttaatgtcgtaagtaattcgttaggaatagtattagttttagtcttaatactctttcactaa

SEQ ID NO: 34 - protein, 261 aa, synthetic construct
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 35 - protein, 273 aa, synthetic construct
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 36 - protein, 698 aa, synthetic construct
SAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 37 - protein, 687 aa, synthetic construct
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 38 - protein, 890 aa, synthetic construct
MSQGILSPPAGLLSDDDVVVSPMFESTAADLGSVVRKNLLSDCSVVSTSLEDKQQVPSEDSMEKVKVYLRVRPLLPSELERQEDQGCVRIENVETLVLQAPKDSFALKSNERGIGQATHRFTFSQIFGPEVGQASFFNLTVKEMVKDVLKGQNWLIYTYGVTNSGKTHTIQGTIKDGGILPRSLALIFNSLQGQLHPTPDLKPLLSNEVIWLDSKQIRQEEMKKLSLLNGGLQEEELSTSLKRSVYIESRIGTSTSFDSGIAGLSSISQCTSSSQLDETSHRWAQPDTAPLPVPANIRFSIWISFFEIYNELLYDLLEPPSQQRKRQTLRLCEDQNGNPYVKDLNWIHVQDAEEAWKLLKVGRKNQSFASTHLNQNSSRSHSIFSIRILHLQGEGDIVPKISELSLCDLAGSERCKDQKSGERLKEAGNINTSLHTLGRCIAALRQNQQNRSKQNLVPFRDSKLTRVFQGFFTGRGRSCMIVNVNPCASTYDETLHVAKFSAIASQLVHAPPMQLGFPSLHSFIKEHSLQVSPSLEKGAKADTGLDDDIENEADISMYGKEELLQVVEAMKTLLLKERQEKLQLEMHLRDEICNEMVEQMQQREQWCSEHLDTQKELLEEMYEEKLNILKESLTSFYQEEIQERDEKIEELEALLQEARQQSVAHQQSGSELALRRSQRLAASASTQQLQEVKAKLQQCKAELNSTTEELHKYQKMLEPPPSAKPFTIDVDKKLEEGQKNIRLLRTELQKLGESLQSAERACCHSTGAGKLRQALTTCDDILIKQDQTLAELQNNMVLVKLDLRKKAACIAEQYHTVLKLQGQVSAKKRLGTNQENQQPNQQPPGKKPFLRNLLPRTPTCQSSTDCSPYARILRSRRSPLLKSGPFGKKY

SEQ ID NO: 39 - protein, 1356 aa, synthetic construct
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 40-49 - skipped (10 entries)

SEQ ID NO: 50 - DNA, 1378 bp, synthetic construct
  source 1..1378: mol_type = other DNA
tctagaacatttgcttctgacacaactgtgttcactagcaacctcaaacagacaccgccgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattc

(19) United States
(12) Patent Application Publication
(10) Pub. No.: US 2024/0350621 A1
(43) Pub. Date: Oct. 24, 2024

(54) DOUBLE STRANDED MRNA VACCINES

(71) Applicant: University of Iowa Research Foundation, Iowa City, IA (US)

(72) Inventor: Kevin G. Rice, Iowa City, IA (US)

(21) Appl. No.: 18/681,741

(22) PCT Filed: Aug. 5, 2022
(86) PCT No.: PCT/US2022/039577

§ 371 Date: Feb. 6, 2024

Related U.S. Application Data
(60) Provisional application No. 63/230,458, filed on Aug. 6, 2021.
(60) Provisional application No. 63/328,559, filed on Apr. 7, 2022.

(51) Int. Cl. A61K 39/215; A61K 9/127; A61K 9/51; A61K 39/00; A61K 39/145; A61P 31/14; C12N 7/00

(52) U.S. Cl. CPC A61K 39/215; A61K 9/1271; A61K 9/5123; A61K 39/145; A61P 31/14; C12N 7/00; A61K 2039/53; A61K 2039/55555; C12N 2760/16134; C12N 2770/20034

(57) ABSTRACT

A composition is provided comprising an amount of double stranded (ds) mRNA encoding a therapeutic or prophylactic gene product, wherein at least one strand of the ds mRNA has a 5′ cap, a start codon, a polyA sequence and encodes the protein, wherein the two strands of the ds mRNA are hydrogen bonded over at least 10 nucleotides, and an amount of a plurality of distinct lipids, as well as methods of using the composition.

### CROSS-REFERENCE TO RELATED APPLICATIONS

This application is a U.S. National Stage Filing under 35 U.S.C. 371 from International Application No. PCT/US2022/039577, filed on Aug. 5, 2022, and published as WO 2023/014974 A1 on Feb. 9, 2023, which application claims the benefit of the filing date of U.S. application No. 63/230,458, filed on Aug. 6, 2021, and U.S. application No. 63/328,559, filed on Apr. 7, 2022, the disclosures of which are incorporated by reference herein.

### INCORPORATION BY REFERENCE OF SEQUENCE LISTING

This application contains a Sequence Listing which has been submitted electronically in ST26 format and is hereby incorporated by reference in its entirety. Said ST26 file, created on May 6, 2024, is named “875223US1.xml” and is 114,198 bytes in size.

### BACKGROUND

The leading SARS-CoV-2 mRNA vaccine (mRNA-1273, encoding S protein) developed by Moderna is in Phase 2 Clinical Trial as of June 2020 (ClinicalTrials.gov Identifier: NCT04283461). The major advantage of this vaccine platform over others is speed. It took only 42 days for Moderna to generate the vaccine for Phase 1 Clinical Trial testing after receiving the DNA sequence (Wu et al., 2020). The Moderna mRNA vaccine relies upon a lipid nanoparticle (LNPs) to achieve cellular delivery of S protein mRNA (Hassett et al., 2019). LNPs are the most widely used in vivo mRNA delivery system at present (Semple et al., 2010). mRNA-LNPs were first demonstrated as efficient delivery systems for mRNA in mice 2015 (Pardi et al., 2015). Since then, multiple vaccine studies have resulted in durable, protective immune responses against multiple infectious pathogens, often after a single dose (Awasthi et al., 2019; Bahl et al., 2017; Jagger et al., 2019; John et al., 2018; Lutz et al., 2017; Meyer et al., 2018; Pardi et al., 2018a; Pardi et al., 2017; Pardi et al., 2018b; Richner et al., 2017; Roth et al., 2019; VanBlargan et al., 2018). The LNP delivery system used in the COVID-19 clinical trial is a precise blend of four lipids mixed with single stranded mRNA in a microfluidic mixer (Hassett et al., 2019). Both the lipid structure and composition dramatically influence the IgG titer following i.m. dosing in mice (Hassett et al., 2019).

All of the currently developed mRNA LNP vaccines are based on using single stranded mRNA. Single stranded mRNA, that possess a 3″ poly A tail and 5′ cap, is metabolically labile due to the action of endogenous RNAse. Encapsulation of single stranded mRNA in a LNP helps protect it from metabolism however, the duration of trans-gene expression from single stranded mRNA LNP is typically 7 days when dosed in muscle (Pardi et al., 2015). The level and duration of trans-gene expression influences the magnitude immune response. mRNA LNP with poor encapsulation or unsuccessful delivery to the cytosol of dendritic cells produce a weaker immune response. Current clinically used single stranded mRNA LNP vaccines all substitute pseudo uridine for uridine to attempt to block the formation of trace quantities of ds RNA biproduct that forms during in vitro translation (IVT). These trace quantities of ds RNA are also removed by HPLC purification.

Double stranded mRNA is a form of metabolically stabilized mRNA. It is approximately 1000-fold more stable than single stranded mRNA when challenged by RNAse digestion. It is generated by preparing a complementary reverse RNA strand that is hybridized with single stranded mRNA. When dosed i.m. and electroporated, double stranded mRNA expression persists for 15 days compared to 7 days for single stranded mRNA. However, the use of double stranded mRNA could trigger an innate immune response, leading to the release of inflammatory cytokines and unwanted side effects, including the unwanted shut down of transgene expression.

### SUMMARY

Double stranded (ds) mRNA is much more metabolically stable than single-stranded (ss) mRNA and so ds mRNA formulations, e.g., lipid nanoparticle (LNP) formulations, as described herein are likewise more stable than corresponding ss mRNA formulations. ds mRNA is also as efficiently translated into protein as single-stranded mRNA. Thus, ds mRNA that includes single-stranded mRNA may be employed in targeted gene delivery system, e.g., systemic delivery, to express prophylactic or therapeutic proteins in animals, e.g., humans. Persistent expression may be achieved by self-amplifying mRNA constructs designed to replicate mRNA in the cytosol and extend its expression.

In one embodiment, a composition comprises ds mRNA LNPs which produce a superior immune response compared to single stranded mRNA LNPs. Surprisingly, ds mRNA LNP vaccines function without causing a serious innate immune response and perform better than single stranded mRNA LNPs.

In particular, the disclosure provides fords mRNA vaccines to treat a variety of diseases. In one embodiment, the ds mRNA is protected from rapid deactivation, thereby improving the stability of the ds mRNA vaccine which, in turn, allows it to be more effective. In one embodiment, the vaccine may be employed to prevent, inhibit or treat pathogen infections including microbial infections, e.g., viral infections such as SARS-CoV2, influenza, hepatitis and measles, and also to prevent, inhibit or treat cancer, dementia, heart disease, diabetes, smoking and any other vaccine treatable disease. In one embodiment, the ds mRNA encodes a COVID 19 spike protein or a portion thereof which induces a protective immune response once administered. In one embodiment, the ds mRNA encodes an antibody, e.g., an IgG, a light chain Ig or a heavy chain Ig, or a scFv or nanobody specific for, for example, TNF-alpha or IL-6 In one embodiment, the ds mRNA encodes influenza hemagglutinin. In one embodiment, the ds mRNA is synthesized from plasmid DNA templates using in vitro transcription (IVT), followed by enzymatic capping and purification.

In one embodiment, the disclosure provides isolated ds mRNA encoding a gene product useful in a vaccine, optionally in combination with a LNP or a peptide conjugate as described herein, which provides for enhanced stability in vivo. At least one strand of the ds mRNA has a 5′ cap, a start codon, and a polyA sequence, and this strand encodes a protein. The two strands of the ds mRNA are hydrogen bonded (Watson Crick) over at least 10 nucleotides and up to the full length of the shortest strand, if the strands are of different lengths. For example, the two strands of the ds mRNA are hydrogen bonded over at least 25, 50, 100, 200, 500, 1000, 2000 or more, e.g., 10,000 nucleotides (or any integer between 25 and 10,000), or over at least 50%, 60%, 70%, 80%, 85%, 90%, 95%, 98% or more of the length of at least one strand. In one embodiment, at least one strand, e.g., the reverse strand, may include one or more non-natural nucleotides, e.g, a nucleotide that has a non-natural sugar, a non-natural nucleotide base, a non-phosphodiester bond between nucleotides, or any combination thereof. In one embodiment, at least one of the strands may be formed using one or more of 2′-fluoro-2′deoxycytidine-5′-triphosphate, 5-iodocytidine-5′-triphosphate, 5-methylcytidine-5-triphosphate, 2′-O-methylcytidine-5′-triphosphate, 7-amino-2′-deoxycytidine-5′-triphosphate, 2-amino-2′-deoxycytidine-5′-triphosphate, 2′-azido-7-deoxycytidine-5′-triphosphate, aracytidine-5′-triphosphate, 2-thiocytidine-5′-triphosphate, 6-azacytidine-5′-triphosphate, 5-bromocytidine-5′-triphosphate, 3′-O-methylcytidine-5′-triphosphate, 5-aminoallylcytidine-5′-triphosphate, pseudoisocytidine-5′-triphosphate, N⁴-methylcytidine-5′-triphosphate, 5-carboxycytidine-5′-triphosphate, 5-formylcytidine-5′-triphosphate, 5-hydroxymethylcytidine-5′-triphosphate, 5-hydroxycytidine-5-triphosphate, 5-methoxycytidine-5′-triphosphate, thienocytidine-5′-triphosphate, cytidine-5′-triphosphate, 3′-deoxycytidine-5′-triphosphate, biotin-16-aminoallylcytidine-5′-triphosphate, cyanine 3-aminoallylcytidine-5′-triphosphate, cyanine 5-aminoallylcytidine-5′-triphosphate or cytidine-5′-O-(1-thiotriphosphate). In one embodiment, at least one of the strands is formed using one or more of 2′-fluoro-2-deoxyuridine-5-triphosphate, 5-iodouridine-5′-triphosphate, 2′-O-methyluridine-5′-triphosphate, pseudouridine-5′-triphosphate, 5-methyluridine-5′-triphosphate, 4-thiouridine-5′-triphosphate, 2′-amino-7-deoxyuridine-5′-triphosphate, 2′-azido-2′-deoxyuridine-5′-triphosphate, 2-thiouridine-5′-triphosphate, arauridine-5′-triphosphate, 5,6-dihydrouridine-5′-triphosphate, 6-azauridine-5′-triphosphate, 2-O-methylpseudouridine-5′-triphosphate, 2′-O-methyl-5-methyluridine-5′-triphosphate, 5-bromouridine-5′-triphosphate, 3-O-methyluridine-5′-triphosphate, 5-aminoallyluridine-5′-triphosphate, N¹-methylpseudouridine-5′-triphosphate, 5,6-dihydro-5-methyluridine-5′-triphosphate, 5-hydroxymethyluridine-5′-triphosphate, 5-formyluridine-5′-triphosphate, 5-carboxyuridine-5′-triphosphate, 5-hydroxyuridine-5′-triphosphate, 5-methoxyuridine-5′-triphosphate, thienouridine-5-triphosphate, 5-carboxymethylesteruridine-5′-triphosphate, uridine-5′-triphosphate, 3′-deoxy-5-methyluridine-5′-triphosphate, 3′-deoxyuridine-5′-triphosphate, biotin-16-aminoallyluridine-5′-triphosphate, desthiobiotin-16-aminoallyl-uridine-5′-triphosphate, cyanine 3-aminoallyluridine-5′-triphosphate, cyanine 7-aminoallyluridine-5′-triphosphate or uridine-5′-O-(1-thiotriphosphate). In one embodiment, at least one of the strands is formed using one or more of 5-aminoallyl-CTP, 2-amino-ATP, 5-Br-UTP, 5-carboxy-CTP, 5-carboxy-UTP, 5-carboxymethyest-UTP, 7-deaza-ATP, 5-formyl-CTP, 5-formyl-UTP, 5-hydroxy-CTP, 5-hydroxy-UTP, 5-hydroxymethyl-CTP, 5-hydroxymethyl-UTP, 5-iodo-UTP, 5-methoxy-CTP, 5-methoxy-UTP, N6-methyl-amino-ATP, N6-methyl-ATP, 5-methyl-CTP, pseudo-UTP, thieno-CTP, thieno-GTP, 1-thio-ATP or 2-thio-UTP. In one embodiment, one of the strands includes 5-formyl cytidine or pseudouridine. In one embodiment, at least 5%, 10%, 20%, 30%, 40%, 50% 60%, 70%, 80%, 90% or more of the nucleotides are non-natural nucleotides, and in one embodiment, the strands are hydrogen bonded over at least 5%, 10%, 20%, 30%, 40%, 50%, 60%, 70%, 80%, 90%, or more of the length of the strands.

In one embodiment, the RNA is at least a partially ds mRNA that is circular, e.g., a circular RNA containing an IRES (internal ribosomal entry site). In one embodiment, only one of the two strands of the ds circular RNA includes one or more non-natural nucleotides. In one embodiment, both of the strands of the ds circular RNA include one or more non-natural nucleotides. In one embodiment, at least one strand of the ds circular RNA has two or more different non-natural nucleotides. In one embodiment, a short RNA anneals with the 5′ cap or IRES and 3′ poly A tail in order to circularize single stranded mRNA, e.g., the overlap that results in ds RNA may be over less than 50%, 40%, 30%, 20%, 10%, 5% or less the full-length mRNA.

In one embodiment, the mRNA is self-amplifying RNA, e.g., generated by hybridization of self-amplifying RNA with a complementary RNA. In one embodiment, only one of the two strands of the ds self-amplifying RNA includes one or more non-natural nucleotides. In one embodiment, both of the strands of the self-amplifying RNA include one or more non-natural nucleotides. In one embodiment, at least one strand of the ds self-amplifying RNA has two or more different non-natural nucleotides. In one embodiment, the mRNA may encode at least one non-structural protein, such as a viral replicase, at least one positive-sense viral protein or at least one alphavirus protein, e.g., an alphavirus replicase such as one from Venezuelan equine encephalitis virus, Semliki forest virus or Sindbis virus, or flock house virus, or the at least one non-structural protein, such as a viral replicase, at least one positive-sense viral protein or at least one alphavirus protein may be provided in trans.

In one embodiment, a composition is provided comprising complexes of the ds mRNA with one or more other molecules that inhibit degradation of the ds mRNA In one embodiment, the composition comprises liposomes, such as a lipid nanoparticle (LNP), and the ds mRNA. In one embodiment, the composition comprises lipid complexes comprising the ds mRNA. In one embodiment, the composition comprises complexes comprising a peptide conjugate, e.g., a PEG-polyacridine peptide, e.g., an oligomer of acridine modified amino acids such as lysine, arginine or histidine, e.g., from about 2 to about 10, such as 3 to 6, acridine modified amino acids which may be the same amino acid of a plurality of different amino acids. In one embodiment, the composition comprises complexes comprising a mannose 9 glycopeptide. In one embodiment, the composition comprises complexes comprising a peptide that facilitates endosomal escape, e.g., mellitin. In one embodiment, the composition comprises complexes comprising a mannose 9 glycopeptide linked to a PEG-polyacridine peptide linked to a peptide that facilitates endosomal escape.

Further provided is a method to prevent, inhibit or treat a disorder in a mammal associated with pathogen infection or cancer. The method includes administering to the mammal an effective amount of a composition comprising one or more distinct ds mRNAs. In one embodiment, the composition may be systemically delivered. In one embodiment, the composition may be locally delivered. In one embodiment, the composition may be intramuscularly (i.m.) delivered, e.g., ds mRNA is expressed more persistently when dosed i.m., e.g., relative to ss mRNA.

Also provided are methods of making a composition comprising ds mRNA encoding a gene product of interest and a LNP or a peptide conjugate. In one embodiment, a strand of mRNA having a 5′ cap, a start codon, a polyA sequence and an open reading frame for the protein and a strand of RNA that has sequence complementarity with the mRNA over at least 10 nucleotides are provided. The mRNA and the RNA with sequence complementarity are allowed to hydrogen bond, thereby providing the ds mRNA. The dsRNA is then combined with a plurality of distinct lipids, e.g., two, three or four different lipids, or a peptide conjugate as described herein. In one embodiment, the strands are provided by transcription of one or more vectors, e.g. a plasmid vector. In one embodiment, the strands are provided by transcription of a single vector that includes an open reading frame for the protein that is flanked by a first promoter positioned to express the strand of mRNA and a second promoter positioned to express the strand of RNA with sequence complementarity. In one embodiment, at least one of the strands includes one or more non-natural nucleotides or nucleotide modifications. In one embodiment, the one or more nucleotide modifications are introduced post-synthesis of at least one of the strands. In one embodiment, the one or more non-natural nucleotides are incorporated during synthesis of at least one of the strands. In one embodiment, the strands are hydrogen bonded over at least 90% of the length of the strands. In one embodiment, the strands are hydrogen bonded over the entire length of the strands. In one embodiment, wherein the strands are not the same length. For example, when hybridized, the 3′ end of the RNA with sequence complementarity overhands the 5′ end of the strand of mRNA, or the 3′ end of the RNA with sequence complementarity is recessed relative to the 5′ end of the strand of mRNA. In one embodiment, the strands are the same length. In one embodiment, at least one of the strands is synthesized in an in vitro transcription reaction. In one embodiment, at least one of the strands is synthesized in a cell.

Further provided is a method of using the compositions comprising ds mRNA, e.g., to express a prophylactic or therapeutic gene product. In one embodiment, a composition comprising a plurality of distinct lipids and a ds mRNA encoding the gene product, wherein at least one strand of the ds mRNA has a 5′ cap, a start codon, a polyA sequence and encodes the protein, wherein the two strands of the ds mRNA are hydrogen bonded over at least 10 nucleotides, is introduced to cells in an amount effective to express the gene product. In one embodiment, a composition comprising a peptide conjugate and a ds mRNA encoding the gene product, wherein at least one strand of the ds mRNA has a 5′ cap, a start codon, a polyA sequence and encodes the protein, wherein the two strands of the ds mRNA are hydrogen bonded over at least 10 nucleotides, is introduced to cells in an amount effective to express the gene product. In one embodiment, the cells are in a mammal for example, the composition is systemically administered to the mammal. In one embodiment, the composition is locally administered to the mammal. In one embodiment, the protein is for cancer immunotherapy. In one embodiment, the protein is a cancer antigen. In one embodiment, the protein is a protein of a pathogen or a microbial protein, for instance, one useful for immunization. In one embodiment, the composition further comprises a carrier protein. In one embodiment, the ds mRNA forms a complex with the plurality of lipids or the peptide conjugate thereby forming a nanoparticle. In one embodiment, the nanoparticle has a diameter of about 50 nm to about 500 nm, about 75 nm to about 250 nm, or about 100 nm to about 200 nm. In one embodiment, the ds mRNA forms a microparticle, e.g., the microparticle has a diameter of about 0.5 μm to about 500 μm, about 10 μm to about 30 μm, or about 20 μm to about 40 μm.

In one embodiment, a vaccine is provided. For example, a vaccine may include lipid nanoparticles (LNPs) comprising an amount of double stranded (ds) mRNA encoding a prophylactic gene product, wherein at least one strand of the ds mRNA has a 5′ cap and/or IRES, a start codon, a polyA sequence and encodes the protein, wherein the two strands of the ds mRNA are hydrogen bonded over at least 10 nucleotides, and a pharmaceutically acceptable carrier. In one embodiment, at least one strand of the ds mRNA encodes a viral protein or an antigenic fragment thereof. In one embodiment, at least one strand of the ds mRNA encodes a coronavirus spike protein or an antigenic portion thereof including the receptor binding domain. In one embodiment, at least one strand of the ds mRNA includes one or more non-natural nucleotides. In one embodiment, at least one of the non-natural nucleotides has a non-natural sugar. In one embodiment, at least one of the non-natural nucleotides has a non-natural nucleobase. In one embodiment, at least one strand of the ds mRNA includes at least one non-phosphodiester bond. In one embodiment, one of the strands includes 5-formyl cytidine or pseudouridine. In one embodiment, at least 5% of the nucleotides are non-natural nucleotides. In one embodiment, the non-natural nucleotide analog is a purine analog. In one embodiment, the strands of the ds mRNA are hydrogen bonded over at least 90% of the length of the strands. In one embodiment, the strands of the ds mRNA are hydrogen bonded over the entire length of the strands. In one embodiment, one of the strands of the ds mRNA is no more than 5 or 10 kb in length. In one embodiment, only one of the two strands of the ds mRNA includes one or more non-natural nucleotides. In one embodiment, both of the strands of the ds mRNA include one or more non-natural nucleotides. In one embodiment, at least one strand of the ds mRNA has two or more different non-natural nucleotides. In one embodiment, the comprises lipid particles have a diameter of about 75 nm to 250 nm. In one embodiment, the LNPs comprise DSPC, cholesterol, PEG-DMA, SM-102, or any combination thereof. In one embodiment, the DSPC is about 5 to about 20 wt %, the cholesterol is about 35 to about 45 wt %, the PEG-DMA is bout 1 to about 2.5 wt %, or the SM-102 is about 40 to about 60 wt %. In one embodiment, the DSPC is about 7.5 to about 13 wt %, the cholesterol is about 35 to about 40 wt %, the PEG-DMA is about 1.25 to about 2 wt %, or the SM-102 is about 45 to about 55 wt %.

Also provided is a method to immunize an animal. The method includes administering to the animal an effective about of the vaccine. In one embodiment, the animal is a mammal. In one embodiment, the animal is a human. In one embodiment, the vaccine is intramuscularly administered. In one embodiment, the vaccine is subcutaneously administered. In one embodiment, an additional dose is administered. In one embodiment, another vaccine to the same gene product is administered.

### BRIEF DESCRIPTION OF FIGURES

FIGS. 1A-1C. Metabolically Stable Double Stranded mRNA. A) The generation of ds mRNA from ss mRNA and reverse mRNA is illustrated B) RNAse A digestion of ss and ds mRNA and polyplexes demonstrates the 1000-fold increase in stability of ds mRNA versus ss mRNA and 1,000,000-fold increase stability of ds mRNA polyplexes that resist digestion even when challenged with 10 μg (50 U) of RNAse A (Poliskey et al., 2018) C) Subcutaneous administration of ds and ss mRNA followed by electroporation results in luciferase expression in the skin for 15 days for ds mRNA compared to 7 days for ss mRNA.

FIG. 2. PEGylated Polyacridine Peptide. The structure of a polyacridine peptide (PAcr) linked by disulfide bond to a 5 kDa polyethylene glycol (PEG) is illustrated. When the PEG-polyacridine peptide-conjugate is combined with ds mRNA, nanoparticles form instantly. Disulfide bond reduction at the cell surface releases PEG causing a rapid rise in nanoparticle charge from +4 mV to +22 mV leading to transfection through charge-mediated pinocytosis.

FIG. 3. High-Mannose N-glycan Targeted Gene Delivery to DCSIGN Expressing CHO Cells. The Man9 glycopeptide (GP) mediates selective gene expression in CHO cells expressing dendritic cell DC-SIGN (+) (Anderson et al., 2010) when compared to peptide alone or polyetheneimine (PEI) (Bousslf et al., 1995). Note that PEI-mediated transfection is more efficient that receptor mediates, but less selective.

FIG. 4. Melittin Mediated Endosomal Escape. The structure of polyacridine-melittin peptide-conjugate (PAcr-Mel) is illustrated. PAcr-Mel is equivalent to polyethyleneimine (PEI) during in vitro transfection of luciferase mRNA in cell culture. However, when dosed in vivo, PAcr-Mel is active and PEI is inactive at luciferase mRNA gene transfer in the skin of mice.

FIGS. 5A-5C. Exemplary Delivery Mechanism for a Peptide-conjugate ds mRNA Nanoparticle Vaccine. A) ds mRNA nanoparticles possess a Man9 ligand that binds to DC-SIGN on dendritic cells. Subsequent reduction of PEG unmasks positively charged nanoparticles that bind to negatively charged glycosaminoglycans (GAG) resulting in pinocytosis. B) ds mRNA nanoparticles are triggered to release melittin that inserts and ruptures the endosomal membrane resulting in mRNA release into the cytosol and translation into COVID-19 spike protein. C) The spike protein is proteolyzed by the proteasome and peptides are loaded onto MHC I and II, then migrate to the cell surface to present antigens to program B and T-cells.

FIG. 6. Peptide-conjugate for mRNA Vaccine Delivery. Steps 1-3 bring together functional components of the peptide-conjugate in a convergent synthesis. Man 9 is tethered to a 5 KDa PEG linker in Step 1. This is reacted with a C-terminal Cys on ds mRNA PAcr binding peptide in Step 2. The thiol-pyridine (TP) functionalized melittin analogue (Mel) is conjugated to the deprotected N-terminal Cys on PAcr in Step 3. Critical disulfide bonds (S—S-1 and S—S-2) are indicated. Inset A shows the PAcr-Mel fragment. Inset B demonstrates RP-HPLC purity of the Man9-PEG-PAcr-Mel peptide-conjugate.

FIG. 7. Plasmid map and gel showing mRNA encoding coronavirus spike protein, the complement thereof, and ds mRNA formed by hybridizing the mRNA and the complement thereof.

FIG. 8. Kinetics of ss and ds mRNA LNP Expression. ss or ds Luc mRNA LNP (5 μg in 20 μl) were dosed i.m. in triplicate mice. At day 1-6, luciferase expression was quantified by Bioluminescence Imaging (BLI).

FIG. 9. mRNA LNP Stability. ss and ds mRNA LNPs were stored at −70° C. or 20° C. for 12 hours, followed by extraction of mRNA and analysis on agarose gel electrophoresis. Integration of the band intensity resulted in (1 vs 2) 15%, (3 vs 4) 45% and (5 vs 6) 85% recovery of mRNA after 12 hours at 20° C.

FIG. 10. Covid-19 Spike ds mRNA. The agarose gel illustrates ss mRNA encoding ½ covid-19 spike protein. The reverse strand, rev, hybridizes with ss to protect the spike coding sequence and forms ds.

FIGS. 11A-11B. Spike ds mRNA LNP Vaccine. A study analyzed the T-cell response to dosing spike ss and ds mRNA LNPs. Panel A compares CD4+ TNFa secreting T-cells. Panel B compared CD8+ TNFa secreting T-cells. N=3, the results were underpowered.

### DETAILED DESCRIPTION

Various non-viral vectors can be used to deliver DNA, mRNA and short double-stranded RNA, including small interfering RNA (siRNA) and microRNA (miRNA) mimics. However, delivery of double stranded RNA (not mRNA, siRNA or miRNA) is highly toxic to cells due to triggering of apoptosis. Moreover, in order to be useful for gene therapy, the vectors need to avoid degradation by serum endonucleases and evade immune detection. They also need to avoid renal clearance from the blood and prevent nonspecific interactions.

A stabilized ds mRNA containing composition is disclosed herein that is useful for prophylactic or therapeutic gene delivery. The compositions may be employed in methods to prevent, inhibit or treat a disorder or disease in a mammal, such as a canine, feline, bovine, porcine, equine, caprine, ovine, or human, which disorder or disease is amenable to treatment with one or more exogenously delivered genes. For example, the disorder or disease may be associated with a decreased amount of a gene product, the absence of a gene product, or the presence of an aberrant gene product, e.g., one having no activity, aberrant activity, reduced activity or increased activity relative to a mammal without the disorder or disease.

mRNA Vaccines

mRNA vaccines have many advantages over traditional vaccines that have been developed (Pardi et al., 2018). Some of these include rapid R&D and production, simultaneous vaccination with multiple immunogens and a high margin of safety (Pardi et al., 2018). One of the most important features is the ability to rapidly generate an mRNA vaccine by substituting new mRNA into an existing delivery vehicle (Gomez-Aguado et al., 2020). This greatly reduces the time normally needed to generate and formulate a viral protein or to develop an attenuated virus, two strategies most often used for traditional vaccine development (L, et al., 2020).

While there are many types of mRNA vaccines, they all use single stranded mRNA to encode an immunogenic viral protein that is packaged, delivered and expressed in muscle or skin cells, leading to a B-cell immune response against the virus (Pardi et al., 2020). Following intra-dermal administration, mRNA nanoparticles are likely taken by fibroblasts and dendritic cells of the dermis (Selmi et al., 2016; Diken et al., 2011). This occurs by either receptor mediated endocytosis or pinocytosis (FIG. 5). Targeted delivery of nanoparticles possessing mannose ligands facilitates dendritic cell entry via DC-SIGN receptor-mediated endocytosis into endosomes (Gao et al., 2020; Le Moignic et al., 2018; Perche et al., 2011; Pichon & Midoux, 2013). Alternatively, the nanoparticle sheds the polyethylene glycol (PEG) layer at the cell surface by reduction of disulfide bonds, leading to charge unmasking and binding to proteoglycans, followed by pinocytosis (FIG. 5). Following cellular endocytosis, mRNA nanoparticles are engineered to exit endosomes into the cytosol by reductive release of pour-forming melittin peptides from the nanoparticle, resulting in melittin lysis of endosomes (FIG. 5B). The intracellular release of mRNA results in binding to ribosomes, and translation of the mRNA into the programed viral protein (COVID-19, FIGS. 5B-C). Viral proteins are proteolyzed by the proteasome and peptide antigens are presented by MHC I on dendritic cell surface to prime for T-cell response (FIG. 5C). Alternatively, transfected fibroblasts secrete viral proteins which are endocytosed by dendritic cells and proteolytically processed, leading to peptide antigen presentation on MHC II to prime the B-cell response (FIG. 5C).

There is intense scientific interest in ways to improve mRNA stability, booster with adjuvants, and direct B and T-cell responses (Pardi et al., 2018; Gomez-Aguado, et al., 2020; Pardi et al., 2020). There is still much debate regarding if it is necessary to target dendritic cells to gain a long lasting T-cell response (Pardi et al., 2020). What is increasingly clear, is that the delivery vehicle and mRNA stability plays a role in determining the magnitude and type of immune response (Pardi, et al., 2020).

The LNP mRNA vaccine includes double stranded mRNA and a plurality of distinct lipid molecules

The peptide-conjugate mRNA vaccine delivery platform 1) contains double stranded mRNA as opposed to single stranded mRNA, 2) contains a peptide-conjugate instead of a lipid nanoparticle, and 3) contains a Man 9 N-glycan to target dendritic cells. The rationale for these changes are that double stranded mRNA is significantly more metabolically stable compared to single stranded mRNA (Poliskey et al., 2018). In addition, the RNA duplex results in a self-adjuvant effect to boost immunogenicity (Uchida et al., 2018). The peptide-conjugate is one of the most sophisticated and well-tested peptide delivery systems in the field which has been systematically optimized to form small stable nanoparticles that release mRNA intracellular (Poliskey et al., 2018; Crowley et al., 2015; Kizzire et al., 2013; Khargharia et al., 2013; Fernandez et al., 2011). The use of a Man 9 targeting ligand to direct increased transfection of dendritic cells in the dermis could influence the quality of the immune response (Gao et al., 2020; Le Moignic et al., 2018; Perche et al., 2011)

### Exemplary Lipids

Numerous lipids which are used in liposome delivery systems may be used. Virtually any lipid or polymer which is used to form a liposome or polymersome may be used. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid according to an embodiment. Often cholesterol is incorporated into lipid particles in order to enhance structural integrity. These lipids are all readily available commercially from Avanti Polar Lipids, Inc. (Alabaster, Alabama, USA). DOPE and DPPE are particularly useful for conjugating (through an appropriate crosslinker) peptides, polypeptides, including antibodies, RNA and DNA through the amine group on the lipid.

In certain embodiments, the lipid is comprised of one or more lipids selected from the group consisting of phosphatidyl-cholines (PCs) and cholesterol.

In certain embodiments, the lipid is comprised of one or more phosphatidyl-cholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

In other embodiments: (a) the lipid is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid nanoparticle is comprised of phospholipid selected from a monoacyl or diacylphosphoglyceride.

In still other illustrative embodiments, the lipid is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In still other embodiments, the lipid comprises one or more PEG-containing phospholipids, for example 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)](ammonium salt) (DSPE-PEG), 1,2-distearoyl-sn-glycero-3-phosphoethanolamine-N-[amino(polyethylene glycol)] (DSPE-PEG-NH₂) (DSPE-PEG). In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N-[amino(polyethylene glycol)-2000](DSPE-PEG₂₀₀₀-NH₂) which can be used to covalent bind a functional moiety to the lipid.

In on embodiment, the lipid particle comprises one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

In one embodiment, pharmaceutical compositions described herein may include, without limitation, lipids such as 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula 1-VI of US Patent Publication No. US20100036115; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl] pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimeth-yl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, [4(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propa-n-2-amine, S—N,N-dimethyl-1-[(9Z,12Z)-octadeca-9,12-dien-1-yloxy]-3-(octy-loxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z-)-oct-5-en-1-yloxy]propan-2-amine, 1-{2-[(9Z,12Z)-octadeca-9,12-dien-1-yloxy]-1-[(octyloxy)methyl]ethyl}azet-idine, (2S)-1-(hexyloxy)-N,N-dimethyl-3-[(9Z,12Z)-octadeca-9,12-dien-1-ylo-xy]propan-2-amine, (2S)-1-(heptyloxy)-N,N-dimethyl-3-[(9Z,12Z)-octadeca-9,12-dien-1-yloxy]pr-opan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-am-ine, (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)pro-pan-2-amine, (2S)-1-(hexyloxy)-3-[(11Z,14Z)-icosa-11,14-dien-1-yloxy-N,N-dimethylprop-an-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)pr-opan-2-amine, (2S)-14(13Z,16Z)-docosa-13,16-dien-1-yloxy]-3-(hexyloxy)-N,N-dimethylpro-pan-2-amine, (2S)-1-[(13Z)-docos-13-en-1-yloxy-3-(hexyloxy)-N,N-dimethylpropan-2-amin-e, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-di-en-1-yloxy]propan-2-amine, N,N-dimethyl-1-(octyloxy)-3-({8-[(1S,2S)-2-{[(1R,2R)-2-pentylcyclopropyl]-methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the LNP may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40.10:48 (see e.g., Geall et al., Nonviral delivery of self-amplifying RNA vaccines, PNAS 2012; PMID: 22908294; herein incorporated by reference in its entirety).

In one embodiment, the LNP may include MC3.

Exemplary Disorders or Diseases for Use with the Compositions

The compositions may be employed to prevent, inhibit or treat a variety of disorders or diseases associated with a deficiency in (or absence of) a protein or an aberrant protein (e.g., with low or no activity or excessive or unregulated activity) (see Table 1 for a list of monogenic disorders). Genes that may be employed include but are not limited to those that prevent, inhibit or treat hemophilia, anemia or other blood disorders, cancer, cardiovascular disease, lysosomal storage diseases, musculoskeletal diseases, neurodegenerative diseases, respiratory disease, and the like. Exemplary genes are shown in Table 2.

TABLE 1
Monogenic disorders
Adrenoleukodystrophy
α-1 antitrypsin deficiency
Becker muscular dystrophy
β-thalassaemia
Canavan disease
Chronic granulomatous disease
Cystic fibrosis
Duchenne muscular dystrophy
Fabry disease
Familial adenomatous polyposis
Familial hypercholesterolaemia
Fanconi anaemia
Galactosialidosis
Gaucher's disease
Gyrate atrophy
Haemophilia A and B
Hurler syndrome
Hunter syndrome
Huntington's chorea
Junctional epidermolysis bullosa
Late infantile neuronal ceroid
lipofuscinosis
Leukocyte adherence deficiency
Limb girdle muscular dystrophy
Lipoprotein lipase deficiency
Mucopolysaccharidosis type VII
Ornithine transcarbamylase
deficiency
Pompe disease
Purine nucleoside phosphorylase
deficiency
Recessive dystrophic epidermolysis
bullosa
Sickle cell disease
Severe combined immunodeficiency
Tay Sachs disease
Wiskott-Aldrich syndrome
Cardiovascular disease
Anaemia of end stage renal disease
Angina pectoris (stable, unstable, refractory)
Coronary artery stenosis
Critical limb ischaemia
Heart failure
Intermittent claudication
Myocardial ischaemia
Peripheral vascular disease
Pulmonary hypertension
Venous ulcers
Infectious disease
Adenovirus infection
Cytomegalovirus infection
Epstein-Barr virus
Hepatitis B and C
HIV/AIDS
Influenza
Japanese encephalitis
Malaria
Paediatric respiratory disease
Respiratory syncytial virus
Tetanus
Tuberculosis
Cancer
Gynaecological - breast, ovary, cervix, vulva
Nervous system - glioblastoma,
leptomeningeal carcinomatosis, glioma, astrocytoma, neuroblastoma,
retinoblastoma
Gastrointestinal - colon, colorectal, liver metastases, post-hepatitis
liver cancer, pancreas, gall bladder
Genitourinary - prostate, renal, bladder, anogenital neoplasia
Skin - melanoma (malignant/metastatic)
Head and neck - nasopharyngeal carcinoma, squamous cell carcinoma,
oesophageal cancer
Lung - adenocarcinoma, small cell/nonsmall cell, mesothelioma
Haematological - leukaemia, lymphoma, multiple myeloma
Sarcoma
Germ cell
Li-Fraumeni syndrome
Thyroid
Neurological diseases
Alzheimer's disease
Amyotrophic lateral sclerosis
Carpal tunnel syndrome
Cubital tunnel syndrome
Diabetic neuropathy
Epilepsy
Multiple sclerosis
Myasthenia gravis
Parkinson's disease
Peripheral neuropathy
Pain
Ocular diseases
Age-related macular degeneration
Diabetic macular edema
Glaucoma
Retinitis pigmentosa
Superficial corneal opacity
Choroideraemia
Leber congenital amaurosis
Inflammatory diseases
Arthritis (rheumatoid, inflammatory, degenerative)
Degenerative joint disease
Degenerative joint disease
Ulcerative colitis
Severe inflammatory disease of the rectum
Other diseases
Chronic renal disease
Erectile dysfunction
Detrusor overactivity
Parotid salivary hypofunction
Oral mucositis
Fractures
Type I diabetes
Diabetic ulcer/foot ulcer
Graft versus host disease/transplant patients

TABLE 2

| Gene Symbol | Protein name | Related Diseases |
|---|---|---|
| BCL2L11 | BCL2-like 11 (apoptosis | Cancer, e.g. human T- |
| | facilitator) | cell acute |
| | | lymphoblastic |
| | | leukemia and |
| | | lymphoma |
| BRCA1 | breast cancer 1, early | Cancer, e.g. breast |
| | onset | cancer, pancreatic |
| | | cancer |
| F8 | coagulation factor VIII, | Hemophilia |
| | procoagulant component | |
| FLI1 | Friend leukemia virus | cancer, e.g. Ewing's |
| | integration 1 | sarcoma, and |
| | | myelodysplasia |
| FMR1 | fragile X mental | Fragile X syndrome |
| | retardation 1 | and premature |
| | | ovarian failure |
| FNDC5 | fibronectin type III | Obesity, Type 2 |
| | domain containing 5 | Diabetes |
| GCK | glucokinase (hexokinase | Obesity, Type 2 |
| | 4) | Diabetes, and |
| | | Hyperinsulinemic |
| | | hypoglycemia |
| GLP1R | glucagon-like peptide 1 | Type 2 Diabetes |
| | receptor | |
| GRN | granulin | autoimmune, |
| | | inflammatory, |
| | | dementia/CNS |
| | | disease, cancer, e.g. |
| | | hepatic cancer |
| HAMP | hepcidin antimicrobial | hemochromatosis, |
| | peptide | thalassemia |
| HPRT1 | hypoxanthine | Lesch-Nyhan disease |
| | phosphoribosyltransferase 1 | and HPRT-related |
| | | gout |
| IDO1 | indoleamine 2,3- | autoimmune and |
| | dioxygenase 1 | inflammatory diseases |
| IGF1 | insulin-like growth factor 1 | metabolic disease, |
| | (somatomedin C) | delayed growth, |
| | | cancer |
| IL10 | interleukin 10 | Autoimmune and |
| | | inflammatory |
| | | diseases, e.g. graft vs. |
| | | host disease and |
| | | rheumatoid arthritis |
| LDLR | low density lipoprotein | dyslipidemias, |
| | receptor | atherosclerosis, and |
| | | hypercholesterolemia |
| NANOG | Nanog homeobox | tissue regeneration |
| PTGS2 | prostaglandin- | inflammation, cancer, |
| | endoperoxide synthase 2 | infectious disease |
| | (prostaglandin G/H | |
| | synthase and | |
| | cyclooxygenase) | |
| RB1 | retinoblastoma 1 | cancer, e.g. bladder |
| | | cancer, osteosarcoma, |
| | | retinoblastoma, small |
| | | cell lung cancer |
| SERPINF1 | serpin peptidase inhibitor, | cancer, choroidal |
| | Glade F (alpha-2 | neovascularization, |
| | antiplasmin, pigment | cardiovascular |
| | epithelium derived | disease, diabetes, and |
| | factor), member 1 | osteogenesis |
| | | imperfecta |
| SIRT1 | sirtuin 1 | Metabolic disease, |
| | | aging |
| SIRT6 | sirtuin 6 | antioxidative |
| | | pathway, anti-NFkB |
| SMAD7 | SMAD family member 7 | Acute kidney injury |
| | | (anti-TGFb), |
| | | colorectal cancer |
| ST7 | suppression of | cancer, e.g. myeloid |
| | tumorigenicity 7 | cancer, head and neck |
| | | squamous cell |
| | | carcinomas, breast |
| | | cancer, colon |
| | | carcinoma, and |
| | | prostate cancer |
| STAT3 | signal transducer and | tissue regeneration |
| | activator of transcription 3 | and Hyper-IgE |
| | (acute-phase response | recurrent infection |
| | factor) | syndrome |
| CFTR | Cystic fibrosis | Cystic fibrosis (CF) |
| | transmembrane | and congenital |
| | conductance regulator | bilateral absence of |
| | | vas deferens |
| | | (CBAVD) |
| PAH | Phenylalanine | Phenylketonuria |
| | hydroxylase | (PKU) |
| CEP290 | Centrosomal protein of | Leber's congenital |
| | 290 kDa | amaurosis (LCA), |
| | | Bardet-Biedl |
| | | syndrome (BBS), |
| | | Joubert syndrome, |
| | | Meckel syndrome, |
| | | Sior-Loken syndrome |
| CD274 | cluster of differentiation | Autoimmune disease, |
| (also | 274 (also known as | transplant rejection, |
| known as | Programmed cell death 1 | allergies or asthma |
| PD-L1) | ligand 1) | |
| ADIPOQ | adiponectin, C1Q and | Obesity and obesity- |
| | collagen domain | linked diseases (e.g., |
| | containing (also known as | hypertension, |
| | adiponectin) | metabolic |
| | | dysfunction, type 2 |
| | | diabetes, |
| | | atherosclerosis, and |
| | | ischemic heart |
| | | disease) |

### Hemophilia-F8, F9, F11, VWF

Hemophilia is a group of hereditary genetic disorders that impair the body's ability to control blood clotting or coagulation, which is used to stop bleeding when a blood vessel is broken. Like most recessive sex-linked, X chromosome disorders, hemophilia is more likely to occur in males than females. For example, Hemophilia A (clotting factor VIII deficiency), the most common form of the disorder, is present in about 1 in 5,000-10,000 male births. Hemophilia B (factor IX deficiency) occurs in around 1 in about 20,000-34,000 male births. Hemophilia lowers blood plasma clotting factor levels of the coagulation factors, e.g. F8, needed for a normal clotting process. Thus when a blood vessel is injured, a temporary scab does form, but the missing coagulation factors prevent fibrin formation, which is necessary to maintain the blood clot. F8, for example, encodes Factor VIII (FVIII), an essential blood clotting protein. Factor VIII participates in blood coagulation, it is a cofactor for factor IXa which, in the presence of Ca⁺² and phospholipids forms a complex that converts factor X to the activated form Xa.

Aspects of the invention disclosed herein provide methods and compositions that are useful for upregulating F8 for the treatment and/or prevention of diseases associated with reduced F8 expression or function such as hemophilia. Aspects of the invention disclosed herein provide methods and compositions that are useful for upregulating F9 for the treatment and/or prevention of diseases associated with reduced F9 expression or function such as hemophilia. Aspects of the invention disclosed herein provide methods and compositions that are useful for upregulating F11 for the treatment and/or prevention of diseases associated with reduced F11 expression or function such as hemophilia. Aspects of the invention disclosed herein provide methods and compositions that are useful for upregulating VWF for the treatment and/or prevention of diseases associated with reduced VFW expression or function such as Von Willebrand's Disease

Thus, in one embodiment, the compositions may be employed to prevent, inhibit or treat hemophilia including but not limited to hemophilia A, characterized by low levels of or the absence of factor 8 (Also called FVIII or factor VIII deficiency), hemophilia B, characterized by low levels of or the absence of factor 9 (Also called FIX or factor IX deficiency), hemophilia C, characterized by low levels of or the absence of factor 11 (Also called FXI or factor XI deficiency), or Von Willebrands Disease, characterized by a deficiency of a blood clotting protein Von Willebrand factor.

### Lysosomal Storage Diseases

In one embodiment, the compositions may be employed to prevent, inhibit or treat a lysosomal storage disease. Lysosomal storage diseases include, but are not limited to, mucopolysacchandosis (MPS) diseases, for instance, mucopolysacchandosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV e.g., mucopolysaccharidosis type IV, e.g., Morquio syndrome (a deficiency of galactosamine-6-sulfate sulfatase or beta-galactosidase); mucopolysaccharidosis type VI, e.g., Maroteaux-Lamy syndrome (a deficiency of arylsulfatase B); mucopolysaccharidosis type II; mucopolysaccharidosis type 11 (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase); mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1 gangliosidosis (type I or type II); Fabry disease (a deficiency in alpha galactosidase); metachromatic leukodystrophy (a deficiency of aryl sulfatase A); Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), ceroid lipofuscinosis, and Gaucher disease (types I, II and III; a deficiency in glucocerebrosidase), as well as disorders such as Hermansky-Pudlak syndrome; Amaurotic idiocy; Tangier disease; aspartylglucosaminuria; congenital disorder of glycosylation, type Ia; Chediak-Higashi syndrome; macular dystrophy, corneal, 1; cystinosis, nephropathic; Fanconi-Bickel syndrome; Farber lipogranulomatosis; fibromatosis; geleophysic dysplasia; glycogen storage disease I; glycogen storage disease Ib; glycogen storage disease Ic; glycogen storage disease III; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis II, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease, infantile sialic acid storage disorder; sialuria; multiple sulfatase deficiency; triglyceride storage disease with impaired long-chain fatty acid oxidation; Winchester disease; Wolman disease (a deficiency of cholesterol ester hydrolase); Deoxyribonuclease I-like 1 disorder; arylsulfatase E disorder; ATPase, H+ transporting, lysosomal, subunit 1 disorder; glycogen storage disease IIb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria.

### Cancer-SERPINF1, BCL2L11, BRCA1, RB1, ST7

In one embodiment, the compositions may be employed to prevent, inhibit or treat cancer. Cancer is a broad group of various diseases, all involving unregulated cell growth. In cancer, cells divide and grow uncontrollably, forming malignant tumors, and invade nearby parts of the body Several genes, many classified as tumor suppressors, are down-regulated during cancer progression, e.g., SERPINF1, BCL2L11, BRCA1, RB1, and ST7, and have roles in inhibiting genomic instability, metabolic processes, immune response, cell growth/cell cycle progression, migration, and/or survival. These cellular processes are important for blocking tumor progression. SERPINF1 encodes an anti-angiogenic factor. BCL2L11 encodes an apoptosis facilitator. BRCA1 encodes a RING finger protein involved in DNA damage repair. RB1 prevents excessive cell growth by inhibiting cell cycle progression until a cell is ready to divide. ST7 suppresses tumor growth in mouse models and is involved in regulation of genes involved in differentiation. Aspects of the invention disclosed herein provide methods and compositions that are useful for upregulating SERPINF1, BCL2L11, BRCA1, RB1, and ST7 for the treatment and/or prevention of diseases associated with reduced SERPINF1, BCL2L11, BRCA1, RB1, and ST7 expression or function such as cancer. For example, aspects of the invention disclosed herein provide methods and compositions that are useful for upregulating BCL2L11 for the treatment or prevention of human T-cell acute lymphoblastic leukemia and lymphoma. In another example, aspects of the invention disclosed herein provide methods and compositions that are useful for upregulating BRCA1 for the treatment or prevention of breast cancer or pancreatic cancer. In another example, aspects of the invention disclosed herein provide methods and compositions that are useful for upregulating RB1 for the treatment or prevention of bladder cancer, osteosarcoma, retinoblastoma, or small cell lung cancer. In another example, aspects of the invention disclosed herein provide methods and compositions that are useful for upregulating ST7 for the treatment or prevention of myeloid cancer, head and neck squamous cell carcinomas, breast cancer, colon carcinoma, or prostate cancer.

Examples of cancer include but are not limited to leukemias, lymphomas, myelomas, carcinomas, metastatic carcinomas, sarcomas, adenomas, nervous system cancers and genito-urinary cancers. In some embodiments, the cancer is adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinemia, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, or Wilms tumor.

### Fragile X Syndrome—FMR1

Fragile X syndrome (FXS) (also known as Martin-Bell syndrome, or Escalante's syndrome) is a genetic syndrome that is the most common known single-gene cause of autism and the most common inherited cause of intellectual disability. It results in a spectrum of intellectual disability ranging from mild to severe as well as physical characteristics such as an elongated face, large or protruding ears, and larger testes (macroorchidism), behavioral characteristics such as stereotypical movements (e.g. hand-flapping), and social anxiety. Fragile X syndrome is associated with the expansion of the CGG trinucleotide repeat affecting the Fragile X mental retardation 1 (FMR1) gene on the X chromosome, resulting reduced expression of the X mental retardation protein (FMRP), which is required for normal neural development. Aspects of the invention disclosed herein provide methods and compositions that are useful for upregulating FMR1 for the treatment and/or prevention of diseases associated with reduced FMR1 expression or function such as Fragile X syndrome.

### Premature Ovarian Failure—FMR1

Premature Ovarian Failure (POF), also known as premature ovarian insufficiency, primary ovarian insufficiency, premature menopause, or hypergonadotropic hypogonadism, is the loss of function of the ovaries before age 40. POF can be associated mutations in the Fragile X mental retardation 1 (FMR1) gene on the X chromosome, resulting reduced expression of the X mental retardation protein (FMRP). Aspects of the invention disclosed herein provide methods and compositions that are useful for upregulating FMR1 for the treatment and/or prevention of diseases associated with reduced FMR1 expression or function such as Premature Ovarian Failure.

### Obesity-FNDC5, GCK, ADIPOQ

Obesity is a medical condition in which excess body fat has accumulated to the extent that it may have an adverse effect on health, leading to reduced life expectancy and/or increased health problems. A person is considered obese when his or her weight is 20% or more above normal weight. The most common measure of obesity is the body mass index or BMI. A person is considered overweight if his or her BMI is between 25 and 29.9; a person is considered obese if his or her BMI is over 30. Obesity increases the likelihood of various diseases, particularly heart disease, type 2 diabetes, obstructive sleep apnea, certain types of cancer, and osteoarthritis. Obesity is most commonly caused by a combination of excessive food energy intake, lack of physical activity, and genetic susceptibility. Overexpression of FNDC5, fibronectin type II containing 5, has been shown in animal models to reduce body weight in obese mice. GCK, glucokinase (hexokinase 4), phosphorylates glucose to produce glucose-6-phosphate, the first step in most glucose metabolism pathways. Mutations in the GCK gene have been found to be associated with obesity in humans. Aspects of the invention disclosed herein provide methods and compositions that are useful for upregulating FNDC5 for the treatment and/or prevention of diseases associated with reduced FNDC5 expression or function such as obesity. Aspects of the invention disclosed herein provide methods and compositions that are useful for upregulating GCK for the treatment and/or prevention of diseases associated with reduced GCK expression or function such as obesity.

Adiponectin, encoded by the ADIPOQ gene, is a hormone that regulates metabolism of lipids and glucose. Adipocytes found in adipose tissue secrete adiponectin into the bloodstream where it self-associates into larger structures by binding of multiple adiponectin trimers to form hexamers and dodecamers. Adiponectin levels are inversely related to the amount of body fat in an individual and positively associated with insulin sensitivity both in healthy subjects and in diabetic patients. Adiponectin has a variety of protective properties against obesity-linked complications, such as hypertension, metabolic dysfunction, type 2 diabetes, atherosclerosis, and ischemic heart disease through its anti-inflammatory and anti-atherogenic properties Specifically with regard to type 2 diabetes, administration of adiponectin has been accompanied by a reduction in plasma glucose and an increase in insulin sensitivity. Aspects of the invention disclosed herein provide methods and compositions that are useful for upregulating ADIPOQ for the treatment and/or prevention of diseases associated with reduced ADIPOQ expression or function such as obesity or an obesity-linked disease or disorders such as hypertension, metabolic dysfunction, type 2 diabetes, atherosclerosis, and ischemic heart disease.

### Type 2 Diabetes—FNDC5, GCK, GLP1R, SIRT1, ADIPOQ

Type 2 diabetes (also called Diabetes mellitus type 2 and formally known as adult-onset diabetes) a metabolic disorder that is characterized by high blood glucose in the context of insulin resistance and relative insulin deficiency. Type 2 diabetes makes up about 90% of cases of diabetes with the other 10% due primarily to diabetes mellitus type 1 and gestational diabetes Obesity is thought to be the primary cause of type 2 diabetes in people who are genetically predisposed to the disease. The prevalence of diabetes has increased dramatically in the last 50 years. As of 2010 there were approximately 285 million people with the disease compared to around 30 million in 1985. Overexpression of FNDC5, fibronectin type II containing 5, has been shown in animal models to improve their insulin sensitivity GCK, glucokinase (hexokinase 4), phosphorylates glucose to produce glucose-6-phosphate, the first step in most glucose metabolism pathways. Mutations in the GCK gene are known to be associated with Type 2 Diabetes. Glucagon-like peptide 1 receptor (GLP1R) is known to be expressed in pancreatic beta cells. Activated GLP1R stimulates the adenylyl cyclase pathway which results in increased insulin synthesis and release of insulin. SIRT1 (Sirtuin 1, also known as NAD-dependent deacetylase sirtuin-1) is an enzyme that deacetylates proteins that contribute to cellular regulation. Sirtuin 1 is downregulated in cells that have high insulin resistance and inducing its expression increases insulin sensitivity, suggesting the molecule is associated with improving insulin sensitivity. Aspects of the invention disclosed herein provide methods and compositions that are useful for upregulating FNDC5 for the treatment and/or prevention of diseases associated with reduced FNDC5 expression or function such as Type 2 Diabetes. Aspects of the invention disclosed herein provide methods and compositions that are useful for upregulating GCK for the treatment and/or prevention of diseases associated with reduced GCK expression or function such as Type 2 Diabetes. Aspects of the invention disclosed herein provide methods and compositions that are useful for upregulating GLP1R for the treatment and/or prevention of diseases associated with reduced GLP1R expression or function such as Type 2 Diabetes. Aspects of the invention disclosed herein provide methods and compositions that are useful for upregulating SIRT1 for the treatment and/or prevention of diseases associated with reduced SIRT1 expression or function such as Type 2 Diabetes Aspects of the invention disclosed herein provide methods and compositions that are useful for upregulating ADIPOQ for the treatment and/or prevention of diseases associated with reduced ADIPOQ expression or function such as Type 2 Diabetes.

### Metabolic Disease—IGF1, SIRT1

Inborn errors of metabolism comprise a large class of genetic diseases involving disorders of metabolism. The majority are due to defects of single genes that code for enzymes that facilitate conversion of various substances (substrates) into others (products). In most of the disorders, problems arise due to accumulation of substances which are toxic or interfere with normal function, or to the effects of reduced ability to synthesize essential compounds. Inborn errors of metabolism are now often referred to as congenital metabolic diseases or inherited metabolic diseases. IGF-1, Insulin growth factor-1, is a hormone similar in molecular structure to insulin. IGF-1 plays an important role in childhood growth and continues to have anabolic effects in adults. Reduced IGF-1 and mutations in the IGF-1 gene are associated with metabolic disease SIRT1 (Sirtuin 1, also known as NAD-dependent deacetylase sirtuin-1) is an enzyme that deacetylates proteins that contribute to cellular regulation. SIRT1 has been shown to de-acetylate and affect the activity of both members of the PGC1-alpha/ERR-alpha complex, which are essential metabolic regulatory transcription factors. Aspects of the invention disclosed herein provide methods and compositions that are useful for upregulating IGF-1 for the treatment and/or prevention of diseases associated with reduced IGF-1 expression or function such as metabolic disease. Aspects of the invention disclosed herein provide methods and compositions that are useful for upregulating SIRT1 for the treatment and/or prevention of diseases associated with reduced SIRT1 expression or function such as metabolic disease.

### Aging/Senescence—SIRT1

Senescence is the state or process of aging Cellular senescence is a phenomenon where isolated cells demonstrate a limited ability to divide in culture, while organismal senescence is the aging of organisms. After a period of near perfect renewal (in humans, between 20 and 35 years of age), organismal senescence/aging is characterised by the declining ability to respond to stress, increasing homeostatic imbalance and increased risk of disease. This currently irreversible series of changes inevitably ends in death. SIRT1 (Sirtuin 1, also known as NAD-dependent deacetylase sirtuin-1) is an enzyme that deacetylates proteins that contribute to cellular regulation. Mice overexpressing SIRT1 present lower levels of DNA damage, decreased expression of the ageing-associated gene p16lnk4a, a better general health and fewer spontaneous carcinomas and sarcomas. Aspects of the invention disclosed herein provide methods and compositions that are useful for upregulating SIRT1 for the treatment and/or prevention of biological processes associated with reduced SIRT1 expression or function such as aging.

### Autoimmune—GRN, IDO1, CD274

Autoimmune diseases arise from an inappropriate immune response of the body against substances and tissues normally present in the body. In other words, the immune system mistakes some part of the body as a pathogen and attacks its own cells. Autoimmune diseases are classified by corresponding types of hypersensitivity: type II, type III, or type IV. Examples of autoimmune disease include, but are not limited to, Ankylosing Spondylitis, Autoimmune cardiomyopathy, Autoimmune hemolytic anemia, Autoimmune hepatitis, Autoimmune inner ear disease, immune lymphoproliferative syndrome, Autoimmune peripheral neuropathy, Autoimmune pancreatitis, Autoimmune polyendocrine syndrome, Autoimmune thrombocytopenic purpura, Celiac disease, Cold agglutinin disease, Contact dermatitis, Crohn's disease, Dermatomyositis, Diabetes mellitus type 1, Eosinophilic fasciitis, Gastrointestinal pemphigoid, Goodpasture's syndrome, Graves' disease, Guillain-Barré syndrome, Hashimoto's encephalopathy, Hashimoto's thyroiditis, Idiopathic thrombocytopenic purpura, Lupus erythematosus, Miller-Fisher syndrome, Myasthenia gravis, Pemphigus vulgaris, Pernicious anaemia, Polymyositis, Primary biliary cirrhosis, Psoriasis, Psoriatic arthritis, Relapsing polychondritis, Rheumatoid arthritis, Sjögren's syndrome, Temporal arteritis, Transverse myelitis, Ulcerative colitis, Undifferentiated connective tissue disease, Vasculitis, Vitiligo, and Wegener's granulomatosis. IDO1 encodes indoleamine 2,3-dioxygenase (IDO)—a heme enzyme that catalyzes the first and rate-limiting step in tryptophan catabolism to N-formyl-kynurenine. This enzyme acts on multiple tryptophan substrates including D-tryptophan, L-tryptophan, 5-hydroxy-tryptophan, tryptamine, and serotonin. This enzyme is thought to play a role in a variety of pathophysiological processes such as antimicrobial and antitumor defense, neuropathology, immunoregulation, and antioxidant activity. Increased catabolism of tryptophan by IDO1 suppresses T cell responses in a variety of diseases or states, including autoimmune disorders. GRN encodes a precursor protein called Progranulin, which is then cleaved to form the secreted protein granulin. Granulin regulates cell division, survival, motility and migration. Granulin has roles in cancer, inflammation, host defense, cartilage development and degeneration, and neurological functions. Downregulation of GRN has been shown to increase the onset of autoimmune diseases like rheumatoid arthritis. Aspects of the invention disclosed herein provide methods and compositions that are useful for upregulating IDO1 for the treatment and/or prevention of diseases associated with reduced IDO1 expression or function such as autoimmune diseases. Aspects of the invention disclosed herein provide methods and compositions that are useful for upregulating GRN for the treatment and/or prevention of diseases associated with reduced GRN expression or function such as autoimmune diseases.

CD274 (also known as PDL1) is a transmembrane protein containing IgV-like and IgC-like extracellular domains expressed on immune cells and non-hematopoietic cells, and is a ligand for the programmed death receptor (PD-1) expressed on lymphocytes and macrophages. PD-1 and CD274 interactions are essential in maintaining the balance of T-cell activation, tolerance, and immune-mediated tissue damage. CD274 is involved in inhibiting the initial phase of activation and expansion of self-reactive T cells, and restricting self-reactive T-cell effector function and target organ injury. More specifically, activation of PD-1 by CD274 inhibits T-cell proliferation, cytokine production, and cytolytic function by blocking the induction of phosphatidylinositol-3-kinase (PI3K) activity and downstream activation of Akt.

Decreased expression of CD274 results in autoimmunity in animal models. For example, mice deficient for the CD274 receptor, PD-1, developed features of late onset lupus. In another instance, blockade of CD274 activity in a mouse model of Type 1 diabetes resulted in accelerated progression of diabetes. In yet another example, CD274 blockade in an animal model of multiple sclerosis resulted in accelerated disease onset and progression.

Increasing expression of CD274 offers a novel approach for treating diseases related to inappropriate or undesirable activation of the immune system, including in the context of translation rejection, allergies, asthma and autoimmune disorders. Aspects of the invention disclosed herein provide methods and compositions that are useful for upregulating CD274 for the treatment and/or prevention of diseases associated with reduced CD274 expression or function such as autoimmune disease, transplant rejection, allergies or asthma.

### Inflammation (Chronic Inflammation)—GRN, IDO1, IL10

Inflammation is part of the complex biological response of vascular tissues to harmful stimuli, such as pathogens, damaged cells, or irritants. Inflammation is a protective attempt by the organism to remove the injurious stimuli and to initiate the healing process. However, chronic inflammation can also lead to a host of diseases, such as hay fever, periodontitis, atherosclerosis, and rheumatoid arthritis. Prolonged inflammation, known as chronic inflammation, leads to a progressive shift in the type of cells present at the site of inflammation and is characterized by simultaneous destruction and healing of the tissue from the inflammatory process. Inflammatory disorder include, but are not limited to, acne vulgaris, asthma, autoimmune diseases, celiac disease, chronic prostatitis, glomerulonephritis, inflammatory bowel diseases, pelvic inflammatory disease, reperfusion injury, rheumatoid arthritis, sarcoidosis, transplantation rejection (graft vs host disease), vasculitis and interstitial cystitis.

GRN encodes a precursor protein called Progranulin, which is then cleaved to form the secreted protein granulin. Granulin regulates cell division, survival, motility and migration. Granulin has roles in cancer, inflammation, host defense, cartilage development and degeneration, and neurological functions. GRN has been shown to alleviate inflammatory arthritis symptoms in mouse models. Indoleamine 2,3-dioxygenase 1 (IDO1 previously referred as IDO or INDO) is the main inducible and rate-limiting enzyme for the catabolism of the amino acid tryptophan through the kynurenine pathway. Increased catabolism of tryptophan by IDO1 suppresses T cell responses in a variety of diseases, such as allograft rejection.

Aspects of the invention disclosed herein provide methods and compositions that are useful for upregulating GRN for the treatment and/or prevention of diseases associated with reduced GRN expression or function such as chronic inflammation. Aspects of the invention disclosed herein provide methods and compositions that are useful for upregulating GRN for the treatment and/or prevention of diseases associated with reduced GRN expression or function such as rheumatoid arthritis. Aspects of the invention disclosed herein provide methods and compositions that are useful for upregulating IDO1 for the treatment and/or prevention of diseases associated with reduced IDO1 expression or function such as chronic inflammation. Aspects of the invention disclosed herein provide methods and compositions that are useful for upregulating IDO1 for the treatment and/or prevention of diseases associated with reduced IDO1 expression or function such as graft vs. host disease.

IL-10 is capable of inhibiting synthesis of pro-inflammatory cytokines such as IFN-γ, IL-2, IL-3, TNFα and GM-CSF made by cells such as macrophages and regulatory T-cells. It also displays a potent ability to suppress the antigen-presentation capacity of antigen presenting cells. Treatment with IL-10 (e.g. as a recombinant protein given to patients) is currently in clinical trials for Crohn's disease. Genetic variation in the IL-10 pathway modulates severity of acute graft-versus-host disease. Mouse models of arthritis have been shown to have decreased levels of IL-10. Aspects of the invention disclosed herein provide methods and compositions that are useful for upregulating GRN for the treatment and/or prevention of diseases associated with reduced GRN expression or function such as chronic inflammation.

Aspects of the invention disclosed herein provide methods and compositions that are useful for upregulating IL-10 for the treatment and/or prevention of diseases associated with reduced IL-10 expression or function such as chronic inflammation. Aspects of the invention disclosed herein provide methods and compositions that are useful for upregulating IL-10 for the treatment and/or prevention of diseases associated with reduced IL-10 expression or function such as rheumatoid arthritis. Aspects of the invention disclosed herein provide methods and compositions that are useful for upregulating IL-10 for the treatment and/or prevention of diseases associated with reduced IL-10 expression or function such as graft vs host disease. Aspects of the invention disclosed herein provide methods and compositions that are useful for upregulating IL-10 for the treatment and/or prevention of diseases associated with reduced IL-10 expression or function such as Crohn's disease.

### Infectious Disease—PTGS2

Infectious diseases, also known as transmissible diseases or communicable diseases comprise clinically evident illness (i.e., characteristic medical signs and/or symptoms of disease) resulting from the infection, presence and growth of pathogenic biological agents in an individual host organism. Infectious pathogens include some viruses, bacteria, fungi, protozoa, multicellular parasites, and aberrant proteins known as prions. A contagious disease is a subset of infectious disease that is especially infective or easily transmitted. Prostaglandin-endoperoxide synthase 2, also known as cyclooxygenase-2 or simply COX-2, is an enzyme that in humans is encoded by the PTGS2 gene. Prostaglandin endoperoxide H synthase, COX 2, converts arachidonic acid (AA) to prostaglandin endoperoxide H2. COX-2 is elevated during inflammation and infection. Aspects of the invention disclosed herein provide methods and compositions that are useful for upregulating PTGS2 for the treatment and/or prevention of diseases associated with reduced PTGS2 expression or function such as infectious disease.

### CNS Disease—IGF1, GRN

Central nervous system (CNS) disease can affect either the spinal cord (myelopathy) or brain (encephalopathy), both of which are part of the central nervous system CNS diseases include Encephalitis, Meningitis, Tropical spastic paraparesis, Arachnoid cysts, Amyotrophic lateral sclerosis, Huntington's disease, Alzheimer's disease, Dementia, Locked-in syndrome, Parkinson's disease, Tourette', and Multiple sclerosis. CNS diseases have a variety of causes including Trauma, Infections, Degeneration, Structural defects, Tumors, Autoimmune Disorders, and Stroke. Symptoms range from persistent headache, loss of feeling, memory loss, loss of muscle strength, tremors, seizures, slurred speech, and in some cases, death. IGF-1, Insulin growth factor-1, is a hormone similar in molecular structure to insulin. IGF-I deficiency is associated with neurodegenerative disease and has been shown to improve survival of neurons both in vitro and in vivo. Aspects of the invention disclosed herein provide methods and compositions that are useful for upregulating IGF1 for the treatment and/or prevention of diseases associated with reduced IGF1 expression or function such as CNS disease.

GRN encodes a precursor protein called Progranulin, which is then cleaved to form the secreted protein granulin. Granulin regulates cell division, survival, motility and migration. Granulin has roles in cancer, inflammation, host defense, cartilage development and degeneration, and neurological functions. Mutations in granulin are associated with dementia. Aspects of the invention disclosed herein provide methods and compositions that are useful for upregulating GRN for the treatment and/or prevention of diseases associated with reduced GRN expression or function such as CNS disease.

### Hemochromatosis—HAMP

Hemochromatosis is the abnormal accumulation of iron in parenchymal organs, leading to organ toxicity. This is the most common inherited liver disease in Caucasians and the most common autosomal recessive genetic disorder. HAMP (hepcidin antimicrobial peptide) encodes the protein hepcidin, which plays a major role in maintaining iron balance in the body. Hepcidin circulates in the blood and inhibits iron absorption by the small intestine when the body's supply of iron is too high. Hepcidin interacts primarily with other proteins in the intestines, liver, and certain white blood cells to adjust iron absorption and storage. At least eight mutations in the HAMP-gene have been identified that result in reduced levels of hepcidin and hemochromatosis. Aspects of the invention disclosed herein provide methods and compositions that are useful for upregulating HAMP for the treatment and/or prevention of diseases associated with reduced HAMP expression or function such as hemochromatosis.

### Acute Kidney Injury—SMAD7

Acute kidney injury (AKI), previously called acute renal failure (ARF), is a rapid loss of kidney function. Its causes are numerous and include low blood volume from any cause, exposure to substances harmful to the kidney, and obstruction of the urinary tract AKI may lead to a number of complications, including metabolic acidosis, high potassium levels, uremia, changes in body fluid balance, and effects to other organ systems. SMAD7 (Mothers against decapentaplegic homolog 7) is a protein that, as its name describes, is a homolog of the Drosophila gene: “Mothers against decapentaplegic”. It belongs to the SMAD family of proteins, which belong to the TGFβ superfamily of ligands. Like many other TGFβ family members, SMAD7 is involved in cell signalling. It is a TGFβ type 1 receptor antagonist. It blocks TGFβ1 and activin associated with the receptor, blocking access to SMAD2. It is an inhibitory SMAD (I-SMAD) and is enhanced by SMURF2. Upon TGF-β treatment, SMAD7 binds to discrete regions of Pellino-1 via distinct regions of the SMAD MH2 domains. The interaction block formation of the IRAK1-mediated IL-1R/TLR signaling complex therefore abrogates NF-κB activity, which subsequently causes reduced expression of pro-inflammatory genes. Overexpression of SMAD7 in the kidney using gene therapy inhibited renal fibrosis and inflammatory pathways. Aspects of the invention disclosed herein provide methods and compositions that are useful for upregulating SMAD7 for the treatment and/or prevention of diseases associated with reduced SMAD7 expression or function such as acute kidney injury.

### Thalassemia—HAMP

Thalassemia is a group of inherited autosomal recessive blood disorders, resulting in a reduced rate of synthesis or no synthesis of one of the globin chains that make up hemoglobin. This can cause the formation of abnormal hemoglobin molecules or reduced numbers of hemoglobin, thus causing anemia, the characteristic presenting symptom of the thalassemias. HAMP (hepcidin antimicrobial peptide) encodes the protein hepcidin, which plays a major role in maintaining iron balance in the body. Hepcidin circulates in the blood and inhibits iron absorption by the small intestine when the body's supply of iron is too high. HAMP expression has been shown to be lower in patients with thalassemia and is associated with iron-overload (sometimes called hemochromatosis) in these patients. Aspects of the invention disclosed herein provide methods and compositions that are useful for upregulating HAMP for the treatment and/or prevention of diseases associated with reduced HAMP expression or function such as thalassemia.

### Lesch-Nyhan Disease—HPRT1

Lesch-Nyhan syndrome (LNS), also known as Nyhan's syndrome, Kelley-Seegmiller syndrome and Juvenile gout, is a rare inherited disorder caused by a deficiency of the enzyme hypoxanthine-guanine phosphoribosyltransferase (HGPRT), produced by mutations in the HPRT gene located on the X chromosome. LNS affects about one in 380,000 live births. The HGPRT deficiency causes a build-up of uric acid in all body fluids. This results in both hyperuricemia and hyperuricosuria, associated with severe gout and kidney problems. Neurological signs include poor muscle control and moderate mental retardation. Aspects of the invention disclosed herein provide methods and compositions that are useful for upregulating HPRT for the treatment and/or prevention of diseases associated with reduced HPRT expression or function such as Lesch-Nyhan syndrome.

### Delayed Growth—IGF-1

Delayed growth is poor or abnormally slow height or weight gains in a child typically younger than age 5, IGF-1, Insulin growth factor-1, is a hormone similar in molecular structure to insulin. IGF-1 plays an important role in childhood growth and continues to have anabolic effects in adults. IGF1 deficiency has been shown to be associated with delayed growth and short stature in humans. Aspects of the invention disclosed herein provide methods and compositions that are useful for upregulating IGF1 for the treatment and/or prevention of diseases associated with reduced IGF1 expression or function such as delayed growth.

### Dyslipidemias and Atherosclerosis—LDLR

Accumulation of lipids in the blood can cause a variety of conditions and diseases, e.g. dyslipidemia and atherosclerosis. Atherosclerosis in particular is the leading cause of death in industrialized societies, making prevention and treatment a high public health concern. Low-density lipoprotein (LDL) is a major transporter of fat molecules, e.g., cholesterol, in the blood stream that delivers fat molecules to cells. High-density lipoprotein (HDL) is another transporter of fat molecules that moves lipids, e.g. cholesterol, from cells to the liver. High levels of LDL are associated with health problems such as dyslipidemia and atherosclerosis, while HDL is protective against atherosclerosis and is involved in maintenance of cholesterol homeostasis.

Dyslipidemia generally describes a condition when an abnormal amount of lipids is present in the blood. Hyperlipidemia, which accounts for the majority of dyslipidemias, refers to an abnormally high amount of lipids in the blood. Hyperlipidemia is often associated with hormonal diseases such as diabetes, hypothyroidism, metabolic syndrome, and Cushing syndrome. Examples of common lipids in dyslipidemias include triglycerides, cholesterol and fat. Abnormal amounts lipids or lipoproteins in the blood can lead to atherosclerosis, heart disease, and stroke.

Atherosclerosis diseases, e.g. coronary artery disease (CAD) and myocardial infarction (MI), involve a thickening of artery walls caused by accumulation of fat in the blood, most commonly cholesterol. This thickening is thought to be the result of chronic inflammation of arteriole walls due to accumulation of LDLs in the vessel walls. LDL molecules can become oxidized once inside vessel walls, resulting in cell damage and recruitment of immune cells like macrophages to absorb the oxidized LDL. Once macrophages internalize oxidized LDL, they become saturated with cholesterol and are referred to as foam cells Smooth muscle cells are then recruited and form a fibrous region. These processes eventually lead to formation of plaques that block arteries and can cause heart attack and stroke. HDL is capable of transporting cholesterol from foam cells to the liver, which aids in inhibition of inflammation and plaque formation.

The LDLR gene encodes the Low-Density Lipoprotein (LDL) Receptor, which is a mosaic protein of about 840 amino acids (after removal of signal peptide) that mediates the endocytosis of cholesterol-rich LDL. It is a cell-surface receptor that recognizes the apoprotein B 100 which is embedded in the phospholipid outer layer of LDL particles. LDL receptor complexes are present in clathrin-coated pits (or buds) on the cell surface, which when bound to LDL-cholesterol via adaptin, are pinched off to form clathrin-coated vesicles inside the cell. This allows LDL-cholesterol to be bound and internalized in a process known as endocytosis. This occurs in all nucleated cells (not erythrocytes), but mainly in the liver which removes about 70% of LDL from the circulation. Aspects of the invention disclosed herein provide methods and compositions that are useful for upregulating LDLR for the treatment and/or prevention of diseases associated with reduced LDLR expression or function such as dyslipidemia or atherosclerosis.

### Tissue Regeneration—NANOG

Regeneration is the process of renewal, restoration, and growth of cells and organs in response to disturbance or damage. Strategies for regeneration of tissue include the rearrangement of pre-existing tissue, the use of adult somatic stem cells and the dedifferentiation and/or transdifferentiation of cells, and more than one mode can operate in different tissues of the same animal. During the developmental process, genes are activated that serve to modify the properties of cells as they differentiate into different tissues. Development and regeneration involves the coordination and organization of populations cells into a blastema, which is a mound of stem cells from which regeneration begins. Dedifferentiation of cells means that they lose their tissue-specific characteristics as tissues remodel during the regeneration process. Transdifferentiation of cells occurs when they lose their tissue-specific characteristics during the regeneration process, and then re-differentiate to a different kind of cell. These strategies result in the re-establishment of appropriate tissue polarity, structure and form. NANOG is a transcription factor critically involved with self-renewal of undifferentiated embryonic stem cells through maintenance of pluripotency. Aspects of the invention disclosed herein provide methods and compositions that are useful for upregulating NANOG for tissue regeneration.

### Oxidative Stress/Antioxidative Pathway—SIRT6

Cells are protected against oxidative stress by an interacting network of antioxidant enzymes. Oxidation reactions can produce superoxides or free radicals. In turn, these radicals can start chain reactions. When the chain reaction occurs in a cell, it can cause damage or death to the cell. Antioxidants terminate these chain reactions by removing free radical intermediates, and inhibit other oxidation reactions. The superoxide released by processes such as oxidative phosphorylation is first converted to hydrogen peroxide and then further reduced to give water. This detoxification pathway is the result of multiple enzymes, with superoxide dismutases catalysing the first step and then catalases and various peroxidases removing hydrogen peroxide. As oxidative stress appears to be an important part of many human diseases, the use of antioxidants in pharmacology is highly attractive. Mono-ADP-ribosyltransferase sirtuin-6 is an enzyme that in humans is encoded by the SIRT6 gene. Sirtuin-6 has been shown to have a protective role against metabolic damage caused by a high fat diet. SIRT6 deficiency is associated with metabolic defects that lead to oxidative stress. Aspects of the invention disclosed herein provide methods and compositions that are useful for upregulating SIRT6 for tissue regeneration. Aspects of the invention disclosed herein provide methods and compositions that are useful for upregulating SIRT6 for the treatment and/or prevention of diseases associated with reduced SIRT6 expression or function such as oxidative stress.

### Choroidal Neovascularization—SERPINF1

Choroidal neovascularization (CNV) is the creation of new blood vessels in the choroid layer of the eye. This is a common symptom of the degenerative maculopathy wet AMD (age-related macular degeneration). Serpin F1 (SERPINF1), also known as Pigment epithelium-derived factor (PEDF), is a multifunctional secreted protein that has anti-angiogenic, anti-tumorigenic, and neurotrophic functions. The anti-angiogenic properties of SERPINF1 allow it to block new blood vessel formation. Aspects of the invention disclosed herein provide methods and compositions that are useful for upregulating SERPINF1 for the treatment and/or prevention of diseases associated with reduced SERPINF1 expression or function such as Choroidal neovascularization.

### Cardiovascular Disease—SERPINF1

Cardiovascular disease is a class of diseases that involve the heart or blood vessels (arteries and veins). Cardiovascular diseases remain the biggest cause of deaths worldwide. Types of cardiovascular disease include, Coronary heart disease, Cardiomyopathy, Hypertensive heart disease, Heart failure, Corpulmonale, Cardiac dysrhythmias, Inflammatory heart disease, Valvular heart disease, Stroke and Peripheral arterial disease. Serpin F1 (SERPINF1), also known as Pigment epithelium-derived factor (PEDF), is a multifunctional secreted protein that has anti-angiogenic, anti-tumorigenic, and neurotrophic functions. SERPINF1 has been shown to have a protective role in atherosclerosis, the main cause of coronary heart disease, myocardial infarction and heart failure due to its anti-inflammatory, antioxidant and antithrombotic effects in the vessel wall and platelets. Additionally SERPINF1 has strong antiangiogenic effects by inducing apoptosis in endothelial cells and by regulating the expression of other angiogenic factors. Aspects of the invention disclosed herein provide methods and compositions that are useful for upregulating SERPINF1 for the treatment and/or prevention of diseases associated with reduced SERPINF1 expression or function such as cardiovascular disease.

### Hyperimmunoglobulin E Syndrome—STAT3

Loss-of-function mutations in the STAT3 gene result in Hyperimmunoglobulin E syndrome, associated with recurrent infections as well as disordered bone and tooth development.

### Leber's Congenital Amaurosis (LCA), Bardet-Biedl Syndrome (BBS), Joubert Syndrome, Meckel Syndrome, Sior-Loken Syndrome—CEP290

Leber's congenital amaurosis (LCA) is a rare autosomal recessive eye disease resulting in a severe form of retinal dystrophy that is present from birth. LCA results in slow or non-existent pupillary responses, involuntary eye movement, and severe loss of vision. LCA is thought to be caused by abnormal photoreceptor cell development or degeneration. Bardet-Biedl syndrome (BBS) is characterized by retinal dystrophy and retinitis pigmentosa. Other manifestations include polydactyly and renal abnormalities. Both LCA and BBS are associated with mutations in Centrosomal protein 290 kDA (CEP290).

CEP290 is a large coiled-coil protein found in the centrosome and cilia of cells. CEP290 modulates ciliary formation and is involved in trafficking ciliary proteins between the cell body and the cilium of a cell. Reduction or abolishment of CEP290 activity, results in retinal and photoreceptor degeneration. This generation is thought to be the result of defects in ciliogenesis. CEP290 is also associated with Joubert syndrome, Meckel syndrome, and Sior-Loken syndrome. Aspects of the invention disclosed herein provide methods and compositions that are useful for upregulating CEP290 for the treatment and/or prevention of diseases associated with reduced CEP290 expression or function such as Leber's congenital amaurosis (LCA), Bardet-Biedl syndrome (BBS), Joubert syndrome, Meckel syndrome, Sior-Loken syndrome.

### Phenylketonuria—PAH

Phenylketonuria (PKU) is an autosomal recessive metabolic disease caused by elevated levels of Phenyalanine (Phe) in the blood. Phe is a large neutral amino acid (LNAA) that interacts with the LNAA transporter in order to cross the blood-brain barrier. When Phe is in excess in the blood, it saturates the LNAA transporter, prevent other essential LNAAs from crossing the blood-brain barrier. This results in depletion of these amino acids in the brain, leading to slowing of the development of the brain and mental retardation. PKU can be managed by strictly controlling and monitoring Phe levels in the diet in infants and children. However, if left untreated, severe mental retardation, irregular motor functions, and behavioral disorders result from Phe accumulation in the blood.

The accumulation in the blood is the result of mutations in the Phenylalanine hydroxylase (PAH) gene, which encodes phenylalanine hydroxylase protein. Phenylalanine hydroxylase is an enzyme that generates tyrosine through hydroxylation of the aromatic side-chain of Phe. Phenylalanine hydroxylase is the rate-limiting enzyme in the degradation of excess Phe. When phenylalanine hydroxylase levels are decreased or enzyme functionality is compromised, Phe begins to accumulate in the blood, resulting in PKU. Aspects of the invention disclosed herein provide methods and compositions that are useful for upregulating PAH for the treatment and/or prevention of diseases associated with reduced PAH expression or function such as PKU.

### Congenital Bilateral Absence of Vas Deferens (CBAVD) and Cystic Fibrosis (CF)—CFTR

CFTR is a cyclic-AMP activated ATP-gated anion channel that transports ions across cell membranes. CFTR is predominantly found in epithelial cells in the lung, liver, pancreas, digestive tract, reproductive tract, and skin. A main function of CFTR is to move chloride and thiocyanate ions out of epithelial cells. In order to maintain electrical balance, sodium ions move with the chloride and thiocyanate ions, resulting in an increase of electrolytes outside of the cell. This increase results in movement of water out of the cell by osmosis, creating bodily fluids such as mucus, sweat, and digestive juices, depending on the organ. When CFTR activity is reduced or abolished, ion transport is affected, resulting in reduced water movement out of cells and abnormally viscous bodily fluids (e.g. sticky and viscous mucus, sweat, or digestives juices).

Mutations in CFTR are associated with congenital bilateral absence of vas deferens (CBAVD) and cystic fibrosis. Males with congenital bilateral absence of the vas deferens often have mutations that result in reduced CFTR activity. As a result of these mutations, the movement of water and salt into and out of cells is disrupted. This disturbance leads to the production of a large amount of thick mucus that blocks the developing vas deferens (a tube that carries sperm from the testes) and causes it to degenerate, resulting in infertility.

Cystic fibrosis (CF) is an autosomal recessive disease characterized by overly viscous secretions in the lungs, pancreas, liver, and intestine. In the lungs, difficulty breathing and frequent infection are common results of mucus build-up Viscous secretions in the pancreas lead to scarring, fibrosis, and cyst formation which can subsequently lead to diabetes. Additionally, absorption of nutrients in the intestine is decreased due to a lack of digestive enzymes provided by the pancreas. Blockage of the intestine is also common due to thickening of the feces. Aspects of the invention disclosed herein provide methods and compositions that are useful for upregulating CFTR for the treatment and/or prevention of diseases associated with reduced CFTR expression or function such CBAVD or CF.

### Exemplary Nucleotide Analogs

Each strand of the ds mRNA molecule can independently include one or more nucleotide analogs, e.g., having modifications to the base, e.g., nucleobases including but not limited to 1,5-dimethyluracil, 1-methyluracil, 2-amino-6-hydroxyaminopurine, 2-aminopurine, 3-methyluracil, 5-(hydroxymethyl)cytosine, 5-bromouracil, 5-carboxycytosine, 5-fluoroorotic acid, 5-fluorouracil, 5-formylcytosine, 8-azaadenine, 8-azaguanine, N⁶-hydroxyadenine, allopurinol, hypoxanthine, orthiouracil, modifications of the sugar group or modifications of the phosphate group. In one embodiment, at least one strand of the ds mRNA molecule includes, but is not limited to, 1-methyladenosine, 2-methylthio-N⁶-hydroxynorvalyl carbamoyladenosine, 2-methyladenosine, 2-O-ribosylphosphate adenosine, N⁶-methyl-N⁶-threonylcarbamoyladenosine, N⁶-acetyladenosine, N⁶-glycinylcarbamoyladenosine, N⁶-isopentenyladenosine, N⁶-methyladenosine, N⁶-threonylcarbamoyladenosine, N⁶, N⁶-dimethyladenosine, N N⁶-(cis-hydroxyisopentenyl)adenosine, N⁶-hydroxynorvalylcarbamoyladenosine, 1,2-O-dimethyladenosine, N⁶,2-O-dimethyladenosine, 2-O-methyladenosine, N⁶, N⁶,O-2-trimethyladenosine, 2-methylthio-N⁶-(cis-hydroxyisopentenyl) adenosine, 2-methylthio-N-methyladenosine, 2-methylthio-N⁶-isopentenyladenosine, 2-methylthio-N⁶-threonyl carbamoyladenosine, 2-thiocytidine, 3-methylcytidine, N⁴-acetylcytidine, 5-formylcytidine, N⁴-methylcytidine, 5-methylcytidine, 5-hydroxymethylcytidine, lysidine, N⁴-acetyl-2-O-methylcytidine, 5-formyl-2-O-methylcytidine, 5,2-O-dimethylcytidine, 2-O-methylcytidine, N⁴,2-O-dimethylcytidine, N⁴, N⁴,2-O-trimethylcytidine, 1-methylguanosine, N²,7-dimethylguanosine, N²-methylguanosine, 2-O-ribosylphosphate guanosine, 7-methylguanosine, under modified hydroxywybutosine, 7-aminomethyl-7-deazaguanosine, 7-cyano-7-deazaguanosine, N², N²-dimethylguanosine, 4-demethylwyosine, epoxyqueuosine, hydroxywybutosine, isowyosine, N²,7,2-O-trimethylguanosine, N²,2-O-dimethylguanosine, 1,2-O-dimethylguanosine, 2-O-methylguanosine, N² N²2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine and 2-O-methylinosine, or any combination thereof.

In one embodiment, at least one strand of the ds mRNA molecule includes, but is not limited to, cytosine arabinoside or fludarabine. In one embodiment, at least one strand of the ds mRNA molecule includes, but is not limited to, cladribine, acyclovir, 2′,3′-dideoxyinosine; 9-β-D-ribofuranosyladenine; .beta.-arabinofuranosylcytosine; arabinosylcytosine; 4-amino-5-fluoro-1-[(2R,5S)-2-(hydroxymethyl)-1,3-oxathiolan-5-yl]-1,2-di-hydropyrimidin-2-one; 2,3′-dideoxy-3′-thiacytidine; 2′-3′-dideoxycytidine; {(1S,4R)-4-[2-amino-6-(cyclopropylamino)-9H-purin-9-yl]cyclopent-2-en-1-y-l}methanol; 2-Amino-9-[(1S,3R,4S)-4-hydroxy-3-(hydroxymethyl)-2-methylidenecyclopenty-l]-6,9-dihydro-3H-purin-6-one; 2′-3′-didehydro-2′-3′-dideoxythymidine; 1-(2-deoxy-.beta.-L-erythro-pentofuranosyl)-5-methylpyrimidine-2,4(1H,3H)-dione; 1-[(2R,4S,5S)-4-azido-5-(hydroxymethyl)oxolan-2-yl]-5-methylpyrimi-dine-2,4-dione; 1-[(2R,4S,5R)-4-hydroxy-5-(hydroxymethyl)oxolan-2-yl]-5-iodo-1,2,3,4-tetr-ahydropyrimidine-2,4-dione; 1-[4-hydroxy-5-(hydroxymethyl)oxolan-2-yl]-5-(trifluoromethyl) pyrimidine-2,4-dione; 5-Fluoro-2′-deoxycytidine; 5-Fluorodeoxycytidine; Floxuridine (5-Fluoro-1-[4-hydroxy-5-(hydroxymethyl)tetrahydrofuran-2-yl]-1H-pyrimidi-ne-2,4-dione); 4-amino-1-(2-deoxy-2,2-difluoro-β-D-erythro-pentofuranosyl)pyrimidin-2(1H)-one; or 2′,2′-difluoro-2′-deoxycytidine; (8R)-3-(2-deoxy-β-D-erythro-pentofuranosyl)-3,4,7,8-tetrahydroimidaz-o[4,5-d][1,3]diazepin-8-ol, or any combination thereof.

In one embodiment, a strand of the ds mRNA may include analogs such as 2′-O-methyl-substituted RNA, locked nucleic acid (LNA) or BNA (Bridged Nucleic Acid), morpholino, or peptide nucleic acid (PNA), or any combination thereof.

In one embodiment, nucleotide analogs include phosphorothioate nucleotides or deazapurine nucleotides and other nucleotide analogs.

In one embodiment, one or more strands of the ds mRNA molecule can independently include a modified nucleotide selected from a deoxyribonucleotide, a dideoxyribonucleotide, an acyclonucleotide, a 3′-deoxyadenosine (cordycepin), a 3′-azido-3′-deoxythymidine (AZT), a 2′,3′-dideoxyinosine (ddl), a 2′,3′-dideoxy-3′-thiacytidine (3TC), a 2′,3′-didehydro-2′,3′-dideoxythymidine (d4T), a monophosphate nucleotide of 3′-azido-3′-deoxythymidine (AZT), a 2′,3′-dideoxy-3′-thiacytidine (3TC) and a monophosphate nucleotide of 2′,3′-didehydro-2′,3′-dideoxythymidine (d4T), a 4-thiouracil, a 5-bromouracil, a 5-iodouracil, a 5-(3-aminoallyl)-uracil, a 2′-O-alkyl ribonucleotide, a 2′-O-methyl ribonucleotide, a 2′-amino ribonucleotide, a 2′-fluoro ribonucleotide, or a locked nucleic acid; or any combination thereof.

In one embodiment, the nucleotide modification includes 2′ modifications, e.g., 2′ F on pyrimidines or 2′ H or 2′ OMe on purines.

In one embodiment, the nucleotide modification includes a phosphate backbone modification selected from a phosphonate, a phosphorothioate, a phosphotriester; a morpholino nucleic acid; or a peptide nucleic acid (PNA).

Sugar modifications in the strand(s) include, but are not limited to, replacing the heteroatoms at the 2′ and 3′ carbons with hydrogen, another heteroatom or an alkyl group; replacing the H's at the 2′ carbon with a heteroatom or alkyl group; replacing the 2′ and 3′ carbons with a heteroatom, most commonly S or O; removing the 2′ and/or 3′ carbons to generate acyclic sugars; replacing the 4′-OH with N, S, or an alkyl group; adding alkyl groups to the 4′-carbon; replacing the 5′-hydroxyl with N or a phosphonate, or interconversion of both the sugar stereochemistry (D vs. L) and anomeric configuration (α vs. β).

Stability of ds mRNA

A codon-optimized firefly luciferase gene with 5′ and 3′ human beta globin untranslated regions (UTRs) was installed onto the pcDNA3.1 plasmid. The firefly luciferase gene was transcribed by in vitro transcription. A 5′ 7-methyl guanosine cap and 3′ poly-A tail was added by enzymatic synthesis. The 5′ m⁷G cap, 3′ poly-A tail, both UTRs, and codon optimization have been shown to dramatically increased luciferase expression in vivo.

Double stranded mRNA was produced by constructing a plasmid with two T7 promoters in reverse orientations, both flanking the codon-optimized luciferase gene. Sense and antisense strands were produced in separate reactions by cutting the plasmid in different positions. The sense strand was capped with 7-methyl guanosine and poly-A tailed. The sense and antisense strands were annealed by heating to 65° C. with slow cooling Uridine was replaced with pseudouridine to reduce the immune response.

The relative stability of ss mRNA and ds mRNA when challenged by digestion with RNase A was compared. ss mRNA and ds mRNA were incubated with increasing amounts of RNase A for 10 minutes at 37° C. and products were immediately separated on an agarose gel. The relatively stability of ds mRNA versus ss mRNA approaches infinity when both are digested with 10 μg of RNAse A.

Serum nucleases degrade RNA. The relative stability of ds mRNA versus ss mRNA was compared when digested with increasing amounts of mouse serum. ss mRNA and ds mRNA were incubated with 0.0008% to 8% vol/vol ratio of mouse serum for 10 minutes at 37° C. then analyzed on an agarose gel. ds mRNA is shown to be highly stable compared to ss mRNA. The relative increase in stability approaches infinity by comparing ss mRNA and ds mRNA digested with 0.8% serum.

The relative translation of ss mRNA and ds mRNA into protein was compared by administering a 1 μg dose of each into mice via the tail vein by direct hydrodynamic injection. The expression of luciferase in the liver was determined at times ranging from 4 to 72 hours by serially measuring the light produced from the liver by in vivo bioluminescence imaging following i.p. dosing of luciferin. The level of luciferase expression for both ss mRNA and ds mRNA peaked at 4 hours and was maintained for 24 hours before declining over 48 and 72 hours. The results demonstrate that ds mRNA and ss mRNA produce equivalent expression of luciferase at times ranging from 4-72 hours.

RNA transcripts (sense strand) may be “tailed” with polyA sequences after being transcribed from the vector or the vector can include sequences that result in polyA tails on transcripts obtained from the vector. Each reverse mRNA was hybridized with forward mRNA to form ds mRNA. The resulting ds mRNAs were then combined with PEG-peptide and a 1 μg dose was administered via the tail vein of triplicate mice. At five minutes post administration, mice were administered a hydrodynamic dose of 1.9 mL of saline in 5 seconds via the tail vein. After 24 hours the mice were dosed i.p. with luciferin and the level of luciferase in liver was determined by quantitative bioluminescence imaging on an IVIS image. The results established that extending the length of the reverse mRNA relative to Xba1 had a negligible result on the level of gene expression. Similar, decreasing the length to fully expose the 5 UTR did not significantly influence the level of gene expression.

Chemically modified reverse mRNA was biosynthesized using 5′ amino allyl modified uridine or cytidine to replace each U or C, and both U and C, to incorporate multiple primary amines in the reverse mRNA strand. Incorporation of 5′aminoallyl uridine and/or cytidine during in vitro transcription is well/tolerated, resulting in full-length (aa-U Rev-, aa-C Rev- or aa-U/C Rev-) RNA with approximately 450 or 900 amines. Reverse strand primary amines May be used as a chemical handle for functionalization with acetyl, maleic acid, succinic acid, thiol-acetate, and PEG. Primary amines were then fully functionalized using anhydrides and N-hydroxysuccinamide esters to generate chemically functionalized reverse mRNA.

Hybridization of chemically functionalized reverse mRNA with forward mRNA resulted in chemically modified ds mRNA. Biological testing of chemically modified ds mRNA included testing for increased metabolic stability and functional translation to express luciferase in vivo.

5′ amino allyl uridine and cytidine modified ds mRNA demonstrated increased RNAse resistance relative to unmodified ds mRNA. However, 5′ amino allyl modified ds mRNA was inactive when tested for translation into luciferase. Alternatively, chemical modification of reverse mRNA with the amino reactive agents in FIG. 9 resulted in ds mRNAs that were partially translationally active in expressing luciferase. The greatest translational activity resulted from modification of 5-aminoallyl uridine with acetyl.

The magnitude of luciferase expression in liver was compared following hydrodynamic dosing of 1 μg of chemically modified ds mRNA into the tail vein of mice. Fully acetylated 100% 5′ amino allyl modified reverse mRNA resulted in a 10-fold decrease of expression relative to control Substitution of 10-50% of reverse mRNA uridine with 5′ amino allyl uridine followed by acetylation resulted in gene expression that was indistinguishable from control.

The results establish that chemical functionalization of ds mRNA can produce translationally active ds mRNA. These or further modifications may produce translationally active ds mRNA with increased metabolic stability.

Exemplary Ds mRNA Vaccines for Use with a Delivery Vehicle

Double stranded mRNA may be produced by constructing a plasmid with two T7 promoters in reverse orientations, both flanking a gene of interest, e.g., one useful for applications including but not limited to cancer immunotherapy, such as Melan-A, tyrosinase, gp100, MAGE-A1, MAGE-A3 or survivin, infectious disease, e.g, a viral or bacterial protein, protein replacement or augmentation, e.g., EPO, IL-10, VEGF-A, surface B protein or Foxp3, somatic reprogramming, or genome editing. Sense and antisense strands may be produced in separate reactions by cutting the plasmid in different positions. The sequences may be codon optimized, e.g., to improve translation or to decrease endonuclease activity, for instance, one or more uridine residues may be replaced with pseudouridine to reduce the immune response, or natural residues may be replaced with other analogs such as 2-thiouridine, 5-methyluridine, 5-methylcytidine or N6-methyl adenosine, or any combination thereof. The sense strand may be capped with 7-methyl guanosine or with cap analogs, and poly-A tailed. The sense and antisense strands are annealed by heating to 65° C. with slow cooling.

For example, for cancer immunotherapy, a double stranded mRNA having a sense strand that encodes a mammalian melanoma antigen recognized by T-cells (MART-1), e.g., where the sense strand has nucleic acid sequences with at least 90%, 92%, 95%, 97%, 98%, 99% or 100% nucleic acid sequence identity to coding sequences in SEQ ID NO:1 or a nucleic acid sequence that encodes a protein with at least 80% amino acid sequence identity to a protein encoded by SEQ ID NO:1; a double stranded mRNA having a sense strand that encodes a mammalian tyrosinase, e.g., where the sense strand has nucleic acid sequences with at least 90%, 92%, 95%, 97%, 98%, 99% or 100% nucleic acid sequence identity to coding sequences in SEQ ID NO:2 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:2; a double stranded mRNA having a sense strand that encodes a mammalian melanoma antigen, e.g., where the sense strand has nucleic acid sequences with at least 90%, 92%, 95%, 97%, 98%, 99% or 100% nucleic acid sequence identity to coding sequences in SEQ ID NO:3 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:3; or a double stranded mRNA having a sense strand that encodes a mammalian survivin, e.g., where the sense strand has nucleic acid sequences with at least 90%, 92%, 95%, 97%, 98%, 99% or 100% nucleic acid sequence identity to coding sequences in SEQ ID NO:4 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:4, may be employed.

(SEQ ID NO: 1)
AGCAGACAGAGGACTCTCATTAAGGAAGGTGTCCTGTGCCCTGACCCTACAAGATGCCAA
GAGAAGATGCTCACTTCATCTATGGTTACCCCAAGAAGGGGCACGGCCACTCTTACACCA
CGGCTGAAGAGGCCGCTGGGATCGGCATCCTGACAGTGATCCTGGGAGTCTTACTGCTCA
TCGGCTGTTGGTATTGTAGAAGACGAAATGGATACAGAGCCTTGATGGATAAAAGTCTTC
ATGTTGGCACTCAATGTGCCTTAACAAGAAGATGCCCACAAGAAGGGTTTGATCATCGGG
ACAGCAAAGTGTCTCTTCAAGAGAAAAACTGTGAACCTGTGGTTCCCAATGCTCCACCTG
CTTATGAGAAACTCTCTGCAGAACAGTCACCACCACCTTATTCACCTTAAGAGCCAGCGA
GACACCTGAGACATGCTGAAATTATTTCTCTCACACTTTTGCTTGAATTTAATACAGACA
TCTAATGTTCTCCTTTGGAATGGTGTAGGAAAAATGCAAGCCATCTCTAATAATAAGTCA
GTGTTAAAATTTTAGTAGGTCCGCTAGCAGTACTAATCATGTGAGGAAATGATGAGAAAT
ATTAAATTGGGAAAACTCCATCAATAAATGTTGCAATGCATGATACTATCTGTGCCAGAG
GTAATGTTAGTAAATCCATGGTGTTATTTTCTGAGAGACAGAATTCAAGTGGGTATTCTG
GGGCCATCCAATTTCTCTTTACTTGAAATTTGGCTAATAACAAACTAGTCAGGTTTTCGA
ACCTTGACCGACATGAACTGTACACAGAATTGTTCCAGTACTATGGAGTGCTCACAAAGG
ATACTTTTACAGGTTAAGACAAAGGGTTGACTGGCCTATTTATCTGATCAAGAACATGTC
AGCAATGTCTCTTTGTGCTCTAAAATTCTATTATACTACAATAATATATTGTAAAGATCC
TATAGCTCTTTTTTTTTGAGATGGAGTTTCGCTTTTGTTGCCCAGGCTGGAGTGCAATGG
CGCGATCTTGGCTCACCATAACCTCCGCCTCCCAGGTTCAAGCAATTCTCCTGCCTTAGC
CTCCTGAGTAGCTGGGATTACAGGCGTGCGCCACTATGCCTGACTAATTTTGTAGTTTTA
GTAGAGACGGGGTTTCTCCATGTTGGTCAGGCTGGTCTCAAACTCCTGACCTCAGGTGAT
CTGCCCGCCTCAGCCTCCCAAAGTGCTGGAATTACAGGCGTGAGCCACCACGCCTGGCTG
GATCCTATATCTTAGGTAAGACATATAACGCAGTCTAATTACATTTCACTTCAAGGCTCA
ATGCTATTCTAACTAATGACAAGTATTTTCTACTAAACCAGAAATTGGTAGAAGGATTTA
AATAAGTAAAAGCTACTATGTACTGCCTTAGTGCTGATGCCTGTGTACTGCCTTAAATGT
ACCTATGGCAATTTAGCTCTCTTGGGTTCCCAAATCCCTCTCACAAGAATGTGCAGAAGA
AATCATAAAGGATCAGAGATTCTG
(SEQ ID NO: 2)
TATTGAGTTCTTCAAACATTGTAGCCTCTTTATGGTCTCTGAGAAATAACTACCTTAAAC
CCATAATCTTTAATACTTCCTAAACTTTCTTAATAAGAGAAGCTCTATTCCTGACACTAC
CTCTCATTTGCAAGGTCAAATCATCATTAGTTTTGTAGTCTATTAACTGGGTTTGCTTAG
GTCAGGCATTATTATTACTAACCTTATTGTTAATATTCTAACCATAAGAATTAAACTATT
AATGGTGAATAGAGTTTTTCACTTTAACATAGGCCTATCCCACTGGTGGGATACGAGCCA
ATTCGAAAGAAAAGTCAGTCATGTGCTTTTCAGAGGATGAAAGCTTAAGATAAAGACTAA
AAGTGTTTGATGCTGGAGGTGGGAGTGGTATTATATAGGTCTCAGCCAAGACATGTGATA
ATCACTGTAGTAGTAGCTGGAAAGAGAAATCTGTGACTCCAATTAGCCAGTTCCTGCAGA
CCTTGTGAGGACTAGAGGAAGAATGCTCCTGGCTGTTTTGTACTGCCTGCTGTGGAGTTT
CCAGACCTCCGCTGGCCATTTCCCTAGAGCCTGTGTCTCCTCTAAGAACCTGATGGAGAA
GGAATGCTGTCCACCGTGGAGCGGGGACAGGAGTCCCTGTGGCCAGCTTTCAGGCAGAGG
TTCCTGTCAGAATATCCTTCTGTCCAATGCACCACTTGGGCCTCAATTTCCCTTCACAGG
GGTGGATGACCGGGAGTCGTGGCCTTCCGTCTTTTATAATAGGACCTGCCAGTGCTCTGG
CAACTTCATGGGATTCAACTGTGGAAACTGCAAGTTTGGCTTTTGGGGACCAAACTGCAC
AGAGAGACGACTCTTGGTGAGAAGAAACATCTTCGATTTGAGTGCCCCAGAGAAGGACAA
ATTTTTTGCCTACCTCACTTTAGCAAAGCATACCATCAGCTCAGACTATGTCATCCCCAT
AGGGACCTATGGCCAAATGAAAAATGGATCAACACCCATGTTTAACGACATCAATATTTA
TGACCTCTTTGTCTGGATGCATTATTATGTGTCAATGGATGCACTGCTTGGGGGATCTGA
AATCTGGAGAGACATTGATTTTGCCCATGAAGCACCAGCTTTTCTGCCTTGGCATAGACT
CTTCTTGTTGCGGTGGGAACAAGAAATCCAGAAGCTGACAGGAGATGAAAACTTCACTAT
TCCATATTGGGACTGGGGGGATGCAGAAAAGTGTGACATTTGCACAGATGAGTACATGGG
AGGTCAGCACCCCACAAATCCTAACTTACTCAGCCCAGCATCATTCTTCTCCTCTTGGCA
GATTGTCTGTAGCCGATTGGAGGAGTACAACAGCCATCAGTCTTTATGCAATGGAACGCC
CGAGGGACCTTTACGGCGTAATCCTGGAAACCATGACAAATCCAGAACCCCAAGGCTCCC
CTCTTCAGCTGATGTAGAATTTTGCCTGAGTTTGACCCAATATGAATCTGGTTCCATGGA
TAAAGCTGCCAATTTCAGCTTTAGAAATACACTGGAAGGATTTGCTAGTCCACTTACTGG
GATAGCGGATGCCTCTCAAAGCAGCATGCACAATGCCTTGCACATCTATATGAATGGAAC
AATGTCCCAGGTACAGGGATCTGCCAACGATCCTATCTTCCTTCTTCACCATGCATTTGT
TGACAGTATTTTTGAGCAGTGGCTCCGAAGGCACCGTCCTCTTCAAGAAGTTTATCCAGA
AGCCAATGCACCCATTGGACATAACCGGGAATCCTACATGGTTCCTTTTATACCACTGTA
CAGAAATGGTGATTTCTTTATTTCATCCAAAGATCTGGGCTATGACTATAGCTATCTACA
AGATTCAGACCCAGACTCTTTTCAAGACTACATTAAGTCCTATTTGGAACAAGCGAGTCG
GATCTGGTCATGGCTCCTTGGGGCGGGGATGGTAGGGGCCGTCCTCACTGCCCTGCTGGC
AGGGCTTGTGAGCTTGCTGTGTCGTCACAAGAGAAAGCAGCTTCCTGAAGAAAAGCAGCC
ACTCCTCATGGAGAAAGAGGATTACCACAGCTTGTATCAGAGCCATTTATAAAAGGCTTA
GGCAATAGAGTAGGGCCAAAAAGCCTGACCTCACTCTAACTCAAAGTAATGTCCAGGTTC
CCAGAGAATATCTGCTGGTATTTTTCTGTAAAGACCATTTGCAAAATTGTAACCTAATAC
AAAGTGTAGCCTTCTTCCAACTCAGGTAGAACACACCTGTCTTTGTCTTGCTGTTTTCAC
TCAGCCCTTTTAACATTTTCCCCTAAGCCCATATGTCTAAGGAAAGGATGCTATTTGGTA
ATGAGGAACTGTTATTTGTATGTGAATTAAAGTGCTCTTATTTT
(SEQ ID NO: 3)
CCCACACTCCCGCCTGTTGCCCTGACCAGAGTCATCATGCCTCTTGAGCAGAGGAGTCAG
CACTGCAAGCCTGAAGAAGGCCTTGAGGCCCGAGGAGAGGCCCTGGGCCTGGTGGGTGCG
CAGGCTCCTGCTACTGAGGAGCAGGAGGCTGCCTCCTCCTCTTCTACTCTAGTTGAAGTC
ACCCTGGGGGAGGTGCCTGCTGCCGAGTCACCAGATCCTCCCCAGAGTCCTCAGGGAGCC
TCCAGCCTCCCCACTACCATGAACTACCCTCTCTGGAGCCAATCCTATGAGGACTCCAGC
AACCAAGAAGAGGAGGGGCCAAGCACCTTCCCTGACCTGGAGTCCGAGTTCCAAGCAGCA
CTCAGTAGGAAGGTGGCCGAGTTGGTTCATTTTCTGCTCCTCAAGTATCGAGCCAGGGAG
CCGGTCACAAAGGCAGAAATGCTGGGGAGTGTCGTCGGAAATTGGCAGTATTTCTTTCCT
GTGATCTTCAGCAAAGCTTTCAGTTCCTTGCAGCTGGTCTTTGGCATCGAGCTGATGGAA
GTGGACCCCATCGGCCACTTGTACATCTTTGCCACCTGCCTGGGCCTCTCCTACGATGGC
CTGCTGGGTGACAATCAGATCATGCCCAAGGCAGGCCTCCTGATAATCGTCCTGGCCATA
ATCGCAAGAGAGGGCGACTGTGCCCCTGAGGAGAAAATCTGGGAGGAGCTGAGTGTGTTA
GAGGTGTTTGAGGGGAGGGAAGACAGTATCTTGGGGGATCCCAAGAAGCTGCTCACCCAA
CATTTCGTGCAGGAAAACTACCTGGAGTACCGGCAGGTCCCCGGCAGTGATCCTGCATGT
TATGAATTCCTGTGGGGTCCAAGGGCCCTCGTTGAAACCAGCTATGTGAAAGTCCTGCAC
CATATGGTAAAGATCAGTGGAGGACCTCACATTTCCTACCCACCCCTGCATGAGTGGGTT
TTGAGAGAGGGGGAAGAGTGAGTCTGAGCACGAGTTGCAGCCAGGGCCAGTGGGAGGGGG
TCTGGGCCAGTGCACCTTCCGGGGCCGCATCCCTTAGTTTCCACTGCCTCCTGTGACGTG
AGGCCCATTCTTCACTCTTTGAAGCGAGCAGTCAGCATTCTTAGTAGTGGGTTTCTGTTC
TGTTGGATGACTTTGAGATTATTCTTTGTTTCCTGTTGGAGTTGTTCAAATGTTCCTTTT
AACGGATGGTTGAATGAGCGTCAGCATCCAGGTTTATGAATGACAGTAGTCACACATAGT
GCTGTTTATATAGTTTAGGAGTAAGGGTCTTGTTTTTTACTCAAATTGGGAAATCCATTC
CATTTTGTGAATTGTGACATAATAATAGCAGTGGTAAAAGTATTTGCTTAAAATTGTGAG
CGAATTAGCAATAACATACATGAGATAACTCAAGAAATCAAAAGATAGTTGATTCTTGCC
TTGTACCTCAATCTATTCTGTAAAATTAAACAAATATGCAAACCAGGATTTCCTTGACTT
CTTTGAGAATGCAAGCGAAATTAAATCTGAATAAATAATTCTTCCTCTTCAAAAAAAAAA
AAAAAAAAAAAAAGGCCACA
(SEQ ID NO: 4)
GTTGGCAGAGGTGGCGGCGGCGGCATGGGTGCCCCGACGTTGCCCCCTGCCTGGCAGCCC
TTTCTCAAGGACCACCGCATCTCTACATTCAAGAACTGGCCCTTCTTGGAGGGCTGCGCC
TGCACCCCGGAGCGGATGGCCGAGGCTGGCTTCATCCACTGCCCCACTGAGAACGAGCCA
GACTTGGCCCAGTGTTTCTTCTGCTTCAAGGAGCTGGAAGGCTGGGAGCCAGATGACGAC
CCCATAGAGGAACATAAAAAGCATTCGTCCGGTTGCGCTTTCCTTTCTGTCAAGAAGCAG
TTTGAAGAATTAACCCTTGGTGAATTTTTGAAACTGGACAGAGAAAGAGCCAAGAACAAA
ATTGCAAAGGAAACCAACAATAAGAAGAAAGAATTTGAGGAAACTGCGAAGAAAGTGCGC
CGTGCCATCGAGCAGCTGGCTGCCATGGATTGAGGCCTCTGGCCGGAGCTGCCTGGTCCC
AGAGTGGCTGCACCACTTCCAGGGTTTATTCCCTGGTGCCACCAGCCTTCCTGTGGGCCC
CTTAGCAATGTCTTAGGAAAGGAGATCAACATTTTCAAATTAGATGTTTCAACTGTGCTC
TTGTTTTGTCTTGAAAGTGGCACCAGAGGTGCTTCTGCCTGTGCAGCGGGTGCTGCTGGT
AACAGTGGCTGCTTCTCTCTCTCTCTCTCTTTTTTGGGGGCTCATTTTTGCTGTTTTGAT
TCCCGGGCTTACCAGGTGAGAAGTGAGGGAGGAAGAAGGCAGTGTCCCTTTTGCTAGAGC
TGACAGCTTTGTTCGCGTGGGCAGAGCCTTCCACAGTGAATGTGTCTGGACCTCATGTTG
TTGAGGCTGTCACAGTCCTGAGTGTGGACTTGGCAGGTGCCTGTTGAATCTGAGCTGCAG
GTTCCTTATCTGTCACACCTGTGCCTCCTCAGAGGACAGTTTTTTTGTTGTTGTGTTTTT
TTGTTTTTTTTTTTTTGGTAGATGCATGACTTGTGTGTGATGAGAGAATGGAGACAGAGT
CCCTGGCTCCTCTACTGTTTAACAACATGGCTTTCTTATTTTGTTTGAATTGTTAATTCA
CAGAATAGCACAAACTACAATTAAAACTAAGCACAAAGCCATTCTAAGTCATTGGGGAAA
CGGGGTGAACTTCAGGTGGATGAGGAGACAGAATAGAGTGATAGGAAGCGTCTGGCAGAT
ACTCCTTTTGCCACTGCTGTGTGATTAGACAGGCCCAGTGAGCCGCGGGGCACATGCTGG
CCGCTCCTCCCTCAGAAAAAGGCAGTGGCCTAAATCCTTTTTAAATGACTTGGCTCGATG
CTGTGGGGGACTGGCTGGGCTGCTGCAGGCCGTGTGTCTGTCAGCCCAACCTTCACATCT
GTCACGTTCTCCACACGGGGGAGAGACGCAGTCCGCCCAGGTCCCCGCTTTCTTTGGAGG
CAGCAGCTCCCGCAGGGCTGAAGTCTGGCGTAAGATGATGGATTTGATTCGCCCTCCTCC
CTGTCATAGAGCTGCAGGGTGGATTGTTACAGCTTCGCTGGAAACCTCTGGAGGTCATCT
CGGCTGTTCCTGAGAAATAAAAAGCCTGTCATTTCAAACACAAAAAAAAAAAAAAAAAAA
AAAAAAAAA

Thus, in one embodiment, double stranded RNA having a sense strand that encodes a cancer antigen, e.g., one that is useful to prevent, inhibit or treat cancer or otherwise enhance the immune system, may be combined with a plurality of distinct lipids or a peptide conjugate and then introduced to a host organism, e.g., a mammal such as a human, optionally with an adjuvant. The double stranded RNA may be directly administered, or by administration of two plasmids, each encoding one of the strands, optionally in conjunction with positively charged polymers such as PEI, cationic polypeptides, e.g., protamine, or dendrimers, or using a delivery vehicle, e.g., a microparticle or nanoparticle, for instance, a liposome. For instance, double stranded RNA having a sense strand that encodes tyrosinase or survivin may be used to treat a melanoma patient, e.g., as an immunotherapeutic.

For infectious disease, a double stranded mRNA having a sense strand that encodes a microbial protein including a protein or glycoprotein specific for a viral pathogen, a bacterial pathogen, an algal pathogen, or a fungal pathogen, for example, a respiratory syncytial virus (RSV) fusion protein, e.g., where the sense strand has nucleic acid sequences with at least 90%, 92%, 95%, 97%, 98%, 99% or 100% nucleic acid sequence identity to coding sequences in SEQ ID NO:5 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:5, may be employed as a vaccine.

(SEQ ID NO: 5)
ATGGAGCTGCTGATCCACAGGTTAAGTGCAATCTTCCTAACTCTTGCTATTAATGCATTG
TACCTCACCTCAAGTCAGAACATAACTGAGGAGTTTTACCAATCGACATGTAGTGCAGTT
AGCAGAGGTTATTTTAGTGCTTTAAGAACAGGTTGGTATACCAGTGTCATAACAATAGAA
TTAAGTAATATAAAAGAAACCAAATGCAATGGAACTGACACTAAAGTAAAACTTATAAAA
CAAGAATTAGATAAGTATAAGAATGCAGTGACAGAATTACAGCTACTTATGCAAAACACA
CCAGCTGCCAACAACCGGGCCAGAAGAGAAGCACCACAGTATATGAACTATACAATCAAT
ACCACTAAAAACCTAAATGTATCAATAAGCAAGAAGAGGAAACGAAGATTTCTGGGCTTC
TTGTTAGGTGTAGGATCTGCAATAGCAAGTGGTATAGCTGTATCCAAAGTTCTACACCTT
GAAGGAGAAGTGAACAAGATCAAAAATGCTTTGTTATCTACAAACAAAGCTGTAGTCAGT
CTATCAAATGGGGTCAGTGTTTTAACCAGCAAAGTGTTAGATCTCAAGAATTACATAAAT
AACCAATTATTACCCATAGTAAATCAACAGAGCTGTCGCATCTCCAACATTGAAACAGTT
ATAGAATTCCAGCAGAAGAACAGCAGATTGTTGGAAATCAACAGAGAATTCAGTGTCAAT
GCAGGTGTAACAACACCTTTAAGCACTTACATGTTAACAAACAGTGAGTTACTATCATTG
ATCAATGATATGCCTATAACAAATGATCAGAAAAAATTAATGTCAAGCAATGTTCAGATA
GTAAGGCAACAAAGTTATTCTATCATGTCTATAATAAAGGAAGAAGTCCTTGCATATGTT
GTACAGCTACCTATCTATGGTGTAATAGATACACCTTGCTGGAAATTACACACATCACCT
CTATGCACCACCAACATCAAAGAAGGATCAAATATTTGTTTAACAAGGACTGATAGAGGA
TGGTATTGTGATAATGCAGGATCAGTATCCTTCTTTCCACAGGCTGACACTTGTAAAGTA
CAGTCCAATCGAGTATTTTGTGACACTATGAACAGTTTGACATTACCAAGTGAAGTCAGC
CTTTGTAACACTGACATATTCAATTCCAAGTATGACTGCAAAATTATGACATCAAAAACA
GACATAAGCAGCTCAGTAATTACTTCTCTTGGAGCTATAGTGTCATGCTATGGTAAAACT
AAATGCACTGCATCCAACAAAAATCGTGGGATTATAAAGACATTTTCTAATGGTTGTGAC
TATGTGTCAAACAAAGGAGTAGATACTGTGTCAGTGGGCAACACTTTATACTATGTAAAC
AAGCTGGAAGGCAAGAACCTTTATGTAAAAGGGGAACCTATAATAAATTACTATGACCCT
CTAGTGTTTCCTTCTGATGAGTTTGATGCATCAATATCTCAAGTCAATGAAAAAATCAAT
CAAAGTTTAGCTTTTATTCGTAGATCTGATGAATTACTACATAATGTAAATACTGGCAAA
TCTACTACAAATATTATGATAACTACAATTATTATAGTAATCATTGTAGTATTGTTATCA
TTAATAGCTATTGGTTTGCTGTTGTATTGCAAAGCCAAAAACACACCAGTTACACTAAGC
AAAGACCAACTAAGTGGAATCAATAATATTGCATTCAGCAAATAG

In one embodiment, double stranded RNA having a sense strand that encodes a microbial antigen, e.g., one that is useful to prevent, inhibit or treat microbial infection, may be combined with a plurality of distinct lipids or a peptide conjugate and then introduced to a host organism, e.g., a mammal such as a human, optionally with an adjuvant. The double stranded RNA may be directly administered, or by administration of two plasmids, each encoding one of the strands, optionally in conjunction with positively charged polymers such as PEI, cationic polypeptides, e.g., protamine, or dendrimers, or using a delivery vehicle, e.g., a microparticle or nanoparticle, e.g., a liposome. For instance, double stranded RNA having a sense strand that encodes a RSV fusion protein may be used as a vaccine.

In one embodiment, for protein replacement or augmentation, a double stranded mRNA having a sense strand that encodes Foxp3, e.g., where the sense strand has nucleic acid sequences with at least 90%, 92%, 95%, 97%, 98%, 99% or 100% nucleic acid sequence identity to coding sequences in SEQ ID NO:6 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:6, or a double stranded mRNA having a sense strand that encodes surfactant protein B (Spb), e.g., where the sense strand has nucleic acid sequences with at least 90%, 92%, 95%, 97%, 98%, 99% or 100% nucleic acid sequence identity to coding sequences in SEQ ID NO:6 or SEQ ID NO:7 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:6 or SEQ ID NO:7, may be employed. The double stranded RNA for protein replacement or augmentation may be combined with a plurality of distinct lipids or a peptide conjugate and then directly administered.

An exemplary sequence for expression is:

(SEQ ID NO: 6)
GTGAGCAGAATCCATGTGCAAGGAGAGCAGGCAGTTCAGGACGAGGGTGAGCTGGTCTCT
GCAGGTTTAGTGCTGTGGCACTGTGCCTGGTATATGCTCCCGGCAACTTCTCCTGACTCT
GCCTTCAGACGAGACTTGGAAGACAGTCACATCTCAGCAGCTCCTCTGCCGTTATCCAGC
CTGCCTCTGACAAGAACCCAATGCCCAACCCTAGGCCAGCCAAGCCTATGGCTCCTTCCT
TGGCCCTTGGCCCATCCCCAGGAGTCTTGCCAAGCTGGAAGACTGCACCCAAGGGCTCAG
AACTTCTAGGGACCAGGGGCTCTGGGGGACCCTTCCAAGGTCGGGACCTGCGAAGTGGGG
CCCACACCTCTTCTTCCTTGAACCCCCTGCCACCATCCCAGCTGCAGCTGCCTACAGTGC
CCCTAGTCATGGTGGCACCGTCTGGGGCCCGACTAGGTCCCTCACCCCACCTACAGGCCC
TTCTCCAGGACAGACCACACTTCATGCATCAGCTCTCCACTGTGGATGCCCATGCCCAGA
CCCCTGTGCTCCAAGTGCGTCCACTGGACAACCCAGCCATGATCAGCCTCCCACCACCTT
CTGCTGCCACTGGGGTCTTCTCCCTCAAGGCCCGGCCTGGCCTGCCACCTGGGATCAATG
TGGCCAGTCTGGAATGGGTGTCCAGGGAGCCAGCTCTACTCTGCACCTTCCCACGCTCGG
GTACACCCAGGAAAGACAGCAACCTTTTGGCTGCACCCCAAGGATCCTACCCACTGCTGG
CAAATGGAGTCTGCAAGTGGCCTGGTTGTGAGAAGGTCTTCGAGGAGCCAGAAGAGTTTC
TCAAGCACTGCCAAGCAGATCATCTCCTGGATGAGAAAGGCAAGGCCCAGTGCCTCCTCC
AGAGAGAAGTGGTGCAGTCTCTGGAGCAGCAGCTGGAGCTGGAAAAGGAGAAGCTGGGAG
CTATGCAGGCCCACCTGGCTGGGAAGATGGCGCTGGCCAAGGCTCCATCTGTGGCCTCAA
TGGACAAGAGCTCTTGCTGCATCGTAGCCACCAGTACTCAGGGCAGTGTGCTCCCGGCCT
GGTCTGCTCCTCGGGAGGCTCCAGACGGCGGCCTGTTTGCAGTGCGGAGGCACCTCTGGG
GAAGCCATGGCAATAGTTCCTTCCCAGAGTTCTTCCACAACATGGACTACTTCAAGTACC
ACAATATGCGACCCCCTTTCACCTATGCCACCCTTATCCGATGGGCCATCCTGGAAGCCC
CGGAGAGGCAGAGGACACTCAATGAAATCTACCATTGGTTTACTCGCATGTTCGCCTACT
TCAGAAACCACCCCGCCACCTGGAAGAATGCCATCCGCCACAACCTGAGCCTGCACAAGT
GCTTTGTGCGAGTGGAGAGCGAGAAGGGAGCAGTGTGGACCGTAGATGAATTTGAGTTTC
GCAAGAAGAGGAGCCAACGCCCCAACAAGTGCTCCAATCCCTGCCCTTGACCTCAAAACC
AAGAAAAGGTGGGGGGGGGAGGGGGCCAAAACCATGAGACTGAGGCTGTGGGGGCAAGGA
GGCAAGTCCTACGTGTACCTATGGAAACCGGGGGATGATGTGCCTGCTATCAGGGCCTCT
GCTCCCTATCTAGCTGCCCTCCTAGATCATATCATCTGCCTTACAGCTGAGAGGGGTGCC
AATCCCAGCCTAGCCCCTAGTTCCAACCTAGCCCCAAGATGAACTTTCCAGTCAAAGAGC
CCTCACAACCAGCTATACATATCTGCCTTGGCCACTGCCAAGCAGAAAGATGACAGACAC
CATCCTAATATTTACTCAACCCAAACCCTAAAACATGAAGAGCCTGCCTTGGTACATTCG
TGAACTTTCAAAGTTAGTCATGCAGTCACACATGACTGCAGTCCTACTGACTCACACCCC
AAAGCACTCACCCACAACATCTGGAACCACGGGCACTATCACACATAGGTGTATATACAG
ACCCTTACACAGCAACAGCACTGGAACCTTCACAATTACATCCCCCCAAACCACACAGGC
ATAACTGATCATACGCAGCCTCAAGCAATGCCCAAAATACAAGTCAGACACAGCTTGTCA
GAACACGCTCGTGTGCACGTACACACATGCAGCCCCTCCACTCTATCTCCTGAGTTCCAT
GAATACACACCGACTCTCCAAGATGTACCCCACGTCTCACTTGCCACTGACCCCAGTTCC
CTACCCACAAGCCCCAATCCATGCCTAAGCGTGGCCCACAGAAGAACTTCTCTTTTATTT
GGGATCCAAGGCCCCTGGCCCCCAGTGCCCATCCAATAAACTGTGGTCAGCTGGACAATC
ACCCTGATCAGATATGGGAACATATAAGCAGACAGCTGGGTTTAAGATCCCAGCAGGAGA
AAGCGGATACCAAATGAAAGAGAGTGCTAGAACAGGTGCCTCAGCACTGTCTCCAGCACC
CCAAATTCCTGCCTGTGGTTAGGAGACATCCATCAGGGCTCTAGGCCTCTCGGACCCGGC
CCAAGAGGCCAGCATTCTCCTGGCGAAGGGCTCGGTAGTCCTCACAGATCTTCTCCAGGT
TGCTCAAAGTCTTCTTGCCCATCTCTGTCTCAATCTAAGAAAACAGGATGCACACTTCTT
CAGCCCCTGCAGGCTGCCCCTCTACTGAACTCCTCCCTGCTCCTCCTATTCCCGTAACAG
CAGCCTGTTCCTTCCCATCACTGGGCTTCTGGGTATGTCCTTCCCTCCACTCCACCTAAA
GCAGCAACTTCTGCCATGGGCTCTGGGAGGCATTAGGAGCCGCAAGCTAAAAGCCAGGGC
TCAGAGTAGGCTACTGGCTAGCTTCAGGTCCCAGGCACAGTGGGCACGAAGGCAAAGCCT
CTAGCTGTTAGTTGTCTGGTTTCAAAGACTCTCAGCGCAAAACAAGGAACTATCCCCTGG
CCTGTCTCCATTCCCCTTACCAGTCCCAGGTCTCACCTGCTCCTCAAGATCTCGAACTTC
CCTCATGATAGTGCCTGTGTCCTCAATGGTCTGGATGAGCTGACTGCAATTCTGGAGACA
GCAAGAATACAAGGCTTGCACCTATGCTGGCCCTCTCCAGCCAACCCACCAGGCACATGG
CTCCCCTCACCTCATGCAGGGCAGCTAGGTACTTGTAGGCTTTCCGAACAGCATCATCCT
TCTTAGCATCCTGATAAGACAAAGGGGATCTCCGAGATATCAGCAAGCCATTCCCCCTTT
TCCACTACTCTATGCCCCTATAAGACCACCCTTTACTAGTACTTTGCCTTCATCCTCCAC
AGAGCAAAGCTAGGCCCCAAGCAACAGTGCACCTAAAGGACTCACAGAGGGGCAGGCAAC
AACTCAGTCCCGCCTCCACCCTCCCGGAGGCCAGCCTGCTCCATACCTTGAACACAAGCT
CATCAGTCACTGCAAATGTCCGGTCGAGCTTCCCAGAGAGAGAGTTGATTTCCTTCTGCA
GTTCCTTTGTGTCCGACAAGATCTGGTAGAAACCAGGGTAACTATCAGTGCACATCTTGG
GCAAGGTAGCTGATCAGTGATAACACTCACGTGCCTATACTTACATCCAGTCAGGGCCCA
TGTCGCTGTGTTGGGGTGACTATTATGTGTTGGAGTGTGCCTGAACAGCTCTGCCTAGTA
GTGAGCATAAAGTCCCTGTGT,
or
(SEQ ID NO: 7)
GGTACCATGTCTATCCTGACCCTAAGATTAGTTCCTCGGGTTTGAGGATTGCAGCAACAC
TGACCGTTCAGGCCCTGGTCAAGGTGGGGCTGCTGCTTCTTCCTTGGCTTCTTTCCAAGG
AGCCACCAAGAGGCAGAAAGAAATGAAGAGACACAAAGCAAGGCAGAATAGCACTTCGGA
TGACACTGTCCGCATTGCCCGACAGATATGGCACTAGACTGCAGCCAAAGGACTCTCTGA
AACTATTAACAAGGTTGTGAGAACTTGTGACCAGTCTGTGAGGTGCTGTGTCTGGGTGTC
ATGTCACTGGGGACATCATCAGTGTCACCAGTGGCACAGTGGAATGCCTGGTGAGCTGAG
ACACACAAATGAGGCAGGCGTGGTGGGCACACACCTGTAATCCCAACGGAACGTAAACCT
GGTATGGCAGTGCTCACCTGTATGTGGTGGCACTCAGGAAGTGGAGGCAGGAGCATTAGG
AGTCTAAGGTCATCCTCAGCTACATTGACAAATCTGAGACCAGCCTGGGCGACATGAGTC
CTTGTGTTAAAAAAAAAAAAAAAAAAAAAAAAAAAAAAAGAGAGAGAGAGAGAGAGAGAG
AGAATCAGACAGTGGTGGTGCATGCTTTTAATCCAGCACTTGGGAGGCAGAGGCAAACAG
ATTACTGTGAGATCAAGGCCAGCCTGGTCTTCAGAGCAAGTTCCAGGACAAGCAGGGCTA
CATAGAAAAAACCCGTCTCTATAAACAAACAAAACAAAACAAAACAAAAAAAAGCAGATC
TCGTGACTCTCTGAAGAAGGCCATTTCCCGCCAGTCCTTGGGGTTAGCCGTAAGTAGCAG
GCTGTAGTGTCTCGAGGCCACAAAAACTAGGAGAACCCTGGGACCACTTCCAGGGTGTCG
TTTTACATCACATGTCCAACTATTTACCTTCATCTTGGGGCTAGCTCCCACCCCATACAG
CCTGTGAGTGCTGGAGGACTTTCTAGGGAGCCTCCGTAGGAAAGGCACTGGCAGGTCTCA
GAAAAGGATCGGGGTCCTGATGGGGGGGGGGGGGTCAGTAGTGCCTAATGCACTCAGACA
AGCACCGGCGCTGCAGCCAGCCCTGAACTGCTTTTTCTCTAAGCCCAGCCAGGTGTGGAC
ATAGCCTCAGAGGACCACGTGTCAGCTGAATCCCATCTCATGCCCAGGAGGGGTGACTGG
GAGAGATGGGCATCTGCTTCTGGGTAAAGCTACCTAAGAGCCACAGGGGACACAGAAATC
TCAGCCTCACAGGGCACTTTCCTGTTTGTCTAATGCTCCTCTCCCTAGCACCAGCCAGGA
GTCTATAGAATCAGAGGATTTTAAAGTAAGGGGGGAGTGGGAGGTCGGTTGGCCCCAGGA
GCACCCTAAGTGTGCCCTTCCGGCACTTACCCTGCGTCAAGAGCCAGGAAGGAAGCTCTC
AAGGGCGTTGCATAAGAGTAGAGGATTGAGAAGCCTGGGGTGGGGCTAGAGAGGCTCATT
CTGACCCCACTCAGCATCCCTTGCACAGTCCAGAGCGTGGGGATCAAACGAGACCCCCTT
GTTTGACGGTGAACAAAGTCAGGCTGAGGGGGTTCGGGAAGGGGGTAAAGGACTAGGAAC
CGACATCGGCCAGCACACGGGAGGTGGACAGGGGTGTCCCTGCTGAGAAGACCTGGAGGG
CTCTCAAGACACAGGCAAACACTGAGGTCAGCCTGTTCCCATGGAGTCCAGCCCCCAGGT
CCTCTCCCCTACTATAAGAGCCCATGACTCAAGTAGGGTACTAAGCAGTAGGCAGCCATG
GCCAAGTCGCACCTACTGCAGTGGCTACTGCTGCTTCCTACCCTCTGCTGCCCAGGTGCA
GGTGAGTCCCCGGCCTCCCTCACAGAGGCCTCTCCAGCACTTACTGAGTCAGCTCCGTGC
CCAGAAAGACCCCAGTCTGCACATAATCCAGAATTTAAACGCCAGTTAGCTGAGGCACAG
AGAAGTCCTAGGGCCTCATCCAAGGTCACAGTTAGTGGATGGATGTTGAAGCAGGAGGAC
TCAGAGCTGCCTGGCAGAAGCAATGGCCACTCCTTTGCAATGAAACTGGGTTGGAGGTGG
GGTGGAGGCAGGGTGCCGAGTGTATGCTGGATCCTGATGAGAGTTGCTCTGACCCCAACT
CCAGCTATCACGTCGGCCTCATCCCTGGAGTGTGCACAAGGCCCTCAATTCTGGTGCCAA
AGCCTGGAGCATGCAGTGCAGTGCAGAGCCCTGGGGCACTGCCTGCAGGAAGTCTGGGGG
CATGCAGGAGCTGTGAGTAGCACCAAGCGGGCACTGGAAATCCAGGGAGGAGGAACTGGG
GTGGATTCTGAGCGGACCTTAGGAAATTGGAGTTCCCACAAGGCTGGGGTGGCAGGGAAT
GATGGAATGGTATAGTGTGACAGGAAATGGTGGGCAGAGTACAATAGAAGGAAACATGGT
GGAATGAGATGAATGGGGTGGGCATGGTGGGTAAGACAGGGTGGATGTGTGGGTAAGACA
GGGTGGATGTGGTGGGTAAGACAGGGTAGATGTGGTGGGTAAGAGGGGGTGAGCATGTGG
GTAAGATGGGGTGGCTGGGGTGAGATGGACAAGATGGAATAGAACAGGGTGGATCAAGTG
GGTGGCACAGAATGGGATGGAATTTGCACAATGGGATGAGATGGGATGATGGGTGGGTAG
CCTTAAGGTACCTGTCAGCCTGTGTCTGAGAAAGCCTCAATCCCTGGAGTTAGGAGCATG
CCCCCAACTCATTAGCCTCACTTGAGACCCTTTCTTCCAGAATGACCTGTGCCAAGAGTG
TGAGGATATTGTCCACCTCCTCACAAAGATGACCAAGGAAGATGCTTTCCAGGTAATGGG
AAACGGTACAGTGTGATCTGGTAGAGGCCTGGCGTCAGGGGACTCTGGTGGGGGCAGACC
TCAGAAAGACCAGGCTAATCCTCCCTTCTCTGCTCTCCCAGGAAGCAATCCGGAAGTTCC
TGGAACAAGAATGTGATATCCTTCCCTTGAAGCTGCTTGTGCCCCGGTGTCGCCAAGTGC
TTGATGTCTACCTGCCCCTGGTTATTGACTACTTCCAGAGCCAGATTGTGAGGACCCTGA
CCTACCTGCCGCACAGTGCATGTGCCTAAGTGGCCACTTACCTATATAAGTGGCACCCCA
ACACATGCACACACACACATACACACCCACAGACGCAATAAGACACACACACACACACGT
ACACACACACACACACACACACACACACACACACACTTCCCACTACAGCCACAGGAAGCT
CAGTCTCTTCATCCAGATACCCAAATCAGAGCCTGCCTGCTCAGCATACTACAGACATTG
AGACCCGCCCTCCATCCCCTCACCCACACATGCCCACATTCTTATTGTCACACAATATGC
TCACACACACTCACTCTTTCCAGACACATGCTCCCAGGCCCTACACAGCCCCATCTCTCT
GTCTTTGTCCCTTTCATAGTGTCCTAAGATGCAGTACTTCACCCAGCCTGCTCCCCATAA
CCCCAGGCTCAAAGACTGTGGCCCTTGTCCCTGAATATGAACCTGGGCAGAGAGGGGTTC
CCTCCTTACCCTAAAACCCCTCACCTGTTCCATGCCCTAGAACCCCAAAGCCATCTGCAA
TCATGTAGGCCTGTGCCCACGTGGGCAGGCTAAGCCAGAACAGAATCCAGGGATGCCGGA
TGCCGTTCCAAACCCTCTGCTGGACAAGCTGGTCCTCCCTGTGCTGCCAGGAGCCCTCTT
GGCAAGGCCTGGGCCTCACACTCAGGTAAGCCAGTCCATTCCCAGCAGCTGCTGGGAATC
CAGAAGGCTAGCATGGCCGCTGAGACGCGTGGGCACCCAGAGAGGCTGAGCTCAAACTAG
GAGGCAGAGATGGCAAGGTCAGGCAAGGTCACACAACCGAGGTAGCTCCCAGCCTAACCA
CACTTCACCGCTTCCTTCCTCAGGACTTCTCTGAGCAACAGCTCCCCATTCCCCTGCCCT
TCTGCTGGCTTTGCAGAACTCTGATCAAGCGGGTTCAAGCCGTGATCCCCAAGGTAAGGA
CCACACAGAGCTCAGAGGGGCCCCCAATAGCTGGCACCTTCCTCCACCTCAACACTCCAA
GAAGGCTGTGAGGAGTTAGATGAGGAGACACCCACACATTGCTCCTACCCAAGGAACCTT
GAGGCTCAGGTATGGGAGGTTAGGTCAGAGCCACCTTCTCTTCCAACAGATCACCATCGG
AAGGCTGAGAAGCACTGGTTGTCACTGTAGGAAAAAAGTACATTAATTTCTCAAAAAAAA
AAAAAAACAGTTCATCAATAGTAAGCATCTCTTCTGTCCTCCAAATCCATGGTAGCCTCT
GCCAGTGCCTTGTCAGATGAGGATTGTTCTCCCCACAAATGGTCATGGCCTATCAACACT
AACACTAAGCCCACATCAGTCATAAAGACAACAGGGCACACAGTCAAGCCTTTCTGAAGC
CTGTGTGATGGAAGGAACGTGCAGACTATAGAGCAGGATGAGCTGAGGGGTCGCACAGAT
AAAAATGGTAACAGACAGGTCAGCCAGGGAGAGGCTCTGAAGAGGGTAACAACTAAGCCA
AGATCTAGGAGAAAACAAGGTCCCCAGGGGCCAAGGACATCCATCCATCAATAAAAAATG
AGCTCAATCAGATGTTGGAGGGAGGGACTCTGTAAGGAGGGACCAGGAGCAGGGGGCAGC
GTTTGGGGTGTAAATGATAGATAAATGCCTTTAAAATGAGCTCAGAGGGCTAGGAAGATG
GCTCGGTGAGTACAGTCCTTGCTGAACCTGAGTTCAGATACTTGCACCCTCATAAAAGTT
GGGGGGTGGGCTGGAGACATGGCTCAGTAGTTAAGAGCACTGACTGCTCTTCTAGAGGTC
CTGAGTTCAATTCCCAGCAACCACTTGGTGGCTCACAACCACCTATAATGGGATCTGATG
CCCTCTTCTGGTGTGTCTGAACTTACATACATAAAATAAAAATAAAAGTTGGGGGTTGCT
CACAGTCAGCTAATGGATGGATCATAGGGCTCCCAATGGAGGAGCTAGAGAAAGTAGCCA
AGGAGCTAAAGGGATCTGCAACCCTATAGGTGGAACAACATTATGAGCTAACCAGTACCC
CGGAGCTCTTGACTCTAGCTGCATATATATCAAAAGATGGCCTAGTCGGCCATCACTGGA
AAGAGAGGCCCATTGGACTTGCAAACTTTATATGCCCCAGTACAGGGGAATACCAGGGCC
AAAAAGGGGGAGTGGGTGGGCAGGGGAGTGGGGGTGGGTGGATATGGGGGACTTTTGGTA
TAGCATTGGAAATGTAAATGAGTTAAATACCTAATAAAAAATGGAAAAAAAAAAAGTTGG
GGGTTAGCAATGAACATTTGTAACCCTACACACTAGGTAGTCAGAAATAGGCAGATCCCT
AGAGCATGCTGGCCAGCCAGTCTAGCCAAATGGATGAGCTTCAGGGTTAGTGTGAGACCT
TGTCTCAAAAAAAAAAAAAAAAAAAAAAAAAATGGACGGCCTGAAGATTCGGATCGACAG
TTAGGAACATTTGCTGCTTTTCAGAAGAGTGAGTTGGGTACCCAGCACCACTGTCAGGCA
GCTCACAACCCCCTGTAACTGCTGCTCTAGGGAATCCAATGCCCTCTTCTGGCAGCCAAG
GGCACCAGCACATATGTGGCATTCATATACTCAGATACACAGACATATGTAAAAATAAAA
ATAAATCTTTAGAAAATAATTAGGTAGGGAGTGAAGTGACTAAGGAAGACACTCAATCTT
GGCTCTGGCCTCCACACACATGTGCACATGTACTTAAACATCTACGTGCAAAACAAACAA
ACAAACACCCAGCCGTATCAATGTGAACATCACTGAGGACCGAAGGCATGAGCAAGACTG
TTAAGAGACAATGTATAGACAGATGGAGATGGCATCAGAATTGCTGAGAGGGGACAGGCA
GCCAACGGGGGACCGTGCTGCATTGCCAGGGAAGCCAAGAGAGAAGGGTGTTTGACTGAT
TGAAAGGCAGCTGAACCATCAGGCAGGGTGAGAGTTAGGCAGGGGATGTGGAAGTGTTCC
AAAAAGGGGAGCAGGCATGGTGAGGCTTCCTAAGGTCAGAAGCCATTCTAGCGTGTTCTC
CAGGCAGCAGGGACCAGAGAGAGGATAAGGCCAGGGAAAGAGGCATGGGTGGAGGTAATC
CAGGAGTGAAGACCATTTCACCAATGAGCAGCTTGGTCATTGACTACAGTGACTATTGAT
TTACATCACCATGACAGGAGAGCCATGTGTGGGTCAATGATAACAGGTGGGTCTCTTAAG
TGAAGTGCCCCATTTGGGAGCCATCACACTCCAGGGGTGTCCATATTCTGAGTCCTCCCC
CTGCCTCAACCTCCTGGCACTGGGGCTAGCTGGTCACATGGGCTGAATAAGGAGTAAAGG
AAAAAGCCACACCCTGGTGACCTCTGTCACCCTTCAGCTAGAGCCTGCTTGGAATTGGAG
TTGAGGTAGGAGATGTGCTGGCTTTCCCAGGGGTTCCAAAAGCCAAAGACATGTCAGCTC
TGGGGGCCAGCAGAAGGAACTGCCTGTCTTCCTGATGCATAAGCATGGGAAGGTAGGTGG
CCCTCGGTCAGGGAATGGGTTTGAATTGGGTCAGGCTGTTAGATGCCATGGCCTTGCAGC
CCCCTTTCAAATGACTCAAGCCTTTAGAGCTAGATCTATATTTGGTGTCAACTGCAGATT
CTCTCAGTGACTCCGGGTGCACCTGAGACCCCTGCTGTCTTGGATGCTCAGTGACCTGTG
GACAGAACTGCTCTTTCCTAGAAGGGAGAAAGGGGATGCATCTGGGGTGCCCACTCAGTT
GGGCACAGTGACATCGTGCCAGAAGAAGGTTCTATGGTTGTCCTTTCTCCACCTTCACCC
CAGGGTGTGCTGGCTGTGGCTGTGTCCCAGGTGTGCCACGTGGTACCCCTGGTGGTGGGT
GGCATCTGCCAGTGCCTGGCTGAGCGCTACACAGTTCTCCTGCTAGACGCACTGCTGGGC
CGTGTGGTGCCCCAGCTAGTCTGTGGCCTTGTCCTCCGATGTTCCACTGAGGATGCCATG
GGCCCTGGTAAGACTTGCCCGTCCCCTCCCCCTCCCCAACTCACATCCCTCCAGTGCACA
TGGGAGGGAACATGGACAAGGTGGGGTTCAGGAACCAACACTTTTTTTAAACTATTTATT
TCTATGGATATGGCTGCTTTTATTTATATAGCTGAGGCTGGCTTTGAACTCCTAATTTCC
CTTCCTCAGCCATTCAAATGTTAGGAAAGGCTAGCAATGACTGTACTCAGCTTCTAGCTC
TCTCCAAGTGGACTTCTCCCAGTTGAGTTAAAGAGTGATGGGGGAGGGGTGGGGAACAGG
GCAGGACCCTGGGAGAAGGCTAAGTTCTTTTTTTGCTCCAGCTTGGACATCTATATACCC
CATGTATGCCTGGCTCCCACAGAGGCCATAAAGGATGTCAAATCCCCTAGAATTGGAATA
ACTGACAGTTATGAGCCATCATGTGGGGCTCTGGGAATCGAACCTCAGCCCTCTGGAAGA
GCAGCCAGTGCTCTTAACCACGGAACCATCTCTCCAGCCCCAGAACCAACACTTGTACAA
GACAGTCCTGGGGGAAAGATTAAAACAGAGTCTTACTACATAGCACAGGTTGGCCTCGAG
CTTGGTGCAATCCTCCTGCCTCAGCCTCTCAAATACTGGCATGACAAGGTATGTGCCTCC
ATACCCAGCTTGCTGGACAATTCTAACTGCTTTCTCTTTAGCCCTCCCTGCTGTGGAGCC
TCTGATAGAAGAATGGCCACTACAGGACACTGAGTGCCATTTCTGCAAGTCTGTGATCAA
CCAGGCCTGGAACACCAGTGAACAGGCTATGCCACAGGCAATGCACCAGGCCTGCCTTCG
CTTCTGGCTAGACAGGCAAAAGGTAGGGGGCCCACGGGTTGGATGTATGTCATATGTGTG
ATGGTGCCGAGCTAGAAGAGACTTTGTAGCTAGACACACGCACGATGCTGGTTCCCAGCC
TGGTGGACAGGCATGTGGGTCAGACAATGATGGGATTGTAACAAATTTAACTGGCTAGGA
GACATCATGGACCCAAGGCTTTGGACTATGGAACATCAGCAGGCCTTCTTTATGGACTAA
GCACAAGAAAAGTCCTGTTAGTCCCAACAGGAAAGGGTCATACTGCCCTTTCTTGGTTTC
ACTCGATGGTGTGTTTGCCACACTGTTCTCCCAGTGTGCCATGTCACCCCCATGATGGGT
GGTAGCATTTGACAGTACCTAGCAGGCACCAGAAAATGAGAAAAGCCAGGGTCAGCTGGA
GCAGAAAAAGAACTTAGCCTTTTCCCAGGGTCCTGTTCTGCCCCACCCTGCTCACTCTGT
AGAAGTCCTGCAGGAGAGAGCTGGAAGCTGGTACCATAGTGCTAGCCTGTAATTCTAACA
TTTGGAAAGGCTGAAGAAGGAGAAATGGGAGTTCAAAGCCAGCCTCAGCTATATAAATAA
TGAGTTCAGGGTCAGCCTGGGCTACATGAGACCCTGTCTGGTGAAAGGAGACAGAGATAG
GAAAGAACATGAGGCTTGGGTAAGGCTCACTGGCATGGCCACAACCAAGTTTGATCCCTG
GGATCCGTATGGTAAACAAAGAGAATCAACTCCTGTAAACTTTCCTTATGAACACACACA
CACACGAAAACATAATTTTGAAGCCAGGCTGTGGTGGTGCACACCTTTAGTCCTAGCCCT
TGGGAGGCAGAAGCAGATGGATCTAAGTTTGAGCCCAGCCTGGTCTACAGTGTGAGCTCC
AGGACAGCCAGGGTTACACAGAGAAACCCTGTCTCACAAAACCAAAAAGAAATCAACAAC
CACAAAGAACTGAACAGATAGTTCCTTAAGCCTGTGATGAATCCCCTCACTACAGTGGGA
CTTTCTTTAGAGAGGGTCCTATGTAACTTAAACCGCCTCCACCTCCTTTGTACTGAGACT
ACAGGCAGGTACCACTACTGAGTTTCATGTAGTTCTGAAGTTGAAACTAAAGGTTTCATG
CATGCTAGGCAACCATGAGACGATGCTAAGCTGCAAGCCTGCTCCAGCTCCAAGGCCCTG
GCTTCCTCCAAAGCCTGGTTTCAGCCAAACTTAGATAGAGTCCCTTTTTTTAAGACTCAT
TTTATTTGTGTTTTTAGTGCATGTATGTATGGACATCATGTGTGTGTGGTGCCGGGGGGA
GGGGGTCAGAAGAGGCCATCAGATTCCCTGGAACTGGAGTTGAGTGGTTATAAGCCGTCC
TTCCTGTCCTCCAAAGAGCAGCAAGTGCCTAACCCCCGAGCCATCAGCCATTCAGCCCTT
CGGTTGAGTCTTTAATGGTCAGCCAGGCACTGATGGAAAAACACAAACCCACAGTCCGGA
GTGGCAGAGTGAGGTAGAACGCCAGATCTGCAGGTTAAGTTCTCTCCTAGAGGGGGGTCT
ACATATTGTGTCTTTCCTCAGTGTGAACAGTTTGTGGAACAGCACATGCCCCAGCTGCTG
GCCCTGGTGCCTAGGAGCCAGGATGCCCACATCACCTGCCAGGTATGCCCACTCTTCAGC
TGGTCCCAGGAGTCCCCTCTGCTCCCACAGTCCCACCCTCCTTGGTCTATGATCCTCAAG
AGCCCCATTTCTTGGATCCAGGAAGCCTAGGGCTCAGAAGCCCAGAACTAAGTGTACCCA
TAGAACAGGCTTTGGACTTGGAGCAGAAAAGAACACATACTGATTAGGTGGGAGGGGCAA
GTTCATGATGGATGGGCAGCTGGGGGCTGGGGTATGATGCTCCTTATTGCATGTGGTGTG
TTTAGTGACCAGTTTGTTCTATGGTGGGGCTATAGTATGAGGTGGGGGTCCCACTAAGTC
CCAAGGCCATTGACTTAGGGAATGGCACAAGGGGTTCTGAAGGTGAAGGTGAAGTGAGAG
TTGTCTCCATAGCCTTGAGAATTAGACGTAGAAAGCTGAGGCCCACGTGCTGTCTCCAAC
AGGCCCTTGGCGTATGTGAGGCCCCGGCTAGCCCTCTGCAGTGCTTCCAAACCCCACACC
TCTGAGAACGCGGTCTCCAGGTGAGTCCAGCCTCCTGGGGAGAGAGAGGAATGGGTCTTT
GCTTGCTAAGGTTTGGGAACAAGATGGTCATCCTGCCCACTTCTGTGGACTGTGTCATCC
TACCTCTGCCAGGCACAGTTCCAGGCTCCTCGGGGTCTCCAGTGGTTCCATCAGGAAAAG
GCAGTCTTTTGGACCTATCGTCACTCCTTGCTCTCCCACCCCATCCAGCCCTCCACAGCT
TCTATCTAAGGCTTCATCACATCTGAGCTGCCTGACCTTAAAGATACTCCATGTTCGAGC
AATGGCCAACATTTCTTACTTCACTGTCTCGGCTGTCTCTCCCTCAGATGCCAGCAGCAC
CATGGTCACCTGACCTCACCCTGCCCAGGCTCCCTGTTTTCTAAGCCAGAAATAGCTCTG
ACACCAGAGTCAGGAAATGACATGGGGAGTGTGGGGCGAGAAAGGCAACAGTCTCTCAAG
TGACCCTGACAGTAATCTGGTCCAGGTCACAATGTACTTAAAGCCAGCGCTCGCTGGGTA
GTCATTTATCCATTTGTTCCCATTTGTGAAAATCTGCTGGTGTGCACAGCTGGCCCACCA
CTTCTAATGCGAGGAAGGACCCCAGCACTGTCACAGCCACTGTGGGCAGAGGGGCACTTC
AAGTCAGTAAGTCCCTTGGGGGCCAATTTAATGTCTCCCCTCCCATCCCCCATCAAGTCC
ATCTGGGTGCGCGAAGGGAGGCAATCCAGGAGTCACCTTTTTCTAGCTCTCAGGGCTCTA
GGCCTTGCCTGCTGAAGAAGGAATTGTGAGAGACTCCCTGAGTTCTGGTCCCAACTCTGC
TATCAACAGTCAGTGGATCCCCCGGGAAAATCGCACAGCCCCCACCCTTTGCGATATCAC
TAAACTAGCTGCAAGTAGCCCAATGAAGGGAACTTCGGCACTTATGAACTGTCACCATCA
CAGTGACAGTGACCCTACTCCCACCAGTAGCTACTCTCCTTGAAAAAGACCTTACCTCCC
ACCCTAATGCTACTTCCTTTCCCACAGCAGCCTGCTCCAGAGGACAAGCCTCAGCCTGCA
CCCTCAGCCCTGTGTCAGTCTGTATGTCCCAGCTCTAACTACAGACCACCACCACCACCA
CCACCACCACCACCACCACCACCACCACCACCACCACCACCACCACCACCACCACAGTGG
TTTCTGGCTCCCCCCGGTGATGGGGGGCGGCAGGCCCACGTCCTCTGGAAGCCTTCAGAA
GGGGCTTCGGGCCTTCGCCTCCACCAGAGCCAAGCCAGCTCCCATAGCTCCCACAGCCCA
CAGGGACTGAGAAGAACTGTTGTGGCTCCAAGAAGACATCGGGTAGAAGCTGGGTATAGC
CACACCAACCCCTTGCTAACATTTCTATGAAATCCAAACTTGAGAAGAATAAAGAATGGG
AACATGGAGCATTATTCTAAGGGCTGTGGGCGAGGCGCAGTGACAGGGCACTTTCCTAGC
AAGCAGGAAACCTGGGTTGGATCC

For somatic reprogramming, a double stranded mRNA having a sense strand that encodes Oct4, e.g., where the sense strand has nucleic acid sequences with at least 90% nucleic acid sequence identity to coding sequences in SEQ ID NO:8 or a nucleic acid sequence that encodes a protein with at least 80% amino acid sequence identity to a protein that is encoded by SEQ ID NO:8; Sox 2, e.g., where the sense strand has nucleic acid sequences with at least 90%, 92%, 95%, 97%, 98%, 99% or 100% nucleic acid sequence identity to coding sequences in SEQ ID NO:8 or SEQ ID NO:9 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:9; Klf4, e.g., where the sense strand has nucleic acid sequences with at least 90%, 92%, 95%, 97%, 98%, 99% or 100% nucleic acid sequence identity to coding sequences in SEQ ID NO:10 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:10, c-myc, e.g., where the sense strand has nucleic acid sequences with at least 90% nucleic acid sequence identity to coding sequences in SEQ ID NO:11 or a nucleic acid sequence that encodes a protein with at least 80%, 82%, 85%, 87%, 90%, 92%, 95%, 97%, 98%, 99% or 100% amino acid sequence identity to a protein that is encoded by SEQ ID NO:11, or any combination thereof, may be employed.

(SEQ ID NO: 8)
ATGGGGGGACACCTGGCTTCGGATTTCGCCTTCTCGCCCCCTCCAGGTGGTGGAGGTGATGGGCCA
GGGGGGCCGGAGCCGGGCTGGGTTGATCCTCGGACCTGGCTAAGCTTCCAAGGCCCTCCTGGAGG
GCCAGGAATCGGGCCGGGGGTTGGGCCAGGCTCTGAGGTGTGGGGGATTCCCCCATGCCCCCCGC
CGTATGAGTTCTGTGGGGGGATGGCGTACTGTGGGCCCCAGGTTGGAGTGGGGCTAGTGCCCCAA
GGCGGCTTGGAGACCTCTCAGCCTGAGGGCGAAGCAGGAGTCGGGGTGGAGAGCAACTCCGATGG
GGCCTCCCCGGAGCCCTGCACCGTCACCCCTGGTGCCGTGAAGCTGGAGAAGGAGAAGCTGGAGC
AAAACCCGGAGGAGTCCCAGGACATCAAAGCTCTGCAGAAAGAACTCGAGCAATTTGCCAAGCTCCT
GAAGCAGAAGAGGATCACCCTGGGATATACACAGGCCGATGTGGGGCTCACCCTGGGGGTTCTATT
TGGGAAGGTATTCAGCCAAACGACCATCTGCCGCTTTGAGGCTCTGCAGCTTAGCTTCAAGAACATG
TGTAAGCTGCGGCCCTTGCTGCAGAAGTGGGTGGAGGAAGCTGACAACAATGAAAATCTTCAGGAG
ATATGCAAAGCAGAAACCCTCGTGCAGGCCCGAAAGAGAAAGCGAACCAGTATCGAGAACCGAGTG
AGAGGCAACCTGGAGAATTTGTTCCTGCAGTGCCCGAAACCCACACTGCAGCAGATCAGCCACATC
GCCCAGCAGCTTGGGCTCGAGAAGGATGTGGTCCGAGTGTGGTTCTGTAACCGGCGCCAGAAGGG
CAAGCGATCAAGCAGCGACTATGCACAACGAGAGGATTTTGAGGCTGCTGGGTCTCCTTTCTCAGG
GGGACCAGTGTCCTTTCCTCTGGCCCCAGGGCCCCATTTTGGTACCCCAGGCTATGGGAGCCCTCA
CTTCACTGCACTGTACTCCTCGGTCCCTTTCCCTGAGGGGGAAGCCTTTCCCCCTGTCTCCGTCACC
ACTCTGGGCTCTCCCATGCATTCAAACTGA
(SEQ ID NO: 9)
ATGTACAACATGATGGAGACGGAGCTGAAGCCGCCGGGCCCGCAGCAAACTTCGGGGGGCGGCGG
CGGCAACTCCACCGCGGGGGGGGCCGGGGGCAACCAGAAAAACAGCCCGGACCGCGTCAACGG
CCCATGAATGCCTTCATGGTGTGGTCCCGCGGGCAGCGGCGCAAGATGGCCCAGGAGAACCCCAA
GATGCACAACTCGGAGATCAGCAAGCGCCTGGGCGCCGAGTGGAAACTTTTGTCGGAGACGGAGAA
GCGGCCGTTCATCGACGAGGCTAAGCGGCTGCGAGCGCTGCACATGAAGGAGCACCCGGATTATAA
ATACCGGCCCCGGCGGAAAACCAAGACGCTCATGAAGAAGGATAAGTACACGCTGCCCGGGGGGC
TGCTGGCCCCCGGGGGCAATAGCATGGCGAGGGGGGTCGGGGTGGGCGCCGGCCTGGGGGGGG
GCGTGAACCAGCGCATGGACAGTTACGCGCACATGAACGGCTGGAGCAACGGCAGCTACAGCATGA
TGCAGGACCAGCTGGGCTACCCGCAGCACCCGGGCCTCAATGCGCACGGCGCAGCGCAGATGCAG
CCCATGCACCGCTACGACGTGAGCGCCCTGCAGTACAACTCCATGACCAGCTCGCAGACCTACATG
AACGGCTCGCCCACCTACAGCATGTCCTACTCGCAGCAGGGCACCCCTGGCATGGCTCTTGGCTCC
ATGGGTTCGGTGGTCAAGTCCGAGGCCAGCTCCAGCCCCCCTGTGGTTACCTCTTCCTCCCACTCC
AGGGCGCCCTGCCAGGCCGGGGACCTCCGGGACATGATCAGCATGTATCTCCCCGGCGCCGAGGT
GCCGGAACCCGCCGCCCCCAGCAGACTTCACATGTCCCAGCACTACCAGAGCGGCCCGGTGCCCG
GCACGGCCATTAACGGCACACTGCCCCTCTCACACATGTGA
(SEQ ID NO: 10)
ATGAGGCAGCCACCTGGCGAGTCTGACATGGCTGTCAGCGACGCGCTGCTCCCATCTTTCTCCACG
TTCGCGTCTGGCCCGGGGGAAGGGAGAAGACACTGCGTCAAGCAGGTGCCCCGAATAACCGCTG
GCGGGAGGAGCTCTCCCACATGAAGCGACTTCCCCCAGTGCTTCCCGGCCGCCCCTATGACCTGGC
GGCGGCGACCGTGGCCACAGACCTGGAGAGCGGCGGAGCCGGTGCGGCTTGCGGCGGTAGCAAC
CTGGCGCCCCTACCTCGGAGAGAGACCGAGGAGTTCAACGATCTCCTGGACCTGGACTTTATTCTCT
CCAATTCGCTGACCCATCCTCCGGAGTCAGTGGCCGCCACCGTGTCCTCGTCAGCGTCAGCCTCCT
CTTCGTCGTCGCCGTCGAGCAGCGGCCCTGCCAGCGCGCCCTCCACCTGCAGCTTCACCTATCCGA
TCCGGGCCGGGAACGACCCGGGCGTGGCGCCGGGGGGCACGGGCGGAGGCCTCCTCTATGGCAG
GGAGTCCGCTCCCCCTCCGACGGCTCCCTTCAACCTGGCGGACATCAACGACGTGAGCCCCTCGG
GCGGCTTCGTGGCCGAGCTCCTGCGGCCAGAATTGGACCCGGTGTACATTCCGCCGCAGCAGCCG
CAGCCGCCAGGTGGCGGGCTGATGGGCAAGTTCGTGCTGAAGGCGTCGCTGAGCGCCCCTGGCAG
CGAGTACGGCAGCCCGTCGGTCATCAGCGTCAGCAAAGGCAGCCCTGACGGCAGCCACCCGGTGG
TGGTGGCGCCCTACAACGGGGGCCGCCGCGCACGTGCCCCAAGATCAAGCAGGAGGCGGTCTCT
TCGTGCACCCACTTGGGCGCTGGACCCCCTCTCAGCAATGGCCACCGGCCGGCTGCACACGACTTC
CCCCTGGGGGGGCAGCTCCCCAGCAGGACTACCCCGACCCTGGGTCTTGAGGAAGTGCTGAGCAG
CAGGGACTGTCACCCTGCCCTGCCGCTTCCTCCCGGCTTCCATCCCCACCCGGGGCCCAATTACCC
ATCCTTCCTGCCCGATCAGATGCAGCCGCAAGTCCCGCCGCTCCATTACCAAGAGCTCATGCCACCC
GGTTCCTGCATGCCAGAGGAGCCCAAGCCAAAGAGGGGAAGACGATCGTGGCCCCGGAAAAGGAC
CGCCACCCACACTTGTGATTACGCGGGCTGCGGCAAAACCTACACAAAGAGTTCCCATCTCAAGGCA
CACCTGCGAACCCACACAGGTGAGAAACCTTACCACTGTGACTGGGACGGCTGTGGATGGAAATTC
GCCCGCTCAGATGAACTGACCAGGCACTACCGTAAACACACGGGGCACCGCCCGTTCCAGTGCCAA
AAATGCGACCGAGCATTTTCCAGGTCGGACCACCTCGCCTTACACATGAAGAGGCATTTTTAA
(SEQ ID NO: 11)
CTGGATTTTTTTCGGGTAGTGGAAAACCAGCAGCCTCCCGCGACGATGCCCCTCAACGTTAGCTTCA
CCAACAGGAACTATGACCTCGACTACGACTCGGTGCAGCCGTATTTCTACTGCGACGAGGAGGAGA
ACTTCTACCAGCAGCAGCAGCAGAGCGAGCTGCAGCCCCCGGCGCCCAGCGAGGATATCTGGAAG
AAATTCGAGCTGCTGCCCACCCCGCCCCTGTCCCCTAGCCGCCGCTCCGGGCTCTGCTCGCCCTCC
TACGTTGCGGTCACACCCTTCTCCCTTCGGGGAGACAACGACGGCGGTGGCGGGAGCTTCTCCACG
GCCGACCAGCTGGAGATGGTGACCGAGCTGCTGGGAGGAGACATGGTGAACCAGAGTTTCATCTGC
GACCCGGACGACGAGACCTTCATCAAAAACATCATCATCCAGGACTGTATGTGGAGCGGCTTCTCGG
CCGCCGCCAAGCTCGTCTCAGAGAAGCTGGCCTCCTACCAGGCTGCGCGCAAAGACAGCGGCAGC
CCGAACCCCGCCCGCGGCCACAGCGTCTGCTCCACCTCCAGCTTGTACCTGCAGGATCTGAGCGCC
GCCGCCTCAGAGTGCATCGACCCCTCGGTGGTCTTCCCCTACCCTCTCAACGACAGCAGCTCGCCC
AAGTCCTGCGCCTCGCAAGACTCCAGCGCCTTCTCTCCGTCCTCGGATTCTCTGCTCTCCTCGACGG
AGTCCTCCCCGCAGGGCAGCCCCGAGCCCCTGGTGCTCCATGAGGAGACACCGCCCACCACCAGC
AGCGACTCTGAGGAGGAACAAGAAGATGAGGAAGAAATCGATGTTGTTTCTGTGGAAAAGAGGCAG
GCTCCTGGCAAAAGGTCAGAGTCTGGATCACCTTCTGCTGGAGGCCACAGCAAACCTCCTCACAGC
CCACTGGTCCTCAAGAGGTGCCACGTCTCCACACATCAGCACAACTACGCAGCGCCTCCCTCCACT
CGGAAGGACTATCCTGCTGCCAAGAGGGTCAAGTTGGACAGTGTCAGAGTCCTGAGACAGATCAGC
AACAACCGAAAATGCACCAGCCCCAGGTCCTCGGACACCGAGGAGAATGTCAAGAGGCGAACACAC
AACGTCTTGGAGCGCCAGAGGAGGAACGAGCTAAAACGGAGCTTTTTTGCCCTGCGTGACCAGATC
CCGGAGTTGGAAAACAATGAAAAGGCCCCCAAGGTAGTTATCCTTAAAAAAGCCACAGCATACATCC
TGTCCGTCCAAGCAGAGGAGCAAAAGCTCATTTCTGAAGAGGACTTGTTGCGGAAACGACGAGAACA
GTTGAAACACAAACTTGAACAGCTACGGAACTCTTGTGCGTAA.

In one embodiment, for genome editing, a double stranded mRNA having a sense strand that encodes a nuclease such as Cas9, e.g., where the sense strand has nucleic acid sequences with at least 90%, 92%, 95%, 97%, 98%, 99% or 100% nucleic acid sequence identity to coding sequences in SEQ ID NO:12 or 13 for a nuclease, or a nucleic acid sequence that encodes a protein with at least 80%, 82%, 85%, 87%, 90%, 92%, 95%, 97%, 98%, 99% or 100% amino acid sequence identity to a nuclease that is encoded by SEQ ID NO:12 or 13, may be employed. The double stranded RNA for a nuclease such as Cas9 may be combined with a plurality of distinct lipids or a peptide conjugate and then directly administered.

(SEQ ID NO: 12)
atggataaga aatactcaat aggcttagat atcggcacaa atagcgtcgg atgggcggtg atcactgatg aatataaggt tccgtctaaa
aagttcaagg ttctgggaaa tacagaccgc cacagtatca aaaaaaatct tataggggct cttttatttg acagtggaga gacagcggaa
gcgactcgtc tcaaacggac agctcgtaga aggtatacac gtcggaagaa tcgtatttgt tatctacagg agattttttc aaatgagatg
gcgaaagtag atgatagttt ctttcatcga cttgaagagt cttttttggt ggaagaagac aagaagcatg aacgtcatcc tatttttgga
aatatagtag atgaagttgc ttatcatgag aaatatccaa ctatctatca tctgcgaaaa aaattggtag attctactga taaagcggat
ttgcgcttaa tctatttggc cttagcgcat atgattaagt ttcgtggtca ttttttgatt gagggagatt taaatcctga taatagtgat
gtggacaaac tatttatcca gttggtacaa acctacaatc aattatttga agaaaaccct attaacgcaa gtggagtaga tgctaaagcg
attctttctg cacgattgag taaatcaaga cgattagaaa atctcattgc tcagctcccc ggtgagaaga aaaatggctt atttgggaat
ctcattgctt tgtcattggg tttgacccct aattttaaat caaattttga tttggcagaa gatgctaaat tacagctttc aaaagatact tacgatgatg
atttagataa tttattggcg caaattggag atcaatatgc tgatttgttt ttggcagcta agaatttatc agatgctatt ttactttcag atatcctaag
agtaaatact gaaataacta aggctcccct atcagcttca atgattaaac gctacgatga acatcatcaa gacttgactc ttttaaaagc
tttagttcga caacaacttc cagaaaagta taaagaaatc ttttttgatc aatcaaaaaa cggatatgca ggttatattg atgggggagc
tagccaagaa gaattttata aatttatcaa accaatttta gaaaaaatgg atggtactga ggaattattg gtgaaactaa atcgtgaaga
tttgctgcgc aagcaacgga cctttgacaa cggctctatt ccccatcaaa ttcacttggg tgagctgcat gctattttga gaagacaaga
agacttttat ccatttttaa aagacaatcg tgagaagatt gaaaaaatct tgacttttcg aattccttat tatgttggtc cattggcgcg
tggcaatagt cgttttgcat ggatgactcg gaagtctgaa gaaacaatta ccccatggaa ttttgaagaa gttgtcgata aaggtgcttc
agctcaatca tttattgaac gcatgacaaa ctttgataaa aatcttccaa atgaaaaagt actaccaaaa catagtttgc tttatgagta
ttttacggtt tataacgaat tgacaaaggt caaatatgtt actgaaggaa tgcgaaaacc agcatttctt tcaggtgaac agaagaaagc
cattgttgat ttactcttca aaacaaatcg aaaagtaacc gttaagcaat taaaagaaga ttatttcaaa aaaatagaat gttttgatag
tcttgaaatt tcaggagttg aagatagatt taatgcttca ttaggtacct accatgattt gctaaaaatt attaaagata aagatttttt
ggataatgaa gaaaatgaag atatcttaga ggatattgtt ttaacattga ccttatttga agatagggag atgattgagg aaagacttaa
aacatatgct cacctctttg atgataaggt gatgaaacag cttaaacgtc gccgttatac tggttgggga cgtttgtctc gaaaattgat
taatggtatt agggataagc aatctggcaa aacaatatta gattttttga aatcagatgg ttttgccaat cgcaatttta tgcagctgat
ccatgatgat agtttgacat ttaaagaaga cattcaaaaa gcacaagtgt ctggacaagg cgatagttta catgaacata ttgcaaattt
agctggtagc cctgctatta aaaaaggtat tttacagact gtaaaagttg ttgatgaatt ggtcaaagta atggggcggc ataagccaga
aaatatcgtt attgaaatgg cacgtgaaaa tcagacaact caaaagggcc agaaaaattc gcgagagcgt atgaaacgaa
tcgaagaagg tatcaaagaa ttaggaagtc agattcttaa agagcatcct gttgaaaata ctcaattgca aaatgaaaag ctctatctct
attatctcca aaatggaaga gacatgtatg tggaccaaga attagatatt aatcgtttaa gtgattatga tgtcgatcac attgttccac
aaagtttcct taaagacgat tcaatagaca ataaggtctt aacgcgttct gataaaaatc gtggtaaatc ggataacgtt ccaagtgaag
aagtagtcaa aaagatgaaa aactattgga gacaacttct aaacgccaag ttaatcactc aacgtaagtt tgataattta
acgaaagctg aacgtggagg tttgagtgaa cttgataaag ctggttttat caaacgccaa ttggttgaaa ctcgccaaat cactaagcat
gtggcacaaa ttttggatag tcgcatgaat actaaatacg atgaaaatga taaacttatt cgagaggtta aagtgattac cttaaaatct
aaattagttt ctgacttccg aaaagatttc caattctata aagtacgtga gattaacaat taccatcatg cccatgatgc gtatctaaat
gccgtcgttg gaactgcttt gattaagaaa tatccaaaac ttgaatcgga gtttgtctat ggtgattata aagtttatga tgttcgtaaa
atgattgcta agtctgagca agaaataggc aaagcaaccg caaaatattt cttttactct aatatcatga acttcttcaa aacagaaatt
acacttgcaa atggagagat tcgcaaacgc cctctaatcg aaactaatgg ggaaactgga gaaattgtct gggataaagg gcgagatttt
gccacagtgc gcaaagtatt gtccatgccc caagtcaata ttgtcaagaa aacagaagta cagacaggcg gattctccaa ggagtcaatt
ttaccaaaaa gaaattcgga caagcttatt gctcgtaaaa aagactggga tccaaaaaaa tatggtggtt ttgatagtcc aacggtagct
tattcagtcc tagtggttgc taaggtggaa aaagggaaat cgaagaagtt aaaatccgtt aaagagttac tagggatcac aattatggaa
agaagttcct ttgaaaaaaa tccgattgac tttttagaag ctaaaggata taaggaagtt aaaaaagact taatcattaa actacctaaa
tatagtcttt ttgagttaga aaacggtcgt aaacggatgc tggctagtgc cggagaatta caaaaaggaa atgagctggc tctgccaagc
aaatatgtga attttttata tttagctagt cattatgaaa agttgaaggg tagtccagaa gataacgaac aaaaacaatt gtttgtggag
cagcataagc attatttaga tgagattatt gagcaaatca gtgaattttc taagcgtgtt attttagcag atgccaattt agataaagtt
cttagtgcat ataacaaaca tagagacaaa ccaatacgtg aacaagcaga aaatattatt catttattta cgttgacgaa tcttggagct
cccgctgctt ttaaatattt tgatacaaca attgatcgta aacgatatac gtctacaaaa gaagttttag atgccactct tatccatcaa
tccatcactg gtctttatga aacacgcatt gatttgagtc agctaggagg tgactga
(SEQ ID NO: 13)
ATGGATAAGAAATACTCAATAGGCTTAGATATCGGCACAAATAGCGTCGGATGGGCGGTGATCACTG
ATGAATATAAGGTTCCGTCTAAAAAGTTCAAGGTTCTGGGAAATACAGACCGCCACAGTATCAAAAAA
AATCTTATAGGGGCTCTTTTATTTGACAGTGGAGAGACAGCGGAAGCGACTCGTCTCAAACGGACAG
CTCGTAGAAGGTATACACGTCGGAAGAATCGTATTTGTTATCTACAGGAGATTTTTTCAAATGAGATG
GCGAAAGTAGATGATAGTTTCTTTCATCGACTTGAAGAGTCTTTTTTGGTGGAAGAAGACAAGAAGCT
GAACGTCATCCTATTTTTGGAAATATAGTAGATGAAGTTGCTTATCATGAGAAATATCCAACTATCTAC
ATCTGCGAAAAAAATTGGTAGATTCTACTGATAAAGCGGATTTGCGCTTAATCTATTTGGCCTTAGCG
CATATGATTAAGTTTCGTGGTCATTTTTTGATTGAGGGAGATTTAAATCCTGATAATAGTGATGTGGAC
AAACTATTTATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTCATTGCTCA
GCTCCCCGGTGAGAAGAAAAATGGCTTATTTGGGAATCTCATTGCTTTGTCATTGGGTTTGACCCCTA
ATTTTAAATCAAATTTTGATTTGGCAGAAGATGCTAAATTACAGCTTTCAAAAGATACTTACGATGATG
ATTTAGATAATTTATTGGCGCAAATTGGAGATCAATATGCTGATTTGTTTTTGGCAGCTAAGAATTTAT
CAGATGCTATTTTACTTTCAGATATCCTAAGAGTAAATACTGAAATAACTAAGGCTCCCCTATCAGCTT
CAATGATTAAACGCTACGATGAACATCATCAAGACTTGACTCTTTTAAAAGCTTTAGTTCGACAACAAC
TTCCAGAAAAGTATAAAGAAATCTTTTTTGATCAATCAAAAAACGGATATGCAGGTTATATTGATGGGG
GAGCTAGCCAAGAAGAATTTTATAAATTTATCAAACCAATTTTAGAAAAAATGGATGGTACTGAGGAAT
TATTGGTGAAACTAAATCGTGAAGATTTGCTGCGCAAGCAACGGACCTTTGACAACGGCTCTATTCCC
CATCAAATTCACTTGGGTGAGCTGCATGCTATTTTGAGAAGACAAGAAGACTTTTATCCATTTTTAAAA
GACAATCGTGAGAAGATTGAAAAAATCTTGACTTTTCGAATTCCTTATTATGTTGGTCCATTGGCGCGT
GGCAATAGTCGTTTTGCATGGATGACTCGGAAGTCTGAAGAAACAATTACCCCATGGAATTTTGAAGA
AGTTGTCGATAAAGGTGCTTCAGCTCAATCATTTATTGAACGCATGACAAACTTTGATAAAAATCTTCC
AAATGAAAAAGTACTACCAAAACATAGTTTGCTTTATGAGTATTTTACGGTTTATAACGAATTGACAAA
GGTCAAATATGTTACTGAAGGAATGCGAAAACCAGCATTTCTTTCAGGTGAACAGAAGAAAGCCATTG
TTGATTTACTCTTCAAAACAAATCGAAAAGTAACCGTTAAGCAATTAAAAGAAGATTATTTCAAAAAAAT
AGAATGTTTTGATAGTGTTGAAATTTCAGGAGTTGAAGATAGATTTAATGCTTCATTAGGTACCTACCA
TGATTTGCTAAAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACATATGCTCAC
CTCTTTGATGATAAGGTGATGAAACAGCTTAAACGTCGCCGTTATACTGGTTGGGGACGTTTGTCTCG
AAAATTGATTAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGACATTCAAAA
AGCACAAGTGTCTGGACAAGGCGATAGTTTACATGAACATATTGCAAATTTAGCTGGTAGCCCTGCTA
TTAAAAAAGGTATTTTACAGACTGTAAAAGTTGTTGATGAATTGGTCAAAGTAATGGGGGGGCATAAG
CCAGAAAATATCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTAAAGAGCATCC
TGTTGAAAATACTCAATTGCAAAATGAAAAGCTCTATCTCTATTATCTCCAAAATGGAAGAGACATGTA
TGTGGACCAAGAATTAGATATTAATCGTTTAAGTGATTATGATGTCGATCACATTGTTCCACAAAGTTT
CCTTAAAGACGATTCAATAGACAATAAGGTCTTAACGCGTTCTGATAAAAATCGTGGTAAATCGGATA
ACGTTCCAAGTGAAGAAGTAGTCAAAAAGATGAAAAACTATTGGAGACAACTTCTAAACGCCAAGTTA
ATCACTCAACGTAAGTTTGATAATTTAACGAAAGCTGAACGTGGAGGTTTGAGTGAACTTGATAAAGC
TGGTTTTATCAAACGCCAATTGGTTGAAACTCGCCAAATCACTAAGCATGTGGCACAAATTTTGGATA
GTCGCATGAATACTAAATACGATGAAAATGATAAACTTATTCGAGAGGTTAAAGTGATTACCTTAAAAT
CTAAATTAGTTTCTGACTTCCGAAAAGATTTCCAATTCTATAAAGTACGTGAGATTAACAATTACCATC
ATGCCCATGATGCGTATCTAAATGCCGTCGTTGGAACTGCTTTGATTAAGAAATATCCAAAACTTGAA
TCGGAGTTTGTCTATGGTGATTATAAAGTTTATGATGTTCGTAAAATGATTGCTAAGTCTGAGCAAGAA
ATAGGCAAAGCAACCGCAAAATATTTCTTTTACTCTAATATCATGAACTTCTTCAAAACAGAAATTACA
CTTGCAAATGGAGAGATTCGCAAACGCCCTCTAATCGAAACTAATGGGGAAACTGGAGAAATTGTCT
GGGATAAAGGGCGAGATTTTGCCACAGTGCGCAAAGTATTGTCCATGCCCCAAGTCAATATTGTCAA
GAAAACAGAAGTACAGACAGGCGGATTCTCCAAGGAGTCAATTTTACCAAAAAGAAATTCGGACAAG
CTTATTGCTCGTAAAAAAGACTGGGATCCAAAAAAATATGGTGGTTTTGATAGTCCAACGGTAGCTTA
TTCAGTCCTAGTGGTTGCTAAGGTGGAAAAAGGGAAATCGAAGAAGTTAAAATCCGTTAAAGAGTTAC
TAGGGATCACAATTATGGAAAGAAGTTCCTTTGAAAAAAATCCGATTGACTTTTTAGAAGCTAAAGGAT
ATAAGGAAGTTAAAAAAGACTTAATCATTAAACTACCTAAATATAGTCTTTTTGAGTTAGAAAACGGTC
GTAAACGGATGCTGGCTAGTGCCGGAGAATTACAAAAAGGAAATGAGCTGGCTCTGCCAAGCAAATA
TGTGAATTTTTTATATTTAGCTAGTCATTATGAAAAGTTGAAGGGTAGTCCAGAAGATAACGAACAAAA
ACAATTGTTTGTGGAGCAGCATAAGCATTATTTAGATGAGATTATTGAGCAAATCAGTGAATTTTCTAA
GCGTGTTATTTTAGCAGATGCCAATTTAGATAAAGTTCTTAGTGCATATAACAAACATAGAGACAAACC
AATACGTGAACAAGCAGAAAATATTATTCATTTATTTACGTTGACGAATCTTGGAGCTCCCGCTGCTTT
TAAATATTTTGATACAACAATTGATCGTAAACGATATACGTCTACAAAAGAAGTTTTAGATGCCACTCT
ATCCATCAATCCATCACTGGTCTTTATGAAACACGCATTGATTTGAGTCAGCTAGGAGGTGACTGA.

Exemplary nucleases include but are not limited to those having SEQ ID NO:14 or 15, or a protein with at least 80%, 82%, 85%, 87%, 90%, 92%, 95%, 97%, 98%, 99% or 100% amino acid sequence identity to a nuclease that is encoded by SEQ ID NO:14 or 15:

mkekyilgld lgitsvgygi infetkkiid agvrlfpean vdnnegrrsk rgsrrlkrrr ihrlervkll lteydlinke qiptsnnpyq irvkglseil skdelaiall hlakrrgihn invssededa snelstkeqi nmnkllkdk yvcevqlqrl kegqirgekn rfkttdilke idqllkvqkd yhnldidfin qykeivetrr eyfegpgqgs pfgwngdlkk wyemlmghct yfpgelrsvk yaysadlfna lndlnnliiq rdnsekleyh ekyhiienvf kqkkkptlkq iakeigvnpe dikgyritks gtpqftefkl yhdlksivfd ksileneail dqiaeiltiy qdeqsikeel nklpeilneq dkaeiaklig yngthrlslk cihlineelw qtsmqmeif nylnikpnkv dlseqnkipk dmvndfilsp vvkrtfiqsi nvinkvieky gipediilel arennsddrk kfinnlqkkn eatrkrinei igqtgnqnak rivekirlhd qqegkclysl esialmdlln npqnyevdhi iprsvafdns ihnkvlvkqi enskkgnrtp yqylnssdak lsynqfkqhi lnlskskdri skkkkdylle erdinkfevq kefinrnivd tryatrelts ylkayfsann mdvkvkting sftnhlrkvw rfdkyrnhgy khhaedalii anadflfken kklqnankil ekptienntk kvtvekeedy nnvfetpklv edikqyrdyk fshrvdkkpn rqlindtlys trmkdehdyi vqtitdiygk dntnlkkqfn knpekflmyq ndpktfekls iimkqysdek kplakyyeet geyttkyskk nngpivkkik llgnkvgnhl dvtnkyenst kklvklsikn yrfdvyltek gykfvtiayl nvfkkdnyyy ipkdkyqelk ekkkikdtdq flasfykndl ikingdlyki igvnsddrni ieldyydiky kdyceinnik geprikktig kktesiekft tdvlgnlylh stekapqlif krgl (SEQ ID NO:14)

mnkpysigld igtnsvgwsi itddykvpak kmrvlgntdk eyikknliga llfdggntas drrlkrtarr rytrrmril ylqeifaeem skvddsffhr ledsflvedd krgskypifa tmqeekdyhe kfptiyhlrk eladkkekad lrlfylalah iikfrghfli eddsfdvrnt diqrqyqafl eifdttfenn hllsqnidve gittdkisks akkdrilaqy pngkstgifa eflklivgnq adfkkhfnle dktplqfakd sydedlenll gqigdefadl fsvakklyds vllsgiltvt dlstkaplsa smiqrydehr edlkqlkqfv kasipekyqe iftdsskdgy agyiegktnq gafykyiskl ftkqegseyf lekiknedfl rkqrtfdngs iphqvhltel kaiirrqsey ypflkenldr iekiltfrip yyvgplarek sdfawmtrkt ddsirpwnfe elvdkeasae afihrmtnnd lylpeekvlp khsliyekft vyneltkvry kneggetyff dsnikqeifd gvfkehrkvs kkklldflak eyeefrivdv igldkenkaf naslgtyhdl kkildkdfld npdnesiled ivqtltlfed remikkrlen ykdlftesql kklyrrhytg wgrlsaklin girdkesqkt ildyliddgk snrnfmqlih ddglsfksii skaqagshsd nikevvgela gspaikkgil qslkivdelv kvmgyepeqi vvemarenqt tnqgrrnsrq rykllddgvk nlasdingni lkeyptdnqa lqnerlflyy lqngrdmytg kaldidnlsq ydidhiipqa fikddsidnr vlvssaknrg ksddvpslei vkdckvfwkk lldaklmsqr kydnltkaer ggltsddkar fiqrqlvetr qitkhvaril derfnnelds kgrrirkvki vtlksnlvsn frkefgfyki revnnyhhah daylnavvak ailtkypqle pefvygdypk ynsyktrksa teklffysni mnffktkvtl adgtvvvkdd ievnndtgei vwdkkkhfat vrkvlsypqv nivkkteiqt ggfskesila hgnsdklipr ktkdiyldpk kyggfdspiv aysvlvvadi kkgkaqklkt vtellgitim ersrfeknps afleskgyln irddklmilp kyslfeleng rrrllasage lqkgnelalp tqfmkflyla srynelkgkp eeieqkqefv vqhvsyfddi lqiindfsnr viladanlek inklyqdnke nisvdelann iinlftftsl gapaafkffd kivdrkryts tkevinstli hqsitglyet ridlgklged (SEQ ID NO:15).

Thus, in one embodiment, the ds mRNA encodes a nuclease such as a Cas9 protein e.g., one having SEQ ID NO:14 or 15, or a protein with at least 80%, 82%, 85%, 87%, 90%, 92%, 95%, 97%, 98%, 99% or 100% amino acid sequence identity to a nuclease that is encoded by SEQ ID NO:14 or 15.

### Exemplary Embodiments

The disclosure provides a composition comprising an amount of double stranded (ds) mRNA encoding a therapeutic or prophylactic gene product, e.g., of a pathogen, wherein at least one strand of the ds mRNA has a 5′ cap, a start codon, a polyA sequence and encodes the protein, wherein the two strands of the ds mRNA are hydrogen bonded over at least 10 nucleotides and a compound comprising a ligand that binds to CD209 linked to a synthetic polymer, e.g., polyethylene glycol (PEG), linked to a peptide comprising at least two amino acids at least one of which is modified with an acridine linked to a molecule that facilitates endosomal escape. In one embodiment, the gene product is an antibody or a fragment thereof. In one embodiment, the ligand that binds to CD209 comprises mannose. In one embodiment, the synthetic polymer has a molecular weight of about 1 kDa to about 10 kDa or about 4 kDa to about 7 kDa. In one embodiment, the synthetic polymer comprises (OCH₂CH₂)n where n is from about 5 to about 150, 10 to 50, 50 to 100, or 100 to 150. In one embodiment, the molecule that facilitates endosomal escape comprises a peptide. In one embodiment, the peptide is mellitin. In one embodiment, the at least one strand of the ds mRNA encodes a viral protein. In one embodiment, at least one strand of the ds mRNA includes one or more non-natural nucleotides. In one embodiment, at least one of the non-natural nucleotides has a non-natural sugar. In one embodiment, at least one of the non-natural nucleotides has a non-natural nucleobase. In one embodiment, at least one strand of the ds mRNA includes at least one non-phosphodiester bond. In one embodiment, the ds mRNA includes 5-formyl cytidine or pseudouridine. In one embodiment, at least 5% of the nucleotides are non-natural nucleotides. In one embodiment, the non-natural nucleotide analog is a purine analog. In one embodiment, the strands of the ds mRNA are hydrogen bonded over at least 90% of the length of the strands. In one embodiment, the strands of the ds mRNA are hydrogen bonded over the entire length of the strands. In one embodiment, one of the strands of the ds mRNA is no more than 5 kb in length. In one embodiment, one of the strands of the ds mRNA is greater than 5 kb in length, e.g., from about 5 kb to about 10 kb, about 10 kb to about 20 kb, about 20 kb to about 30 kb, about 30 kb to about 40 kb, about 40 kb to about 50 kb, or more. In one embodiment, only one of the two strands of the ds mRNA includes one or more non-natural nucleotides. In one embodiment, both of the strands of the ds mRNA include one or more non-natural nucleotides. In one embodiment, at least one strand of the ds mRNA has two or more different non-natural nucleotides. In one embodiment, the composition comprises nanoparticles having a diameter of about 100 nm to about 200 nm. In one embodiment, the RNA is a circular RNA. In one embodiment, the RNA is a self-amplifying RNA

Also provided is a method of expressing a therapeutic or prophylactic gene product, comprising: providing the composition disclosed herein; and introducing the composition to cells in an amount effective to express the gene product. In one embodiment, the cells are in a mammal. In one embodiment, the composition is systemically administered to the mammal. In one embodiment, the composition is locally administered to the mammal. In one embodiment, the gene product is a viral protein. In one embodiment, the viral protein is influenza HA In one embodiment, the viral protein is coronavirus spike. In one embodiment, the gene product is a bacterial protein. In one embodiment, the mammal is a human, bovine, equine, swine, caprine, feline or canine. In one embodiment, the synthetic polymer comprises PEG. In one embodiment, the gene product is a cancer antigen.

### Exemplary Embodiments

In one embodiment, the disclosure provides a composition comprising an amount of double stranded (ds) mRNA encoding a therapeutic or prophylactic gene product, wherein at least one strand of the ds mRNA has a 5′ cap, a start codon, a polyA sequence and encodes the protein, wherein the two strands of the ds mRNA are hydrogen bonded over at least 10 nucleotides, and an amount of a plurality of distinct lipids. In one embodiment, the at least one strand of the ds mRNA encodes a viral protein. In one embodiment, the at least one strand of the ds mRNA encodes a coronavirus spike protein or an antigenic portion thereof including the receptor binding domain. In one embodiment, the at least one strand of the ds mRNA encodes a bacterial protein. In one embodiment, the at least one strand of the ds mRNA encodes a cancer antigen. In one embodiment, at least one strand of the ds mRNA includes one or more non-natural nucleotides. In one embodiment, at least one of the non-natural nucleotides has a non-natural sugar. In one embodiment, at least one of the non-natural nucleotides has a non-natural nucleobase. In one embodiment, at least one strand of the ds mRNA includes at least one non-phosphodiester bond. In one embodiment, the ds mRNA includes 5-formyl cytidine or pseudouridine. In one embodiment, at least 5% of the nucleotides are non-natural nucleotides. In one embodiment, the non-natural nucleotide analog is a purine analog. In one embodiment, the strands of the ds mRNA are hydrogen bonded over at least 90% of the length of the strands. In one embodiment, the strands of the ds mRNA are hydrogen bonded over the entire length of the strands. In one embodiment, one of the strands of the ds mRNA is no more than 5 or 10 kb in length. In one embodiment, only one of the two strands of the ds mRNA includes one or more non-natural nucleotides. In one embodiment, both of the strands of the ds mRNA include one or more non-natural nucleotides. In one embodiment, at least one strand of the ds mRNA has two or more different non-natural nucleotides. In one embodiment, the lipid particles have a diameter of about 50 nm to 500 nm.

Further provided is a method of expressing a therapeutic or prophylactic gene product, comprising introducing the composition to mammalian cells in an amount effective to express the gene product. In one embodiment, the cells are in a mammal. In one embodiment, the composition is systemically administered to the mammal. In one embodiment, the composition is locally administered to the mammal. In one embodiment, the gene product is a viral protein, e. g., an influenza HA or a coronavirus spike protein. In one embodiment, the gene product is a bacterial protein. In one embodiment, the gene product is a cancer antigen. In one embodiment, the mammal is a human, bovine, equine, swine, caprine, feline or canine.

The disclosure also provided a composition comprising an amount of double stranded (ds) mRNA encoding a therapeutic or prophylactic gene product, wherein at least one strand of the ds mRNA has a 5′ cap, a start codon, a polyA sequence and encodes the protein, wherein the two strands of the ds mRNA are hydrogen bonded over at least 10 nucleotides, and an amount of a compound comprising a ligand that binds to CD209 linked to a synthetic polymer peptide conjugate linked to a molecule that facilitates endosomal escape, wherein the peptide comprises at least two amino acids at least one of which is modified with an acridine. In one embodiment, the ligand that binds to CD209 comprises mannose. In one embodiment, the synthetic polymer has a molecular weight of about 1 kDa to about 10 kDa or about 4 kDa to about 7 kDa. In one embodiment, the synthetic polymer comprises (OCH₂CH₂)n where n is from about 5 to about 150. In one embodiment, the molecule that facilitates endosomal escape comprises a peptide. In one embodiment, the peptide is mellitin. In one embodiment, the at least one strand of the ds mRNA encodes a viral protein. In one embodiment, at least one strand of the ds mRNA includes one or more non-natural nucleotides. In one embodiment, at least one of the non-natural nucleotides has a non-natural sugar. In one embodiment, at least one of the non-natural nucleotides has a non-natural nucleobase. In one embodiment, at least one strand of the ds mRNA includes at least one non-phosphodiester bond. In one embodiment, one of the strands includes 5-formyl cytidine or pseudouridine. In one embodiment, at least 5% of the nucleotides are non-natural nucleotides. In one embodiment, the non-natural nucleotide analog is a purine analog. In one embodiment, the strands of the ds mRNA are hydrogen bonded over at least 90% of the length of the strands. In one embodiment, the strands of the ds mRNA are hydrogen bonded over the entire length of the strands. In one embodiment, one of the strands of the ds mRNA is no more than 5 or 10 kb in length. In one embodiment, only one of the two strands of the ds mRNA includes one or more non-natural nucleotides. In one embodiment, both of the strands of the ds mRNA include one or more non-natural nucleotides. In one embodiment, at least one strand of the ds mRNA has two or more different non-natural nucleotides. In one embodiment, the composition comprises nanoparticles having a diameter of about 100 nm to about 200 nm comprising the ds mRNA and the compound.

Also provided is a method of expressing a therapeutic or prophylactic gene product, comprising: introducing the composition having the peptide conjugate to mammalian cells in an amount effective to express the gene product. In one embodiment, the cells are in a mammal. In one embodiment, the composition is systemically administered to the mammal. In one embodiment, the composition is locally administered to the mammal. In one embodiment, the gene product is a viral protein. In one embodiment, the viral protein is influenza HA In one embodiment, the viral protein is a coronavirus spike protein. In one embodiment, the gene product is a bacterial protein. In one embodiment, the mammal is a human, bovine, equine, swine, caprine, feline or canine. In one embodiment, the synthetic polymer comprises PEG. In one embodiment, the gene product is a cancer antigen.

The invention will be described by the following non-limiting examples.

### Example I

The efficacy of single stranded and double stranded mRNA is tested by intramuscular electroporation mediated gene delivery in mice. Three types of ssmRNA and dsmRNA are prepared, which include 1) light chain IgG, 2) heavy chain IgG and 3) hemagglutinin. Each ssmRNA and dsmRNA is synthesized from plasmid DNA templates using in vitro transcription (IVT), followed by enzymatic capping and purification on a Qiagen mRNA membrane. Each ss and ds mRNA is quantified by absorbance and characterized for purity and molecular weight by agarose gel electrophoresis.

In particular, the plasmid contains a forward and reverse T7 promoter, a 5′ and 3′ UTR (untranslated region from human b-globin mRNA) flanking the transgene, and a 80A (poly A tail) sequence following the 3′UTR. In addition, a forward (Bsm BI) and reverse (Bsa I) restriction site were inserted just upstream of the 5′ Cap or downstream of the 3′ 80A tail to allow linearization of plasmid prior to IVT. The reverse strand contains a 17 base 3′ overhang to allow efficient T7 IVT and a 3 base 5′ overhang which is necessary with Bsa I restriction.
20 μg of each plasmid is used to prepare the following mRNAs.
200 μg of HA ss mRNA
400 μg of HA ds mRNA (ds composed of 200 μg of forward mRNA and 200 μg of reverse mRNA) 400 μg of ss light chain IgG mRNA
400 μg of ss heavy chain IgG mRNA
800 μg of ds light chain IgG mRNA (composed of 400 μg of forward and 400 μg of reverse mRNA) 800 μg of ds heavy chain IgG mRNA (composed of 400 μg of forward and 400 μg of reverse mRNA).

### Example II

The packaging of mRNA encoding a viral protein and delivery to the skin results in immunization which gives individuals the ability to fight off subsequent infection by the virus and stops the spread of disease in the community. Some of the current vaccines include a lipid nanoparticle (LNP) single stranded mRNA vaccine for COVID-19. In order for a vaccine to be effective and durable it will likely need to invoke both a humoral and cellular immunity response. The substitution of LNP single stranded mRNA with double stranded mRNA likely results in more durable humoral and cellular immunity. Increased metabolic stability of ds mRNA likely results in longer expression and an increased level and durability of the immune response. As described below, the lipid portion of the LNP may be substituted with a synthetic peptide-conjugate to package double stranded mRNA into stable nanoparticles that target and transfect dendritic cells. Dendritic cell targeting with peptide-conjugate double stranded COVID-19 mRNA nanoparticles may enhance the humoral and cellular immune response in mice. The use of a peptide-conjugate double stranded mRNA nanoparticles provides an alternative to single stranded mRNA LNP which may produce more durable immunity.

### Example III

DNA binding peptides, PEG-peptides and glycopeptides have been employed for gene delivery. For example, a synthetic peptide of 18 lysine residues or longer was able to package and delivery DNA in vitro (Wadhwa et al., 1997). Gene delivery N-linked glycopeptides (Collard et al., 2000a) and PEG-peptides (Kwok et al., 1999) have been described and employed in vivo (Collard et al., 2000b).

Sulfhydryl cross/inking peptides that cage DNA and undergo triggered intracellular release have been described (see, e.g., sulfhydryl cross-linking peptides (McKenzie et al., 2000; McKenzie et al., 2000; Chen et al., 2006) and a sulfhydryl releasable melittin peptide that mediates potent in vitro gene delivery (Chen et al., 2007; Baumhover et al., 2010)). A poly-melittin prepared by disulfide bond crosslinking has been reported (Chen et al., 2006).

Polyintercalating PEG-peptides stabilize DNA in the circulation. To increase DNA binding affinity polyacridine peptides were developed (Fernandez et al., 2010; Fernandez et al., 2011; Kizzire et al., 2013 Khargharia et al., 20133). A chemical method to control sulfhydryl crosslinking by iterative reducible ligation has been reported (Ericson & Rice, 2012; Ericson & Rice 2013). Representative publications are included.

Double stranded mRNA nanoparticles contain mRNA that is much more metabolically stable and retains transfection activity (Crowley et al., 2015; Poliskey et al., 2018). Circulatory stable PEG-PolyAcr DNA nanoparticles were used to investigate uptake by the scavenger receptor on Kupffer cells. Heat shrinking may be used to control the particle size of DNA nanoparticles (Crowley & Rice, 2015; Matthew et al., 2020). Kupffer cell binding in the liver can be avoided (Khargharia et al., 2014; Baumhover et al., 2015; Allen et al., 2018; Matthew et al., 2020).

### Overview

Peptide-conjugate double stranded mRNA nanoparticles are a rapid deployment vaccine platform that outperforms LNPs. The disclosed peptide-conjugate gene delivery system is composed of a short amino acid synthetic polyacridine peptide (PAcr) that binds with high affinity to double stranded mRNA PAcr is precisely conjugated through reversible disulfide bond to an endosomal lytic melittin peptide analogue (Mel) that boosts mRNA release into the cytosol (Baumhover et al., 2010). The peptide-conjugate is further modified with a polyethylene glycol (PEG) linked to a high-mannose N-linked glycan (Man9) targeting ligand that binds to DC-SIGN, a cell surface lectin on dendritic cells, to direct receptor mediated endocytosis. These nanoparticles are prepared by mixing of ds mRNA and the peptide-conjugate. The RNAse resistant nanoparticles are stable during long term storage, may be freeze dried and reconstitution, and produce small, stable, particle sizes when prepared in saline at high concentrations needed for i.m. or intra-dermal injection. The peptide-conjugate mRNA vaccine achieves a greater T-cell response by targeting mRNA nanoparticles to dendritic cells to increase T-cell priming. The efficacy of COVID-19 double stranded mRNA delivered as a peptide-conjugate or LNP vaccine is determined. Ligand targeted peptide-conjugates are used to steer the T-cell response into a longer lasting immunity. Specifically, the magnitude and duration of the B-cell and T-cell response for single stranded and double stranded (COVID spike) mRNA delivered as either a lipid nanoparticle, peptide-conjugate nanoparticle or by electroporation are determined. B-cell and T-cell responses are measured for targeted peptide-conjugate double stranded mRNA nanoparticles.

Exemplary Viral Antigen Encoding ds mRNA

The leading SARS-CoV-2 mRNA vaccine (mRNA-1273, encoding S protein) developed by Moderna is in Phase 3 Clinical Trial as of July 2020 (ClinicalTrials.gov Identifier: NCT04283461). The major advantage of this vaccine platform over others is speed. It took only 42 days for Moderna to generate the vaccine for Phase 1 Clinical Trial testing after receiving the DNA sequence (Wu et al., 2020). The Moderna mRNA vaccine relies upon a lipid nanoparticle (LNPs) to achieve cellular delivery of S protein mRNA (Hassett et al., 2019). LNPs are the most widely used in vivo mRNA delivery system at present (Semple et al., 2010). mRNA-LNPs were first demonstrated as efficient delivery systems for mRNA in mice 2015 (Pardi et al., 2015). Since then, multiple vaccine studies have resulted in durable, protective immune responses against multiple infectious pathogens, often after a single dose (Awasthi et al., 2019; VanBlargan et al., 2018). The LNP delivery system used in the COVID-19 clinical trial is a precise blend of four lipids mixed with single stranded mRNA in a microfluidic mixer (Hassett et al., 2019). Both the lipid structure and composition dramatically influences the IgG titer following i.m. dosing in mice (Hassett et al., 2019). Double stranded mRNA may enhance the potency and efficacy of LNP-mRNA, however, it is not certain if B-cell immunity is sufficient or if T-cell immunity is also necessary to achieve durable immunity (Corey et al., 2020).

Peptide-conjugate delivery vehicle. An exemplary peptide-conjugate mRNA vaccine delivery platform described herein includes 1) double stranded mRNA, 2) a peptide-conjugate, and 3) a Man9 N-glycan to target dendritic cells. The double stranded mRNA is significantly more metabolically stable compared to single stranded mRNA (Poliskey et al., 2018). The RNA duplex results in a self-adjuvant effect to boost immunogenicity (Uchida et al., 2018). The peptide-conjugate forms small stable nanoparticles that release mRNA intracellularly (Poliskey et al., 2018; Crowley et al., 2015; Fernandez et al., 2011). The Man9 targeting ligand directs increased transfection of dendritic cells, e.g., in the dermis, which could influence the quality of the immune response (Gao et al., 2020; Perche e al., 2011).

mRNA Vaccines

mRNA vaccines have many advantages over traditional vaccines that have been developed (Pardi et al., 2018). Some of these include rapid R&D and production, simultaneous vaccination with multiple immunogens and a high margin of safety (Pardi et al., 2018). One of the most important features is the ability to rapidly generate an mRNA vaccine by substituting new mRNA into an existing delivery vehicle (Gomez-Aguado et al., 2020). This greatly reduces the time normally needed to generate and formulate a viral protein or to develop an attenuated virus, two strategies most often used for traditional vaccine development (L, et al., 2020).

While there are many types of mRNA vaccines, they all use single stranded mRNA to encode an immunogenic viral protein that is packaged, delivered and expressed in muscle or dermis, leading to a B-cell immune response against the virus (Pardi et al., 2020). Here we propose the following mechanism for peptide-conjugate mRNA vaccines described in this proposal. Following intra-dermal administration, mRNA nanoparticles are likely taken by fibroblasts and dendritic cells of the dermis (Selmi et al., 2016; Diken et al., 2011). This occurs by either receptor mediated endocytosis or pinocytosis (FIG. 5). Targeted delivery of nanoparticles possessing mannose ligands is proposed to facilitate dendritic cell entry via DC-SIGN receptor-mediated endocytosis into endosomes (Gao et al., 2020; Le Moignic et al., 2018; Perche et al., 2011a, Perche et al., 2011b; Pichon et al., 2013). Alternatively, the nanoparticle sheds the polyethylene glycol (PEG) layer at the cell surface by reduction of disulfide bonds, leading to charge unmasking and binding to proteoglycans, followed by pinocytosis (FIG. 5A). Following cellular endocytosis, mRNA nanoparticles are engineered to exit endosomes into the cytosol by reductive release of pour-forming melittin peptides from the nanoparticle, resulting in melittin lysis of endosomes (FIG. 5B). The intracellular release of mRNA results in binding to ribosomes and translation of the mRNA into the programed viral protein (COVID-19, FIGS. 56-C). Viral proteins are proteolyzed by the proteasome and peptide antigens are presented by MHC I on dendritic cell surface to prime for T-cell response (FIG. 5C). Alternatively, transfected fibroblasts secrete viral proteins which are endocytosed by dendritic cells and proteolytically processed, leading to peptide antigen presentation on MHC II to prime the B-cell response (FIG. 5C).

The delivery vehicle likely plays a role in determining the magnitude and type of immune response (L et al., 2020).

### Results

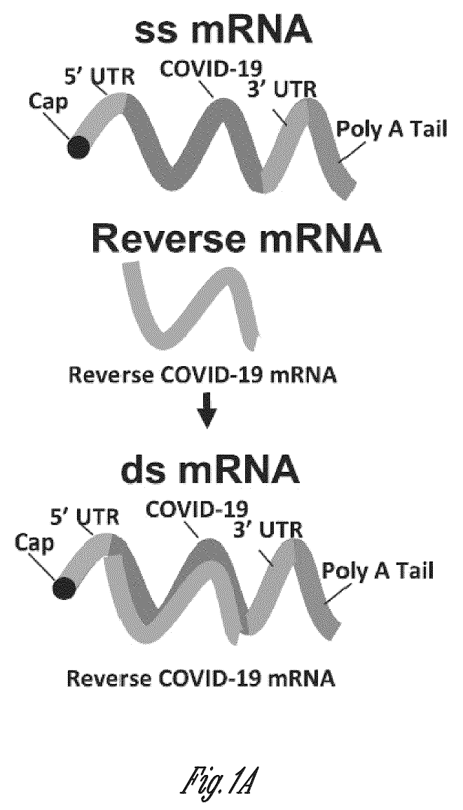

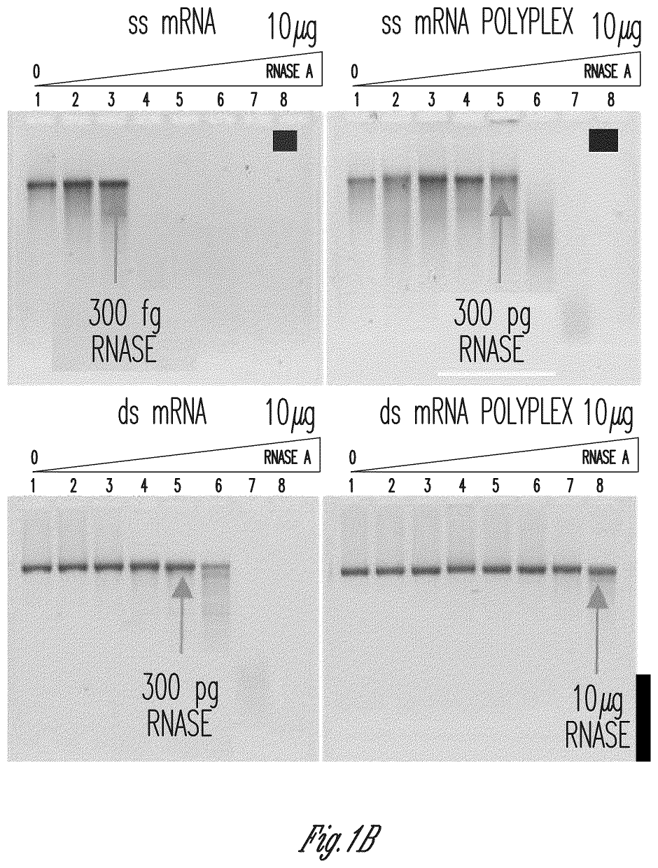

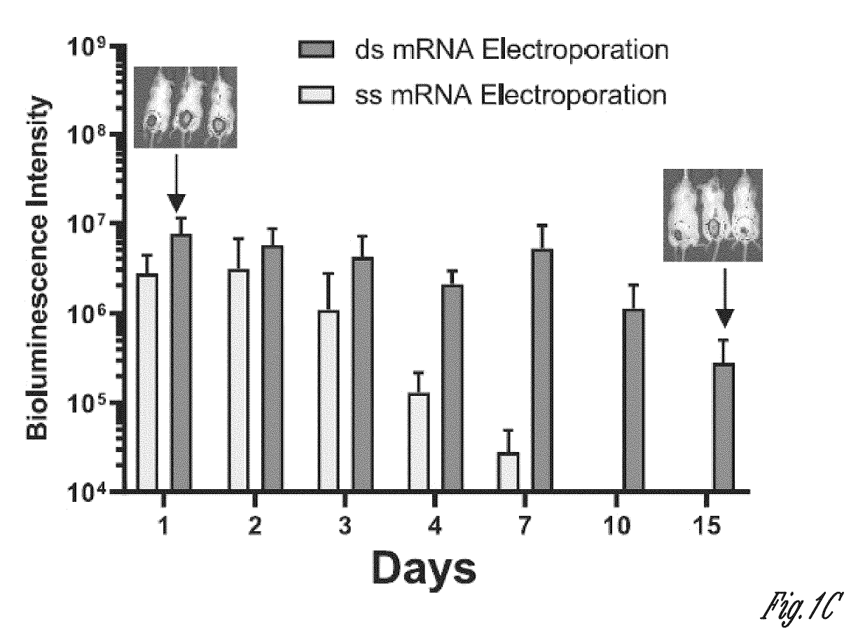

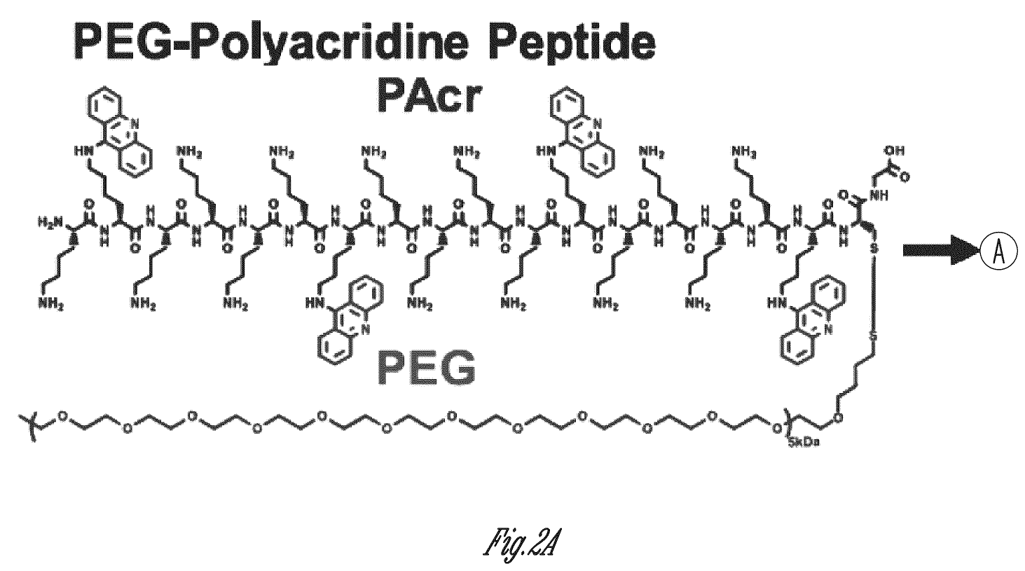

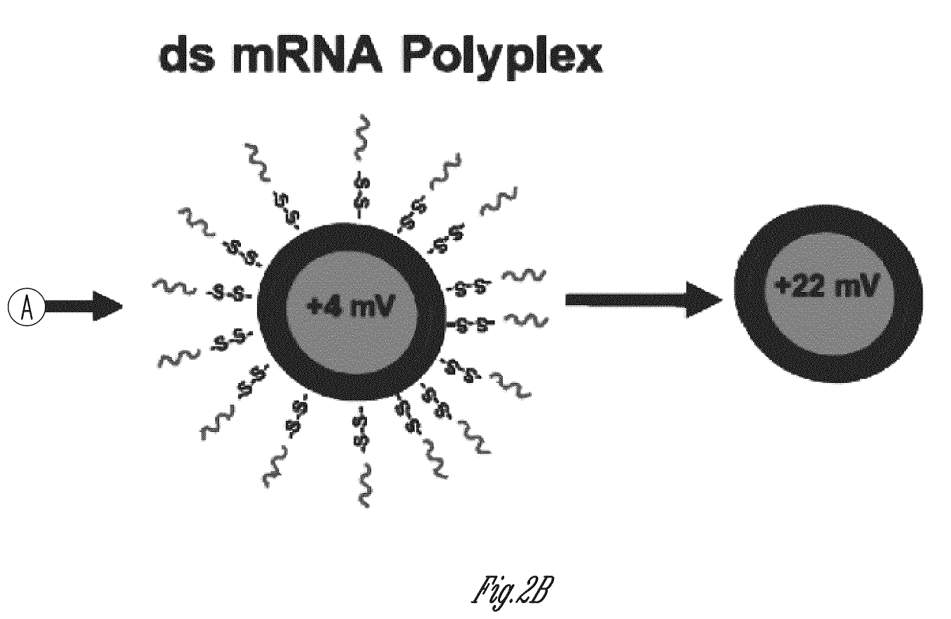

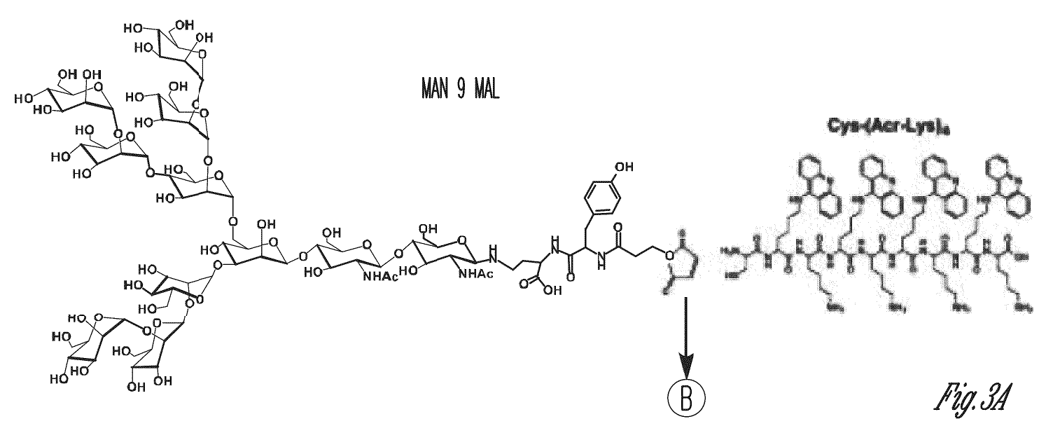

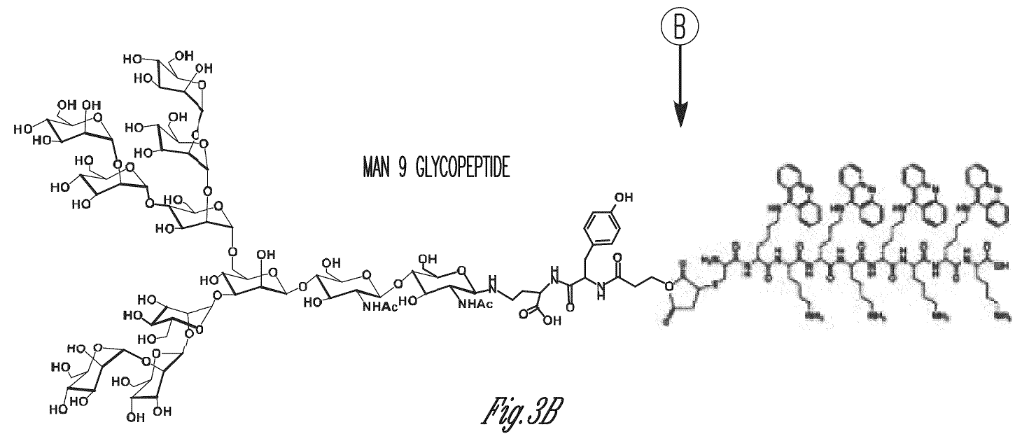

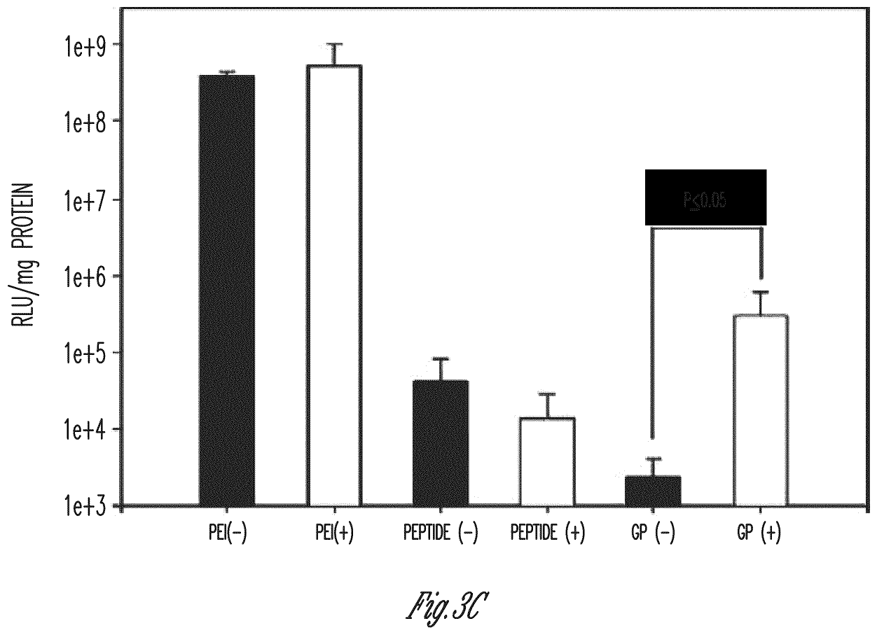

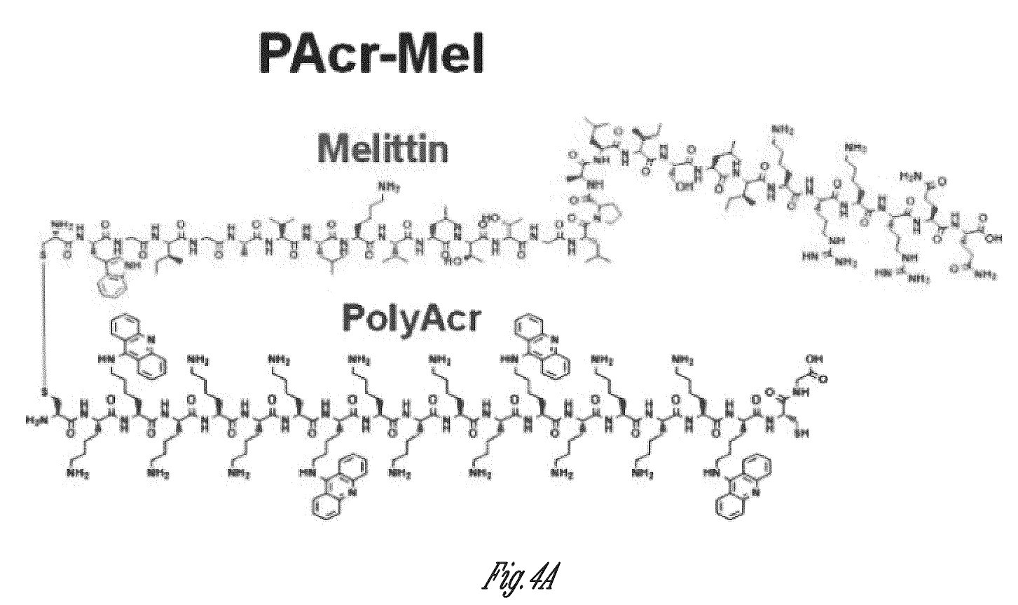

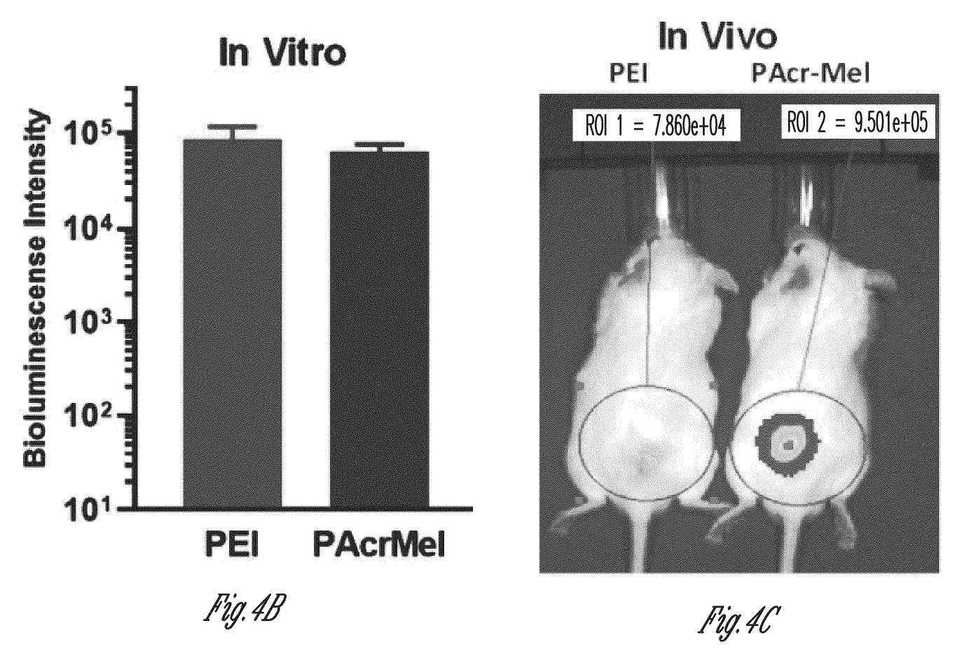

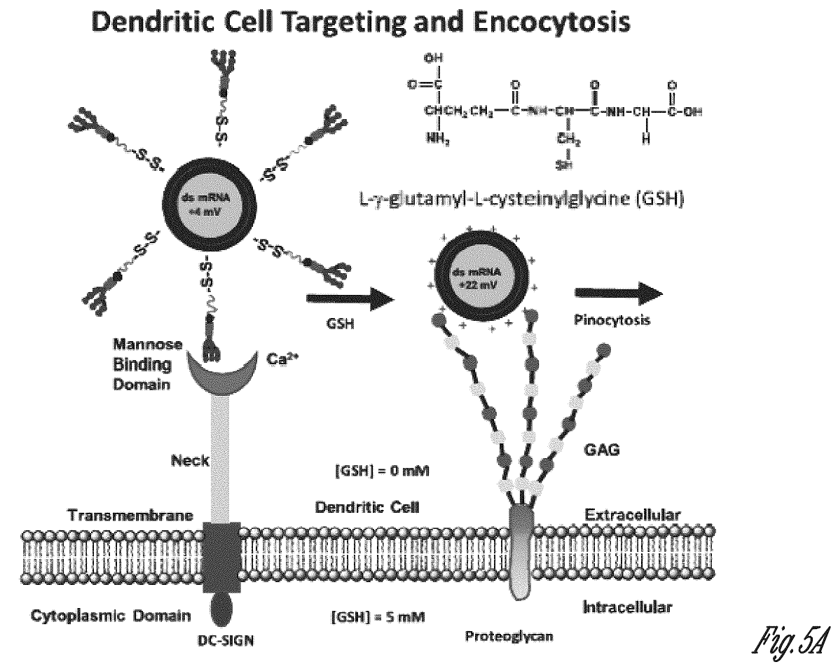

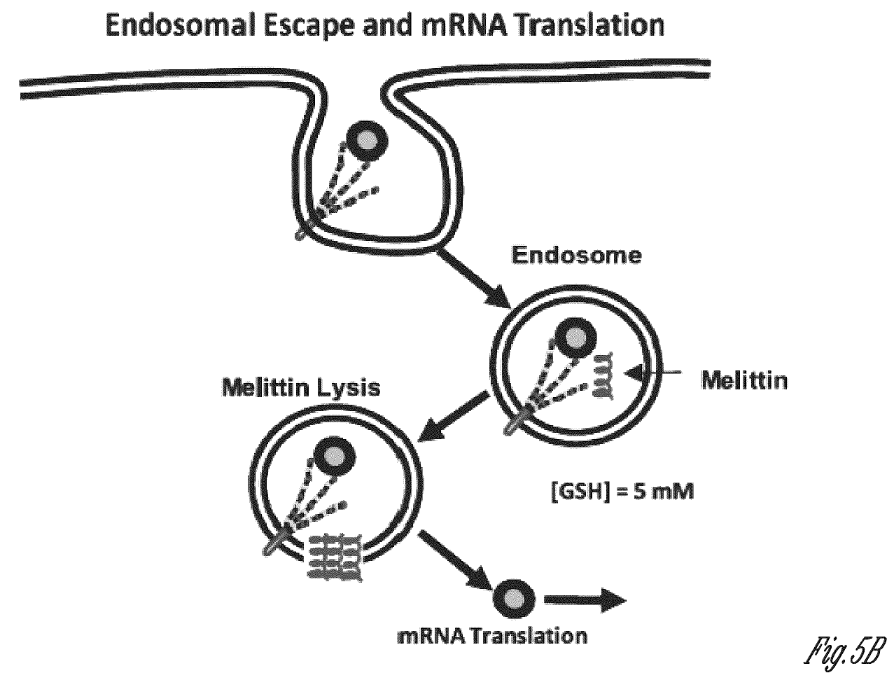

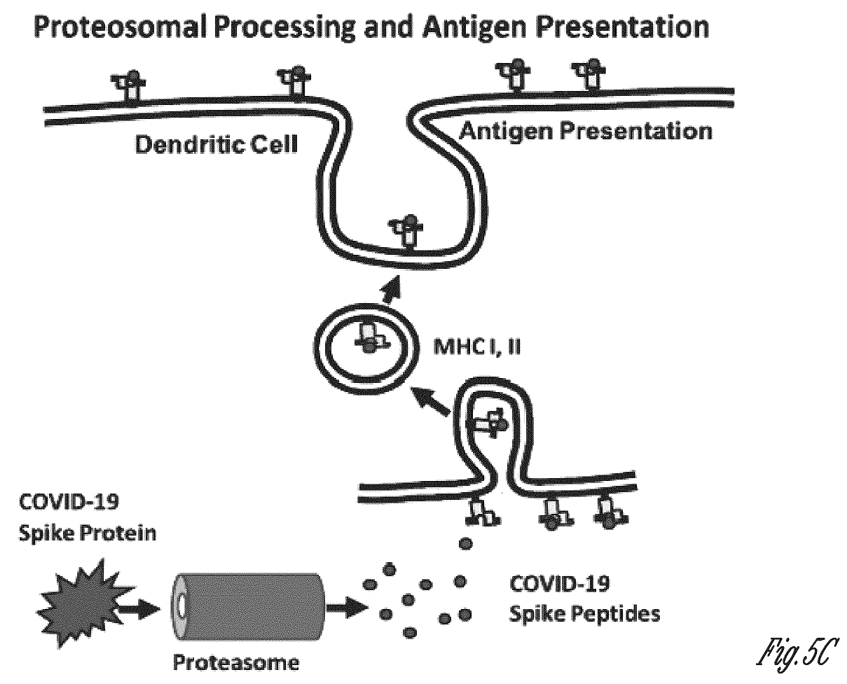

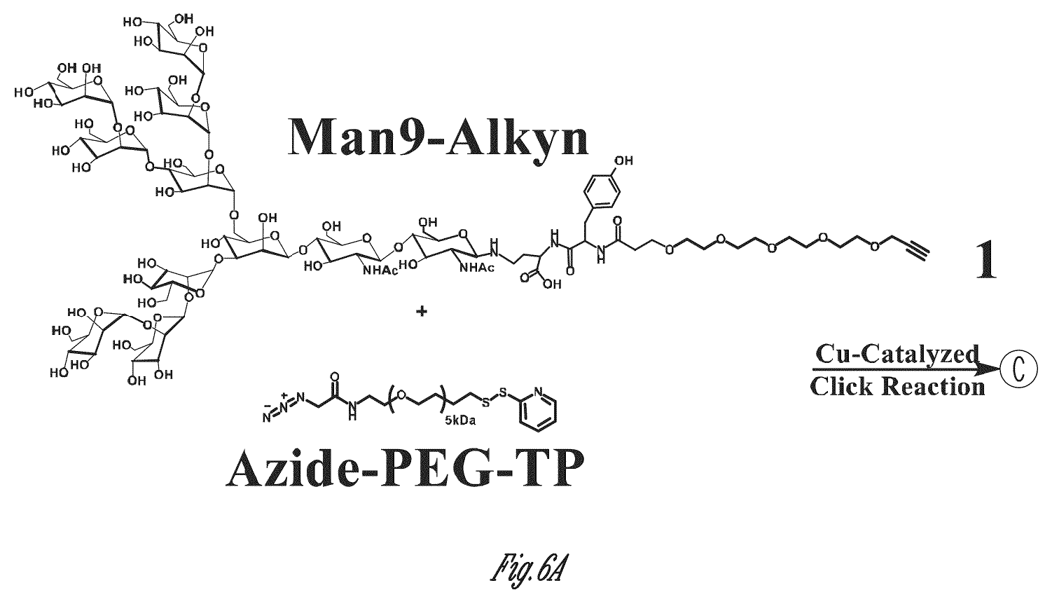

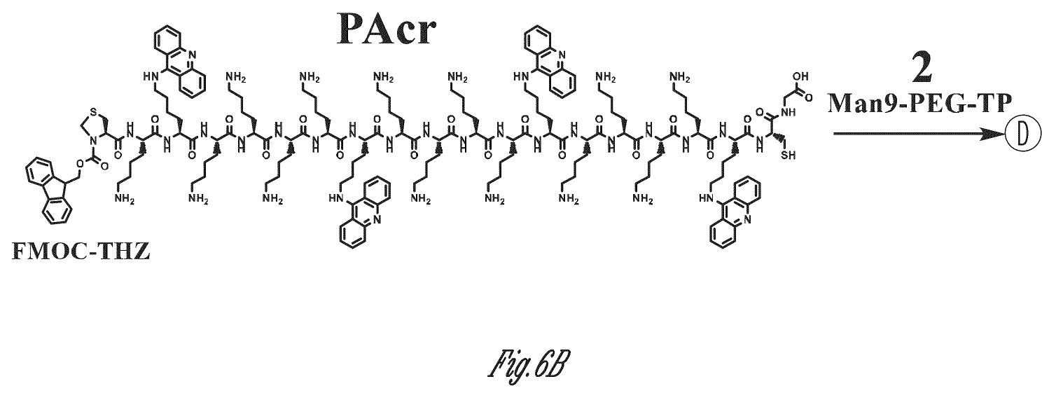

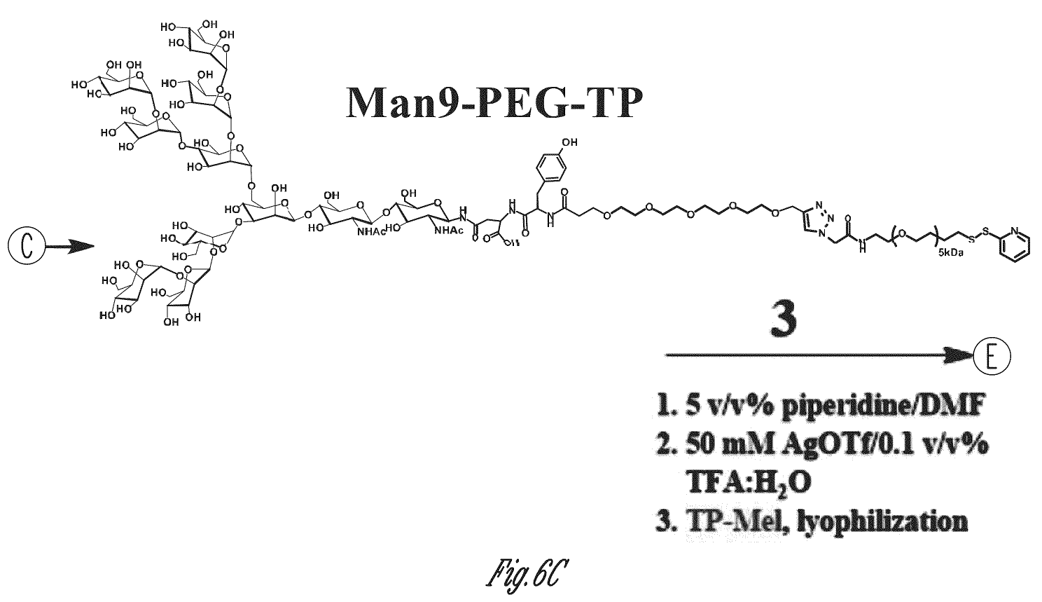

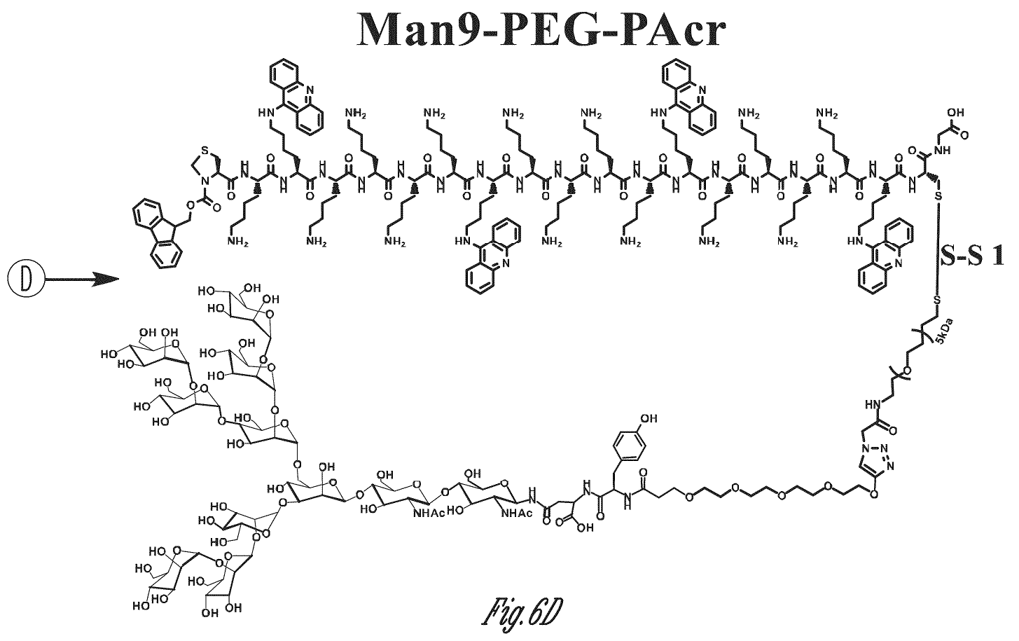

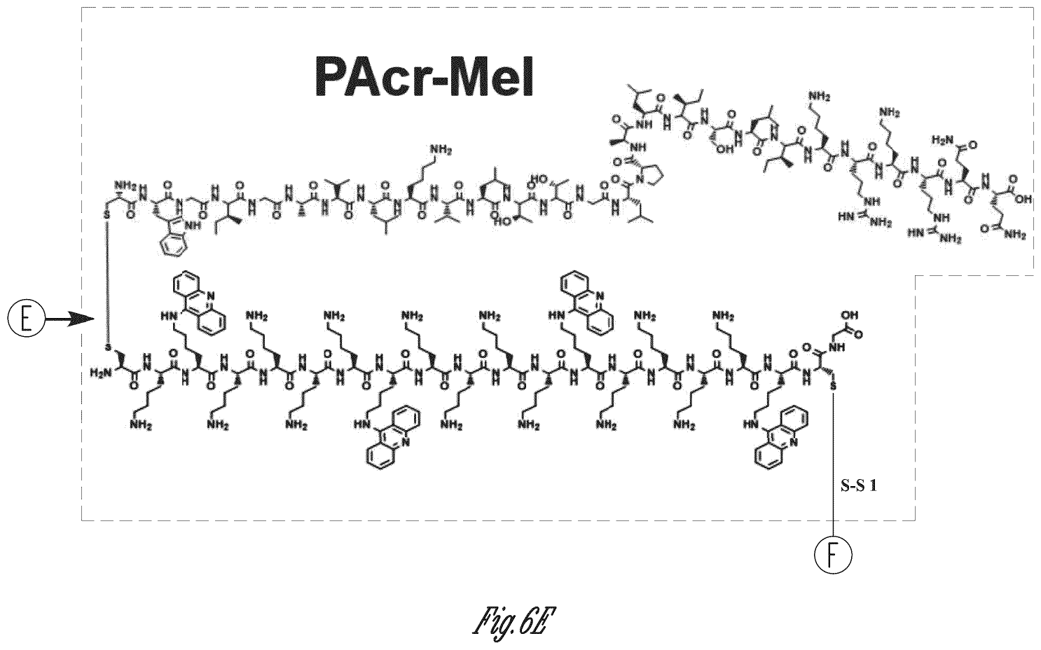

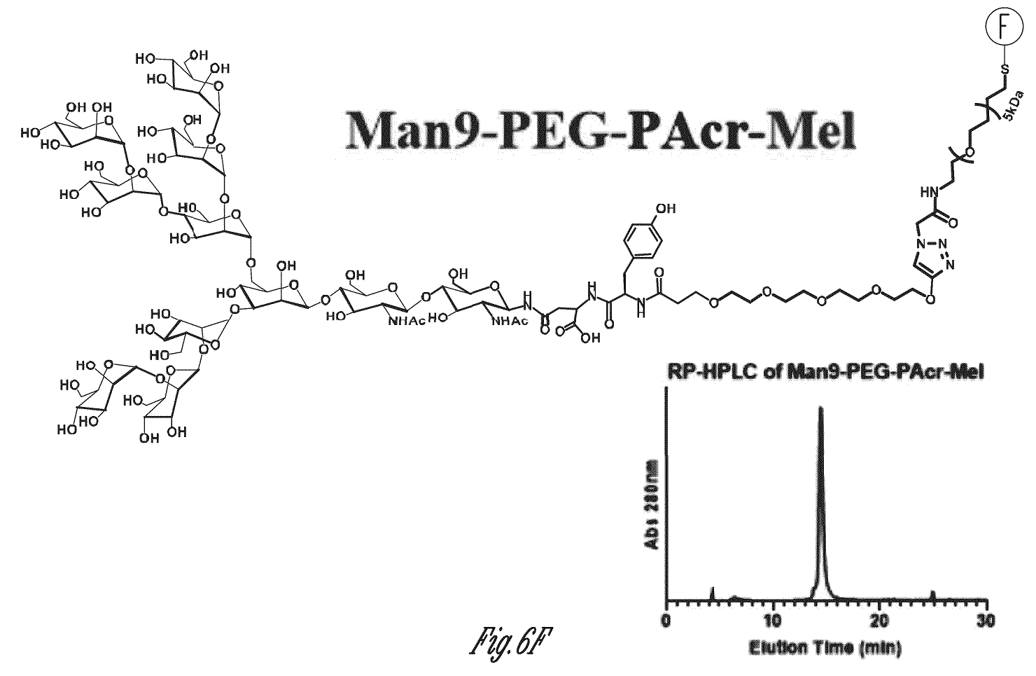

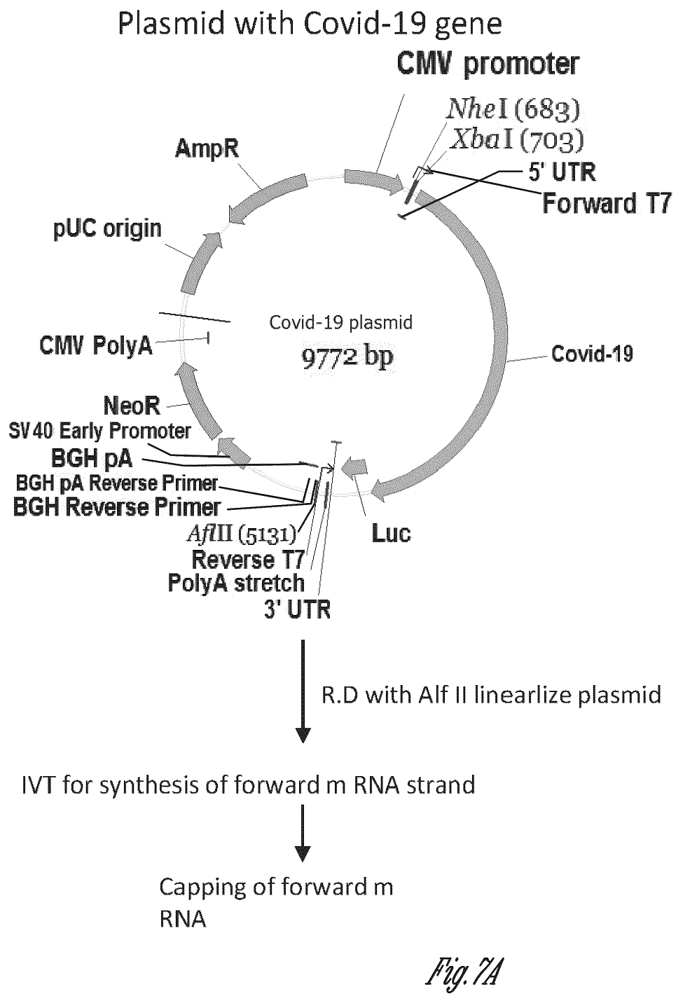

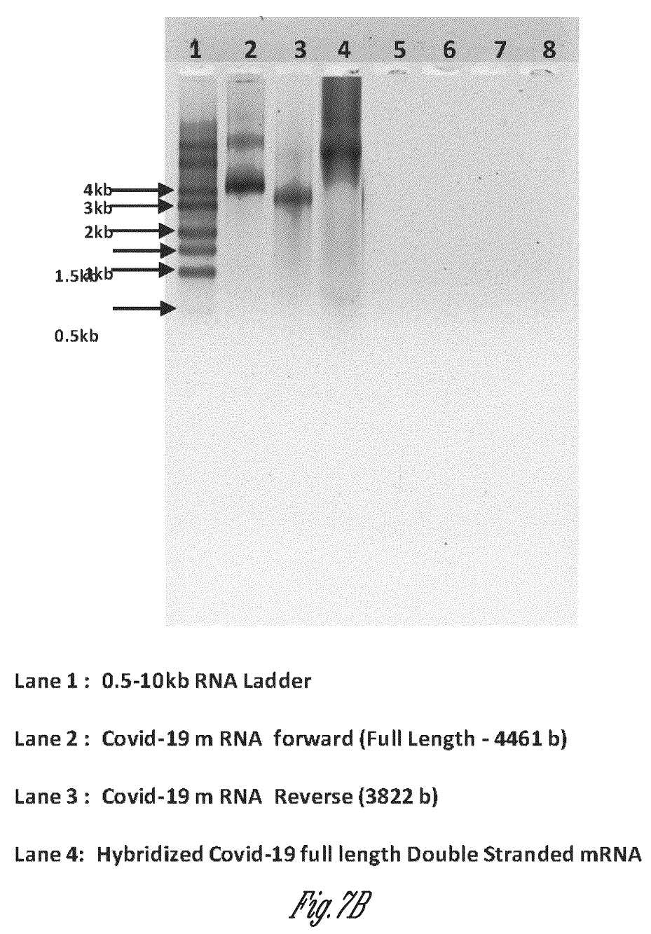

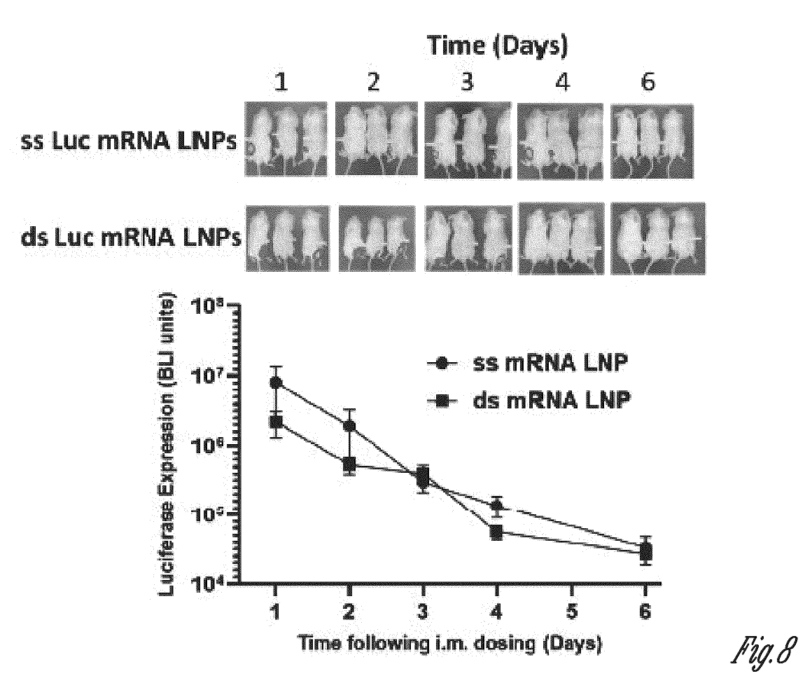

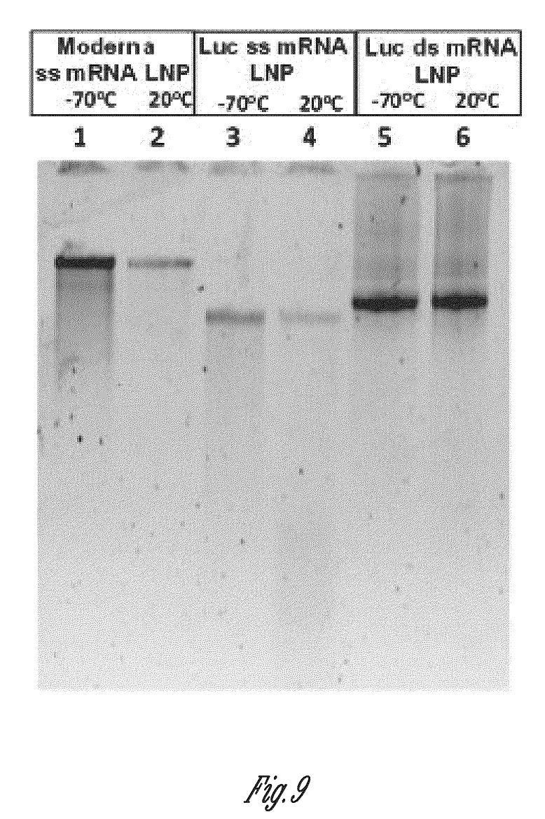

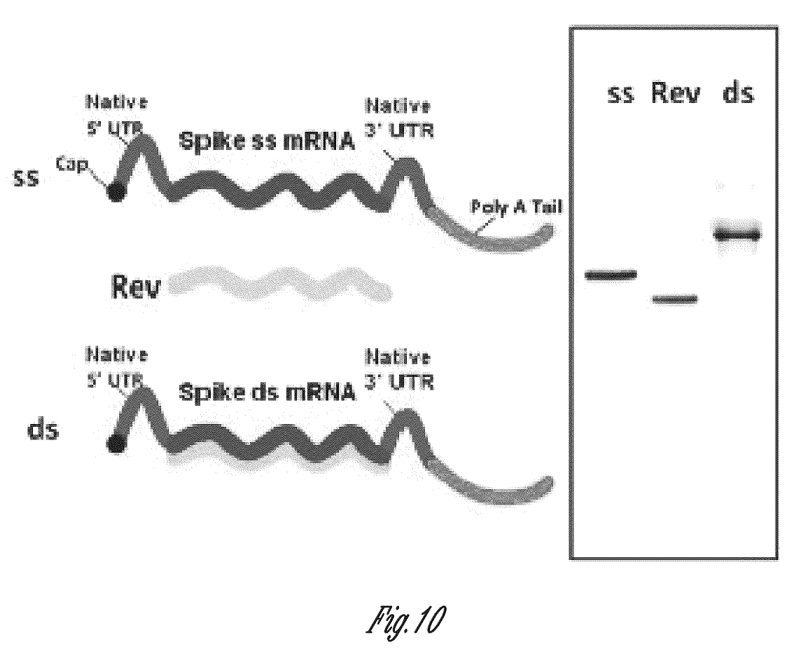

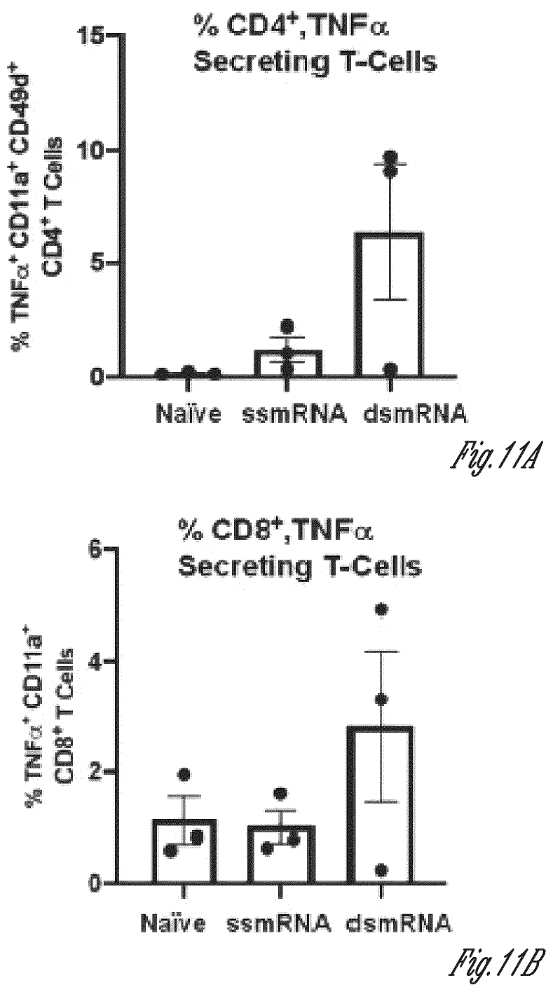

Double Stranded mRNA ds mRNA increases the metabolic stability of mRNA without decreasing its potency (Poliskey et al., 2018). It is derived by in vitro transcription (IVT) using T7 polymerase to drive transcription of two RNAs, complementary forward and reverse, from the same plasmid. The reverse strand is shorter and binds to the transgene sequence of the forward, without disrupting the 5′ and 3′ UTR, to generate metabolically stable ds mRNA (FIG. 1A). Double stranded mRNA possesses dramatically increased resistance to digestion with RNAse as demonstrated in the gels illustrated in FIG. 1B (Poliskey et al., 2018). Forward mRNA remains fully translationally competent when the reverse strand hybridizes to the transgene inside of the 5′ and 3′ UTR, which must remain native to gain full potency. The luciferase expression over fifteen days following intradermal dosing and electroporation of double stranded and single stranded mRNA in mice is illustrated in FIG. 1C. The improved stability of double stranded mRNA results in longer expression relative to single stranded mRNA. When combined with a self-adjuvant effect of double stranded mRNA7, we anticipate this will lead to a robust immune response, resulting in higher antibody titers for an equivalent dose of single stranded mRNA vaccine.

Peptide-Conjugate mRNA Nanoparticles. Peptide-conjugate mRNA nanoparticles offer an alternative and highly versatile vaccine platform to LNPs that might provide the opportunity to steer the Band T-cell response (Corey et al., 2020). A single peptide-conjugate can be used to package and stabilize mRNA, target mRNA to bind to and enter dendritic cells and release mRNA from endosomes into the cytosol to undergo translation. Thereby, peptide-conjugate mRNA nanoparticles offer a potential solution to anticipated difficulties in scale-up and cold storage of LNP-mRNA vaccines (Corey et al., 2020).

Gene delivery peptides may stabilize mRNA during delivery and release mRNA into the cytosol (Fernandez et al., 2011; Mathew et al., 2020; Crowley et al., 215; Khargharia et al., 2014; Retig & Rice, 2007; Chen et al., 2007; Kwok et al., 2001; McKenzie et al., 2000; Adami, 1999; Wadhwa et al., 1997; Wadhwa et al., 1995). Polyacridine peptides (FIG. 2) that bind to ds mRNA through a combination of ionic and hydrophobic interaction result in much more stable nanoparticles when dosed i.v. (Poliskey et al., 2018; Crowley et al., 2015; Kizzire et al., 2013. Khargharia et al., 2013; Fernandez et al., 2011, Mathew et al., 2020; Khargharia et al., 2014).

Incorporation of four Lys-Acr residues (FIG. 2) into a short polylysine peptide dramatically increases the binding affinity to ds mRNA (Poliskey et al., 2018; Kizzire et al., 2013). Polyacridine peptides may be designed to possess a Cys residue to allow conjugation of a single polyethylene glycol (PEG) per peptide Using this design a peptide sequence, PEG length, linkage and location were selected to generate PEGylated polyacridine peptides that form small, highly stable, mRNA nanoparticles of controlled size and charge (Poliskey et al., 2018, Crowley et al., 2015, Kizzire et al., 2013, Khargharia et al., 2013; Fernandez et al., 2011, Mathew et al., 2020; Fernandez et al., 2010). These have been dosed i.v. in mice and proven to stabilize both DNA and double stranded mRNA in the circulation (Poliskey et al., 2018; Crowley et al., 2015; Kizzire et al., 2013; Khargharia et al., 2013; Fernandez et al., 2011; Mathew et al., 2020; Fernandez et al., 2010). This well-developed ds mRNA binding polyacridine peptide will be used to generate nanoparticles to transfect both fibroblasts and dendritic cells follow target intradermal dosing.

### Man9 Targeting Ligand

Dendritic cells endocytose and proteolytically process viral proteins and present viral peptides on MHCII, resulting in B-cell programing and the humoral response (FIG. 5). DC-SIGN is a cell surface lectin found on dendritic cells that binds to Man9 N-glycans found on viruses (Alvarez et al., 2002; Engering et al., 2002; Feinberg et al., 2007; Lue et al., 2002). We previously purified Man9 and prepared a peptide-conjugate used to transfect DNA nanoparticles (FIG. 3) (Anderson et al., 2010; Evers et al., 1998). Man9 peptide mediated gene transfer was demonstrated in Chinese hamster ovary (CHO) cells that stably express DC-SIGN (FIG. 3, GP (−) and GP (+)) (Anderson et al., 2010). As described below, the Man9 N-glycan ligand is incorporated into a peptide-conjugate which is turn is used to generate a double stranded mRNA nanoparticle.

### Melittin Endosomal Escape Peptide

The ability to release mRNA into the cytosol of cells is fundamental to achieving translation and B and T-cell response. Melittin can be incorporated into DNA and mRNA nanoparticles (Baumhover et al., 2010; Chen et al., 2006). Melittin is a 26 amino acid amphiphilic peptide derived from bee venom (Ogris et al., 2001; Boeckle et al., 2006). When released from the nanoparticle, melittin oligomerizes to generate membrane pores that facilitate endosomal escape into the cytosol (Baumhover et al., 2010; Chen et al., 2006; Yang et al., 2001). Incorporation of melittin into nanoparticles results in a large (1000-fold) increase in gene transfer efficiency (Baumhover et al., 2010, Chen et al., 2006). Melittin-PolyAcr was as efficient as polyethylenimine (PEI) when used to transfect DNA nanoparticles in vitro (Baumhover et al., 2010). Melittin's membrane lytic activity is dependent on its release from the polyacridine peptide through disulfide bond reduction, which is likely achieved by intracellular glutathione (GSH) (Read et al., 2005). Substitution of this disulfide linkage with a redox stable maleimide linkage inactivates melittin release and blocks gene expression (Baumhover et al., 2010). Peptide-conjugates of melittin linked to an optimized polyacridine peptide (PAcr-Mel) possess potent in vitro gene transfer efficiency equivalent to PEI (Boussif et al., 1995) (FIG. 4). However, only PAcr-Mel ds mRNA nanoparticles mediate luciferase expression in vivo when dosed intradermally in mice, whereas PEI ds mRNA is inactive (FIG. 4). These results reflect a difference in the mechanism of endosomal escape for peptide-conjugate mRNA and PEI-mRNA nanoparticles.

Peptide-Conjugate mRNA Vaccine

The overall design incorporates delivery concepts of packaging, targeting and endosomal escape illustrated in FIGS. 1-5 into a single mRNA delivery conjugate (FIG. 6). Man9-PEG and the melittin are linked to the C and N terminal Cys residues of a polyacridine peptide by reversible disulfide bonds. The method to chemically install two distinct disulfide bonds was pioneered by my group using FMOC-thiazolidene (FMOC-THZ) as a protected Cys residue (Ericson & Rice, 2013a; Ericson & Rice, 2013b; Ericson et al., 1999). The peptide-conjugate modular design allows replacement of elements to optimize performance for each new gene delivery application. The PEG length, density and chemical linkage to the surface of ds mRNA nanoparticles are all important to optimizing physical and biological performance in each new application (Kizzire et al., 2013; Khargharia et al., 2013). While PAcr-Mel is a potent in vitro gene transfer peptide-conjugate, it has limited use in vivo due to it charge. The attachment of PEG to each peptide masks the nanoparticle charge and blocks physical aggregation, allowing formulation of nanoparticles of 1 mg/ml or higher needed for intradermal dosing (Kwok et al., 1999). Positioning of Man9 on the end of PEG improves nanoparticle binding to DC-SIGN on dendritic cells (Crowley & Rice, 2015). The release of PEG from the surface of the nanoparticle by disulfide bond reduction at the cell surface is proposed to cascade in the release of disulfide bonded melittin in the endosome (Scheme 10). Substitution with a shorter PEG will expose nanoparticle positive charge and might influence the transfection ratio of fibroblasts versus dendritic cells, thereby influence B and T-cell response. Substitution of Cys disulfide bonds with more reductively stable (Pen) bonds will likely influence the persistence of mRNA expression (Ericson & Rice, 2013b). Peptide-conjugate mRNA nanoparticles are formulated by simple one-step mixing in normal saline and can be freeze dried and reconstituted to retain full activity (Kwok et al., 2000). They offer a clear formulation advantage over lipid nanoparticles that require complex microfluidic mixing and have temperature stability concerns (Le et al., 2020).

### COVID-19 Antigen Selection

SARS-CoV-2 is a positive-strand RNA virus possessing a genome of approximately 29,700 nucleotides that shares 79.5% sequence identity with SARS-CoV9 (Guo et al., 2020). The genome encodes four major structural proteins identified as the spike (S) protein, nucleocapsid (N) protein, membrane (M) protein, and the envelope (E) protein (Phan, 2020). Spike protein (S protein) is the most promising antigen for vaccination against SARS-CoV-2. Its surface exposure allows direct recognition by the host immune system. The monomer S protein from SARS-CoV-2 contains 1273 amino acids, with a molecular weight of approximately 140 kDa that self-assembles into a homo-trimer. The S protein is composed of two subunits (S1 and S2) (Wu et al., 2020). The S1 subunit is further divided into the N-terminal domain (NTD) and the C-terminal domain (CTD) with the receptor binding domain (RBD) located in the CTD. The S2 subunit contains the basic elements required for membrane fusion (Wu et al., 2020; Zhang et al., 2020).

A plasmid (Luc80A) was used to prepare poly A tailed mRNA and a complementary reverse mRNA of any length (Poliskey et al., 2018). The plasmid encodes for optimized 13-globin 5′ and 3′ UTRs that flank the Luc transgene (Crowley et al., 2015). Using the published COVID-19 RNA sequence 9 the S-protein coding sequence was substituted for the Luc transgene and COVID-19 ss and ds mRNA were prepared by in vitro transcription. In one embodiment of the ds mRNA, the reverse strand hybridizes with only the transgene, leaving the 5′ and 3′ UTR in their native stem-loop folded conformation (Crowley et al., 2015).

Compare the Magnitude and Duration of the B-Cell and T-Cell Response for Single Stranded and Double Stranded (COVID Spike) mRNA Delivered as Either a Hold Nanoparticle, Peptide-Conjugate Nanoparticle or by Electroporation.

Double Stranded mRNA Vaccine Formulation and Testing. Antibody titer and cellular immunity are measured in mice vaccinated with either single or double stranded COVID-19 spike mRNA delivered by either peptide-conjugate, lipid nanoparticle or electroporation Peptide-conjugate mRNA nanoparticle vaccine will be prepared by adding Man9-PEG-PAcr-Mel (FIG. 6) to single or double stranded mRNA. These are administered to C57BL6 mice in a 50 μl dose given intra-dermally in normal saline. Likewise, ss mRNA and ds mRNA lipid nanoparticles (LNP) are prepared according to the procedure described by Hassett (2019) and dosed intra-dermally. Similarly, electroporation is conducted by dosing ss mRNA and ds mRNA intra-dermally followed by applying an optimized pulse sequence (FIG. 1C).

Groups of six male and female C57BL/6 mice weighing approximately 20 g are administered an escalating dose of 1, 5, and 10 μg of mRNA (peptide-conjugate nanoparticles, LNP, or electroporated) followed by an equal booster dose administered on day 28. Control mice (6 male and female for each formulation) are administered inactive ss or ds COVID-19 mRNA prepared by omitting the capping step (Crowley et al., 2015). Mice are weighed at the time of blood draw. Blood is removed serially every five days (Day 1-60) from the tail vein and analyzed for antibody titer.

Blood samples from vaccinated mice are analyzed for specific IgG using an ELISA assay (Stadlbauer et al., 2020). The assay is developed using commercially available recombinant COVID-19 spike protein (S1) and an anti-spike polyclonal antibody. The assay is validated for precision, accuracy, linearity and limit-of-detection. The assay is cross referenced with other commercially available serological assays for COVID-19 anti-spike protein. Cellular immunity is evaluated at day 60. Splenocytes are collected and cultured, then analyzed by ELISpot assay following incubation with Covid-19 spike protein for 24 hours to measure INF-γ (Hassett et al., 2019).

B-Cel1 and T-Cel1 Response for Targeted Peptide-Conjugate Double Stranded mRNA Nanoparticles.

The durability of the immune response may be dependent upon optimizing both the humeral and cellular immunity. Peptide-conjugate targeted expression of COVID-19 proteins in dendritic cells may improve cellular immunity. The influence of Man9 targeted binding to DC-SIGN on dendritic cells can be directly compared by substituting Man9-PEG-PAcr-Mel with PEG-PAcr-Mel in double stranded mRNA nanoparticles (FIG. 6).

The disulfide linkage used to join Man9-PEG and PAcr-Mel (FIG. 6) may be designed to undergo reduction, resulting in PEG-shedding and subsequent charge-mediated pinocytosis and transfection of both dendritic cells and fibroblasts (FIG. 5). Replacing the disulfide bond with a more stable Pen-Pen disulfide bond or reductively stable maleimide linkage will slow or block PEG shedding and subsequent pinocytosis, and thereby may favor receptor-medicated endocytosis via DC-SIGN, leading to increased cellular immunity. Likewise, the particle size of ds mRNA nanoparticles (170 nm) can be systematically decreased to less than 100 nm diameter by a heat shrinking method (Mathew et al., 2020). Decreasing the particle size to less than 100 nm may improve DC-SIGN receptor mediated uptake and potentially improve cellular immunity.

In one embodiment, an animal may be vaccinated with nanoparticles possessing ds mRNAs from both spike (S) and nucleocapsid (N) COVID-19 proteins, to invoke both a B-cell response against the spike and a long lasting T-cell response to the nucleocapsid. Blended S&N ds mRNA peptide-conjugate nanoparticles are studied for increased duration of cellular immunity. In addition, the immune response to I chimeric mRNA encoding S or N and chimerized with a highly immunogenic keyhole limpet hemocyanin protein (KLH) (Stadlbauer et al., 2020). Is determined A portion of KLH is cloned into a plasmid vector to generate a chimeric mRNA that translates into either COVID-19 S-KLH or N-KLH protein. This potentially increases the immunogenicity of S and N by boosting via the hapten effect with KLH (Marato et al., 2005).

### Example IV

Double Stranded mRNA ds mRNA is derived by in vitro transcription (IVT) using T7 polymerase to drive transcription of two RNAs, complementary forward and reverse, from the same plasmid. The reverse strand is shorter and binds to the transgene sequence of the forward, without disrupting the 5′ and 3′ UTR, to generate metabolically stable ds mRNA (FIG. 1). Double stranded mRNA possesses dramatically increase resistance to digestion with RNAse as demonstrated in the published gels illustrated in FIG. 1B (Poliskey et al., 2018). Forward mRNA remains fully translationally competent when the reverse strand hybridizes to the transgene inside of the 5′ and 3′ UTR, which must remain native to gain full potency. The luciferase expression over fifteen days following intradermal dosing and electroporation of double stranded and single stranded mRNA in mice is illustrated in FIG. 1C. The improved stability of double stranded mRNA results in longer expression relative to single stranded mRNA. When combined with a self-adjuvant effect of double stranded mRNA (Uchida et al., 2018), this will lead to a robust immune response, resulting in higher titers of antibody for an equivalent dose of single stranded mRNA vaccine.

Peptide-Conjugate mRNA Nanoparticles

Peptide-conjugate mRNA nanoparticles offer an alternative and highly versatile vaccine platform to LNPs that might provide the opportunity to steer the Band T-cell response (Corey et al., 2020). A single peptide-conjugate can be used to package and stabilize mRNA, target mRNA to bind to and enter dendritic cells and release mRNA from endosomes into the cytosol to undergo translation. Thereby, peptide-conjugate mRNA nanoparticles offer a solution to anticipated difficulties in scale-up and cold storage of LNP-mRNA vaccines (Corey et al., 2020).

Gene delivery peptides are prepared with the goal of stabilize mRNA during delivery and releasing mRNA into the cytosol (Mathew et al., 2020; Khargharia et al., 2014; Fernandez et al., 2011; Crowley & Rice, 2015; Rettig & Rice, 2007; Chen et al., 2007; Kwok et al., 2001; McKenzie et al., 2000; Adami, 1999; Wadhwa et al., 1997; Wadhwa et al., 1995). Polyacridine peptides (FIG. 2) bind to ds mRNA through a combination of ionic and hydrophobic interaction and resulted in much more stable nanoparticles when dosed i.v. (Poliskey et al., 2018; Mathew et al., 2020; Crowley et al., 2015; Khargharia et al., 2014; Kizzire et al., 2013; Khargharia et al., 2013, Fernandez 2011).

Incorporation of four Lys-Acr residues (FIG. 2) into a short polylysine peptide dramatically increases the binding affinity to ds mRNA (Poliskey et al., 2018; Kizzire et al., 2013). Polyacridine peptides are designed to possess a Cys residue to allow conjugation of a single polyethylene glycol (PEG) per peptide Using this design, the peptide sequence, PEG length, linkage and location were selected to generate PEGylated polyacridine peptides that form small, highly stable, mRNA nanoparticles of controlled size and charge (Poliskey et al., 2018; Mathew et al., 2020; Crowley et al., 2015; Kizzire et al., 2013; Khargharia et al., Fernandez et al., 2011). These have been dosed i.v. in mice and proven to stabilize both DNA and double stranded mRNA in the circulation (Poliskey et al., 2018; Mathew et al., 2020; Crowley et al., 2015; Kizzire et al., 2013; Khargharia et al., 2013; Fernandez et al., 2011; Fernandez et al., 2010). This ds mRNA binding polyacridine peptide issued to generate nanoparticles to transfect both fibroblasts and dendritic cells follow target intradermal dosing.

### Man 9 Targeting Ligand

Dendritic cells endocytose and proteolytically process viral proteins and present viral peptides on resulting in B-cell programing and the MHCII, humeral response (FIG. 5). DC-SIGN is a cell surface lectin found on dendritic cells that binds to Man 9 N-glycans found on viruses (Alvarez et al., 2002; Engering et al., 2002; Feinberg et al., 2007; Lue et al., 2002). Man 9 is purified and a peptide-conjugate with Man 9 is prepared and used to transfect DNA nanoparticles (FIG. 3) (Anderson et al., 2010; Evers et al., 1998). Successful Man 9 peptide mediate gene transfer was demonstrated in Chinese hamster ovary (CHO) cells that stably express DC-SIGN (FIG. 3, GP (−) and GP (+)) (Anderson et al., 2010). As described below, the Man 9 N-glycan ligand is incorporated into a peptide-conjugate to generate a double stranded mRNA nanoparticle.

### Melittin Endosomal Escape Peptide

The ability to release mRNA into the cytosol of cells is fundamental to achieving translation and B and T-cell response. We have developed a successful strategy of incorporating melittin into DNA and mRNA nanoparticles (Baumhover et al., 2010; Chen et al., 2006). Melittin is a 26 amino acid amphiphilic peptide derived from bee venom (Ogris et al., 2001; Boeckle et al., 2006). When released from the nanoparticle, melittin oligomerizes to generate membrane pores that facilitate endosomal escape into the cytosol (Baumhover et al., 2010; Chan et al., 2006; Yang et al., 2001). Incorporation of melittin into nanoparticles results in a large (1000-fold) increase in gene transfer efficiency (Baumhover et al., 2010; Chan et al., 2006). Melittin-PolyAcr was as efficient as polyethylenimine (PEI) when used to transfect DNA nanoparticles in vitro (Baumhover et al., 2010). Melittin's membrane lytic activity is dependent on its release from the polyacridine peptide through disulfide bond reduction, which is likely achieved by intracellular glutathione (GSH) (Read et al., 2005). Substitution of this disulfide linkage with a redox stable maleimide linkage inactivates melittin release and blocks gene expression (Baumhover et al., 2010). Peptide-conjugates of melittin linked to a polyacridine peptide (PAcr-Mel) possess potent in vitro gene transfer efficiency equivalent to PEI (FIG. 4). However, only PAcr-Mel ds mRNA nanoparticles mediate luciferase expression in vivo when dosed intradermally in mice, whereas PEI ds mRNA is inactive (FIG. 4). These results reflect a difference in the mechanism of endosomal escape for peptide-conjugate mRNA and PEI-mRNA nanoparticles.

Peptide-Conjugate mRNA Vaccine

The delivery vehicle influences the magnitude, type and durability of the immune response to an mRNA vaccine. Different mRNA vaccine platforms are compared to determine how to improve efficacy.

The overall design includes a Man9-PEGylated polyacridine peptide disulfide linked to melittin. This design incorporates delivery concepts of packaging, targeting and endosomal escape illustrated in FIGS. 1-5 into a single mRNA delivery conjugate (FIG. 6). Man9-PEG and the melittin are linked to the C and N terminal Cys residues of a polyacridine peptide by reversible disulfide bonds. The method to chemically install two unique disulfide bonds employs FMOC-thiazolidene (FMOC-THZ) as a protected Cys residue (Ericson & Rice, 2013a; Ericson & Rice, 2013b; Ericson & Rice, 2012).

The Man9-PEG-PAcr-Mel conjugate is assembled is three chemical steps as illustrated in FIG. 6. The assembly of the Man9-PEG-TP by the Click reaction (Step 1) allows its conjugation to a C-terminal Cys on PAcr (Step 2). The preparation of polyacridine peptide (PAcr) by solid phase peptide synthesis 6⋅ 8 includes installation of FMOC-THZ at the N-terminus, to direct selective conjugation of the C-terminal Cys with Man9-PEG-TP (Step 2, TP refers to thiol-pyridine). Removal of FMOC-THZ exposes the N-terminal Cys for reaction with TP-Cysmelittin (Step 3) resulting in Man9-PEG-PAcr-Mel. RP-HPLC analysis of the purified conjugate is illustrated in inset B. The synthesis is flexible in its design allowing for variations in each of the polyacridine peptide, melittin peptide, PEG length, Cys linkages and targeting ligand.

Double Stranded mRNA Vaccine Formulation and Testing

A published RNA sequence of COVID-19 was used to design mRNA that encodes the spike protein (Wu et al., 2020, which is incorporated by reference herein). mRNA encoding the entire spike protein or shorter fragments of the spike protein (Zhang et al., 2020). In one embodiment, an animal is immunized using nanoparticles possessing ds mRNAs from both spike (S) and nucleocapsid (N) COVID-19 proteins, to invoke both a B-cell response against the spike and a long lasting T-cell response the nucleocapsid. The immunogenicity of chimeric mRNA that encodes S or N linked to the highly immunogenic keyhole limpet hemocyanin protein (KLH) (Swaminathan et al., 2014) is also evaluated as this may increase the immunogenicity of S and/or N by boosting via the hapten effect with KLH (Marcato et al., 2005).

Antibody titer is compared in mice vaccinated with either single or double stranded mRNA delivered by either peptide-conjugate, lipid nanoparticle or electroporation. The peptide-conjugate mRNA nanoparticle vaccine is prepared by adding Man9-PEG-PAcr-Mel or PAcr-Mel, or blends of the two to COVID-19 double stranded mRNA to investigate the influence of targeted and non-targeted delivery on B and T-cell response. Nanoparticles are administered to ICR mice in a 50 μl dose delivered intra-dermally in normal saline. Groups of six male and female ICR mice weighing approximately 20 g are administered an escalating dose of 1, 10 and 50 μg of mRNA nanoparticles followed by a booster dose administered on day 30. Control mice are administered peptide-conjugate, LNP or electroporation alone. Mice are weighed at the time of blood draw (50 μl), removed serially every five days (Day 1-60) from the tail vein and analyzed for antibody titer.

Blood samples from vaccinated mice are analyzed for specific IgG using an ELISA assay (Stadlbauer et al., 2020). The assay is developed using commercially available recombinant COVID-19 spike protein (S1) and an anti-spike polyclonal antibody. The assay is validated for precision, accuracy, linearity and limit-of-detection. The assay is cross referenced with other commercially available serological assays for COVID-19 anti-spike protein. Detailed immunological studies to determine activation of dendritic cells and T-cell response are conducted.

### Example V

Exemplary Viral Antigen Encoding ds mRNA

The leading SARS-CoV-2 mRNA vaccine (mRNA-1273, encoding S protein) developed by Moderna is in Phase 3 Clinical Trial as of July 2020 (ClinicalTrials.gov Identifier: NCT04283461). The major advantage of this vaccine platform over others is speed. It took only 42 days for Moderna to generate the vaccine for Phase 1 Clinical Trial testing after receiving the DNA sequence (Wu et al., 2020). The Moderna mRNA vaccine relies upon a lipid nanoparticle (LNPs) to achieve cellular delivery of S protein mRNA (Hassett et al., 2019). LNPs are the most widely used in vivo mRNA delivery system at present (Semple et al., 2010). mRNA-LNPs were first demonstrated as efficient delivery systems for mRNA in mice 2015 (Pardi et al., 2015). Since then, multiple vaccine studies have resulted in durable, protective immune responses against multiple infectious pathogens, often after a single dose (Awasthi et al., 2019; VanBlargan et al., 2018). The LNP delivery system used in the COVID-19 clinical trial is a precise blend of four lipids mixed with single stranded mRNA in a microfluidic mixer (Hassett et al., 2019). Both the lipid structure and composition influence the IgG titer following i.m. dosing in mice (Hassett et al., 2019). Double stranded mRNA may enhance the potency and efficacy of LNP-mRNA, however, it is not certain if B-cell immunity is sufficient or if T-cell immunity is also necessary to achieve durable immunity (Corey et al., 2020).

mRNA Vaccines

mRNA vaccines have many advantages over traditional vaccines that have been developed (Pardi et al., 2018). Some of these include rapid R&D and production, simultaneous vaccination with multiple immunogens and a high margin of safety (Pardi et al., 2018). One of the most important features is the ability to rapidly generate an mRNA vaccine by substituting new mRNA into an existing delivery vehicle (Gomez-Aguado et al., 2020). This greatly reduces the time normally needed to generate and formulate a viral protein or to develop an attenuated virus, two strategies most often used for traditional vaccine development (L. et al., 2020).

While there are many types of mRNA vaccines, they all use single stranded mRNA to encode an immunogenic viral protein that is packaged, delivered and expressed in muscle or dermis, leading to a B-cell immune response against the virus (Pardi et al., 2020). Following intra-dermal administration, mRNA nanoparticles are likely taken by fibroblasts and dendritic cells of the dermis (Selmi et al., 2016; Diken et al., 2011). The intracellular release of mRNA results in binding to ribosomes and translation of the mRNA into the programed viral protein. Viral proteins are proteolyzed by the proteasome and peptide antigens are presented by MHC I on dendritic cell surface to prime for T-cell response. Alternatively, transfected fibroblasts secrete viral proteins which are endocytosed by dendritic cells and proteolytically processed, leading to peptide antigen presentation on MHC II to prime the B-cell response. mRNA stability and the delivery vehicle likely play a role in determining the magnitude and type of immune response. response (Le et al., 2020).

### Results

Double Stranded mRNA. ds mRNA increases the metabolic stability of mRNA without decreasing its potency (Poliskey et al., 2018). It is derived by in vitro transcription (IVT) using T7 polymerase to drive transcription of two RNAs, complementary forward and reverse, from the same plasmid. The reverse strand is shorter and binds to the transgene sequence of the forward, without disrupting the 5′ and 3′ UTR, to generate metabolically stable ds mRNA (FIG. 1A). Double stranded mRNA possesses dramatically increased resistance to digestion with RNAse as demonstrated in the gels illustrated in FIG. 1B (Poliskey et al., 2018). Forward mRNA remains fully translationally competent when the reverse strand hybridizes to the transgene inside of the 5′ and 3′ UTR, which must remain native to gain full potency. The luciferase expression over fifteen days following intradermal dosing and electroporation of double stranded and single stranded mRNA in mice is illustrated in FIG. 1C. The improved stability of double stranded mRNA results in longer expression relative to single stranded mRNA. When combined with a self-adjuvant effect of double stranded mRNA7, we anticipate this will lead to a robust immune response, resulting in higher antibody titers for an equivalent dose of single stranded mRNA vaccine.

### COVID-19 Antigen Selection

SARS-CoV-2 is a positive-strand RNA virus possessing a genome of approximately 29,700 nucleotides that shares 79.5% sequence identity with SARS-CoV9 (Guo et al., 2020). The genome encodes four major structural proteins identified as the spike (S) protein, nucleocapsid (N) protein, membrane (M) protein, and the envelope (E) protein (Phan, 2020). Spike protein (S protein) is the most promising antigen for vaccination against SARS-CoV-2. Its surface exposure allows direct recognition by the host immune system. The monomer S protein from SARS-CoV-2 contains 1273 amino acids, with a molecular weight of approximately 140 kDa that self-assembles into a homo-trimer. The S protein is composed of two subunits (S1 and S2) (Wu et al., 2020). The S1 subunit is further divided into the N-terminal domain (NTD) and the C-terminal domain (CTD) with the receptor binding domain (RBD) located in the CTD. The S2 subunit contains the basic elements required for membrane fusion (Wu et al., 2020; Zhang et al., 2020).

A plasmid (Luc80A) was used to prepare poly A tailed mRNA and a complementary reverse mRNA of any length (Poliskey et al., 2018). The plasmid encodes for optimized 13-globin 5′ and 3′ UTRs that flank the Luc transgene (Crowley et al., 2015). Using the published COVID-19 RNA sequence the S-protein coding sequence was substituted for the Luc transgene and COVID-19 ss and ds mRNA were prepared by in vitro transcription. In one embodiment of the ds mRNA, the reverse strand hybridizes with only the transgene, leaving the 5′ and 3′ UTR in their native stem-loop folded conformation (Crowley et al., 2015).

Double Stranded mRNA Vaccine Formulation and Testing

A published RNA sequence of COVID-19 was used to design mRNA that encodes the spike protein (Wu et al., 2020, which is incorporated by reference herein). mRNA encoding the entire spike protein or shorter fragments of the spike protein (Zhang et al., 2020). In one embodiment, an animal is immunized using nanoparticles possessing ds mRNAs from both spike (S) and nucleocapsid (N) COVID-19 proteins, to invoke both a B-cell response against the spike and a long lasting T-cell response the nucleocapsid. The immunogenicity of chimeric mRNA that encodes S or N linked to the highly immunogenic keyhole limpet hemocyanin protein (KLH) (Swaminathan et al., 2014) is also evaluated as this may increase the immunogenicity of S and/or N by boosting via the hapten effect with KLH (Marcato et al., 2005).

Antibody titer is compared in mice vaccinated with either single or double stranded mRNA delivered by lipid nanoparticle. Nanoparticles are administered to ICR mice in a 50 μl dose delivered intra-dermally in normal saline. Groups of six male and female ICR mice weighing approximately 20 g are administered an escalating dose of 1, 10 and 50 μg of mRNA nanoparticles followed by a booster dose administered on day 30. Control mice are administered peptide-conjugate, LNP or electroporation alone Mice are weighed at the time of blood draw (50 μl), removed serially every five days (Day 1-60) from the tail vein and analyzed for antibody titer.

Blood samples from vaccinated mice are analyzed for specific IgG using an ELISA assay (Stadlbauer et al., 2020). The assay is developed using commercially available recombinant COVID-19 spike protein (S1) and an anti-spike polyclonal antibody. The assay is validated for precision, accuracy, linearity and limit-of-detection. The assay is cross referenced with other commercially available serological assays for COVID-19 anti-spike protein. Detailed immunological studies to determine activation of dendritic cells and T-cell response are conducted.

B-cell and T-cell response for double stranded mRNA nanoparticles. The durability of the immune response may be dependent upon optimizing both the humeral and cellular immunity. In one embodiment, an animal may be vaccinated with nanoparticles possessing ds mRNAs from both spike (S) and nucleocapsid (N) COVID-19 proteins, to invoke both a B-cell response against the spike and a long lasting T-cell response to the nucleocapsid. Blended S&N ds mRNA peptide-conjugate nanoparticles are studied for increased duration of cellular immunity. In addition, the immune response to I chimeric mRNA encoding S or N and chimerized with a highly immunogenic keyhole limpet hemocyanin protein (KLH) (Stadlbauer et al., 2020). Is determined A portion of KLH is cloned into a plasmid vector to generate a chimeric mRNA that translates into either COVID-19 S-KLH or N-KLH protein. This potentially increases the immunogenicity of S and N by boosting via the hapten effect with KLH (Marato t al., 2005).

Other studies were performed with mRNA lipid nanoparticles using a luciferase read-out in mice after i.m. dosing, and vaccinating mice with Covid Spike single stranded mRNA lipid nanoparticles and double stranded mRNA lipid nanoparticles.

### Example VI

### Exemplary Viral Templates for ds RNA Vaccines

A ds RNA may be prepared based on the sequence for Varicella Zoster Virus glycoprotein E, or an antigenic portion thereof, e.g., a dsRNA that encodes

(SEQ ID NO: 20)
MGTVNKPVVGVLMGFGIITGTLRITNPVRASVLRYDDFHIDEDKLDTNSVYEPYYHSDHAESSWVNRGESSRKAYDHN
SPYIWPRNDYDGFLENAHEHHGVYNQGRGIDSGERLMQPTQMSAQEDLGDDTGIHVIPTLNGDDRHKIVNVDQRQYGD
VFKGDLNPKPQGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELDPPEIEPGVLKVLRTEKQYLGVYIWNMRGSDGTSTYAT
FLVTWKGDEKTRNPTPAVTPQPRGAEFHMWNYHSHVFSVGDTFSLAMHLQYKIHEAPFDLLLEWLYVPIDPTCQPMRL
YSTCLYHPNAPQCLSHMNSGCTFTSPHLAQRVASTVYQNCEHADNYTAYCLGISHMEPSFGLILHDGGTTLKFVDTPE
SLSGLYVEVVYFNGHVEAVAYTVVSTVDHFVNAIEERGFPPTAGQPPATTKPKEITPVNPGTSPLLRYAAWTGGLAAV
VLLCLVIFLICTAKRMRVKAYRVDKSPYNQSMYYAGLPVDDFEDSESTDTEEEFGNAIGGSHGGSSYTVYIDKTR,

or a polypeptide having at least 80%, 82%, 85%, 86%, 88%, 90%, 92%, 94%, 95%, 97%, 98% or 99% amino acid sequence identity thereto, such as a dsRNA comprising mRNA sequences having an open reading (ORF) corresponding to an ORF in DNA comprising:

(SEQ ID NO: 21)
atggggacag ttaataaacc tgtggtgggg gtattgatgg ggttcggaat tatcacggga
acgttgcgta taacgaatcc ggtcagagca tccgtcttgc gatacgatga ttttcacatc
gatgaagaca aactggatac aaactccgta tatgagcctt actaccattc agatcatgcg
gagtcttcat gggtaaatcg gggagagtct tcgcgaaaag cgtacgatca taactcacct
tatatatggc cacgtaatga ttatgatgga tttttagaga acgcacacga acaccatggg
gtgtataatc agggccgtgg tatcgatagc ggggaacggt taatgcaacc cacacaaatg
tctgcacagg aggatcttgg ggacgatacg ggcatccacg ttatccctac gttaaacggc
gatgacagac ataaaattgt aaatgtggac caacgtcaat acggtgacgt gtttaaagga
gatcttaatc caaaacccca aggccaaaga ctcattgagg tgtcagtgga agaaaatcac
ccgtttactt tacgcgcacc gattcagcgg atttatggag tccggtacac cgagacttgg
agctttttgc cgtcattaac ctgtacggga gacgcagcgc ccgccatcca gcatatatgt
ttaaaacata caacatgctt tcaagacgtg gtggtggatg tggattgcgc ggaaaatact
aaagaggatc agttggccga aatcagttac cgttttcaag gtaagaagga agcggaccaa
ccgtggatty ttgtaaacac gagcacactg tttgatgaac tcgaattaga cccccccgag
attgaaccgg gtgtcttgaa agtacttcgg acagaaaaac aatacttggg tgtgtacatt
tggaacatgc gcggctccga tggtacgtct acctacgcca cgtttttggt cacctggaaa
ggggatgaaa aaacaagaaa ccctacgccc gcagtaactc ctcaaccaag aggggctgag
tttcatatgt ggaattacca ctcgcatgta ttttcagttg gtgatacgtt tagcttggca
atgcatcttc agtataagat acatgaagcg ccatttgatt tgctgttaga gtggttgtat
gtccccatcg atcctacatg tcaaccaatg cggttatatt ctacgtgttt gtatcatccc
aacgcacccc aatgcctctc tcatatgaat tccggttgta catttacctc gccacattta
gcccagcgtg ttgcaagcac agtgtatcaa aattgtgaac atgcagataa ctacaccgca
tattgtctgg gaatatctca tatggagcct agctttggtc taatcttaca cgacgggggc
accacgttaa agtttgtaga tacacccgag agtttgtcgg gattatacgt ttttgtggtg
tattttaacg ggcatgttga agccgtagca tacactgttg tatccacagt agatcatttt
gtaaacgcaa ttgaagagcg tggatttccg ccaacggccg gtcagccacc ggcgactact
aaacccaagg aaattacccc cgtaaacccc ggaacgtcac cacttctacg atatgccgca
tggaccggag ggcttgcagc agtagtactt ttatgtctcg taatattttt aatctgtacg
gctaaacgaa tgagggttaa agcctatagg gtagacaagt ccccgtataa ccaaagcatg
tattacgctg gccttccagt ggacgatttc gaggactcgg aatctacgga tacggaagaa
gagtttggta acgcgattgg agggagtcac gggggttcga gttacacggt gtatatagat
aagacccggt ga

or a nucleotide sequence having at least 80%, 82%, 85%, 86%, 88%, 90%, 92%, 94%, 95%, 97%, 98% or 99% nucleic acid sequence identity thereto.

A ds RNA may be prepared based on a sequence encoding Ebolavirus glycoprotein, or an antigenic portion thereof, e.g., a dsRNA that encodes

(SEQ ID NO: 22)
MGVTGILQLPRDRFKRTSFFLWVIILFQRTESIPLGVIHNSTLQ
VSDVDKLVCRDKLSSTNQLRSVGLNLEGNGVATDVPSATKRWGFRSGVPPKVVNYEAG
EWAENCYNLEIKKPDGSECLPAAPDGIRGFPRCRYVHKVSGTGPCAGDFAFHKEGAFF
LYDRLASTVIYRGTTFAEGVVAFLILPQAKKDFFSSHPLREPVNATEDPSSGYYSTTI
RYQATGFGTNETEYLFEVDNLTYVQLESRFTPQELLOLNETRYTSGKRSNTTGKLIWK
VNPEIDTTIGEWAFWETKKNLTRKIRSEELSFTAVSNRAKNISGQSPARTSSDPGINT
TTEDHKIMASENSSAMVOVHSQGREAAVSHLTTLATISTSLRPPITKPGPDNSTHNTB
VYKLDISEATQVEQHHRRTDNASTTSDTPPATTAAGPLKAENTNTSKGTDLLDPATTT
SPQNHSETAGNNNTHHODTGEESASSGKLGLITNTIAGVAGLITGGRRTRREAIVNAQ
PKCNPNLHYWTTQDEGAAIGLAWIPYFGPAAEGIYIEGLMHNQDGLICGLRQLANETT
QALQLFIRATTELRTFSILNRKAIDFLLQRWGGTCHILGPDCCIEPHDWTKNITDKID
QIIHDFVDKTLPDQGDNDNWWTGWRQWIPAGIGVIGVIIAVIALFCICKFVF

or a polypeptide having at least 80%, 82%, 85%, 86%, 88%, 90%, 92%, 94%, 95%, 97%, 98% or 99% amino acid sequence identity thereto, such as a dsRNA comprising mRNA sequences having an open reading (ORF) corresponding to an ORF in DNA comprising:

(SEQ ID NO: 23)
gcgatgaaga ttaagccgac agtgagcgta atcttcatct ctcttagatt atttgtcctc
cagagcaggg atcgtcaggt tcttttcaat cgtataacca aaataaactt cactagaagg
atattgtggg gcaacaacac aatgggtgtt acaggaatat tgcagttacc tcgtgatcga
ttcaagagga catcattctt tctttgggta attatccttt tccaaagaac attttccatc
ccacttggag tcatccacaa tagcacatta caggttagtg atgtcgacaa actggtttgc
cgtgacaaac tgtcatccac gaatcaattg agatcagttg gactgaatct cgaagggaat
ggagtggcaa ctgacgtgcc atctgcaact aaaagatggg gcttcaggtc cggtgtccca
ccaaaagtgg tcaattatga agctggtgaa tgggctgaaa actgctacaa tcttgaaatc
aaaaaacctg acgggagtga gtgtctacca gcagcgccag acgggattcg gggcttcccc
cggtgccggt atgtgcacaa agtatcagga acgggaccgt gtgccggaga ctttgccttc
cacaaagagg gtgctttctt cctgtatgat cgacttgctt ccacagttat ctaccgagga
acgactttcg ctgaaggtgt cgtggcattt ctgatactgc cccaagctaa gaaggacttc
ttcagctcac accccttgag agagccggtc aatgcaacgg aggacccgtc tagtggctac
tattctacca caattagata tcaggctacc ggctttggaa ccaatgagac agagtatttg
ttcgaggttg acaatttgac ctacgtccaa cttgaatcaa gattcacacc acagtttctg
ctccagctga atgagacaag atatacaagt gggaaaagga gcaataccac gggaaaacta
atttggaagg tcaaccccga aattgataca acaatcgggg agtgggcctt ctgggaaact
aaaaaaacct cactagaaaa attcgcagtg aagagttgtc tttcacagct gtatcaaaca
gagccaaaaa catcagtggt cagagtccgg cgcgaacttc ttccgaccca gggaccaaca
caacaactga agaccacaaa atcatggctt cagaaaattc ctctgcaatg gttcaagtgc
acagtcaagg aagggaagct gcagtgtcgc atctgacaac ccttgccaca atctccacga
gtcttcgacc ccccataacc aaaccaggtc cggacaacag cacccacaat acacccgtgt
ataaacttga catctctgag gcaactcaag ttgaacaaca tcaccgcaga acagacaacg
ccagcacaac ctccgacact ccccccgcca cgaccgcagc cggaccccta aaagcagaga
acaccaacac gagcaagggc actgacctcc tggaccccgc caccacaaca agtccccaaa
accacagcga gaccgctggc aacaacaaca ctcatcacca agataccgga gaagagagtg
ccagcagcgg gaagctaggc ttaattacca atactattgc tggagtcgca ggactgatca
caggcgggag aagaactcga agagaagcaa ttgtcaatgc tcaacccaaa tgcaacccta
atttacatta ctggactact caggatgaag gtgctgcaat cggactggcc tggataccat
atttcgggcc agcagccgag ggaatttaca tagaggggct gatgcacaat caagatggtt
taatctgtgg gttgagacag ctggccaacg agacgactca agctcttcaa ctattcctga
gagccacaac cgagctacgc accttttcaa tcctcaaccg taaggcaatt gatttcttgc
tgcagcgatg gggcggcaca tgccacattt tgggaccgga ctgctgtatc gaaccacatg
attggactaa gaacataacg gacaaaattg atcagattat tcatgatttt gttgataaaa
cccttccgga ccagggggac aatgacaatt ggtggacagg atggagacag tggataccgg
caggtattgg agttacaggc gttataattg cagttatcgc tttattctgt atatgcaaat
ttgtctttta gtttttcttc agattgcttc atggcaaagc tcagcctcaa atcaatgaaa
ccaggattta attatatgga ttacttgaat ctaagattac ttgacaaatg ataatataat
acactggagc tttaaacata gccaatgtga ttctaactct tttaaactca cagttaatca
taaataaggt ttgacatcaa tctagttatc tctttgagaa tgataaactt gatgaagatt
aagaaaaagg taatctttcg attatcttta atcttcatcc ttgatt

or a nucleotide sequence having at least 80%, 82%, 85%, 86%, 88%, 90%, 92%, 94%, 95%, 97%, 98% or 99% nucleic acid sequence identity thereto.

A ds RNA may be prepared based on a sequence that encodes SARS-Covid-Spike protein, or an antigenic portion thereof, e.g., a dsRNA encoding

(SEQ ID NO: 24)
MFVFLVLLPLVSSQCVNLTTRTQLPPAYTNSFTRGVYYPDKVFR
SSVLASTQDLFLPFFSNVTWFHAIHVSGINGIKRFDNPVLPENDGVYFASTEKSNIIR
GWIFGTTLDSKTQSLLIVNNATNVVIKVCEFQFCNDPFLGVYYHKNNKSWMESEFRVY
SSANNCTFEYVSQPFLMDLEGKQGNEKNLREFVEKNIDGYFKIYSKHTPINLVRDLPQ
GFSALEPLVDLPIGINITRFQTLLALHRSYLTPGDSSSGWTAGAAAYYVGYLQPRTFL
LKYNENGTITDAVDCALDPLSETKCTLKSFTVEKGIYQTSNERVQPTESIVREPNITN
LCPFGEVENATREASVYAWNRKRISNCVADYSVLYNSASFSTFKCYGVSPTKINDLCF
TNVYADSEVIRGDEVRQIAPGQTGKIADYNYKLPDDETGCVIAWNSNNLDSKVGGNYN
YLYRLFRKSNLKPFERDISTEIYQAGSTPCNGVEGENCYFPLQSYGFQPINGVGYQPY
RVVVLSFELLHAPATVCGPKKSTNLVKNKCVNENENGLIGTGVLTESNKKELPFQQFG
RDIADITDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFG
GFNFSQILPDPSKPSKRSFIEDLLENKVTLADAGFIKQYGDCLGDIAARDLICAQKFN
GLTVLPPLLTDEMIAQYTSALLAGTITSGWTFGAGAALQIPFAMQMAYRENGIGVTQN
VLYENQKLIANQFNSAIGKIQDSLSSTASALGKLQDVVNQNAQALNTLVKQLSSNFGA
ISSVINDILSRLDKVEAEVQIDRLITGRLQSLQTYVTQQLIRAAEIRASANLAATKMS
ECVLGQSKRVDFCGKGYHLMSFPQSAPHGVVELHVTYVPAQEKNFTTAPAICHDGKAH
FPREGVFVSNGTHWFVTQRNFYEPQIITTDNTEVSGNCDVVIGIVNNTVYDPLQPELD
SFKEELDKYEKNHTSPDVDLGDISGINASVVNIQKEIDRINEVAKNINESLIDLQELG
KYEQYIKWPWYIWLGFIAGLIAIVMVTIMLCCMTSCCSCLKGCCSCGSCCKFDEDDSE
PVLKGVKLHYT

or a polypeptide having at least 80%, 82%, 85%, 86%, 88%, 90%, 92%, 94%, 95%, 97%, 98% or 99% amino acid sequence identity thereto, such as a dsRNA comprising mRNA sequences having an open reading (ORF) corresponding to an ORF in DNA comprising:

(SEQ ID NO: 25)
tgtgcaactg tacatacagc taataaatgg gatctcatta ttagtgatat gtacgaccct
aagactaaaa atgttacaaa agaaaatgac tctaaagagg gttttttcac ttacatttgt
gggtttatac aacaaaagct agctcttgga ggttccgtgg ctataaagat aacagaacat
tcttggaatg ctgatcttta taagctcatg ggacacttcg catggtggac agcctttgtt
actaatgtga atgcgtcatc atctgaagca tttttaattg gatgtaatta tcttggcaaa
ccacgcgaac aaatagatgg ttatgtcatg catgcaaatt acatattttg gaggaataca
aatccaattc agttgtcttc ctattcttta tttgacatga gtaaatttcc ccttaaatta
aggggtactg ctgttatgtc tttaaaagaa ggtcaaatca atgatatgat tttatctctt
cttagtaaag gtagacttat aattagagaa aacaacagag ttgttatttc tagtgatgtt
cttgttaaca actaaacgaa caatgtttgt ttttcttgtt ttattgccac tagtctctag
tcagtgtgtt aatcttacaa ccagaactca attaccccct gcatacacta attctttcac
acgtggtgtt tattaccctg acaaagtttt cagatcctca gttttacatt caactcagga
cttgttctta cctttctttt ccaatgttac ttggttccat gctatacatg tctctgggac
caatggtact aagaggtttg ataaccctgt cctaccattt aatgatggtg tttattttgc
ttccactgag aagtctaaca taataagagg ctggattttt ggtactactt tagattcgaa
gacccagtcc ctacttattg ttaataacgc tactaatgtt gttattaaag tctgtgaatt
tcaattttgt aatgatccat ttttgggtgt ttattaccac aaaaacaaca aaagttggat
ggaaagtgag ttcagagttt attctagtgc gaataattgc acttttgaat atgtctctca
gccttttctt atggaccttg aaggaaaaca gggtaatttc aaaaatctta gggaatttgt
gtttaagaat attgatggtt attttaaaat atattctaag cacacgccta ttaatttagt
gcgtgatctc cctcagggtt tttcggcttt agaaccattg gtagatttgc caataggtat
taacatcact aggtttcaaa ctttacttgc tttacataga agttatttga ctcctggtga
ttcttcttca ggttggacag ctggtgctgc agcttattat gtgggttatc ttcaacctag
gacttttcta ttaaaatata atgaaaatgg aaccattaca gatgctgtag actgtgcact
tgaccctctc tcagaaacaa agtgtacgtt gaaatccttc actgtagaaa aaggaatcta
tcaaacttct aactttagag tccaaccaac agaatctatt gttagatttc ctaatattac
aaacttgtgc ccttttggtg aagtttttaa cgccaccaga tttgcatctg tttatgcttg
gaacaggaag agaatcagca actgtgttgc tgattattct gtcctatata attccgcatc
attttccact tttaagtgtt atggagtgtc tcctactaaa ttaaatgatc tctgctttac
taatgtctat gcagattcat ttgtaattag aggtgatgaa gtcagacaaa tcgctccagg
gcaaactgga aagattgctg attataatta taaattacca gatgatttta caggctgcgt
tatagcttgg aattctaaca atcttgattc taaggttggt ggtaattata attacctgta
tagattgttt aggaagtcta atctcaaacc ttttgagaga gatatttcaa ctgaaatcta
tcaggccggt agcacacctt gtaatggtgt tgaaggtttt aattgttact ttcctttaca
atcatatggt ttccaaccca ctaatggtgt tggttaccaa ccatacagag tagtagtact
ttcttttgaa cttctacatg caccagcaac tgtttgtgga cctaaaaagt ctactaattt
ggttaaaaac aaatgtgtca atttcaactt caatggttta acaggcacag gtgttcttac
tgagtctaac aaaaagtttc tgcctttcca acaatttggc agagacattg ctgacactac
tgatgctgtc cgtgatccac agacacttga gattcttgac attacaccat gttcttttgg
tggtgtcagt gttataacac caggaacaaa tacttctaac caggttgctg ttctttatca
ggatgttaac tgcacagaag tccctgttgc tattcatgca gatcaactta ctcctacttg
gcgtgtttat tctacaggtt ctaatgtttt tcaaacacgt gcaggctgtt taataggggc
tgaacatgtc aacaactcat atgagtgtga catacccatt ggtgcaggta tatgcgctag
ttatcagact cagactaatt ctcctcggcg ggcacgtagt gtagctagtc aatccatcat
tgcctacact atgtcacttg gtgcagaaaa ttcagttgct tactctaata actctattgc
catacccaca aattttacta ttagtgttac cacagaaatt ctaccagtgt ctatgaccaa
gacatcagta gattgtacaa tgtacatttg tggtgattca actgaatgca gcaatctttt
gttgcaatat ggcagttttt gtacacaatt aaaccgtgct ttaactggaa tagctgttga
acaagacaaa aacacccaag aagtttttgc acaagtcaaa caaatttaca aaacaccacc
aattaaagat tttggtggtt ttaatttttc acaaatatta ccagatccat caaaaccaag
caagaggtca tttattgaag atctactttt caacaaagtg acacttgcag atgctggctt
catcaaacaa tatggtgatt gccttggtga tattgctgct agagacctca tttgtgcaca
aaagtttaac ggccttactg ttttgccacc tttgctcaca gatgaaatga ttgctcaata
cacttctgca ctgttagcgg gtacaatcac ttctggttgg acctttggtg caggtgctgc
attacaaata ccatttccta tgcaaatggc ttataggttt aatggtattg gagttacaca
gaatgttctc tatgagaacc aaaaattgat tgccaaccaa tttaatagtg ctattggcaa
aattcaagac tcactttctt ccacagcaag tgcacttgga aaacttcaag atgtggtcaa
ccaaaatgca caagctttaa acacgcttgt taaacaactt agctccaatt ttggtgcaat
ttcaagtgtt ttaaatgata tcctttcacg tcttgacaaa gttgaggctg aagtgcaaat
tgataggttg atcacaggca gacttcaaag tttgcagaca tatgtgactc aacaattaat
tagagctgca gaaatcagag cttctgctaa tcttgctgct actaaaatgt cagagtgtgt
acttggacaa tcaaaaagag ttgatttttg tggaaagggc tatcatctta tgtccttccc
tcagtcagca cctcatggtg tagtcttctt gcatgtgact tatgtccctg cacaagaaaa
gaacttcaca actgctcctg ccatttgtca tgatggaaaa gcacactttc ctcgtgaagg
tgtctttgtt tcaaatggca cacactggtt tgtaacacaa aggaattttt atgaaccaca
aatcattact acagacaaca catttgtgtc tggtaactgt gatgttgtaa taggaattgt
caacaacaca gtttatgatc ctttgcaacc tgaattagac tcattcaagg aggagttaga
taaatatttt aagaatcata catcaccaga tgttgattta ggtgacatct ctggcattaa
tgcttcagtt gtaaacattc aaaaagaaat tgaccgcctc aatgaggttg ccaagaattt
aaatgaatct ctcatcgatc tccaagaact tggaaagtat gagcagtata taaaatggcc
atggtacatt tggctaggtt ttatagctgg cttgattgcc atagtaatgg tgacaattat
gctttgctgt atgaccagtt gctgtagttg tctcaagggc tgttgttctt gtggatcctg
ctgcaaattt gatgaagacg actctgagcc agtgctcaaa ggagtcaaat tacattacac
ataaacgaac ttatggattt gtttatgaga atcttcacaa ttggaactgt aactttgaag

or a nucleotide sequence having at least 80%, 82%, 85%, 86%, 88%, 90%, 92%, 94%, 95%, 97%, 98% or 99% nucleic acid sequence identity thereto, or in Accession No. MG772933.

Ads RNA may be prepared based on sequences encoding Dengue virus envelope and/or premembrane proteins of at least one of 4 serotypes, or an antigenic portion thereof, e.g., a dsRNA encoding envelope and/or premembrane protein in the following polyprotein:

(SEQ ID NO: 26)
MINQRKKTGRPSFNMLKRARNRVSTVSQLAKRFSKGLLSGQGPMKLVMA
FIAFLRFLAIPPTAGILARWGSFKKNGAIKVLRGFKREISNMLNIMNRR
KRSVTMLLMLLPTALAFHLTTRGGEPHMIVSKQERGKSLLFKTSAGVNM
CTLIAMDLGELCEDTMTYKCPRITEAEPDDVDCWCNATDTWVTYGTCSQ
TGEHRRDKRSVALAPHVGLGLETRAETWMSSEGAWKQIQKVETWALRHP
GFTVIALFLAHAIGTSITQKGIIFILLMLVTPSMAMRCVGIGNRDFVEG
LSGATWVDVVLEHGSCVTTMAKNKPTLDIELLKTEVTNPAVLRKLCIEA
KISNTTTDSRCPTQGEATLVEEQDANFVCRRTFVDRGWGNGCGLFGKGS
LITCAKFKCVTKLEGKIAQYENLKYSVIVTVHTGDQHQVGNETTEHGTT
ATITPQAPTSEIQLTDYGTLTLDCSPRTGLDENEMVLLTMKKKSWLVHK
QWFLDLPLPWTSGALTSQETWNRQDLLVTFKTAHAKKQEVVVLGSQEGA
MHTALTGATEIQTSGTTTIFAGHLKCRLKMDKLTLKGMSYVMCTGSFKL
EKEVAETQHGTVLVQVKYEGTDAPCKIPFSTQDEKGATQNGRLITANPI
VTDKEKPVNIEAEPPFGESYIVVGAGEKALKLSWFKKGSSIGKMFEATA
RGARRMAILGDTAWDFGSIGGVFTSMGKLVHQVFGTAYGVLFSGVSWTM
KIGIGILLTWLGLNSRNTSLSVMCIAVGMVTLYLGVMVQADSGCVINWK
GRELKCGSGIFVTNEVHTWTEQYKFQADSPKRLSAAIGKAWEEGVCGIR
SATRLENIMWKQISNELNHILLENDMKFTVVVGDVSGILAQGKKMIRPQ
PMEHKYSWKSWGKAKIIGADVQNTTFIIDGPNTPECPDNQRAWNIWEVE
DYGFGIFTTNIWLKLRDSYTQVCDHRLMSAAIKDSKAVHADMGYWIESE
KNETWKLARASFIEVKTCIWPKSHTLWSNGVLESEMIIPKIYGGPISQH
NYRPGYFTQTAGPWHLGKLELDFDFCEGTTVVVDEHCGNRGPSLRTTTV
TGKIIHEWCCRSCTLPPLRFKGEDGCWYGMEIRPVKDKEENLVKSMVSA
GSGEVDSFSLGLLCISIMIEEVMRSRWSKKMLMTGTLAVFLLLIMGQLT
WSDLIRLCIMVGANASDKMGMGTTYLALMATFKMRPMFAVGLLFRRLTS
REVLLLTIGLSLVASVELPSSLEELGDGLAIGIMMLKLLTDFQSHQLWA
TLLSLTFIKTTFSLHYAWKTMAMVLSIVSLFPLCLSTTSQKTTWLPVLL
GSLGCKPLPMFLITENKIWGRKSWPLNEGIMAVGIVSILLSSLLKNDVP
LAGPLIAGGMLIACYVISGSSADLSLEKAAEVSWEEEAEHSGASHNILV
EVQDDGTMKIKDEERDDTLTILLKATLLAVSGVYPMSIPATLFVWYFWQ
KKKQRSGVLWDTPSPPEVERAVLDDGIYRILQRGLLGRSQVGVGVFQEG
VFHTMWHVTRGAVLMYQGKRLEPSWASVKKDLISYGGGWRFQGSWNTGE
EVQVIAVEPGKNPKNVQTTPGTFKTPEGEVGAIALDFKPGTSGSPIVNR
EGKIVGLYGNGVVTTSGTYVSAIAQAKASQEGPLPEIEDKVFRKRNLTI
MDLHPGSGKTRRYLPAIVREAIKRKLRTLILAPTRVVASEMAEALKGVP
IRYQTTAVKSEHTGKEIVDLMCHATFTMRLLSPVRVPNYNMIIMDEAHF
TDPASIAARGYISTRVGMGEAAAIFMTATPPGSVEAFPQSNAIIQDEER
DIPERSWNSGYDWITDFPGKTVWFVPSIKSGNDIANCLRKNGKRVIQLS
RKTFDTEYQKTKNNDWDYVVTTDISEMGANFRADRVIDPRRCLKPVILK
DGPERVILAGPMPVTVASAAQRRGRIGRNQNKEGDQYIYMGQPLNNDED
HAHWTEAKMLLDNINTPEGIIPALFEPEREKSAAIDGEYRLRGEARKTF
VELMRRGDLPVWLSYKVASEGFQYSDRRWCFDGERNNQVLEENMDVEIW
TKEGERKKLRPRWLDARTYSDPLALREFKEFAAGRRSVSGDLILEIGKL
PQHLTQRAQNALDNLVMLHNSEQGGKAYRHAMEELPDTIETLMLLALIA
VLTGGVTLFFLSGRGLGKTSIGLLCVMASSALLWMASVEPHWIAASIIL
EFFLMVLLIPEPDRQRTPQDNQLAYVVIGLLFVILTVAANEMGLLETTK
KDLGIGHVAAENHHHATMLDVDLHPASAWTLYAVATTIITPMMRHTIEN
TTANISLTAIANQAAILMGLDKGWPISKMDIGVPLLALGCYSQVNPLTL
IAAVLMLVAHYAIIGPGLQAKATREAQKRTAAGIMKNPTVDGIVAIDLD
PVVYDAKFEKQLGQIMLLILCTSQILLMRTTWALCESITLATGPLTTLW
EGSPGKFWNTTIAVSMANIFRGSYLAGAGLAFSLMKSLGGGRRGTGAQG
ETLGEKWKRQLNQLSKSEFNTYKRSGIMEVDRSEAKEGLKRGETTKHAV
SRGTAKLRWFVERNLVKPEGKVIDLGCGRGGWSYYCAGLKKVTEVKGYT
KGGPGHEEPIPMATYGWNLVKLHSGKDVFFIPPEKCDTLLCDIGESSPN
PTIEEGRTLRVLKMVEPWLRGNQFCIKILNPYMPSVVETLEQMQRKHGG
MLVRNPLSRNSTHEMYWVSCGTGNIVSAVNMTSRMLLNRFTMAHRKPTY
ERDVDLGAGTRHVAVEPEVANLDIIGQRIENIKNEHKSTWHYDEDNPYK
TWAYHGSYEVKPSGSASSMVNGVVRLLTKPWDVIPMVTQIAMTDTTPFG
QQRVFKEKVDTRTPKAKRGTAQIMEVTARWLWGFLSRNKKPRICTREEF
TRKVRSNAAIGAVFVDENQWNSAKEAVEDERFWELVHRERELHKQGKCA
TCVYNMMGKREKKLGEFGKAKGSRAIWYMWLGARFLEFEALGEMNEDHW
FSRENSLSGVEGEGLHKLGYILRDISRIPGGNMYADDTAGWDTRITEDD
LQNEAKITDIMEPEHALLATSIFKLTYQNKVVRVQRPAKNGTVMDVISR
RDQRGSGQVGTYGLNTFTNMEAQLIRQMESEGIFLPSELETPNLAGRVL
DWLEKYGVERLKRMAISGDDCVVKPIDDRFATALTALNDMGKVRKDIPQ
WEPSKGWNDWQQVPFCSHHFHQLIMKDGREIVVPCRNQDELVGRARVSQ
GAGWSLRETACLGKSYAQMWQLMYFHRRDLRLAANAICSAVPVDWVPTS
RTTWSIHAHHQWMTTEDMLSVWNRVWIEENPWMEDKTHVSSWEEVPYLG
KREDQWCGSLIGLTARATWATNIQVAINQVRRLIGNENYLDYMTSMKRF
KNESDPEGALW

or a polypeptide having at least 80%, 82%, 85%, 86%, 88%, 90%, 92%, 94%, 95%, 97%, 98% or 99% amino acid sequence identity thereto, e.g., to residues 437 to 934 or residues 935 to 2419, such as a dsRNA corresponding to RNA having an open reading (ORE) corresponding to an ORE in DNA comprising:

(SEQ ID NO: 27)
agttgttagt ctacgtggac cgacaagaac agtttcgaat cggaagcttg cttaacgtag
ttctaacagt tttttattag agagcagatc tctgatgatc aaccaacgaa aaaagacggg
tcgaccgtct ttcaatatgc tgaaacgcgc gagaaaccgc gtgtcaactg tttcacagtt
ggcgaagaga ttctcaaaag gattgctctc aggccaagga cccatgaaat tggtgatggc
tttcatagca ttcttaagat ttctagccat acccccaaca gcaggaattt tggctagatg
gggctcattc aagaagaatg gagcgattaa agtgttacgg ggtttcaaga gagaaatctc
aaacatgcta aacataatga acaggaggaa aagatccgtg accatgctcc ttatgctgct
gcccacagcc ctggcgttcc atctgacgac acgaggggga gagccgcata tgatagttag
caagcaggaa agaggaaagt cacttttgtt caagacctct gcaggtgtca acatgtgcac
cctcattgcg atggatttgg gagagttgtg tgaggacacg atgacctaca aatgcccccg
gatcactgag gcggaaccag atgacgttga ctgttggtgc aatgccacgg acacatgggt
gacctatgga acgtgctctc aaactggcga acaccgacga gacaaacgtt ccgtcgcatt
ggccccacac gtggggcttg gcctagaaac aagagccgaa acgtggatgt cctctgaagg
tgcttggaaa cagatacaaa aagtagagac ttgggctctg agacatccag gattcacggt
gatagccctt tttctagcac atgccatagg aacatccatc acccagaaag ggatcatttt
cattttgctg atgctggtaa caccatctat ggccatgcga tgcgtgggaa taggcaacag
agacttcgtg gaaggactgt caggagcaac atgggtggat gtggtactgg agcatggaag
ttgcgtcacc accatggcaa aaaacaaacc aacactggac attgaactct tgaagacgga
ggtcacaaac cctgcagttc tgcgtaaatt gtgcattgaa gctaaaatat caaacaccac
caccgattcg agatgtccaa cacaaggaga agccacactg gtggaagaac aagacgcgaa
ctttgtgtgc cgacgaacgt tcgtggacag aggctggggc aatggctgtg ggctattcgg
aaaaggtagt ctaataacgt gtgccaagtt taagtgtgtg acaaaactag aaggaaagat
agctcaatat gaaaacctaa aatattcagt gatagtcacc gtccacactg gagatcagca
ccaggtggga aatgagacta cagaacatgg aacaactgca accataacac ctcaagctcc
tacgtcggaa atacagctga ccgactacgg aaccettaca ttagattgtt cacctaggac
agggctagat tttaacgaga tggtgttgct gacaatgaaa aagaaatcat ggcttgtcca
caaacagtgg tttctagact taccactgcc ttggacctct ggggctttaa catcccaaga
gacttggaac agacaagatt tactggtcac atttaagaca gctcatgcaa agaagcagga
agtagtcgta ctaggatcac aagaaggagc aatgcacact gcgctgactg gagcgacaga
aatccaaacg tcaggaacga caacaatttt cgcaggacac ctaaaatgca gactaaaaat
ggacaaacta actttaaaag ggatgtcata tgtgatgtgc acaggctcat tcaagttaga
gaaagaagtg gctgagaccc agcatggaac tgttctggtg caggttaaat atgaaggaac
agacgcacca tgcaagattc ccttttcgac ccaagatgag aaaggagcaa cccagaatgg
gagattaata acagccaacc ccatagtcac tgacaaagaa aaaccagtca atattgaggc
agaaccaccc tttggtgaga gctacatcgt ggtaggagca ggtgaaaaag ctttgaaact
aagctggttc aagaaaggaa gcagcatagg gaaaatgttt gaagcaactg cccgaggagc
acgaaggatg gccattctgg gagacaccgc atgggacttc ggttctatag gaggagtgtt
cacgtctatg ggaaaactgg tacaccaggt ttttggaact gcatatggag ttttgtttag
cggagtttct tggaccatga aaataggaat agggattctg ctgacatggc taggattaaa
ttcaaggaac acgtcccttt cggtgatgtg catcgcagtt ggcatggtca cactgtacct
aggagtcatg gttcaggcag attcgggatg tgtaatcaac tggaaaggca gagaacttaa
atgtggaagc ggcatttttg tcactaatga agttcacact tggacagagc aatacaaatt
ccaggctgac tcccccaaga gactatcagc agccattggg aaggcatggg aggagggtgt
gtgtggaatc cgatcagcca ctcgtctcga gaacatcatg tggaaacaaa tatcaaatga
attgaaccac atcctacttg aaaatgacat gaaatttaca gtggtcgtgg gagacgttag
tggaatcttg gcccaaggga aaaaaatgat taggccacaa cccatggaac acaaatactc
gtggaaaagc tggggaaaag ctaaaatcat aggagcggat gtacagaaca ccaccttcat
catcgacggc ccaaacaccc cagaatgccc tgacaatcaa agagcatgga atatttggga
agtagaggac tatggatttg ggattttcac gacaaacata tggttgaaat tgcgtgactc
ctacacccaa gtatgtgacc accggctgat gtcagctgcc attaaggaca gcaaggcagt
ccatgctgac atggggtact ggatagaaag tgaaaagaac gagacatgga agttggcgag
agcctccttt atagaagtta agacatgcat ctggccaaaa tcccacactc tatggagcaa
tggagttctg gaaagtgaaa tgataattcc aaagatatat ggaggaccaa tatctcagca
caactacaga ccaggatatt tcacacaaac agcagggccg tggcacctag gcaagttgga
actagatttc gatttttgtg aaggtaccac agttgttgtg gatgaacatt gtggaaatcg
aggaccatct ctcagaacca caacagtcac aggaaagata atccatgaat ggtgctgcag
atcttgtacg ctaccccccc tacgtttcaa aggggaagac gggtgttggt acggcatgga
aatcagacca gtgaaggaca aggaagagaa cctggtcaag tcaatggtct ctgcagggtc
aggagaagtg gacagctttt cactaggact gctatgcata tcaataatga ttgaagaagt
gatgagatcc agatggagca aaaaaatgct gatgactgga acactggctg tgttcctcct
tcttataatg ggacaattga catggagtga tctgatcagg ttatgtatta tggttggagc
caacgcttca gacaagatgg ggatgggaac aacgtaccta gctttaatgg ccactttcaa
aatgagacca atgttcgccg tcgggctatt atttcgcaga ctaacatcta gagaagttct
tcttcttaca attggcttga gcctggtggc atccgtggag ctaccaagtt ccctagagga
gctgggggat ggacttgcaa taggcatcat gatgttgaaa ttattgactg attttcagtc
acaccagcta tgggctactc tgctatcctt gacatttatt aaaacaactt tttcattgca
ctatgcatgg aagacaatgg ctatggtact gtcaattgta tctctcttcc ctttatgcct
gtccacgacc tctcaaaaaa caacatgget tccggtgctg ttgggatctc ttggatgcaa
accactaccc atgtttctta taacagaaaa caaaatctgg ggaaggaaga gttggcccct
caatgaagga attatggctg ttggaatagt tagtattcta ctaagttcac ttttaaaaaa
tgatgtgccg ctagccggcc cattaatagc tggaggcatg ctaatagcat gttatgtcat
atccggaagc tcagctgatt tatcactgga gaaagcggct gaggtctcct gggaggaaga
agcagaacac tcaggcgcct cacacaacat actagtagag gttcaagatg atggaaccat
gaagataaaa gatgaagaga gagatgacac gctcaccatt ctccttaaag caactctgct
ggcagtctca ggggtgtacc caatgtcaat accagcgacc ctttttgtgt ggtatttttg
gcagaaaaag aaacagagat caggagtgct atgggacaca cccagccccc cagaagtgga
aagagcagtt cttgatgatg gcatctatag aattttgcaa agaggactgt tgggcaggtc
ccaagtagga gtaggagttt tccaagaagg cgtgttccac acaatgtggc acgtcactag
gggagctgtc ctcatgtatc aaggaaaaag gctggaacca agctgggcca gtgtcaaaaa
agacttgatc tcatatggag gaggttggag gtttcaagga tcctggaaca cgggagaaga
agtacaggtg attgctgttg aaccgggaaa aaaccccaaa aatgtacaaa caacgccggg
taccttcaag acccctgaag gcgaagttgg agccatagcc ttagacttta aacctggcac
atctggatct cccatcgtaa acagagaggg aaaaatagta ggtctttatg gaaatggagt
ggtgacaaca agcggaactt acgttagtgc catagctcaa gctaaggcat cacaagaagg
gcctctacca gagattgagg acaaggtgtt taggaaaaga aacttaacaa taatggacct
acatccagga tcgggaaaaa caagaagata ccttccagcc atagtccgtg aggccataaa
aaggaagctg cgcacgctaa tcctagetcc cacaagagtt gtcgcttctg aaatggcaga
ggcactcaag ggagtgccaa taaggtatca gacaacagca gtgaagagtg aacacacagg
aaaggagata gttgacctta tgtgccacgc cactttcacc atgcgcctcc tgtctcccgt
gagagttccc aattataaca tgattatcat ggatgaagca cacttcaccg atccagccag
catagcagcc agagggtaca tctcaacccg agtgggtatg ggtgaagcag ctgcgatctt
tatgacagcc actcccccag gatcggtgga ggcctttcca cagagcaatg caattatcca
agatgaggaa agagacattc ctgagagatc atggaactca ggctatgact ggatcactga
ttttccaggt aaaacagtct ggtttgttcc aagcatcaaa tcaggaaatg acattgccaa
ctgtttaaga aaaaacggga aacgggtgat ccaattgagc agaaaaacct ttgacactga
gtaccagaaa acaaaaaaca acgactggga ctatgtcgtc acaacagaca tttccgaaat
gggagcaaat ttccgggccg acagggtaat agacccaagg cggtgtctga aaccggtaat
actaaaagat ggtccagagc gcgtcattct agccggaccg atgccagtga ctgtggccag
tgccgcccag aggagaggaa gaattggaag gaaccaaaac aaggaaggtg atcagtatat
ttacatggga cagcctttaa acaatgatga ggaccacgct cattggacag aagcaaagat
gctccttgac aatataaaca caccagaagg gattatccca gccctctttg agccggagag
agaaaagagt gcagctatag acggggaata cagactgcgg ggtgaagcaa ggaaaacgtt
cgtggagctc atgagaagag gggatctacc agtctggcta tcctacaaag ttgcctcaga
aggcttccag tactccgaca gaaggtggtg cttcgatggg gaaaggaaca accaggtgtt
ggaggagaac atggacgtgg agatctggac aaaagaagga gaaagaaaga aactacgacc
tcgctggttg gacgccagaa catactctga cccactggct ctgcgcgagt ttaaagagtt
tgcagcagga agaagaagcg tctcaggtga cctaatatta gaaataggga aacttccaca
acatttgacg caaagggccc agaatgcttt ggacaacttg gtcatgttgc acaattccga
acaaggagga aaagcctata gacatgctat ggaagaactg ccagacacaa tagaaacgtt
gatgctccta gccttgatag ctgtgttgac tggtggagtg acgctgttct tcctatcagg
aagaggtcta ggaaaaacat ctatcggctt actctgcgtg atggcctcaa gcgcactgtt
atggatggcc agtgtggagc cccattggat agcggcctcc atcatactgg agttctttct
gatggtactg cttattccag agccagacag acagcgcact ccacaggaca accagctagc
atatgtggtg ataggtctgt tattcgtgat attgacagtg gcagccaatg agatgggatt
attggaaacc acaaagaaag acctggggat tggccatgta gctgctgaaa accaccacca
tgctacaatg ctggacgtag acctacatcc agcttcagcc tggaccctct atgcagtggc
cacaacaatc atcactccta tgatgagaca cacaattgaa aacacaacgg caaatatttc
cctgacagcc atcgcaaacc aagcagctat attgatggga cttgacaagg gatggccaat
atcgaagatg gacataggag ttccacttct cgccttgggg tgctattccc aagtgaatcc
gctgacactg atagcggcag tattgatgct agtagctcat tacgccataa ttggacctgg
actgcaagca aaagctacta gagaagctca aaaaagaaca gcggctggaa taatgaaaaa
tccaactgtc gacgggattg ttgcaataga cttagatccc gtggtttacg atgcaaaatt
tgaaaaacag ctaggccaaa taatgttgtt gatactttgc acatcacaga ttcttttgat
gcggactaca tgggccttgt gtgaatccat cacattggct actggacctc tgaccactct
ttgggaggga tctccaggaa aattctggaa caccacaata gcggtatcca tggcaaacat
tttcaggggg agttatctag caggagcagg tctggccttc tcattaatga aatctctagg
aggaggtagg agaggcacgg gagcccaagg ggaaacactg ggagaaaaat ggaaaagaca
actaaaccaa ctgagcaagt cagaattcaa tacttacaag aggagtggga ttatggaggt
ggatagatcc gaagccaaag agggactgaa aagaggagaa acaaccaaac acgcagtatc
gagaggaacg gccaaactga ggtggttcgt ggagaggaac cttgtgaaac cagaagggaa
agtcatagac ctcggttgtg gaagaggtgg ctggtcatat tattgcgctg ggctgaagaa
agtcacagaa gtgaaaggat acacaaaagg aggacctgga catgaggaac caatcccaat
ggcgacctat ggatggaacc tagtaaagct gcactccgga aaagatgtat tttttatacc
acctgagaaa tgtgacaccc ttttgtgtga tattggtgag tectctccga acccaactat
agaggaagga agaacgttac gtgttctgaa aatggtggaa ccatggctca gaggaaacca
attttgcata aaaattctaa atccctatat gccgagcgtg gtagaaactc tggaacaaat
gcaaagaaaa catggaggaa tgctagtgcg aaacccactc tcaagaaatt ccacccatga
aatgtactgg gtttcatgtg gaacaggaaa cattgtgtca gcagtaaaca tgacatctag
aatgttgcta aatcggttca caatggctca caggaagcca acatatgaaa gagacgtgga
cttaggcgct ggaacaagac atgtggcagt agaaccagag gtagccaacc tagatatcat
tggccagagg atagagaata taaaaaatga acataagtca acatggcatt atgatgagga
caatccatac aaaacatggg cctatcatgg atcatatgag gttaagccat caggatcggc
ctcatccatg gtcaatggcg tggtgagatt gctcaccaaa ccatgggatg ttatccccat
ggtcacacaa atagccatga ctgataccac accctttgga caacagaggg tgtttaaaga
gaaagttgac acgcgcacac caaaagcaaa acgtggcaca gcacaaatta tggaagtgac
agccaggtgg ttatggggtt tcctttctag aaacaaaaaa cccagaattt gcacaagaga
ggagtttaca agaaaagtta ggtcaaacgc agctattgga gcagtgttcg ttgatgaaaa
tcaatggaac tcggcaaaag aagcagtgga agacgaacgg ttctgggaac ttgtccacag
agagagggag cttcataaac aggggaaatg tgccacgtgt gtctacaata tgatggggaa
gagagagaaa aaattaggag agttcggaaa ggcaaaagga agtcgtgcaa tatggtacat
gtggttggga gcacgcttcc tagagtttga agcccttggt ttcatgaatg aagatcactg
gttcagtaga gagaattcac tcagtggagt ggaaggagaa ggactccaca aacttggata
catactcaga gacatatcaa ggattccagg ggggaacatg tatgcagatg acacagccgg
atgggacaca agaataacag aggatgatct ccagaatgag gctaaaatca ctgacatcat
ggagcccgaa catgccctgc tggctacgtc aatctttaag ctgacctacc aaaataaggt
ggtaagggtg cagagaccag caaaaaatgg aaccgtgatg gatgttatat ccagacgtga
ccagagaggc agtggacagg ttggaactta tggcttaaac actttcacca acatggaggc
ccaactgata agacaaatgg agtctgaggg aatcttttta cccagcgaat tggaaacccc
aaatctagcc ggaagagttc tcgactggtt ggaaaaatat ggtgtcgaaa ggctgaaaag
aatggcaatc agcggagatg actgtgtggt gaaaccaatt gatgacaggt tcgcaacagc
cttaacagct ttgaatgaca tgggaaaagt aagaaaagac ataccacaat gggaaccttc
aaaaggatgg aatgattggc aacaagtgcc tttctgttca caccacttcc accagctaat
tatgaaggat gggagggaga tagtggtgcc atgccgcaac caagatgaac ttgtggggag
ggccagagta tcacaaggcg ccggatggag cctgagagaa accgcatgcc taggcaagtc
atatgcacaa atgtggcagc tgatgtattt ccacaggaga gacctgagac tggcggctaa
cgctatttgt tcagccgttc cagttgattg ggtcccaacc agccgcacca cctggtcgat
ccatgcccat caccaatgga tgacaacaga agacatgtta tcagtatgga atagggtctg
gatagaggaa aacccatgga tggaggataa gactcatgtg tccagttggg aagaagttcc
atacctagga aagagggaag atcagtggtg tggatccctg ataggcttaa cagcaagggc
cacctgggcc actaatatac aagtggccat aaaccaagtg agaaggctca ttgggaatga
gaattatcta gattacatga catcaatgaa gagattcaag aatgagagtg atcccgaagg
ggcactctgg taagtcaaca cattcacaaa ataaaggaaa ataaaaaatc aaatgaggca
agaagtcagg ccagattaag ccatagtacg gtaagagcta tgctgcctgt gagccccgtc
caaggacgta aaatgaagtc aggccgaaag ccacggtttg agcaagccgt gctgcctgtg
gctccatcgt ggggatgtaa aaacccggga ggctgcaacc catggaagct gtacgcatgg
ggtagcagac tagtggttag aggagacccc tcccaagaca caacgcagca gcggggccca
acaccagggg aagctgtacc ctggtggtaa ggactagagg ttagaggaga ccccccgcgt
aacaataaac agcatattga cgctgggaga gaccagagat cctgctgtct ctacagcatc
attccaggca cagaacgcca gaaaatggaa tggtgctgtt gaatcaacag gttct

or a nucleotide sequence having at least 80%, 82%, 85%, 86%, 88%, 90%, 92%, 94%, 95%, 97%, 98% or 99% nucleic acid sequence identity thereto.

A ds RNA may be prepared based on sequences encoding HIV envelope proteins (gp), or an antigenic portion thereof, e.g., a dsRNA encoding:

(SEQ ID NO: 28)
MKVMGRRRNCQQWWELSSLCLWVVIIGSVAGNLWVTVYYGVPVW
KEAKTTLFCASDAKAYKKEVHNIWATHACVPTDPSPQEILLENV
TENFNMWKNDMVDQMHEDVVSLWDQSLKPCVKLTPLCVTLHCVN
ATYKIVNDTKVNATYNDTGVGELKNCSFNMTTEVRDQKQTRYAL
FYKLDIVPLSNDHNHSNYSNHSNYRLISCKTSTLTQACPKVTFD
PLPIHYCAPAGYAILKCNNKTFNGTGPCTNVSTVQCTHGIKPVV
STQLLINGSLSEGEIIIRSENLTNNVKTIIVHLNVSVEINCSRP
NNNTRKSVRIGPGQTFYAMGDIIGDIRAAHCKISDKAWSETLQN
VTKKLREHFPNKTIKFEPSSGGDLEITTHSFNCKGEFFYCNTSK
LFNRTYWPNGTHWSNSTDNSSIIMLPCRIKQIINMWQKVGRAMY
APPIAGKLTCNSNITGLLLVRDGGRENNTNNTETFRPGGGNMKD
NWRSELYKYKVVEIKPLGVAPTQARRMVVTREKRAVGIGAVLLG
FLGAAGSTMGAASITLTAQARQVLSGIVQQQSNLLRAIEAQQHL
LQLTVWGIKQLQTRVLAIERYLKDQQLLGLWGCSGKLICTTSVP
WNSSWSNKSQTDIWNNMTWMQWDREISNYTSTIYKLLEDSQIQQ
DKNEKDLLALDSWKNLWSWFDISNWLWYIKIFIMIVGGLIGLRI
VFAVLSIVNRVRQGYSPLSFQTLTPNQIPGGLDRLGRIEEEGGE

or a polypeptide having at least 80%, 82%, 85%, 86%, 88%, 90%, 92%, 94%, 95%, 97%, 98% or 99% amino acid sequence identity thereto, such as a dsRNA comprising mRNA sequences having an open reading (ORF) corresponding to an ORF in DNA comprising:

(SEQ ID NO: 29)
atgaaagtga tggggagacg gaggaattgt caacaatggt
gggaattgag cagcttatgc ttgtgggtgg tgataattgg
tagtgtggca ggaaacttgt gggtcacagt ctattatggg
gtacctgtgt ggaaagaagc aaaaactact ctattctgtg
catcagatgc taaagcctat aagaaagaag tgcataatat
atgggctaca catgcctgtg tacccacaga ccccagccca
caagaaatac tcttggaaaa tgtaacagag aattttaaca
tgtggaaaaa tgacatggtg gatcagatgc atgaggatgt
agttagttta tgggatcaaa gtctaaagcc atgtgtaaag
ttaactccac tctgtgtcac tttacactgt gtcaatgcta
catacaaaat tgtcaatgac actaaagtca atgctacata
caatgataca ggggttggag aactaaaaaa ttgctctttc
aatatgacaa cagaagtaag agatcagaaa cagactaggt
atgcactttt ttataaactt gatatagtac cacttagtaa
tgaccataac catagtaact atagtaacca tagcaactat
agattaataa gttgtaagac ctcaacccta acacaagcct
gtccaaaggt cacttttgac ccacttccta tacattattg
tgctccagct ggttatgcga ttctaaaatg taataataag
acattcaatg ggacaggacc atgcactaat gtcagcacag
tacaatgtac acatggaatt aagccagtgg tatcaactca
attactgtta aatggtagcc tatcagaagg agagataata
attagatctg aaaatttgac aaacaatgtc aaaacaataa
tagtacatct taatgtatct gtagaaatta attgctcaag
gcccaacaat aatacaagga aaagtgtaag gataggacca
ggacaaacat tttatgcaat gggtgacata ataggagata
taagagcagc acattgtaaa attagcgaca aagcatggtc
tgaaacttta caaaatgtaa caaaaaaatt aagagaacac
ttccctaata agacaataaa atttgaacca tcctcaggag
gggacctaga aattacaaca catagcttta attgtaaggg
agaatttttc tattgtaata catcaaagct gtttaatagg
acatactggc ctaatggcac acactggtct aatagtactg
ataactcctc catcatcatg ctcccatgca gaataaaaca
aattataaac atgtggcaga aggtagggcg agcaatgtat
gcccctccca ttgcagggaa actaacatgt aattcaaata
tcacaggact actattagta cgtgatggag gaagagaaaa
caacacaaac aacacagaga cattcagacc tggaggagga
aatatgaagg ataattggag aagtgaatta tacaaatata
aagtggtaga aattaagcca ttgggagtag cacccaccca
ggcacggagg atggtggtga cgagggaaaa aagagcagta
ggaataggag ctgtactcct tgggttcttg ggagctgcag
gaagcactat gggcgcggcg tcaataacgc tgacggcaca
ggccagacaa gtattgtctg gtatagtgca acagcaaagc
aatttgctga gagctataga ggcgcaacaa catctgttgc
aactcacagt ctggggcatt aaacagctcc agacaagagt
cctggctata gaaagatacc taaaggatca acagctccta
gggctttggg gctgctctgg aaagctcatc tgcaccacct
ctgtgccttg gaactccagt tggagtaata aatctcaaac
agacatttgg aataacatga cctggatgca gtgggataga
gaaattagta actacacaag tacaatatac aagctacttg
aagactcgca aatccagcag gataaaaatg aaaaagactt
attagcattg gacagttgga aaaatctgtg gagttggttt
gacatatcaa actggctgtg gtatataaaa atattcatca
tgatagtagg aggtttgata ggtttgagaa tagtttttgc
tgtactgtct atagtgaata gagttaggca gggatactca
cctttgtcgt ttcagaccct taccccgaac cagatcccgg
gaggactcga caggctcgga agaatcgaag aagaaggtgg
agagtaagac agaaggagat ccattcgatt agtgagcgga
ttcttgtctc ttgcctggga agacttgagg agcctgtgcc
tcttcagcta ccaccgattg agagacttcg cattgattgt
aacgagagca gtggaacttc tgggacgcag cagcctcagg
ggactacaga gggggtggga ggcccttaaa tatctgggag
gcctcgtgca gtattggggt ctggaactaa aaaagagtgc
tattagtctg cttaatacca tagcaatagt agtagctgaa
ggaacagata ggattataga actagtacaa agaatttgta
gggctatcct caatatacct agaagagtaa gacaaggctt
tgaagccgcg ttactataa

or a nucleotide sequence having at least 80%, 82%, 85%, 86%, 88%, 90%, 92%, 94%, 95%, 97%, 98% or 99% nucleic acid sequence identity thereto.

### Exemplary Bacterial Templates for ds RNA Vaccines

A ds RNA may be prepared based on sequences encoding Bordetella pertussis pertactin, or an antigenic portion thereof, e.g., a dsRNA encoding:

(SEQ ID NO: 30)
MNMSLSRIVKAAPLRRTTLAMALGALGAAPAAHADWNNQSIVKT
GERQHGIHIQGSDPGGVRTASGTTIKVSGRQAQGILLENPAAEL
QFRNGSVTSSGQLFDDGIRRFLGTVTVKAGKLVADHATLANVGD
TWDDDGIALYVAGEQAQASIADSTLQGAGGVQIERGANVTVQRS
AIVDGGLHIGALQSLQPEDIPPSRVVLRDINVTAVPASGAPAAV
SVEGASELTLDGGHITGGRAAGVAAMQGAVVHLQRATIRRGDAP
AGGGVIGGAVPGGAVPGGAVPGGFGPGGFGPVLDGWYGVDVSGS
SVELAQSIVEAPELGAAIRVGRGARVTVSGGSLFAPHGNVIETG
GARRFAPQAAPLSITLQAGAHAQGKALLYRVLPEPVKLTLTGGA
DAQGDIVATELPSIPGTSIGPLDVALASQARWTGATRAVDSLSI
DNATWVMTDNSNVGALRLASDGSVDFQQPAEAGRFKVLTVNTLA
GSGLERMNVFADLGLSDKLVVMQDASGQHRLWVRNSGSEPASAN
TLLLVQTPRGSAATFTLANKDGKVDIGTYRYRLAANGNGQWSLV
GAKAPPAPKPAPQPGPQPPQPQPEAPAPQPPAGRELSAAANAAV
NTGGVGLASTLWYAESNALSKREGELRLNPDAGGAWGRGFAQRQ
QLDNRAGRREDQKVAGFELGADHAVAVAGGRWHLGGLAGYTRGD
RGFTGDGGGHTDSVHVGGYATYIADSGFYLDATLRASRLENDFK
VAGSDGYAVKGKYRTHGVGASLEAGRRFTHADGWFLEPQAELAV
FRAGGGAYRAANGLRVRDEGGSSVLGRLGLEVGKRIELAGGRQV
QPYIKASVLQEFDGAGTVRTNGIAHRTELRGTRAELGLGMAAAL
GRGHSLYASYEYSKGPKLAMPWTFHAGYRYSW

or a polypeptide having at least 80%, 82%, 85%, 86%, 88%, 90%, 92%, 94%, 95%, 97%, 98% or 99% amino acid sequence identity thereto, such as a dsRNA comprising mRNA sequences having an open reading (ORF) corresponding to an ORF in DNA comprising:

(SEQ ID NO: 31)
atgaacatgt ctctgtcacg cattgtcaag gcggcgcccc
tgcgccgcac cacgctggcc atggcgctgg gcgcgctggg
cgccgccccg gcggcgcatg ccgactggaa caaccagtcc
atcgtcaaga ccggtgagcg ccagcatggc atccatatcc
agggctccga cccgggcggc gtacggaccg ccagcggaac
caccatcaag gtaagcggcc gtcaggccca gggcatcctg
ctagaaaatc ccgcggccga gctgcagttc cggaacggca
gtgtcacgtc gtcgggacag ttgttcgacg atggcatccg
gcgctttctg ggcaccgtca ccgtcaaggc cggcaagctg
gtcgccgatc acgccacgct ggccaacgtc ggcgacacct
gggacgacga cggcatcgcg ctctatgtgg ccggcgaaca
ggcccaggcc agcatcgccg acagcaccct gcagggcgct
ggcggcgtgc agatcgagcg cggcgccaat gtcacggtcc
aacgcagcgc catcgtcgac gggggcttgc atatcggcgc
cctgcagtca ttgcagccgg aagaccttcc gcccagccgg
gtggtgctgc gcgacaccaa cgtgaccgcc gtgcccgcca
gcggcgcgcc cgcggcggtg tctgtgttgg gggccagtga
gcttacgctc gacggcgggc acatcaccgg cgggcgggca
gcgggggtgg cggccatgca aggggcggtc gtgcatctgc
agcgcgcgac gatacggcgc ggggacgcgc ctgccggcgg
tggggttccc ggcggtgcgg ttcccggcgg tgcggttccc
ggtggtgcgg ttcccggcgg cttcggtccc ggcggcttcg
gtcccgtcct cgacggctgg tatggcgtgg acgtatcggg
ctccagcgtg gagctcgccc agtcgatcgt cgaggcgccg
gagctgggcg ccgcaatccg ggtgggccgc ggcgccaggg
tgacggtgtc gggcggcagc ttgttcgcac cgcacggcaa
tgtcatcgag accggcggcg cgcgtcgctt tgcgcctcaa
gccgcgcccc tgtcgatcac cttgcaggcc ggcgcgcatg
cccaggggaa agcgctgctg taccgggtcc tgccggagcc
cgtgaagctg acgctgaccg ggggcgccga tgcgcagggc
gacatcgtcg cgacggagct gccctctatt cccggcacgt
cgatcgggcc gctcgacgtg gcgctggcca gccaggcccg
atggacgggc gctacccgcg cggtcgactc gctgtccatc
gacaacgcca cctgggtcat gacggacaac tcgaacgtcg
gtgcgctacg gctggccagc gacggcagcg tcgatttcca
gcagccggcc gaagctgggc ggttcaaggt cctgacggtc
aatacgctgg cgggttcggg gctgttccgc atgaatgtct
tcgcggacct ggggctgagc gacaagctgg tcgtcatgca
ggacgccagc ggccagcaca ggctgtgggt ccgcaacagc
ggcagcgagc cggccagcgc caacaccctg ctgctggtgc
agacgccacg aggcagcgcg gcgaccttta cccttgccaa
caaggacggc aaggtcgata tcggtaccta tcgctatcga
ttggccgcca acggcaatgg gcagtggagc ctggtgggcg
cgaaggcgcc gccggcgccc aagcccgcgc cgcagccggg
tccccagccg ccgcagccgc agccggaagc gccggcgccg
caaccgccgg cgggcaggga gttgtccgcc gccgccaacg
cggcggtcaa cacgggtggg gtgggcctgg ccagcacgct
ctggtacgcc gaaagcaatg cgttgtccaa gcgcctgggc
gagttgcgcc tgaatccgga cgccggcggc gcctggggcc
gcggcttcgc gcaacgccag cagctggaca accgcgccgg
gcggcgcttc gaccagaagg tggccggctt cgagctgggc
gccgaccacg cggtggcggt ggccggcgga cgctggcacc
tgggcgggct ggccggctat acgcgcggcg accgcggctt
caccggcgac ggcggcggcc acaccgacag cgtgcatgtc
gggggctatg ccacatatat cgccgacagc ggtttctacc
tggacgcgac gctgcgcgcc agccgcctgg agaatgactt
caaggtggcg ggcagcgacg ggtacgcggt caagggcaag
taccgcaccc atggggtggg cgcctcgctc gaggcgggcc
ggcgctttac ccatgccgac ggctggttcc tcgagccgca
ggccgagctg gcggtattcc gggccggcgg cggtgcgtac
cgcgcggcca acggcctgcg ggtgcgcgac gaaggcggca
gctcggtgct gggtcgcctg ggcctggagg tcggcaagcg
catcgaactg gcaggcggca ggcaggtgca gccatacatc
aaggccagcg tgctgcagga gttcgacggc gcgggtacgg
tacgcaccaa cggcatcgcg caccgcaccg aactgcgcgg
cacgcgcgcc gaactgggcc tgggcatggc cgccgcgctg
ggccgcggcc acagcctgta tgcctcgtac gagtactcca
agggcccgaa gctggccatg ccgtggacct tccacgcggg
ctaccggtac agctggtaa

or a nucleotide sequence having at least 80%, 82%, 85%, 86%, 88%, 90%, 92%, 94%, 95%, 97%, 98% or 99% nucleic acid sequence identity thereto.

### Exemplary Protozoa Templates for ds RNA Vaccines

A ds RNA may be prepared based on sequences encoding Plasmodium circumsporozoite protein, or an antigenic portion thereof, e.g., a dsRNA encoding:

(SEQ ID NO: 32)
KKLSVLAISSFLIVDFLFPGYHHNSNSTKSRNLSELCYNNVDT
KLFNELEVRYSTNQDHFYNYNKTIRLLNENNNEKDGNVTNERK
KKPTKAVENKLKQPPGDDDGAGNDAGNDAGNAAGNAAGNAAGN
AAGNAAGNDAGNAAGNAAGNAAGNAAGNAAGNAAGNDAGNAAG
NAAGNAAGNAAGNAAGNDAGNAAGNAAGNAAGNAAGNAAGNAA
GNAAGNAAGNAAGNAAGNDAGNAAGNAAGNAAGNAAGNAAGNA
AGNAAGNAAGNAAGNAAGNAAGNAAGNAAGNAAGNAAGNAAGN
AAGNAAGNAAGNAAGNEKAKNKDNKVDANTNKKDNQEENNDSS
NGPSEEHIKNYLESIRNSITEEWSPCSVTCGSGIRARRKVDAK
NKKPAELVLSDLETEICSLDKCSSTFNVVSNSLGIVLVLVLIL
FH,

or a polypeptide having at least 80%, 82%, 85%, 86%, 88%, 90%, 92%, 94%, 95%, 97%, 98% or 99% amino acid sequence identity thereto, such as a dsRNA comprising mRNA sequences having an open reading (ORF) corresponding to an ORF in DNA comprising:

(SEQ ID NO: 33)
atgaagaagt tatctgtctt agcaatatcc tcttttttaa
ttgttgattt cctcttccct ggatatcatc acaactcaaa
ttccaccaag tcaagaaatt taagtgagtt gtgttacaat
aatgtggaca ctaaattatt taatgagtta gaagtcagat
atagcacgaa tcaagatcat ttctataact ataataagac
aatcagatta cttaatgaaa ataacaatga aaaagatgga
aatgtgacca atgaaagaaa aaaaaaaccc acaaaagctg
ttgaaaataa attgaaacaa ccccccggag atgatgatgg
cgcaggaaat gatgcaggaa atgatgcagg aaatgcagca
ggaaatgcag caggaaatgc agcaggaaat gcagcaggaa
atgcagcagg aaatgatgca ggaaatgcag caggaaatgc
agcaggaaat gcagcaggta acgcagcagg aaatgcagca
ggaaatgcag caggaaatga tgcaggaaat gcagcaggta
acgcagcagg aaatgcagca ggaaatgcag caggaaatgc
agcaggaaat gatgcaggaa atgcagcagg aaatgcagca
ggaaatgcag caggtaacgc agcaggaaat gcagcaggaa
atgcagcagg taacgcagca ggtaacgcag caggaaatgc
agcaggaaat gcagcaggaa atgatgcagg aaatgcagca
ggtaacgcag caggaaatgc agcaggaaat gcagcaggaa
atgcagcagg taacgcagca ggtaacgcag caggaaatgc
agcaggaaat gcagcaggaa atgcagcagg aaatgcagca
ggtaacgcag caggaaatgc agcaggaaat gcagcaggta
acgcagcagg aaatgcagca ggaaatgcag caggtaacgc
agcaggaaat gcagcaggaa atgcagcagg aaatgaaaaa
gcgaaaaata aggataataa agtggatgca aatacgaata
aaaaggacaa ccaggaagaa aataatgatt cgtctaatgg
tccatctgaa gaacatataa agaattattt agaaagtatt
cgtaatagta ttacggagga atggtcacca tgtagtgtaa
cttgtggaag tggtataagg gctagaagaa aggttgatgc
aaaaaataag aaacctgcag aattagtttt aagtgacctt
gaaactgaaa tttgttcact agataaatgc tccagtacat
ttaatgtcgt aagtaattcg ttaggaatag tattagtttt
agtcttaata ctctttcact aa

or a nucleotide sequence having at least 80%, 82%, 85%, 86%, 88%, 90%, 92%, 94%, 95%, 97%, 98% or 99% nucleic acid sequence identity thereto.

### Exemplary Tumor Antigen Templates for ds RNA Vaccines

A ds RNA may be prepared based on sequences encoding a cancer associated antigen, or an antigenic portion thereof, e.g., a dsRNA encoding:

(SEQ ID NO: 34)
MWVPVVFLTLSVTWIGAAPLILSRIVGGWECEKHSQPWQVLVAS
RGRAVCGGVLVHPQWVLTAAHCIRNKSVILLGRHSLFHPEDTGQ
VFQVSHSFPHPLYDMSLLKNRFLRPGDDSSHDLMLLRLSEPAEL
TDAVKVMDLPTQEPALGTTCYASGWGSIEPEEFLTPKKLQCVDL
HVISNDVCAQVHPQKVTKFMLCAGRWTGGKSTCSGDSGGPLVCN
GVLQGITSWGSEPCALPERPSLYTKVVHYRKWIKDTIVANP,

or a polypeptide having at least 80%, 82%, 85%, 86%, 88%, 90%, 92%, 94%, 95%, 97%, 98% or 99% amino acid sequence identity thereto.

A ds RNA may be prepared based on sequences encoding a cancer associated antigen, or an antigenic portion thereof. e.g., a dsRNA encoding:

(SEQ ID NO: 35)
MTPGTQSPFFLLLLLTVLTVVTGSGHASSTPGGEKETSATQRSS
VPSSTEKNALSTGVSFFFLSFHISNLQFNSSLEDPSTDYYQELQ
RDISEMFLQIYKQGGFLGLSNIKFRPGSVVVQLTLAFREGTINV
HDVETQFNQYKTEAASRYNLTISDVSVSDVPFPFSAQSGAGVPG
WGIALLVLVCVLVALAIVYLIALAVCQCRRKNYGQLDIFPARDT
YHPMSEYPTYHTHGRYVPPSSTDRSPYEKVSAGNGGSSLSYTNP
AVAATSANL,

or a polypeptide having at least 80%, 82%, 85%, 86%, 88%, 90%, 92%, 94%, 95%, 97%, 98% or 99% amino acid sequence identity thereto.

A ds RNA may be prepared based on sequences encoding a cancer associated antigen, or an antigenic portion thereof, e.g., a dsRNA encoding:

(SEQ ID NO: 36)
SAPPHRWCIPWQRLLLTASLLTFWNPPTTAKLTIESTPFNVAEG
KEVLLLVHNLPQHLFGYSWYKGERVDGNRQIIGYVIGTQQATPG
PAYSGREIIYPNASLLIQNIIQNDTGFYTLHVIKSDLVNEEATG
QFRVYPELPKPSISSNNSKPVEDKDAVAFTCEPETQDATYLWWV
NNQSLPVSPRLQLSNGNRTLTLFNVTRNDTASYKCETQNPVSAR
RSDSVILNVLYGPDAPTISPLNTSYRSGENLNLSCHAASNPPAQ
YSWFVNGTFQQSTQELFIPNITVNNSGSYTCQAHNSDTGLNRTT
VTTITVYAEPPKPFITSNNSNPVEDEDAVALTCEPEIQNTTYLW
WVNNQSLPVSPRLQLSNDNRTLTLLSVTRNDVGPYECGIQNELS
VDHSDPVILNVLYGPDDPTISPSYTYYRPGVNLSLSCHAASNPP
AQYSWLIDGNIQQHTQELFISNITEKNSGLYTCQANNSASGHSR
TTVKTITVSAELPKPSISSNNSKPVEDKDAVAFTCEPEAQNTTY
LWWVNGQSLPVSPRLQLSNGNRTLTLFNVTRNDARAYVCGIQNS
VSANRSDPVTLDVLYGPDTPIISPPDSSYLSGANLNLSCHSASN
PSPQYSWRINGIPQQHTQVLFIAKITPNNNGTYACFVSNLATGR
NNSIVKSITVSASGTSPGLSAGATVGIMIGVLVGVALI,

or a polypeptide having at least 80%, 82%, 85%, 86%, 88%, 90%, 92%, 94%, 95%, 97%, 98% or 99% amino acid sequence identity thereto.

A ds RNA may be prepared based on sequences encoding a cancer associated antigen, or an antigenic portion thereof, e.g., a dsRNA encoding:

(SEQ ID NO: 37)
MVSYWDTGVLLCALLSCLLLTGSSSGSKLKDPELSLKGTQHIMQ
AGQTLHLQCRGEAAHKWSLPEMVSKESERLSITKSACGRNGKQF
CSTLTLNTAQANHTGFYSCKYLAVPTSKKKETESAIYIFISDTG
RPFVEMYSEIPEIIHMTEGRELVIPCRVTSPNITVTLKKFPLDT
LIPDGKRIIWDSRKGFIISNATYKEIGLLTCEATVNGHLYKTNY
LTHRQTNTIIDVQISTPRPVKLLRGHTLVLNCTATTPLNTRVQM
TWSYPDEKNKRASVRRRIDQSNSHANIFYSVLTIDKMQNKDKGL
YTCRVRSGPSFKSVNTSVHIYDKAFITVKHRKQQVLETVAGKRS
YRLSMKVKAFPSPEVVWLKDGLPATEKSARYLTRGYSLIIKDVT
EEDAGNYTILLSIKQSNVFKNLTATLIVNVKPQIYEKAVSSFPD
PALYPLGSRQILTCTAYGIPQPTIKWFWHPCNHNHSEARCDFCS
NNEESFILDADSNMGNRIESITQRMAIIEGKNKMASTLVVADSR
ISGIYICIASNKVGTVGRNISFYITDVPNGFHVNLEKMPTEGED
LKLSCTVNKFLYRDVTWILLRTVNNRTMHYSISKQKMAITKEHS
ITLNLTIMNVSLQDSGTYACRARNVYTGEEILQKKEITIRGEHC
NKKAVFSRISKFKSTRNDCTTQSNVKH,

or a polypeptide having at least 80%, 82%, 85%, 86%, 88%, 90%, 92%, 94%, 95%, 97%, 98% or 99% amino acid sequence identity thereto.

A ds RNA may be prepared based on sequences encoding a cancer associated antigen, or an antigenic portion thereof, e.g., a dsRNA encoding:

MQSKVLLAVALWLCVETRAASVGLPSVSLDLPRLSIQKDILTIK
ANTTLQITCRGQRDLDWLWPNNQSGSEQRVEVTECSDGLFCKTL
TIPKVIGNDTGAYKCFYRETDLASVIYVYVQDYRSPFIASVSDQ
HGVVYITENKNKTVVIPCLGSISNLNVSLCARYPEKRFVPDGNR
ISWDSKKGFTIPSYMISYAGMVFCEAKINDESYQSIMYIVVVVG
YRIYDVVLSPSHGIELSVGEKLVLNCTARTELNVGIDFNWEYPS
SKHQHKKLVNRDLKTQSGSEMKKFLSTLTIDGVTRSDQGLYTCA
ASSGLMTKKNSTFVRVHEKPFVAFGSGMESLVEATVGERVRIPA
KYLGYPPPEIKWYKNGIPLESNHTIKAGHVLTIMEVSERDTGNY
TVILTNPISKEKQSHVVSLVVYVPPQIGEKSLISPVDSYQYGTT
QTLTCTVYAIPPPHHIHWYWQLEEECANEPSQAVSVTNPYPCEE
WRSVEDFQGGNKIEVNKNQFALIEGKNKTVSTLVIQAANVSALY
KCEAVNKVGRGERVISFHVTRGPEITLQPDMQPTEQESVSLWCT
ADRSTFENLTWYKLGPQPLPIHVGELPTPVCKNLDTLWKLNATM
FSNSTNDILIMELKNASLQDQGDYVCLAQDRKTKKRHCVVRQLT
VLERVAPTITGNLENQTTSIGESIEVSCTASGNPPPQIMWFKDN
ETLVEDSGIVLKDGNRNLTIRRVRKEDEGLYTCQACSVLGCAKV
EAFFIIEGAQEKTNLEIIILVGTAVIAMFFWLLLVIILRTVKRA
NGGELKTGYLSIVMDPDELPLDEHCERLPYDASKWEFPRDRLKL
GKPLGRGAFGQVIEADAFGIDKTATCRTVAVKMLKEGATHSEHR
ALMSELKILIHIGHHLNVVNLLGACTKPGGPLMVIVEFCKFGNL
STYLRSKRNEFVPYKTKGARFRQGKDYVGAIPVDLKRRLDSITS
SQSSASSGFVEEKSLSDVEEEEAPEDLYKDFLTLEHLICYSFQV
AKGMEFLASRKCIHRDLAARNILLSEKNVVKICDFGLARDIYKD
PDYVRKGDARLPLKWMAPETIFDRVYTIQSDVWSFGVLLWEIFS
LGASPYPGVKIDEEFCRRLKEGTRMRAPDYTTPEMYQTMLDCWH
GEPSQRPTFSELVEHLGNLLQANAQQDGKDYIVLPISETLSMEE
DSGLSLPTSPVSCMEEEEVCDPKFHYDNTAGISQYLQNSKRKSR
PVSVKTFEDIPLEEPEVKVIPDDNQTDSGMVLASEELKTLEDRT
KLSPSFGGMVPSKSRESVASEGSNQTSGYQSGYHSDDTDTTVYS
SEEAELLKLIEIGVQTGSTAQILQPDSGTTLSSPPV

A ds RNA may be prepared based on sequences encoding a cancer associated antigen, or an antigenic portion thereof, e.g., a dsRNA encoding:

(SEQ ID NO: 38)
MSQGILSPPAGLLSDDDVVVSPMFESTAADLGSVVRKNLLSDCS
VVSTSLEDKQQVPSEDSMEKVKVYLRVRPLLPSELERQEDQGCV
RIENVETLVLQAPKDSFALKSNERGIGQATHRFTFSQIFGPEVG
QASFFNLTVKEMVKDVLKGQNWLIYTYGVTNSGKTHTIQGTIKD
GGILPRSLALIFNSLQGQLHPTPDLKPLLSNEVIWLDSKQIRQE
EMKKLSLLNGGLQEEELSTSLKRSVYIESRIGTSTSFDSGIAGL
SSISQCTSSSQLDETSHRWAQPDTAPLPVPANIRFSIWISFFEI
YNELLYDLLEPPSQQRKRQTLRLCEDQNGNPYVKDLNWIHVQDA
EEAWKLLKVGRKNQSFASTHLNQNSSRSHSIFSIRILHLQGEGD
IVPKISELSLCDLAGSERCKDQKSGERLKEAGNINTSLHTLGRC
IAALRQNQQNRSKQNLVPFRDSKLTRVFQGFFTGRGRSCMIVNV
NPCASTYDETLHVAKFSAIASQLVHAPPMQLGFPSLHSFIKEHS
LQVSPSLEKGAKADTGLDDDIENEADISMYGKEELLQVVEAMKT
LLLKERQEKLQLEMHLRDEICNEMVEQMQQREQWCSEHLDTQKE
LLEEMYEEKLNILKESLTSFYQEEIQERDEKIEELEALLQEARQ
QSVAHQQSGSELALRRSQRLAASASTQQLQEVKAKLQQCKAELN
STTEELHKYQKMLEPPPSAKPFTIDVDKKLEEGQKNIRLLRTEL
QKLGESLQSAERACCHSTGAGKLRQALTTCDDILIKQDQTLAEL
QNNMVLVKLDLRKKAACIAEQYHTVLKLQGQVSAKKRLGTNQEN
QQPNQQPPGKKPFLRNLLPRTPTCQSSTDCSPYARILRSRRSPL
LKSGPFGKKY,

or a polypeptide having at least 80%, 82%, 85%, 86%, 88%, 90%, 92%, 94%, 95%, 97%, 98% or 99% amino acid sequence identity thereto.

### Example VII

Lipid nanoparticles (LNP) were prepared using the ionic lipid SM-102 (Witzigmann et al., 2020; Cullis et al., 2017). LNPs are composed of DSPC (10 wt %), ChoI (38.5 wt %), PEG-DMA (1.5 wt %) and SM-102 (50 wt %) (Verbeke et al., 2021). This formulation was used to prepare ss and ds mRNA LNPs expressing luciferase (FIG. 8). The lipids (70 vol % in ethanol) were rapidly mixed with mRNA (30 vol % in sodium citrate buffer pH 4) under turbulent flow (Hirota et al., 1999). mRNA LNPs were dialyzed to exchange buffer to phosphate buffered saline pH 7.4, then concentrated using a centrifugation concentrator. The entire procedure used RNAse free buffers and plastic ware, and the resulting ss and ds mRNA LNPs possessed a particle size of 100-120 nm and an mRNA encapsulation of 80%.

### Example VIII

To establish the ability of ds mRNA to substitute for ss mRNA in a LNP vaccine, Covid-19 spike ss and ds mRNAs were prepared (FIG. 10). A shorter spike mRNA was prepared representing 1898 bases from the N299 terminus (see below) due to ease of cloning the template DNA into pLuc80A to replace luciferase.

(SEQ ID NO: 50)
TCTAGAACATTTGCTTCTGACACAACTGTGTTCACTAGCAACCTCAAA
CAGACACCGCCGCCACCATGTTTGTTTTTCTTGTTTTATTGCCACTAG
TCTCTAGTCAGTGTGTTAATCTTACAACCAGAACTCAATTACCCCCTG
CATACACTAATTCTTTCACACGTGGTGTTTATTACCCTGACAAAGTTT
TCAGATCCTCAGTTTTACATTCAACTCAGGACTTGTTCTTACCTTTCT
TTTCCAATGTTACTTGGTTCCATGCTATACATGTCTCTGGGACCAATG
GTACTAAGAGGTTTGATAACCCTGTCCTACCATTTAATGATGGTGTTT
ATTTTGCTTCCACTGAGAAGTCTAACATAATAAGAGGCTGGATTTTTG
GTACTACTTTAGATTCGAAGACCCAGTCCCTACTTATTGTTAATAACG
CTACTAATGTTGTTATTAAAGTCTGTGAATTTCAATTTTGTAATGATC
CATTTTTGGGTGTTTATTACCACAAAAACAACAAAAGTTGGATGGAAA
GTGAGTTCAGAGTTTATTCTAGTGCGAATAATTGCACTTTTGAATATG
TCTCTCAGCCTTTTCTTATGGACCTTGAAGGAAAACAGGGTAATTTCA
AAAATCTTAGGGAATTTGTGTTTAAGAATATTGATGGTTATTTTAAAA
TATATTCTAAGCACACGCCTATTAATTTAGTGCGTGATCTCCCTCAGG
GTTTTTCGGCTTTAGAACCATTGGTAGATTTGCCAATAGGTATTAACA
TCACTAGGTTTCAAACTTTACTTGCTTTACATAGAAGTTATTTGACTC
CTGGTGATTCTTCTTCAGGTTGGACAGCTGGTGCTGCAGCTTATTATG
TGGGTTATCTTCAACCTAGGACTTTTCTATTAAAATATAATGAAAATG
GAACCATTACAGATGCTGTAGACTGTGCACTTGACCCTCTCTCAGAAA
CAAAGTGTACGTTGAAATCCTTCACTGTAGAAAAAGGAATCTATCAAA
CTTCTAACTTTAGAGTCCAACCAACAGAATCTATTGTTAGATTTCCTA
ATATTACAAACTTGTGCCCTTTTGGTGAAGTTTTTAACGCCACCAGAT
TTGCATCTGTTTATGCTTGGAACAGGAAGAGAATCAGCAACTGTGTTG
CTGATTATTCTGTCCTATATAATTCCGCATCATTTTCCACTTTTAAGT
GTTATGGAGTGTCTCCTACTAAATTAAATGATCTCTGCTTTACTAATG
TCTATGCAGATTCATTTGTAATTAGAGGTGATGAAGTCAGACAAATCG
CTCCAGGGCAAACTGGAAAGATTGCTGATTATAATTATAAATTACCAG
ATGATTTTACAGGCTGCGTTATAGCTTGGAATTC

The studies employed ds mRNA that encodes the full length spike mRNA (4461 bases). ss mRNA was enzymatically capped prior to generation of ds mRNA by hybridizing ss and Rev as illustrated in FIG. 10. Spike ss and ds mRNA LNPs were generated using SM-102. Both ss and ds mRNA LNPs (5 μg of total mRNA in 20 μl) were dosed i.m. in triplicate ICR male mice and compared to triplicate naïve control mice. After two weeks the mice received a second identical dose of ss or ds mRNA LNP administered i.m. After three additional weeks the mice were euthanized, and the blood and spleen were harvested. The blood was processed into plasma and analyzed for anti-spike IgG using an ELISA.

The ELISA identified specific anti spike IgG titers of 2-3 ug/ml for mice dosed with ss and ds mRNA LNP, relative to background derived for naive mouse plasma. The spleens were processed to harvest splenocytes enriched in T-cells from each mouse Splenocytes were stimulated with spike protein to evoke selective T-cell proliferation. The T-cells were permeabilized and incubated with fluorescent antibodies (anti-CD11a, CD49d) to select for CD4+ and CD8+ T-cell markers, and intracellular TNF-alpha, and INF-gamma (FIG. 11). The percent of stimulated CD4+ and CD8+ T-cells secreting TNF-alpha increased 2-3-fold for ds mRNA versus ss mRNA (FIG. 11). This study established that both ds mRNA LNPs invoked an antigen specific T-cell response in mice. Moreover, it was surprising that the T-cell response with ds mRNA was more effective. Previous studies have determined the harmful effects of delivering ds RNA to human dendritic cells in culture, leading to the release of cytokines and apoptosis

The internalization of mRNA LNPs into dendritic cells is sensed by innate immune sensors that are localized in the endosomes and cytosol Chen et al., 2017). Tatematsu et al., 2018).). The detection of mRNA by the endosomal TLR8 leads to the expression of type I IFNs (IFN-alpha and IFN-beta) (Linares-Fernandez et al. 2020). dsRNA contaminants and native stem-loop structures in ss mRNA can activate RIG-1 and MDA5 antiviral signaling pathways that lead to the transcriptional up-regulation of the type I and III interferons (Linares-Fernandez et al. 2020). Type I IFNs stimulate autocrine or paracrine receptors that regulate antiviral immunity. This includes the expression of MHC-I and co-stimulatory molecules needed for T cell responses as well as antiviral proteins involved with undesirable anti-RNA responses (Linares-Fernandez et al 2020). Substitution of uridine with pseudo-uridine, the removal of dsRNA fragments, and sequence-engineering, have been used to minimize type I IFN activity of mRNA (Verbeke et al. 2021). The concern that mRNA stimulation of the innate immune response will suppress transgene expression is based on the work of Kariko and Weissman who reported that mRNA substitution with pseudo-uridine decreased secretion of INF-alpha and increase gene expression (Karikó, et al., 2008; Kariko et al., 2005). These conclusions are derived from in vitro transfection data using mRNA delivered with lipofectamine (Kariko et al., 2005. For this reason, all current mRNA LNP vaccines remove trace quantities of ds RNA biproducts generated by IVT, over concern that they will stimulate the innate immune response to cause dendritic cell apoptosis and an inactive vaccine mRNA (Verbeke et al. 2021). However, the Anderson group has since reported no change in expression when substituting uridine for pseudo uridine in mRNA LNPs (Kaufmann et al., 2016). The expression levels of hydrodynamically dosed mRNA in the liver are not influenced by pseudo-undine substitution (Poliskey et al., 2018) A recent study described promoter less T7 transcription during IVT resulting in a 10% by-product composed of random ds RNA detected by native PAGE (Mu et al., 2018). Substitution with pseudo-uridine decreased the amount of ds RNA by-product. Importantly, it is only the uncapped ds RNA by-products, and not capped mRNA, that pose a concern of signaling through the innate immune sensors to block expression (Nallagatla et al., 2007; Zust et al., 2011). There is only one report of capped ds mRNA stimulating the expression of INF in cell culture (Kato et al., 2008), and no reports of ds mRNA invoking a type I INF mediated innate immune response in vivo.

Given the structural similarity of ds RNA and ds mRNA, it was therefore unexpected that ds mRNA LNPs not only prime dendritic cell immunity to produce a B-cell response, but unexpectedly primed an increased T-cell response compared to ss mRNA LNPs (FIG. 11). The use of ds mRNA LNPs to prime a greater T-cell response is an unexpected finding in view of the current understanding of how dendric cells process ds mRNA LNPs that are sensed by the innate immune response to generate robust B and T cell immune responses.

### Example IX

Most LNP delivery systems have been utilized for the intravenous delivery of siRNA into the liver. Recently, significant advancements have been made to optimize this delivery system for intramuscular use, such as the Moderna COVID-19 vaccine used to deliver single-stranded spike protein mRNA. Despite its overwhelming success, concerns have arisen regarding the thermostability of this formulation during transportation. Therefore, the disclosed LNP gene delivery system was compared to double-stranded mRNA. By utilizing a small-scale PLEXER device, LNPs encapsulating single- and double-stranded Luciferase mRNA were assembled by turbulently mixing 4 lipid components with the mRNA: an ionizable cationic lipid (SM-102), a phospholipid (DSPC), cholesterol, and a pegylated lipid (DMG-PEG 2000). This apparatus allowed us the generation LNPs of comparable particle size (˜100 nm) to those found in the existing vaccines as determined through dynamic light scattering measurements. After incubation at room temperature (20° C.) for 12 hours, the mRNA was extracted out of the LNPs. Band intensity from agarose gel analysis indicated that the RNA in the current Moderna COVID-19 vaccine and our single-stranded mRNA formulation degraded by 82% and 55% respectively, whereas the double-stranded mRNA formulation demonstrated no degradation. Furthermore, double-stranded mRNA remained intact after harsher incubation conditions at 37° C. for 12 hours whereas the other two formulations were destroyed. Indeed, single-stranded mRNA consistently degraded over multiple incubation experiments while double-stranded mRNA remained resilient. To discern the nature of this degradation, extractions were repeated in the presence of RNAse OUT, an RNAse inhibitor. Nearly all the RNA in each formulation was recovered, suggesting that the instability is caused by enzymatic degradation by RNAse when not stored under optimal conditions. This data demonstrates the enhanced stability of double-stranded mRNA as a superior candidate for vaccines over single-stranded mRNA.

Thus, double-stranded mRNA LNP vaccines display enhanced thermostability compared to single-stranded mRNA formulations that were tested.

### REFERENCES

Adami, J. Pharm. Sci., 88:739 (1999).
Al Dosari et al., Hydrodynamic Delivery, in Advances in Genetics. Academic Press. p. 65 (2005).
Allen et al., Mol. Pharm., 15:3881 (2018).
Alvarez et al., J. Virology, 76:6841 (2002).
Anderson et al., Bioconjugate Chemistry 21:1479 (2010).
Andrianaivo et al., J. Gene Med., 6:877 (2004).
Avci-Adali et al., J. Biol. Eng., 8:8 (2014).
Awasthi et al., Sci. Immunol. 4: (2019)
Bahl et al., Mol Ther., 25:1316 (2017).
Baumhover et al., Bioconjug. Chem., 21:74 (2010)
Baumhover et al., Mol. Pharm., 12:4321 (2015).
Bayat et al., Intn. Immunopharm., 97:107679 (2021).
Boeckle et al., J. Control Release, 112:240 (2006).
Boussif et al., Proc. Nat. Acad. Sci. USA. 92:7297 (1995).
Chen et al., Bioconj. Chem., 18:371 (2007).
Chen et al., Bioconjugate Chem. 17:1057 (2006)
Chen et al., Intn. Union Biochem. Mol. Biol., 9:297 (2017).
Chen et al., IUBMB Life, 66:297 (2017)).
Cheng et al., Biomaterials, 2:6868 (2012).
Chuah et al., J. Thromb. Haemost, 11:99 (2013) w
Collard et al., Carbohydr. Res., 22176 (2000)
Collard et al., J. Pharm. Sci 89:499 (2000).
Corey et al., Science, 368:948 (2020).
Crowley & Rice, J. Control Release, 219:457 (2015).
Crowley et al., Gene Ther., 22.993 (2015).
Cullis et al., Mol. Ther., 2:1467 (2017).
Debus et al., J. Control Release, 148:334 (2010).
Deering et al., Expert Opin. Drug Deliv., 11:885 (2014)
Diken et al., Gene Ther., 18:702 (2011).
Duskey & Rice, AAPS PharmSci., 51345 (2014).
Engering t al., J. Immunol., 168:2118 (2002).
Ericson & Rice, Peptide Sci., 98:510 (2012).
Ericson & Rice, Tetrahedron Letters, 54:3440 (2013b).
Ericson & Rice, Tetrahedron Letters, 54:4746 (2013a).
Ericson et al., Adv in Gene Therapy, Imperial College Press, Daniel Scherman editor, pg. 207 (2014)
Evers et al., Anal. Biochem., 265:313 (1998).
Feinberg et al., J. Biol. Chem., 282:4202 (2007).
Fernandez et al., Bioconjugate Chem., 21.723 (2010).
Fernandez et al., Gene Ther., 18:23 (2011).
Gao et al., Carbohydr. Res., 487:107877 (2020).
Geall et al., Proc. Natl. Acad Sci. USA 109:14604 (2012).
Gebre et al., Cell, 184:1589 (2021).
Gomez-Aguado et al., Nanomaterials (Basel), 10: (2020).
Guo et al., Mil Med Res 2020, 7, 11.
Hassett et al., Ther. Nucleic Acids, 15:1 (2019).
Hirota et al., Biotechnigues, 27:286 (1999).
Hodges et al., Expert Opin. Biol. Ther., 3:911 (2003).
Hu et al., ACS Nano, 7:5376 (2013).
Huysmans et al., Mol. Ther. Nucleic Acids, 17:867 (2019).
Jagger et al., J. Infect. Dis., 220:1577 (2019).
John et al., Vaccine 2018, 36, 1689-1699.
Kariko et al., Mol. Ther., 16:1833 (2008).
Kariko et al., Mol. Ther., 20:948 (2012).
Kariko et al., Immunity 23:165 (2005).
Kato et al., JEM, 205:1601 (2005).
Kato et al., Exp Med., 205:1601 (2008).
Kauffman et al., Biomaterials, 109:78 (2016).
Khargharia et al., Gene Therapy, 21:1021 (2014).
Khargharia et al., J. Control Release, 170:325 (2013).
Kizzire et al., Gene Ther., 20:407 (2013).
Kormann et al., Nat. Biotechnol., 29:154 (2011).
Kwok et al., Current Op. Mol. Ther., 3:142 (2001).
Kwok et al., Int. J. Pharm., 203:81 (2000).
Kwok et al., J. Pharm. Sci., 88:996 (1999).
L, Science, 668:1949 (2020).
Le Moignic, et al., J. Control Release, 278:110 (2018).
Lenter et al., Pharmaceutical Research, 21:683 (2004).
Li et al., Analytical Biochemistry, 470:14 (2015).
Linares-Fernandez et al., Trends Mol. Med., 26:311 (2020).
Liu et al., Gene Ther., 6:1258 (1999).
Lue et al., J. Virol., 76:10299 (2002).
Lutz et al., NPJ Vaccines, 2:29 (2017).
Malone et al., Proc. Natl. Acad. Sci. USA, 86:6077 (1989).
Marcato et al., Infect. Immun., 73:6523 (2005)
Mathew et al., Gene Ther., 27:196 (2020)
McCaffrey et al., Mol. Ther., 5:676 (2002).
McKenzie et al., Abstracts of Papers of the Am. Chem. Soc., 219:U6 (2000).
McKenzie et al., Bioconj. Chem., 11:901 (2000).
McKenzie et al., J. Biol. Chem., 275:9970 (2000).
Meyer et al., J. Infect. Dis., 217:451 (2018).
Mu et al., NAR, 46:5239 (2018).
Nallagatla et al., Science. 318:1455 (2007).
Ogris et al., J. Biol. Chem., 276:47550 (2001).
Pardi et al., Curr. Opin. Immunol., 65:14 (2020).
Pardi et al., J. Control Release, 217:345 (2015)
Pardi et al., J. Exp. Med., 21:1571 (2018).
Pardi et al., Nat. Rev. Drug Discov.: (2018).
Pardi et al., Nature. 543:248 (2017)1.
Pardi, et al., Nat. Commun., 9:3361 (2018).
Park et al., Intn. J. Bio. Sci., 17:446 (2021).
Perche et al., J. Drug Target, 19:315 (2011).
Perche et al., Nanomedicine, 7:445 (2011).
Phan, Infect. Genet. Evol., 79:104211 (2020).
Phua et al., J. Control Release, 166:227 (2013)
Phua et al., Sci Rep., 4:5128 (2014).
Pichon et al., Methods Mol. Biol., 969, 247 (2013).
Poliskey et al., Gene Ther., 25:473 (2018)
Pollard et al., Mol. Ther., 21:251 (2013).
Potter et al., Curr. Protoc. Mol. Biol., 121:931 (2018)
Pun et al., Bioconjugate Chemistry, 13:630 (2002).
Read et al., in Advances in Genetics, J. C. Hall, J. C. Dunlap, T. Friedmann, and V. van Heyningen, Editors. Academic Press. p. 19-46 (2005).
Read et al., Nucleic Acids Res., 33:e86 (2005).
Rettig & Rice, Expert Opin. Biol Ther., 7, 799 (2007).
Richard et al., Gene Ther., 16:746 (2009).
Richner et al., Cell, 169:176 (2017)
Richner et al., Cell, 170:273 (2017).
Roth et al., Front Immunol., 10:1424 (2019).
Sahin et al., Nat. Rev. Drug Discov., 13:759 (2014).
Schlake et al., RNA Biol., 9:1319 (2012).
Selmi et al., Cancer Immunol. Immunother., 65:1075 (2016).
Semple et al., Nat. Biotechnol., 28:172 (2010).
Stadlbauer et al., Curr. Protoc. Microbiol., 57:el00 (2020).
Swaminathan et al., Br. J. Clin. Pharmacol., 78:1135 (2014).
Tatematsu et al., J. Innate Immun., 10:398 (2018).
Teijaro et al., Nat. Rev. Immunol., 21:195 (2021).
Uchida et al., Biomaterials, 150:162 (2018)
Uchida et al., PLoS One, 8:e56220 (2013).
VanBlargan et al., Cell Rep., 25:3382 (2018).
Verbeke et al., J. Cont. Rel., 333:511 (2021).
Wadhwa et al., Bioconjug. Chem., 6:283 (1995).
Wadhwa et al., Bioconjug. Chem., 8:81 (1997).
Wang et al., Mol. Ther., 21:358 (2013).
Wesselhoeft et al., Nat. Commun., 9:2629 (2018).
Wilber et al., Mol. Ther., 13:625 (2006).
Wiseman et al., Immunity, 23:165 (2005).
Wiseman et al., NAR, 38:5884 (2010).
Witzigmann et al., Adv Drug Deliv Rev., 159:344 (2020)
Wolff et al., Science 247:1465 (1990)
Wooddell et al., Mol. Ther., 21:973 (2013)
Wu et al., J. Biol. Chem., 263:14621 (1988)
Wu et al., Nature. 579:265 (2020).
Yang et al., Biohys. J. 81:1475 (2001).
Zangi et al., Nat. Biotechnol., 31:898 (2013).
Zhang et al., Gene Ther., 11:675 (2004).
Zhang et al., Vaccines (Basel), 8: (2020).
Zust et al., Nat Immunol., 12:137 (2011).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising an amount of double stranded (ds) mRNA encoding a therapeutic or prophylactic gene product, wherein at least one strand of the ds mRNA has a 5′ cap, a start codon, a polyA sequence and encodes the protein, wherein the two strands of the ds mRNA are hydrogen bonded over at least 10 nucleotides, and an amount of a plurality of distinct lipids.

2. The composition of claim 1 wherein the at least one strand of the ds mRNA encodes a viral protein, a bacterial protein or a cancer antigen.

3. The composition of claim 2 wherein the at least one strand of the ds mRNA encodes a coronavirus spike protein or an antigenic portion thereof including the receptor binding domain.

4-5. (canceled)

6. The composition of claim 1 wherein at least one strand of the ds mRNA includes one or more non-natural nucleotides.

7-8. (canceled)

9. The composition of claim 1 wherein at least one strand of the ds mRNA includes at least one non-phosphodiester bond.

10-16. (canceled)

17. The composition of claim 6 wherein both of the strands of the ds mRNA include one or more non-natural nucleotides.

18. The composition of claim 16 wherein at least one strand of the ds mRNA has two or more different non-natural nucleotides.

19. (canceled)

20. The composition of claim 1 wherein the RNA is circular RNA, resulting in ds circular mRNA.

21. The composition of claim 1 wherein the RNA is self-amplifying RNA, resulting in ds self-amplifying RNA.

22-23. (canceled)

24. A method of expressing a therapeutic or prophylactic gene product, comprising: introducing the composition of claim 1 to mammalian cells in an amount effective to express the gene product.

25-27. (canceled)

28. The method of claim 24 wherein the gene product is a viral protein, is a bacterial protein or is a cancer antigen.

29. The method of claim 28 wherein the viral protein is influenza HA.

30. The method of claim 28 wherein the viral protein is a coronavirus spike protein.

31-34. (canceled)

35. The composition of claim 1 which is a vaccine.

36. The composition of claim 35 wherein the gene product is a coronavirus spike protein or an antigenic portion thereof.

37. A vaccine comprising lipid nanoparticles (LNPs) comprising an amount of double stranded (ds) mRNA encoding a prophylactic gene product, wherein at least one strand of the ds mRNA has a 5′ cap and/or IRES, a start codon, a polyA sequence and encodes the protein, wherein the two strands of the ds mRNA are hydrogen bonded over at least 10 nucleotides, and a pharmaceutically acceptable carrier.

38. The vaccine of claim 37 wherein the at least one strand of the ds mRNA encodes a viral protein.

39. The vaccine of claim 38 wherein the at least one strand of the ds mRNA encodes a coronavirus spike protein or an antigenic portion thereof including the receptor binding domain.

40. The vaccine of claim 37 wherein at least one strand of the ds mRNA includes one or more non-natural nucleotides.

41-42. (canceled)

43. The vaccine of claim 37 wherein at least one strand of the ds mRNA includes at least one non-phosphodiester bond.

44-50. (canceled)

51. The vaccine of claim 40 wherein both of the strands of the ds mRNA include one or more non-natural nucleotides.

52. The vaccine of claim 50 wherein at least one strand of the ds mRNA has two or more different non-natural nucleotides.

53. The vaccine of claim 37 wherein the RNA is ds circular RNA.

54. The vaccine of claim 37 wherein the RNA is ds self-amplifying RNA.

55. (canceled)

56. The vaccine of claim 37 wherein the LNPs comprise DSPC, cholesterol, PEG-DMA, SM-102, or any combination thereof.

57. The vaccine of claim 56 wherein the DSPC is about 5 to about 20 wt %, the cholesterol is about 35 to about 45 wt %, the PEG-DMA is about 1 to about 2.5 wt %, or the SM-102 is about 40 to about 60 wt %.

58. (canceled)

59. A method to immunize an animal, comprising administering to the animal an effective about of the vaccine of claim 37.

60-63. (canceled)
